(19) United States
(12) Patent Application Publication
(10) Pub. No.: US 2024/0427838 A2
(43) Pub. Date: Dec. 26, 2024

(54) SYSTEMS AND METHODS FOR ENCODING, DECODING, AND MATCHING SIGNALS USING SSM MODELS

(71) Applicants: Alexander Stoytchev, Ames, IA (US); Volodymyr Sukhoy, Huxley, IA (US)

(72) Inventors: Alexander Stoytchev, Ames, IA (US); Volodymyr Sukhoy, Huxley, IA (US)

(73) Assignee: Iowa State University Research Foundation, Inc., Ames, IA (US)

(21) Appl. No.: 18/326,517

(22) Filed: May 31, 2023

Related U.S. Application Data
(62) Division of application No. 16/112,179, filed on Aug. 24, 2018.
(60) Provisional application No. 62/550,223, filed on Aug. 25, 2017.

(51) Int. Cl. G06F 17/15; G06F 17/10; G06F 17/16; G06F 17/17; G16B 30/00

(52) U.S. Cl. CPC G06F 17/153; G06F 17/10; G06F 17/16; G06F 17/17; G16B 30/00; G06F 30/00; G16B 50/00; G16B 99/00

(57) ABSTRACT

Disclosed herein are embodiments of methods for encoding, decoding, and matching patterns in collections of signals. These methods use weighting functions to scale the signals. This scaling enables the use of signals of arbitrary duration, wherein the signals may include discrete sequences and spike trains. In the most general case, the signals can be represented using functionals, which extends the expressive power of the methods. Further disclosed herein are embodiments of a system that performs these methods.

### CROSS-REFERENCE TO RELATED PATENT APPLICATIONS

This patent application is a divisional of co-pending U.S. patent application Ser. No. 16/112,179, filed Aug. 24, 2018, which claims the benefit of U.S. Provisional Patent Application No. 62/550,223, filed Aug. 25, 2017, the entire teachings and disclosure of which are incorporated herein by reference thereto.

### FIELD OF THE INVENTION

This invention generally relates to data correlation, data association, signal processing, and, more particularly, to systems and methods for encoding signals into SSM Models, decoding signals from encoded SSM Models, and matching signals to a plurality of SSM Models.

### BACKGROUND OF THE INVENTION

In many contexts, it is helpful to associate a data input with a previously received or encoded data input in order to perform a responsive action or address an error. Addi-tionally, it may be helpful to encode a data input for a first time so that it can be used with future associations. These associations are used in a variety of fields, including pattern and sequence recognition, robotics, artificial intelligence, machine learning, etc. However, many conventional methods of performing these encoding and decoding operations have limita-tions in terms of computational complexity, sequence length, discrete vs. continuous signal operation, and robustness to noise.

Embodiments of the present disclosure address the limitations associated with conventional methods of encoding, decoding, and matching data inputs. These and other advantages of the invention, as well as additional inventive features, will be apparent from the description of the invention provided herein.

### BRIEF SUMMARY OF THE INVENTION

This disclosure describes a biologically-inspired representation for associating data inputs and a family of algorithms that encode and decode this representation. After encoding, this representation can be used to recall one data input given another data input, even if the second data input is not identical to the one used during encoding. This representation can also be used for matching of data inputs to previously encoded models based on the length of the decoded sequence or based on the similarity of the decoded output to one of the data inputs. This representation generalizes and extends the SSM Sequence Model (SSM) that was described in U.S. Pat. No. 10,007,662, entitled “Systems and Methods for Recognizing, Classifying, Recalling and Analyzing Information Utilizing SSM Sequence Models,” filed on Jan. 9, 2015, the entirety of which is hereby incorporated by reference thereto.

The extended SSM model described here generalizes the SSM model to work with weighted sequences. This generalization is done for both discrete-time and continuous-time signals. The properties of the model are both explained and proved using the theory behind the z-transform and the Laplace transform. Emphasis is placed on deriving sufficient conditions for accurate decoding. Two new families of algorithms are introduced: the ZUV family for discrete sequences and the SUV family for continuous spike trains. The ZUV family of algorithms utilizes the unilateral z-transform with parameter z and weighting functions u and v, and the SUV family of algorithms utilizes the Laplace transform with parameter s and weighting functions u and v.

As will be described more fully in the paragraphs below, present herein is an overview of the encoding and decoding algorithms for discrete sequences that were introduced in U.S. Pat. No. 10,007,662, including aspects of the present disclosure that build upon the previous disclosure. Also provided herein is a theoretical model for the discrete-time representation that follows from the concatenation theorem for the unilateral z-transform, which is stated and proven in the present disclosure. The discrete-time model is then extended to work with weighted sequences and the ZUV family of algorithms is introduced. It also proves sufficient conditions under which the ZUV decoding algorithm can decode SSM Models for sequences of arbitrary length. The discrete model is then applied to sequences that may contain gaps, and the ZUV algorithms are extended to work with these types of sequences.

The present disclosure also proves the concatenation theorem for the Laplace transform and uses it to describe a continuous-time model that works with spike trains. In the continuous-time model, the timing of the spikes is not constrained to be at discrete intervals, i.e., spikes can come in at any time. The continuous-time model is also extended to work with weighted spike trains, particularly in the form of the SUV family of algorithms. The properties of the SUV decoding algorithm are described, and its robustness to noise is demonstrated. This model is then generalized to work with functionals. That is, the spike-based model becomes a special case of the general functional-based model when the functionals are set to shifted Dirac's deltas.

The properties of the ZUV and SUV models allow both the encoding and the decoding to be performed in parallel on multiple computational units. This enables embodiments in which the encoding and decoding time is commensurate with the duration of the signals.

In further embodiments, the representations described herein can be distributed and replicated over a plurality of computational units so that each of these units holds only a subset of the SSM model. Thus, the encoding or decoding process can continue even if some computational units fail.

This disclosure enables using weighting functions to encode collections of signals of arbitrary length into SSM models and decode collections of signals of arbitrary length from SSM models. In embodiments, the decoding process may end early or become quiescent if the collection of signals used to decode does not fit the model sufficiently well. In other embodiments, the signals decoded from a model can be compared to the signals available during the decoding and a match can be detected if there is sufficient similarity between them. In other embodiments, pattern matching is implemented by analyzing the lengths of decoded collections of signals, wherein the lengths are used as a similarity measure. These properties of the extended SSM model enable a new class of distributed systems and representations. In embodiments, this is used to implement pattern matching in a way that does not require comparing the elements of SSM matrices.

Contexts and applications for these various algorithms and models are presented herein. Embodiments of the present disclosure use weighting functions during encoding and decoding. The models and algorithms can be utilized for approximate pattern matching, pattern completion, and pattern association. In these embodiments, the patterns can be represented using collections of signals. Additionally, the models and algorithms can be used in robotics, speech and sound recognition, and computer vision. In robotics, embodiments of the present disclosure perform interactive object recognition, learn affordances of objects and detect these affordances across sensory modalities. In the field of computer vision, further embodiments perform object recognition, including recognition of partially occluded objects, and face recognition. In other embodiments, the models and algorithms can be used to build, search, and update an associative memory using a collection of signals. In addition, the models can be used for predicting, completing, and correcting biological sequences, which may include both DNA sequences and protein sequences. It should be noted that these contexts and applications for use of the algorithm and models are exemplary only, and the algorithms and models are not limited strictly thereto.

Other aspects, objectives, and advantages of the invention will become more apparent from the following detailed description when taken in conjunction with the accompanying drawings.

### BRIEF DESCRIPTION OF THE DRAWINGS

The accompanying drawings incorporated in and forming a part of the specification illustrate several aspects of the present invention and, together with the description, serve to explain the principles of the invention. The following paragraphs provide a brief description of each figure.

FIG. 1. Depicts two sequences: S′=βαγβ and S″=ABAB.

FIG. 2. Shows how to map a letter sequence to a number sequence. The mapping in this case is based on the alphabetical order of the characters in the Greek alphabet.

FIG. 3. Shows the histogram for the sequence S′=βαγβ.

FIG. 4. Shows the histogram for the sequence S″=ABAB.

FIG. 5. Shows a visualization of the incremental computation of the histogram for the sequence S′=βαγβ. The sequence characters are processes one at a time. As each new character becomes available, the histogram and its vector representation are updated to reflect this new information.

FIG. 6. Incremental computation of the histogram for the sequence S″=ABAB.

FIG. 7. Shows the open bigrams for the sequence pair S′=βαγβ and S″=ABAB.

FIG. 8. Constructing an SSM matrix from the sequences S′=βαγβ and S″=ABAB.

FIG. 9. Constructing an SSM matrix from the sequences S″=ABAB and S′=βαγβ.

FIG. 10. Illustration of the encoding algorithm. The two character sequences in this example are S′=βαγβ and S″=ABAB. Each row corresponds to a different encoding iteration. The components that are added or modified during the fourth iteration are highlighted in different colors.

FIG. 11. The encoding SSM model for the sequence pair (S′=βαγβ, S″=ABAB). The encoding model has three components: the histogram h′ for the sequence S′, the matrix M, and the histogram h″ for the sequence S″. Their values are the same as in the last row in FIG. 10.

FIG. 12. The decoding SSM model for the sequences S′=βαγβ and S″=ABAB. The components of the model are the matrix M and the histogram h″ for the sequence S″. Note that the histogram h′ for the sequence S′ is not part of the model because it is not needed during decoding.

FIG. 13. Illustration of the decoding task. The box in the middle represents the SSM model, which consists of the matrix M(S′, S″) and the histogram vector h″. Given the sequence S″ at run time, the goal is to decode the sequence S′ from the model.

FIG. 14. Illustration of the decoding algorithm. Each row corresponds to a different decoding iteration. The matrix in this example was encoded from the pair of sequences S′=βαγβ and S″=ABAB. Given the sequence S″ the algorithm decodes the sequence S′ using the matrix and h″. The components that are modified during the last iteration are highlighted in red and green.

FIG. 15. All four possible matrices for sequences of length one.

FIG. 16. The histograms for the English sequences shown in FIG. 15.

FIG. 17. All 16 possible matrices for sequences of length two.

FIG. 18. The histograms for the English sequences shown in FIG. 17.

FIG. 19. Example of aliasing. The sequence pairs (αββ, ABA) and (βαα, ABA) map to the same matrix. Because the second sequence is the same in both pairs they also map to the same second histogram. Thus, the decoding algorithm has to work with the same SSM model for both pairs.

FIG. 20. Given the input sequence ABA and the SSM model shown in FIG. 19 it is possible to decode two different output sequences: αββ and βαα. In other words, this example demonstrates that the decoding process could be ambiguous for sequences of length three.

FIG. 21. All 64 possible matrices for sequences of length three.

FIG. 22. The histograms for the English sequences shown in FIG. 21.

FIG. 23. The number of possible sequence pairs as a function of M′, M″, and T.

FIG. 24. The eight boxes in this figure illustrate the possible outcomes after encoding a model from the sequence pair (S₁, S₂) and then attempting to decode this model given only the sequence S₂ at run time. Double arrows represent encoding, which takes two sequences and produces a model (i.e., a matrix M and a histogram vector h″). Single arrows represent decoding, which takes one sequence and uses the model to output another sequence.

FIG. 25. Classification of the decoding outcomes for M′=M″=2 and T=1, 2, . . . , 10. Each subplot corresponds to one of the 8 cases from FIG. 24.

FIG. 26. Encoding example with exponential decay. The two input sequences in this example are S′=βαγβ and S″=ABAB. The elements that are added or modified during the last iteration are highlighted in red and green.

FIG. 27. The encoding SSM model for this example. The values of the three components are the same as the ones in the last row of FIG. 26. The vector h′ is not used by the decoding algorithm and can be discarded at the end of the encoding process.

FIG. 28. Visualization of the decoding algorithm with exponential decay. The two sequences from which the matrix was encoded are S′=βαγβ and S″=ABAB. Given the sequence S″ at run time, this example shows how to decode the sequence S′ using the matrix M and the vector h″.

FIG. 29. In the exponential case the matrix for the pair of sequences S′=βαα and S″=ABA is not deterministically decodable because the algorithm can take two different steps during the first iteration. a) If it picks the first row, then it gets stuck during the second iteration. b) If it selects the second row, then it can successfully decode the Greek sequence.

FIG. 30. Classification of the decoding outcomes for M′=M″=2 and T=1, 2 . . . , 10.

FIG. 31. Summary of the convolution and cross-correlation theorems for the bilateral z-transform. The formulas in the cross-correlation column come from Theorem 3.15 and Theorem 3.16.

FIG. 32. Summary of the convolution and cross-correlation theorems for the unilateral z-transform. The two theorems in the cross-correlation column are described in Section 3.4. Two special cases of the theorem in the lower-right corner provide the mathematical justification for the encoding and the decoding algorithm.

FIG. 33. Example of a two-sided infinite sequence.

FIG. 34. Example of a right-sided infinite sequence.

FIG. 35. Example of a two-sided finite sequence.

FIG. 36. Example of a right-sided finite sequence.

FIG. 37. The decimal number 2147.514 represented as a two-sided finite sequence.

FIG. 38. The decimal number 2147.514 from FIG. 37 represented as a two-sided infinite sequence. The left tail and the right tail of this sequence are padded with infinitely many zeros.

FIG. 39. The number 1101.101 expressed as a finite two-sided sequence of digits. For each digit there is a corresponding power of z that is also shown in this figure. If we pick a value for z, then we can compute the value of the bilateral z-transform of this sequence evaluated at z by simply multiplying each digit with its corresponding power of z and adding all products.

FIG. 40. Visualization of the bilateral z-transform of the two-sided finite sequence b that is shown in FIG. 39. This plot is only for real z. The blue circles indicate the value of the transform at z=−2.5, z=0.4, and z=2. The transform has a singularity at z=0.

FIG. 41. The elements of the sequence b=(b₀, b₁, b₂) and their corresponding powers of z.

FIG. 42. Same as FIG. 41 but the concrete sequence is now b=(1, 4, 2).

FIG. 43. Visualization of the unilateral z-transform of the sequence b=(1, 4, 2) in the range z∈[−5, 5]. The transform has a singularity at z=0. This plot is only for real z.

FIG. 44. Computing the elements (a*b)ₙ of the convolution sequence for different values of n.

FIG. 45. The elements (a*b)ₙ of the convolution of a=(a₀, a₁, a₂) and b=(b₀, b₁, b₂).

FIG. 46. Numerical example of convolution. The sequences in this example are: a=(2, 2, 1) and b=(1, 2, 3). Notice that the sequence b must be reversed before performing this operation.

FIG. 47. Computing the cross-correlation of a=(a₀, a₁, a₂) and b=(b₀, b₁, b₂).

FIG. 48. The elements (a*b)ₙ of the cross-correlation of a and b for different values of n.

FIG. 49. Numerical example of cross-correlation. The two sequences in this example are: a=(2, 2, 1) and b=(1, 2, 3). Because in this case the sequence a contains only real numbers there is no need to conjugate them before they are multiplied with the elements of b.

FIG. 50. Computing the cross-correlation of b=(b₀, b₁, b₂) and a=(a₀, a₁, a₂).

FIG. 51. The elements (b*a)ₙ of the cross-correlation of a and b for different values of n.

FIG. 52. The elements (a*b)ₙ of the cross-correlation sequence for different values of n. When computing the unilateral z-transform of the cross-correlation of a and b the left tail of this sequence is ignored. The ignored elements, which have negative indices, are shown in gray.

FIG. 53. Computing the elements (a*b)ₙ of the cross-correlation of a and b for n≥0.

FIG. 54. The elements (a*b)ₙ of the cross-correlation sequence for n≥0.

FIG. 55. Summary of the six different formulas for [symbol] _{a*b}⁺(z), expressed as two nested sums. Each row of the table corresponds to the index of the outer sum and each column corresponds to the index of the inner sum. The indices in each formula iterate over two of the following three options: 1) the negative powers of z; 2) the elements of a; and 3) the elements of b.

FIG. 56. The six formulas for [symbol] _{a*b}⁺(z), expressed using the Heaviside function. Each row correspond to the index of the outer sum. Each column correspond to the index of the inner sum.

FIG. 57. The two sequences of length five used in this example.

FIG. 58. The same two sequences as in FIG. 57, but now each is split into two parts.

FIG. 59. Visualization of the representation that is used for the sequences a=(a₀, a₁, a₂, a₃, a₄) and b=(b₀, b₁, b₂, b₃, b₄). Each sequence is equal to the elementwise sum of a “prefix” sequence and a “suffix” sequence that are padded with the appropriate number of zeros.

FIG. 60. Computing the elements (a*b)ₙ of the cross-correlation of a and b for n≥0.

FIG. 61. Computing the elements (a′*b′), of the cross-correlation of a′ and b′ for n≥0.

FIG. 62. Computing the elements (a″*b″), of the cross-correlation of a″ and b″ for n≥0.

FIG. 63. Computing the elements (a′ *b″), of the cross-correlation of a′ and b″. This figure also shows how elements with negative indices are computed. Because the non-zero elements of a′ and b″ don't overlap for n<0, however, the left tail of the resulting cross-correlation sequences contains only zeros. As a consequence of this, the bilateral z-transform of a′*b″ is equal to the unilateral z-transform of a′*b″.

FIG. 64. Computing the elements (a″ *b′)ₙ of the cross-correlation of a″ and b′ for n≥0. Note that the right tail of the resulting cross-correlation sequence contains only zeros.

FIG. 65. Illustration of the first special case of the concatenation theorem in which each of the two suffixes consists of only a single element.

FIG. 66. Illustration of the second special case of the concatenation theorem in which each of the two prefixes consists of only a single element.

FIG. 67. Summing the terms of [symbol] _{a*b}⁺(z) along the diagonals.

FIG. 68. Summing the terms of [symbol] _{a*b}⁺(z) along the columns.

FIG. 69. Summing the terms of [symbol] _{a*b}⁺(z) along the rows.

FIG. 70. Visualization of the mapping of the character sequence S′=ααβ to two exponentially weighted sequences a=(1.0, 0.5, 0.0) and A=(0.0, 0.0, 0.25).

FIG. 71. Mapping the character sequence S″=ABA to two exponentially weighted sequences A=(1, 0, 4) and B=(0, 2, 0).

FIG. 72. Formulas for the three components returned by the ZUV encoding algorithm.

FIG. 73. Illustration of the incremental computation of the three helper variables \hat{z}, û, and \hat{v} by the ZUV encoding algorithm. The integer index in the square brackets after each variable corresponds to a specific iteration number, e.g., \hat{z}[2] is the value of z during the 2-nd iteration.

FIG. 74. Numerical example of ZUV encoding. The two character sequences from which the matrix is constructed are S′=ααβ and S″=ABA. In this case z=2, u=1, and v=1. Because in this example both u=1 and v=1 this encoding corresponds to the traditional exponential case that has only one argument, i.e., z=2.

FIG. 75. Numerical example of ZUV encoding. The two character sequences from which the matrix is constructed are S′=ααβ and S″=ABA. In this case z=2, u=2, and v=1.

FIG. 76. Numerical example of ZUV encoding. The two input sequences are S′=ααβ and S″=ABA. In this example z=1, u=2, and v=0.5. Because in this case z=1, the elements of h′ don't decay over time.

FIG. 77. Numerical example of ZUV encoding. The two input sequences are S′=ααβ and S″=ABA. In this example z=2, u=4, and v=0.5.

FIG. 78. Numerical example of ZUV decoding. The two sequences from which the matrix was encoded are S′=ααβ and S″=ABA. Given the sequence S″ at run time, this example shows how to decode the sequence S′ using the matrix and the vector h″. In this case the values of the three parameters are: z=2, u=1, and v=1. Since both u and v are equal to one, this example reduces to the special case of exponential decoding in which there is only one argument, i.e., z=2.

FIG. 79. Numerical example of ZUV decoding. The two sequences from which the matrix was encoded are S′=ααβ and S″=ABA. Given the sequence S″ at run time, this example shows how to decode the sequence S′ using the matrix and the vector h″. In this case the values of the three parameters are: z=2, u=2, and v=1.

FIG. 80. Numerical example of ZUV decoding. The two sequences from which the matrix was encoded are S′=ααβ and S″=ABA. Given the sequence S″ at run time, this example shows how to decode the sequence S′ using the matrix and the vector h″. In this case the values of the three parameters are: z=1, u=2, and v=0.5. Note that because v<1, the value of v grows exponentially from 1 to 2 to 4, which reflects what is subtracted from h″ during each iteration.

FIG. 81. Numerical example of ZUV decoding. The two sequences from which the matrix was encoded are S′=ααβ and S″=ABA. Given the sequence S″ at run time, this example shows how to decode the sequence S′ using the matrix and the vector h″. In this case the values of the three parameters are: z=2, u=4, and v=0.5. Note that because v<1, the value of v grows exponentially from 1 to 2 to 4, which reflects what is subtracted from h″ during each iteration.

FIG. 82. The four test cases used in the experiments and how they relate to the two sufficient conditions for deterministic ZUV decoding.

FIG. 83. Classification of the ZUV decoding outcomes for z=2, u=1, v=1, M′=M″=2, and T=1, 2, . . . , 10.

FIG. 84. Classification of the ZUV decoding outcomes for z=2, u=2, v=1, M′=M″=2, and T=1, 2, . . . , 10.

FIG. 85. Classification of the ZUV decoding outcomes for z=1, u=2, v=0.5, M′=M″=2, and T=1, 2, . . . , 10.

FIG. 86. Classification of the ZUV decoding outcomes for z=2, u=4, v=0.5, M′=M″=2, and T=1, 2, . . . , 10.

FIG. 87. Visualization of the input sequences S′=γ_αβ and S″=BA_B, which are used in the examples. Each sequence contains one gap, which is indicated with the underscore character.

FIG. 88. Visualization of how the two character sequences S′ and S″ can be represented with a set of binary sequences. The Greek sequence S′=γ_αβ is split into three binary sequences: α=(0, 0, 1, 0), β=(0, 0, 0, 1), and γ=(1, 0, 0, 0). The gap in S′ is at index 1 and is represented with a zero at that index in all three binary sequences. Similarly, the English sequence S″=BA_B is jointly represented by two other binary sequences: A=(0, 1, 0, 0) and B=(1, 0, 0, 1). The gap in the character sequence S″ is represented with a zero at index 2 in both binary sequences.

FIG. 89. Abstract values for the three outputs of the encoding algorithm. The Greek alphabet in this example has three letters, i.e., Γ′={α, β, γ}, and thus h′ is a column vector of size 3. The English alphabet is Γ″={A, B}, and thus h″ is a row vector of size 2. The matrix is of size 3×2.

FIG. 90. The numerical values for h′, h″, and M shown in FIG. 89. These numbers were computed by the encoding algorithm using the sequences shown in FIG. 88. The value of z was equal to 2 in this case.

FIG. 91. Encoding example with exponential decay for sequences with gaps. The two input sequences in this example are S′=γ_αβ and S″=BA_B. The underscores indicate the locations of the gaps. Note that the matrix is the same after the second and the third iteration. The reason for this is that the incoming character on S″ during the third iteration is a gap, which suppresses the matrix update. The vector h′, however, is updated at that time as its elements decay by a factor of z=2 at each iteration. The elements that are added or updated during the fourth iteration are highlighted in the last row of this figure.

FIG. 92. Illustration of the decoding algorithm for sequences with gaps. The matrix in this example is encoded from the sequences S′=γ_αβ and S″=BA_B. This figure shows how given the sequence S″ at run time the algorithm can decode the sequence S′ from the matrix. Note that during the second iteration it is not possible to subtract the vector h″ from any row of the matrix. Therefore, the matrix update is suppressed and the output character for that iteration is a gap, which is indicated with the ‘_’ symbol. The components that are updated during the fourth iteration are highlighted in the last row of the figure. This example assumes that z is equal to 2.

FIG. 93. The four sets of parameter values and their mapping to the two sufficient conditions for deterministic decoding.

FIG. 94. Classification of the ZUV decoding outcomes for z=2, u=1, v=1, M′=M″=2, and T=1, 2, . . . , 10. Both S′ and S″ may contain gaps.

FIG. 95. Classification of the ZUV decoding outcomes for z=2, u=2, v=1, M′=M″=2, and T=1, 2, . . . , 10. Both S′ and S″ may contain gaps.

FIG. 96. Classification of the ZUV decoding outcomes for z=1, u=2, v=0.5, M′=M″=2, and T=1, 2, . . . , 10. Both S′ and S″ may contain gaps.

FIG. 97. Classification of the ZUV decoding outcomes for z=2, u=4, v=0.5, M′=M″=2, and T=1, 2, . . . , 10. Both S′ and S″ may contain gaps.

FIG. 98. The five test cases used in the experiments and how they map to the two sufficient conditions for deterministic decoding (first set) and the aliasing conditions for h″ (second set).

FIG. 99. Classification of the ZUV decoding outcomes for z=2, u=1, v=1, M′=M″=2, and T=1, 2, . . . , 10. Both S′ and S″ may contain gaps, but S″ can't end with a gap.

FIG. 100. Classification of the ZUV decoding outcomes for z=2, u=2, v=1, M′=M″=2, and T=1, 2, . . . , 10. Both S′ and S″ may contain gaps, but S″ can't end with a gap. The aliased/aliased plot shows that the condition uv≥2 is no longer sufficient for the case with gaps.

FIG. 101. Classification of the ZUV decoding outcomes for z=1, u=2, v=0.5, M′=M″=2, and T=1, 2, . . . , 10. Both S′ and S″ may contain gaps, but S″ can't end with a gap. In this case the decoding is perfect because u≥2z.

FIG. 102. Classification of the ZUV decoding outcomes for z=2, u=4, v=0.5, M′=M″=2, and T=1, 2, . . . , 10. Both S′ and S″ may contain gaps, but S″ can't end with a gap. In this case vz=1, which leads to aliasing of h″. This aliasing does not affect the decoding results as long as the sequence S″ from which the matrix was encoded is provided at run time.

FIG. 103. Classification of the ZUV decoding outcomes for z=2, u=4, v=1, M′=M″=2, and T=1, 2, . . . , 10. Both S′ and S″ may contain gaps, but S″ can't end with a gap. Because the condition u≥2z is satisfied, the decoding is perfect. Unlike FIG. 102, there is no h″ aliasing in this case because vz≥2.

FIG. 104. A plot of the template function δₙ(t) for n<<∞. The area under this curve is equal to 1 for any n, i.e.,

$$( \frac{1}{2 n} + \frac{1}{2 n} ) n = 1 .$$

FIG. 105. A plot of the template function δₙ(t−t₀) for n<<∞. This curve is shifted to the right by t₀ relative to the curve shown in FIG. 104, i.e., the center is at to and the right edge is at

$${t}_{0} + \frac{1}{2 n} .$$

The area under the curve is still equal to 1.

FIG. 106. Visualization of the sequence of functions that model a shifted Dirac's delta, where the shift is equal to 1. As the value of n increases the curves for the template functions δₙ(t−1) become more narrow and more peaked. The last plot shows an idealized impulse as n→∞.

FIG. 107. An example of a spike train a=(a₁, a₂, a₃, a₄, a_{b}) that contains five spikes and is represented with the function a^{(m)}(t). This function, in turn, is represented as the following sum: δₘ(t−a₁)+δₘ(t−a₂)+δₘ(t−a₃)+δₘ(t−a₄)+δₘ(t−a₅). In this example, the value of m is 2 and the spikes occur at times a₁=1, a₂=3, a₃=4, a₄=6, and a₅=9.

FIG. 108. An example of a spike train b=(b₁, b₂, b₃, b₄) that has four spikes and is modeled with the function b⁽ⁿ⁾(t). This is similar to FIG. 107, but now n=3 and the spikes occur at times b₁=2.1, b₂=4.9, b₃=7.4, and b₄=9.2. Note that these times are no longer integers.

FIG. 109. Illustration of the interaction of two Heaviside step functions. The first two plots show the graphs for H(t₁−t) and H(t₂−t). The third plot shows the product of the first two.

FIG. 110. The three components of the SSM model for M′=M″=2. This figure summarizes the notation for each element of the matrix M and the two vectors h′ and h″.

FIG. 111. The three components of the SSM model for M′=M″=2. Each element is expressed as the Laplace transform of a spike train or as the Laplace transform of the cross-correlation of two spike trains. All transforms are evaluated at only one point, i.e., at s.

FIG. 112. Summary of the notation for the values of the three components of the SSM model and each of their elements at time t during encoding.

FIG. 113. Summary of the notation for the components of the SSM model and each of their elements at time t during decoding. The vector h′ is not used during decoding.

FIG. 114. Summary of the formulas, stated using the Laplace transform notation.

FIG. 115. Summary of the encoding formulas for a common timeline. If two spikes from a and b coincide, then the spike that comes from a is processed first.

FIG. 116. The state of the SSM model after iteration i in the common timeline. In two of the formulas the right truncation bracket is round (highlighted in red).

FIG. 117. Summary of the decoding verification formulas for a common timeline. For pairs of coincident spikes, it is assumed that the spike from a is processed before the spike from b.

FIG. 118. The state of the SSM model at the end of the (i+1)-st verification iteration. Note that three of the truncation brackets are round, not square (highlighted in red).

FIG. 119. Summary of the four special cases. Each case examines the segments of the spike trains a and b between cᵢ and cᵢ₊₁. Depending on the temporal order of the two spikes, these four cases will be referred to as case aa, ab, ba, and bb. By the construction of the common timeline, coincidences are possible only in the case ab, because if two spikes coincide, then precedence is given to the spike from a.

FIG. 120. Visualization of the effect of multiplying the shifted template function by a real scalar. a) Plot of the original shifted template function δₙ(t−t₀). b) Plot of the same template function after it has been multiplied by the real scalar c. The resulting function is cδₙ(t−t₀).

FIG. 121. Notation for the three components of the SUV model for M′=M″=2.

FIG. 122. The elements of the SUV model expressed using the Laplace transform notation.

FIG. 123. Notation for the three components of the SUV model during encoding.

FIG. 124. Notation for the components of the SUV model during decoding.

FIG. 125. Summary of the SUV formulas using the Laplace transform notation.

FIG. 126. Summary of the SUV encoding formulas for a common timeline. If two spikes on a and b coincide, then the spike from a is processed before the spike from b.

FIG. 127. The state of the SUV model after the i-th iteration of the encoding algorithm. Note that two of the truncation brackets are not square but round (highlighted in red).

FIG. 128. Summary of the decoding verification formulas for a common timeline. If a spike from a coincides with a spike from b, then the spike from a is processed first.

FIG. 129. The state of the SUV model at the end of the (i+1)-st iteration of the decoding verification algorithm. Note that three of the truncation brackets are round (highlighted in red).

FIG. 130. This figure illustrates an example where the matrix is encoded from the spike train ^{e}α and the collection of spike trains A=(A⁽¹⁾, A⁽²⁾, A⁽³⁾). The list t″ stores the sorted times of all spikes in A. The list c″ stores the origin of each spike in t″, e.g., a value of 2 indicates that the spike came from A⁽²⁾. The sequence ψ stores the candidate decoding times for the output spikes. In this case, the time in ψ is uniformly discretized in 0.5 increments. The decoded spike train ^{d}α is shown at the bottom of the figure. In this case, ^{e}α=^{d}α.

FIG. 131. A counter-example that shows that a model encoded with a=(a₁, a₂) and b=(b₁) where a₁, a₂≤b₁ can lead to decoding a single spike at time tᵢ<a₁.

FIG. 132. Example of non-interleaving. The spikes on A⁽¹⁾ occur in two different inter-spike intervals of α.

FIG. 133. Example of non-interleaving. Both A⁽¹⁾ and A⁽²⁾ have spikes that occur in two different inter-spike intervals of α.

FIG. 134. Example of non-interleaving. A⁽¹⁾ has spikes in all three inter-spike intervals of α. A⁽²⁾ has spikes in two inter-spike intervals of α.

FIG. 135. Example of insufficient interleaving. No spikes from either A⁽¹⁾ or A⁽²⁾ fall in the last interval of α, i.e, :008 α₂, ∞).

FIG. 136. Example of insufficient interleaving. The middle interval [α₁, α₂) contains no spikes from A⁽¹⁾ or A⁽²⁾.

FIG. 137. Example of insufficient interleaving. The middle interval [α₁, α₂) contains all spikes from both A⁽¹⁾ and A⁽²⁾.

FIG. 138. Example of insufficient interleaving. The interval [α₁, α₂) does not contain any spikes from A⁽¹⁾, A⁽²⁾, or A⁽³⁾. This is also true for the interval [α₃, ∞).

FIG. 139. Example of minimally sufficient interleaving.

FIG. 140. Example of minimally sufficient interleaving.

FIG. 141. Example of minimally sufficient interleaving.

FIG. 142. Example of minimally sufficient interleaving.

FIG. 143. Example of sufficient but not minimally sufficient interleaving. If A⁽¹⁾ is removed, then this example becomes minimally sufficient.

FIG. 144. Example of sufficient but not minimally sufficient interleaving. If A⁽¹⁾ or A⁽²⁾ is removed, but not both, then this example becomes minimally sufficient.

FIG. 145. Example of sufficient but not minimally sufficient interleaving. If A⁽¹⁾ is removed, then this example becomes minimally sufficient.

FIG. 146. Example of sufficient interleaving between two collections of spike trains. Both α⁽¹⁾ and α⁽²⁾ sufficiently interleave A=(A⁽¹⁾, A⁽²⁾).

FIG. 147. Example of insufficient interleaving between two collections of spike trains. In this case, only α⁽¹⁾ sufficiently interleaves A=(A⁽¹⁾, A⁽²⁾). The spike train α⁽²⁾ does interleave A, but the interleaving is insufficient because the interval [α₂⁽²⁾, ∞) contains no spikes from A⁽¹⁾ or A⁽²⁾.

FIG. 148. Example of insufficient interleaving between two collections of spike trains. This example is similar to FIG. 147, however, in this example it is the interval [α₁⁽²⁾, α₂⁽²⁾) that contains no spikes from A⁽¹⁾ or A⁽²⁾.

FIG. 149. Example of computing the projection spike train r from α⁽¹⁾ and α⁽²⁾. In this case, r₁=α₁⁽¹⁾, r₂=α₁⁽²⁾, r₃=α₂⁽¹⁾, and r₄=α₂⁽²⁾.

FIG. 150. Example of sufficient interleaving. In this case, the projected spike train r sufficiently interleaves the collection A=(A⁽¹⁾, A⁽²⁾, A⁽³⁾, A⁽⁴⁾).

FIG. 151. Example of sufficient interleaving between two collections of spike trains. That is, the collection a=(α⁽¹⁾, α⁽²⁾) sufficiently interleaves the collection A=(A⁽¹⁾, A⁽²⁾, A⁽³⁾, A⁽⁴⁾).

FIG. 152. Example of perfect decoding in the presence of noise (advance a spike).

FIG. 153. Example of perfect decoding in the presence of noise (delay a spike).

FIG. 154. Example of perfect decoding in the presence of noise (delete a spike).

FIG. 155. Example of perfect decoding in the presence of noise (add an early spike).

FIG. 156. Example of perfect decoding in the presence of noise (add a late spike).

FIG. 157. Example of perfect decoding in the presence of noise (triple a spike).

FIG. 158. Example of perfect decoding in the presence of noise (delay both spikes).

FIG. 159. Example of perfect decoding in the presence of noise (advance both spikes).

FIG. 160. Example of perfect decoding in the presence of noise (double both spikes).

FIG. 161. Example of perfect decoding in the presence of noise (delete the second spike).

FIG. 162. Example of perfect decoding in the presence of noise (delay both spikes).

FIG. 163. Example of perfect decoding in the presence of noise (advance both spikes).

FIG. 164. Example of perfect decoding in the presence of noise (double both spikes).

FIG. 165. Example of perfect decoding in the presence of noise (delay over inters-pike boundary).

FIG. 166. Imperfect decoding in the presence of noise (advance over inter-spike boundary).

FIG. 167. Imperfect decoding in the presence of noise (advance over inter-spike boundary).

While the invention will be described in connection with certain preferred embodiments, there is no intent to limit it to those embodiments. On the contrary, the intent is to cover all alternatives, modifications and equivalents as included within the spirit and scope of the invention as defined by the appended claims.

### DETAILED DESCRIPTION OF THE INVENTION

### 2 Review of Encoding and Decoding Algorithms

This section provides a quick overview of the encoding and decoding algorithms for discrete SSM Sequence Models. The focus of this disclosure is on identifying the shortcomings of these algorithms that motivated the extensions and generalizations described in this disclosure.

### 2.1 Sequences

The encoding algorithms work with a pair of sequences. In other words, they take two different sequences as input arguments. Because the order of the two sequences matters, we will use S′ to denote the first sequence and S″ to denote the second sequence. To further distinguish between these two sequences, we will use Greek letters to spell the sequence S′ and English letters to spell the sequence S″. This convention will be used throughout this disclosure.

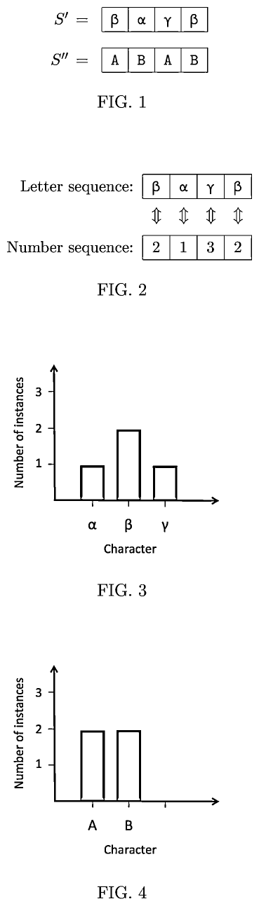

FIG. 1 shows the sequences S′=βαγβ and S″=ABAB that will be used in several of the examples described below. The first sequence is spelled with three unique letters that are drawn from an abbreviated Greek alphabet. We will use Γ″ to denote the alphabet of S′ and M′ to denote its size. In this example Γ′={α, β, γ} and M′=3. Similarly, the sequence S″ is spelled with letters from an abbreviated English alphabet, which will be denoted with Γ″ and its size with M″. For the sequence S″=ABAB the alphabet is Γ″={A, B} and M″=2. Finally, we will use T to denote the length of a sequence. Both sequences in FIG. 1 are of length T=4.

A sequence of letters can be easily converted into a sequence of numbers, and vice versa. One way to perform this conversion is to use a lookup table. For example, the ASCII table is one commonly used method in computer applications. FIG. 2 shows one way to map the sequence S′=βαγβ to a number sequence. A similar mapping can be performed for sequences spelled with English letters. The examples described below use letter sequences; the algorithms use number sequences.

### 2.2 The Histogram of a Sequence

By counting the number of times that each character appears in a given sequence, we can compute the histogram for the sequence. For example, the sequence S′=βαγβ has one α, two β, and one γ. FIG. 3 shows the histogram for this sequence as a bar chart. The height of each bar represents the number of instances of the corresponding character in the sequence.

This bar chart is useful for visualizing the histogram, but it is not very convenient for working with it. Instead, the same information will be represented with a vector. For the sequence S′=βαγβ this vector is h′=[1, 2, 1]. In other words, the values of the histogram bin counters become the elements of the vector h′. In general, this vector is of size M′, where M′ is the size of the alphabet V′.

Similarly, the histogram for the second sequence S″=ABAB can be represented with the vector h″=[2,2] because the sequence contains two A's and two B's. This vector is of size M″, which is the size of the abbreviated English alphabet Γ″ in this example. FIG. 4 visualizes this histogram as a bar chart.

### 2.3 The Histogram of a Sequence Over Time

By definition, the histogram of a sequence is computed for the entire sequence. For some applications, however, it may be useful to compute a histogram only for a prefix of the sequence. The encoding algorithm described later in this chapter incrementally computes the histograms for all possible prefixes of the Greek sequence.

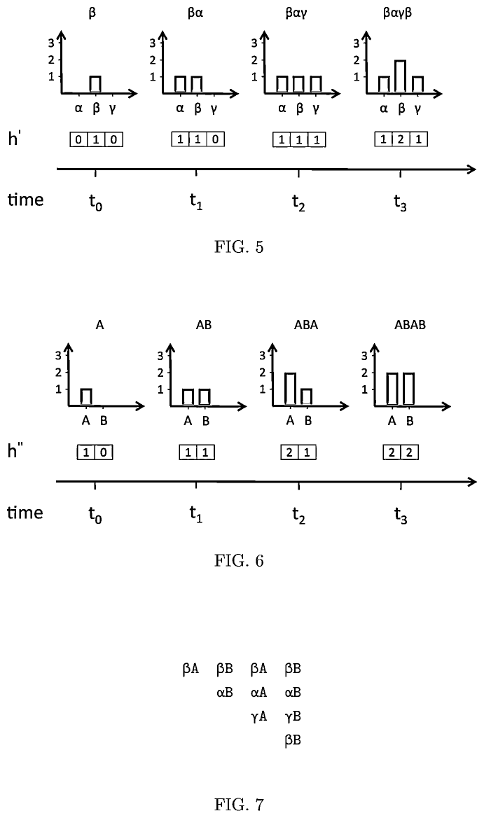

FIG. 5 gives an example with the sequence S′=βαγβ. At time t₀ only the character p is available and the histogram vector is h′=[0,1,0]. At time tᵢ the character α is added to the sequence and the vector is updated to h′=[1, 1, 0]. And so on. At the end of this process h′=[1, 2, 1], which is the histogram for the entire sequence. This computation is performed in place, i.e., all intermediate results are stored in the vector h′. The histogram vector for the sequence S″=ABAB can also be computed incrementally. This process is shown in FIG. 6.

### 2.4 Open Bigrams

Two characters that occur one after another in a sequence form a bigram. For example, the sequence S′=βαγβ has three bigrams: βα, αγ, and γβ. In general, a sequence of length T has T−1 bigrams. Bigrams have a long history in machine learning and artificial intelligence, but we will not use them. Instead, we will use open bigrams.

An open bigram can be formed between any two characters as long as the first character occurs temporally before the second one. In other words, it is no longer required for the two characters to be adjacent in the sequence. For the sequence S′=βαγβ the open bigrams are: βα, βγ, ββ, αγ, αβ, and γβ. In a further extension of this idea, we allow each character to form an open bigram with itself. The reasons for this will become clear later, but for now this adds four additional open bigrams to the list: ββ, αα, γγ, and ββ. Thus, for this sequence there are 10 open bigrams. In general, for a sequence of length T, there are T(T+1)/2 open bigrams. Therefore, a list of open bigrams is a much more dense sequence representation than a list of regular bigrams.

### 2.5 Cross-Sequence Open Bigrams

If we have two different sequences that unfold in parallel over time, then we can generalize the concept of open bigram to cross-sequence open bigram. The principles for forming one of these are similar to the previous case, but now the first character in the cross-sequence open bigram can only come from the first sequence and the second character can only come from the second sequence. The temporal restriction still applies, i.e., the second character cannot be temporally before the first character. A character is no longer allowed to form an open bigram with itself, but it can form an open bigram with the character in the same position in the second sequence. For a pair of sequences, each of which is of length T, there are T(T+1)/2 cross-sequence open bigrams. The rest of this document uses only cross-sequence open bigrams, which will be called open bigrams for the sake of brevity.

FIG. 7 lists all open bigrams for the pair of sequences S′=βαγβ and S″=ABAB. These open bigrams are arranged in an upper triangular grid such that the Greek character in each row is the same and the English character in each column is also the same. This arrangement has some really interesting properties that are used by the encoding and the decoding algorithms.

### 2.6 The SSM Matrix

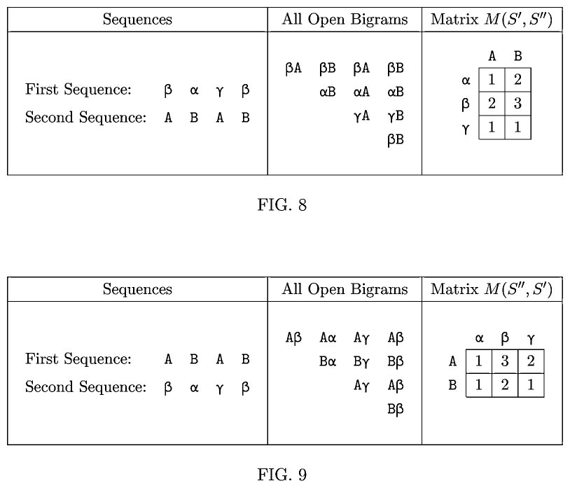

Given two sequences S′ and S″, the open bigrams formed between their characters can be organized in a matrix M, which is called the SSM matrix. FIG. 8 shows an example with the pair of sequences (S′=βαγβ, S″=ABAB). The first column shows the two sequences, which are aligned vertically to denote that they unfold in parallel over time. The middle column shows all open bigrams. The third column shows the matrix. The rows of the matrix are labeled with Greek letters. Its columns are labeled with English letters. Each element of the matrix can be interpreted as a counter that counts the number of open bigrams of a given type. For example, the element in row a and column B is equal to 2, which indicates that the open bigram αB occurs twice in the list of open bigrams. Similarly, the element in row y and column A is equal to one because the open bigram γA appears only once in the list.

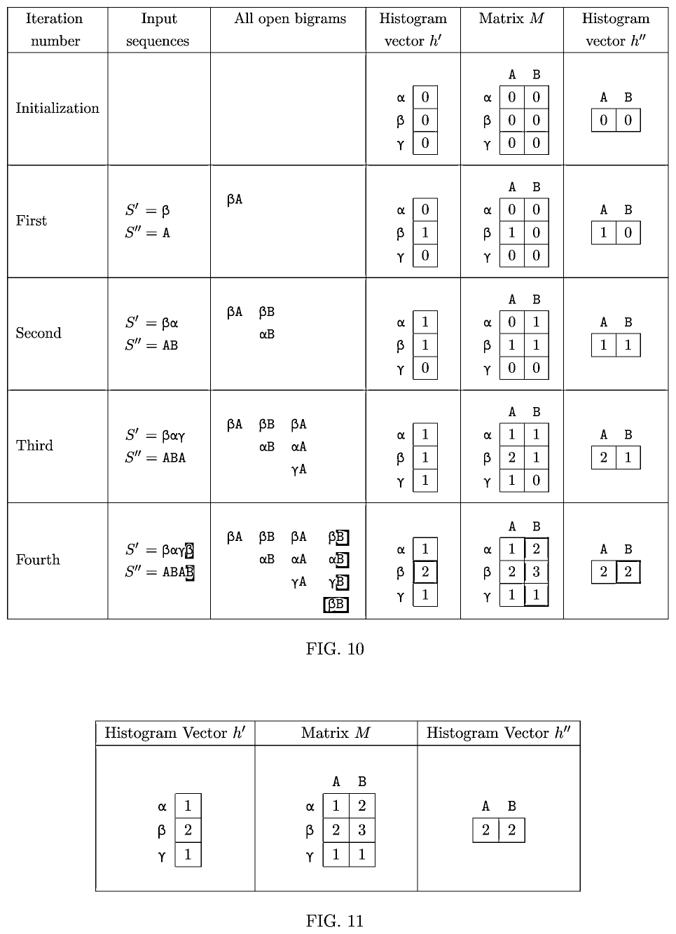

When constructing a matrix, the order of the two sequences matters. To illustrate this, FIG. 9 gives another example with the pair of sequences (S″=ABAB, S′=βαγβ). Now the English sequence S″ is first and the Greek sequence S′ is second. Because the first character in each open bigram now comes from the English alphabet and the second one comes form the Greek alphabet, the list of open bigrams is completely different from the previous example. The matrix is also different. Its rows are now labeled with English letters and its columns are labeled with Greek letters. Each element of the matrix, however, can still be interpreted as a counter for the number of instances of a particular open bigram. For example, the element in row A and column p is equal to 3 because the open bigram Aβ occurs three times in the list. Thus, given two sequences S′ and S″, there are two different matrices that can be constructed. To distinguish between them, we will denote the first one with M(S′, S″) and the second one with M(S″, S′). Unless stated otherwise, all matrices in this document will be of the type M(S′, S″) and they will be denoted with M.

### 2.7 Encoding Example

The encoding algorithm is an efficient way of counting the open bigrams in a pair of sequences and arranging the resulting counts in a matrix format. This section gives a quick overview of this computational procedure.

FIG. 10 gives a step-by-step example that illustrates how the encoding algorithm works. The two sequences in this example are S′=βαγβ and S″=ABAB. Each row of this figure corresponds to one encoding iteration. The second column of the figure shows the prefix of each sequence that has been observed by the algorithm up to that point. The third column shows the open bigrams that have been constructed from these prefixes. The last three columns show the contents of the histogram vector h′, the matrix M, and the histogram vector h″ at the end of each iteration. Because h′ is updated incrementally, it can be interpreted as the histogram of the currently observed prefix of the sequence S′. Similarly, h″ is the histogram of the currently observed prefix of S″.

The last row of FIG. 10 shows the updates performed by the algorithm during the fourth iteration. The elements that are added or modified are highlighted in different colors. We will use this row to explain how the algorithm works. The incoming character from the sequence S′ is p, which is highlighted in red. Therefore, the corresponding bin of the first histogram h′, which is also highlighted in red, is incremented by one. The incoming character from the second sequence S″ is B. Thus, the algorithm adds the contents of the vector h′ to the matrix column that corresponds to B (this is the green column in the figure). The incoming character from S″ also selects which bin of h″ should be incremented by one (the B-th bin in this case, which is highlighted in green).

FIG. 10 may imply that the algorithm has access to the prefixes of both sequences, but in practice it needs only the most recent character from each sequence to perform the calculations for each iteration. Thus, there is no need to store the sequences and the encoding can be performed with a single pass through both sequences, without the need to go back and look at any previous characters. The computational complexity of the encoding algorithm is O(TM′), where T is the length of the sequences and M′ is the alphabet size of the first sequence. In other words, during each of the T iterations the algorithm updates only one column of the matrix, which has M′ elements.

The number of open bigrams that need to be counted grows with each iteration. There is only 1 during the first iteration, 2 during the second, 3 during the third, and 4 during the fourth. In total there are 10 of them in this example. This begs the question: How can the algorithm keep up with this ever increasing number of open bigrams and still maintain its computational complexity?

The last row of FIG. 10 helps explain this. During the fourth iteration the algorithm needs to account for 4 open bigrams: βB, αB, γB, and βB. The second character in all four of these is B (highlighted in green in the figure). This character corresponds to the current character from S″, and also to the matrix column that needs to be updated. The first character in the fourth open bigram is β and it corresponds to the current character from S′ (highlighted in red). The first character in the other three open bigrams corresponds to one of the three characters in the prefix of S′. Note that even though there are four open bigrams, two of them are the same. That is, there are two instances of the open bigram βB. Also, note that the value of the vector h′ at this time is h′=[1, 2, 1]^{T}. This can be interpreted as one α, two β, and one γ. By adding this vector to the B-th column of the matrix the algorithm can account for all open bigrams at this iteration. The repeated instance of βB is correctly accounted for because the p-th bin of h′ is equal to 2. This explains why 4 open bigrams can be accounted for in the matrix using only 3 additions.

In other words, the algorithm uses the vector h′ to perform the computation more efficiently. It uses the fact that, no matter how many open bigrams need to be counted at each iteration, there will be at most M′ unique ones. That is, the first alphabet has a finite and fixed size, and therefore there will be at most that many unique open bigrams at each iteration (recall that the second character in each of these open bigrams is always the same). Furthermore, the value of the histogram h′ can be reused from one iteration to the next, after incrementing only one of its bin counters. In other words, the histogram is computed incrementally—it does not need to be recomputed from scratch during each iteration.

To summarize, during each encoding iteration, the current character from S′ indicates which bin counter of h′ will be incremented by one. The current character from S″ selects the matrix column to which h′ must be added. The current character from S″ also determines which bin of h″ should be incremented. Thus, during each iteration, the algorithm needs to update only one bin of h′, only one column of the matrix, and only one bin of h″. Note that the vector h″ is computed by the encoding algorithm, but it is not used to update the matrix. Instead, it is used at a later time by the decoding algorithm.

### 2.8 The SSM Model

This section defines the SSM model, which is used by the decoding algorithm and the evaluation script described later in this chapter.

The encoding SSM model for the sequence pair (S′, S″) is defined as the matrix M and the vectors h′ and h″ that are computed by the encoding algorithm. To give a concrete example, we will use the sequences S′=βαγβ and S″=ABAB from the previous section. The matrix in this case is of size 3×2. The vector h′, which represents the histogram for the sequence S′, is a column vector of size 3. The histogram vector h″ for the sequence S″ is a row vector of size 2. FIG. 11 shows the final values for all three, which are the same as in the last row of FIG. 10.

In general, the size of the computed matrix is M′×M″, where M′ is the alphabet size for the sequence S′ and M″ is the alphabet size for the sequence S″. The vectors h′ and h″ are of size M′ and M″, respectively. Once again, all three of these are computed by the encoding algorithm. However, the decoding algorithm, which is described in the next section, needs only the matrix and the second histogram. That is, the decoding algorithm does not need h′ in order to decode S′ from the matrix. Therefore, the first histogram can be discarded after the encoding is done.

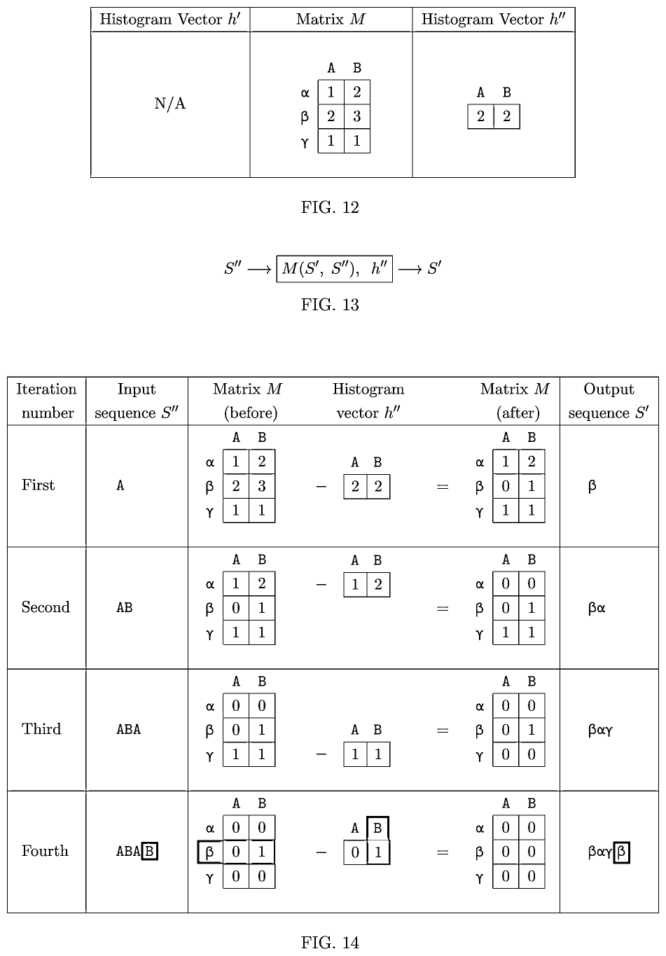

The decoding SSM model for the sequence pair (S′, S″) is defined as the matrix M and the vector h″ that are computed by the encoding algorithm. FIG. 12 shows the decoding model for the sequence pair (S′=βαγβ, S″=ABAB). The histogram vector h′ for the first sequence is used by the encoding algorithm to compute the matrix M, but it is not included in the decoding model. In other words, h′ can be viewed as a helper array that can be discarded at the end of the encoding process. The rest of this chapter uses the word model or SSM model to refer to this decoding model. By default, SSM model refers to a decoding SSM model. Also, for the purposes of aliasing detection (which is defined below) this is the default model as well.

### 2.9 Decoding Example

This section gives an example that illustrates the decoding algorithm. FIG. 13 visualizes the decoding task as a flow diagram. The box in the middle represents the SSM model after the end of encoding. This model consists of the matrix M(S′, S″) and the histogram vector h″. In other words, this box can be viewed as an abbreviated notation for the contents of FIG. 12. Given the sequence S″ at run time, the decoding algorithm tries to decode the sequence S′ from the model. The arrows indicate the input and the output of this process.

FIG. 14 gives a step-by-step example of the decoding process. The model in this case was computed from the sequences S′=βαγβ and S″=ABAB, which are the same two sequences that were used in the encoding example in the previous section. Each row of the figure corresponds to one decoding iteration. During each iteration the algorithm tries to find one row of the matrix from which it can subtract the vector h″. A precondition for this operation, however, is that after the subtraction none of the matrix elements can be negative. If such a row can be found, then the subtraction is performed and the matrix is updated. The Greek letter that corresponds to this row is then added to the output sequence. Before the next iteration the bin counter of h″ that corresponds to the current character from S″ is decremented by one.

The last row of FIG. 14 shows the updates that are performed during the fourth decoding iteration. In this case, h″ can only be subtracted from the second row of the matrix without any elements becoming negative. This row corresponds to the Greek letter p, which is added to the output sequence and is highlighted in red in the figure. The incoming character on S″ at this time is B (highlighted in green) and therefore, the B-th bin of h″ will be decremented by one (also highlighted in green). At the end of the decoding process, both the matrix and the vector h″ contain only zeros.

The computational complexity of this algorithm is O(TM″). This is comparable to the complexity of the encoding algorithm, which is O(TM′).

### 2.10 Decoding Limitations of Regular Matrices

This section analyzes the decoding properties of SSM matrices. The analysis shows that for some pairs of sequences of length three these matrices are not uniquely decodable. The analysis also shows that these decoding limitations increase as the sequence length increases.

Without loss of generality, all examples in this section use pairs of sequences that are constructed from an abbreviated Greek alphabet with only two letters and an abbreviated English alphabet with only two letters as well.

### 2.10.1 Sequences of Length 1

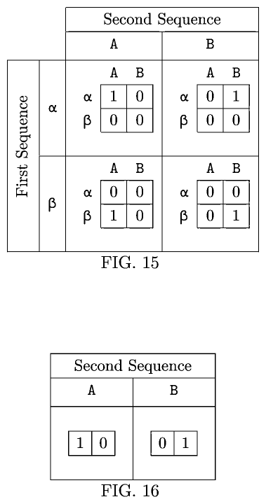

When the two sequences are of length one, there are only four pairs of sequences from which a matrix can be constructed. These sequence pairs are: (α, A), (α, B), (β, A), and (β, B). FIG. 15 shows the four matrices that correspond to these sequence pairs. The histograms that correspond to the English sequences are shown in FIG. 16. It is easy to verify that all four matrices are uniquely decodable given the original English sequence at run time.

### 2.10.2 Sequences of Length 2

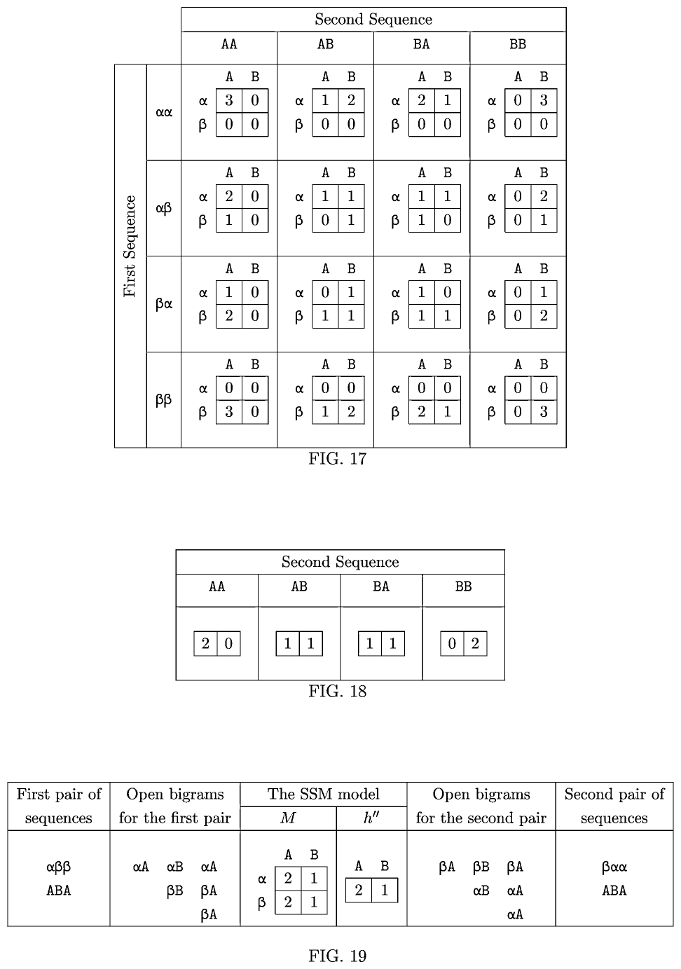

There are only four possible Greek sequences of length two that can be constructed from a two-letter alphabet: αα, αβ, βα, and ββ. Similarly, there are only four possible English sequences: AA, AB, BA, and BB. Thus, in this case, there are 16 possible combinations of a Greek sequence and an English sequence. All 16 matrices for these sequence pairs are shown in FIG. 17.

It is relatively easy to verify that all 16 matrices are uniquely decodable. Once again, it is assumed that the English sequence that was used to encode the matrix is available at run time. The histograms for the English sequences are shown in FIG. 18.

### 2.10.3 Sequences of Length 3

If the input sequences are at least three characters long, then the mapping from sequence pairs to matrices is no longer unique. In other words, when T=3 there are at least two different pairs of sequences that map to the same matrix. For example, both (αββ, ABA) and (βαα, ABA) map to the matrix shown in FIG. 19. Because the English sequence S″=ABA is the same in both pairs they also have the same histogram vector h″=[2,1]. Sequence pairs like these will be called aliased because they map to the same matrix and the same second histogram, i.e., they have the same SSM model.

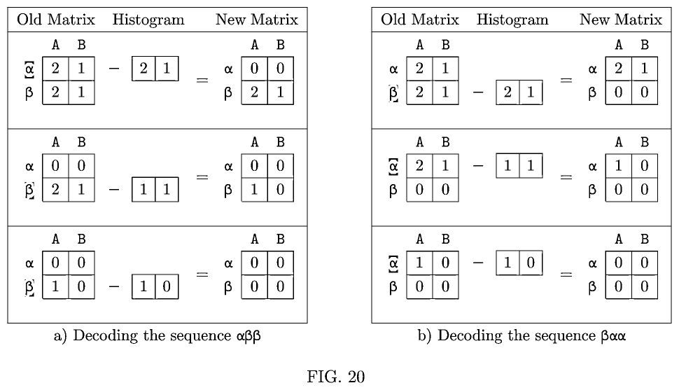

FIG. 20 shows that it is possible to decode the two aliased Greek sequences from this model, given the same English sequence at run time. Thus, the ambiguity limit for decoding of dual matrices is T=3.

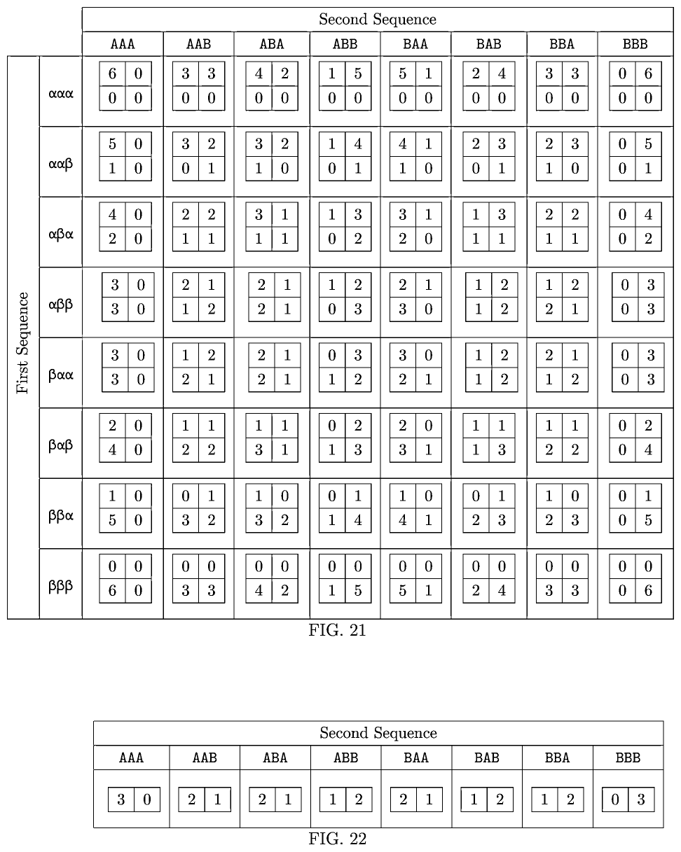

FIG. 19 showed only one example of aliasing. Are there any other examples? To answer this question, we can use exhaustive enumeration to list all 64 possible sequence pairs and construct a model for each pair. FIG. 21 shows the 64 possible matrices. FIG. 22 shows the histograms for the English sequences as vectors. Because there are only 8 possible S″ sequences, there are only 8 possible h″ vectors. In other words, the matrices in each column of FIG. 21 have the same h″ vector, which is shown in the corresponding column of FIG. 22.

Visual inspection shows that there are four groups of aliased models (their matrices are highlighted in gray in FIG. 21). The four groups are encoded from the following sequence pairs: 1) (αββ, AAA) and (βαα, AAA); 2) (αββ, ABA) and (βαα, ABA); 3) (αββ, BAB) and (βαα, BAB); and 4) (αββ, BBB) and (βαα, BBB). That is, the sequence pairs in each group map to the same M and h″. The decoding algorithm described in Section 2.9, however, always decodes the S′ sequence associated with the first pair. The reason is that given a choice the algorithm always subtracts h″ from the matrix row that is first in alphabetical order. For the example shown in FIG. 20 the algorithm will always choose the first row of the matrix and decode opp. Even though the sequence pair (βαα, AAA) maps to the same matrix, the algorithm will never output βαα given AAA at run time. Thus, the decoding will be wrong for only 4 of the 64 possible models.

To summarize, for T=3 there are 64 possible sequence pairs. Each pair maps to a matrix and a histogram vector h″. Thus, there are 64 models. Of these, 56 are unique and 8 are aliased. The aliased models can be split into 4 groups of 2, such that in each group both M and h″ are the same. For the first sequence pair in each group, the decoding algorithm returns the correct S′ sequence because it picks the Greek letter that is first in alphabetical order. For the second pair the decoding algorithm returns the aliased S′ sequence, i.e., the one that belongs to the other pair. For the 56 non-aliased sequence pairs the decoding algorithm always returns the correct S′ sequence. Thus, for T=3 there are 4 (or 6.25%) wrong decoding outcomes (i.e., an aliased S′ is decoded) and 60 (or 93.75%) correct outcomes. The correct outcomes, however, can be split into two groups. The first group contains 56 sequence pairs that are uniquely mapped to an SSM model. The second group contains 4 sequence pairs that have an aliased mapping, but for which the decoding algorithm returns the correct S′ because of the way it does tie breaking.

### 2.10.4 Sequences of Length Up to 10

This section extends the decoding evaluation from the previous section to sequences of length up to 10. As the sequences become longer, the number of sequence pairs grows exponentially. If the sizes of the two alphabets are M′ and M″, then there are (M′)^{T}×(M″)^{T} possible sequence pairs of length T. For example, when M′=M″=2 and T=10 there are 2¹⁰×2¹⁰=1,048,576 possible sequence pairs. FIG. 23 shows how the number of sequence pairs (and models) grows as a function of M′, M″ and T.

At this point it should be obvious that evaluation by hand is neither feasible nor desirable. To get around this problem, we used a computer script. The script takes M′, M″, and T as parameters and then exhaustively enumerates all possible (M′)^{T}×(M″)^{T} sequence pairs. For each pair, the script runs the encoding algorithm and computes a matrix and a histogram for the second sequence. The script then evaluates both the encoding outcomes and the decoding outcomes as described below.

To characterize the encoding outcomes, the script compares the SSM model of each sequence pair against the models of all other sequence pairs. If there is no match, then the encoding is counted as unique. If it finds a match, then the encoding is counted as aliased. That is, there are at least two different sequence pairs that map to the same M and h″. Once this check is done for all pairs, the script reports the percentage of unique and aliased sequence pairs.

For the decoding outcomes, the script attempts to decode each model after it is encoded. To explain this process, let (S₁, S₂) be a sequence pair for which a model was computed. The script then calls the decoding algorithm with S₂ as a parameter and compares the decoded sequence to S₁ (i.e., the same one that was used to encode the matrix). There are four possible outcomes of this process: 1) the decoded sequence is the same as the sequence S₁ that was used during encoding; 2) the decoded sequence is different from S₁, but it is equal to one of the sequences in the aliased pairs; 3) the decoded sequence is of length T, but it is neither the correct sequence nor an aliased sequence; and 4) the decoded sequence is wrong and its length is shorter than T. The last case corresponds to a decoding process that got “stuck”, i.e., the algorithm reached a point at which it couldn't subtract h″ from any row of the matrix without some matrix elements becoming negative in the process.

Thus, there are two encoding outcomes and four decoding outcomes. Combining these outcomes leads to eight different cases that are summarized in FIG. 24. The top row of the figure is for unique encoding outcomes; the bottom row is for aliased outcomes. Each cell contains a diagram that illustrates each of these eight outcomes. The second cell in the first row is denoted with N/A because this particular encoding-decoding combination is impossible. In other words, it is not possible for the encoding algorithm to produce a unique model and for the decoding algorithm to produce an aliased sequence.

Consider the diagram in the upper-left corner in FIG. 24. The input in this case is the sequence pair (S₁, S₂). The double arrow indicates the encoding process, which takes two sequences and produces an SSM model. Thus, the encoding is represented by (S₁, S₂)⇒Model. The rest of the diagram is for the decoding process, which takes one sequence as input and produces one sequence as output. In this case the input is the sequence S₂, which is connected with a regular arrow to the model. The output is S₁, which is connected with a regular arrow as well. Thus, S₂→Model→S₁ captures the decoding process. The other diagrams in the first row of FIG. 24 are similar. Because they represent a failed decoding process, however, the output sequence is indicated with either S_{w} (i.e., wrong sequence) or S_{ws} (i.e., wrong short sequence).

The diagrams in the bottom row of the figure are for aliased encoding. In this case two (or more) sequence pairs map to the same model. This is indicated with the sequence pairs (S₁, S₂), . . . , (Sₚ, S_{q}) that are connected with double arrows to the model. As described above, these aliasing effects are detected by the script using exhaustive enumeration. For evaluation purposes, however, only one of these sequence pairs is considered the main one during a particular testing iteration. For the sake of explanation, let (S₁, S₂) be that pair. Thus, when testing the decoding outcome, the script will provide the sequence S₂ as input and compare the output sequence to S₁. If the decoded sequence is equal to S₁ (see the 1-st column in the figure), then the decoding is considered correct. In some cases the decoded sequence is from one of the aliased pairs (e.g., Sₚ as in the 2-nd column). In the remaining two cases the decoded sequence is wrong (indicated with S_{w} in the 3-rd column) or wrong and short (indicated with S_{ws} in the 4-th column).

FIG. 25 shows the evaluation results for M′=M″=2 and for T=1, 2, . . . , 10. The eight plots in this figure correspond to one of the eight cases shown in FIG. 24. The impossible case is represented with a plot that is always at 0%. The results in these plots are expressed as a percentage of the number of sequence pairs for each T. As shown in FIG. 23, the number of these pairs grows exponentially as T increases. For example, for T=2 there are 16 pairs while for T=5 there are 1024 pairs. In the first case 16 (or 100%) are uniquely decodable, while in the second case only 330 (or 32%) are uniquely decodable. Thus, even though there are more decodable matrices for T=5, they represent a lower percentage of the total number for that sequence length.

There are several interesting things to note here. First, the performance drops quite rapidly as T increases. The plot in the upper-left cell in FIG. 25 is probably the most informative one. Second, there is aliasing for T≥3. Third, as T increases the percentage of wrong sequences during decoding also increases. Another interesting property is that the decoding algorithm either returns an aliased sequence or it gets stuck. It never returns a wrong sequence of the same length T (i.e., the plots in the 3-rd column are always at 0%).

These results indicate that the performance of the decoding algorithm drops quite rapidly as the sequence length increases. The next two sections describe one possible extension of the SSM model, and its associated algorithms, that performs better according to these metrics.

2.11 Encoding Example with Exponential Decay

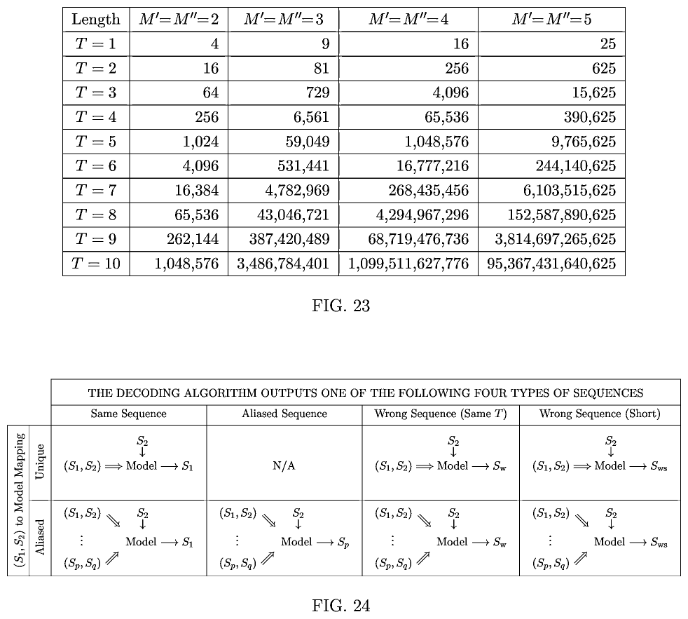

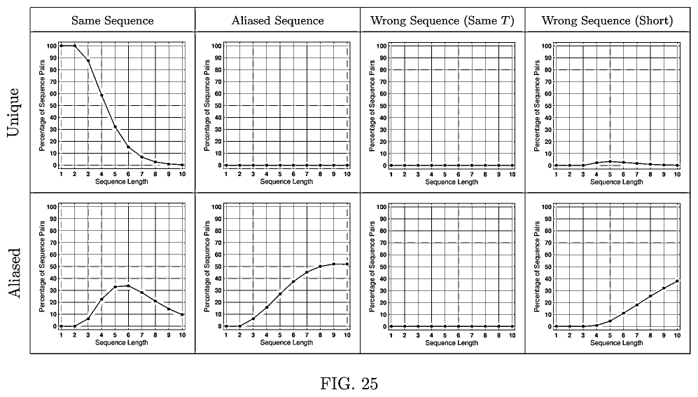

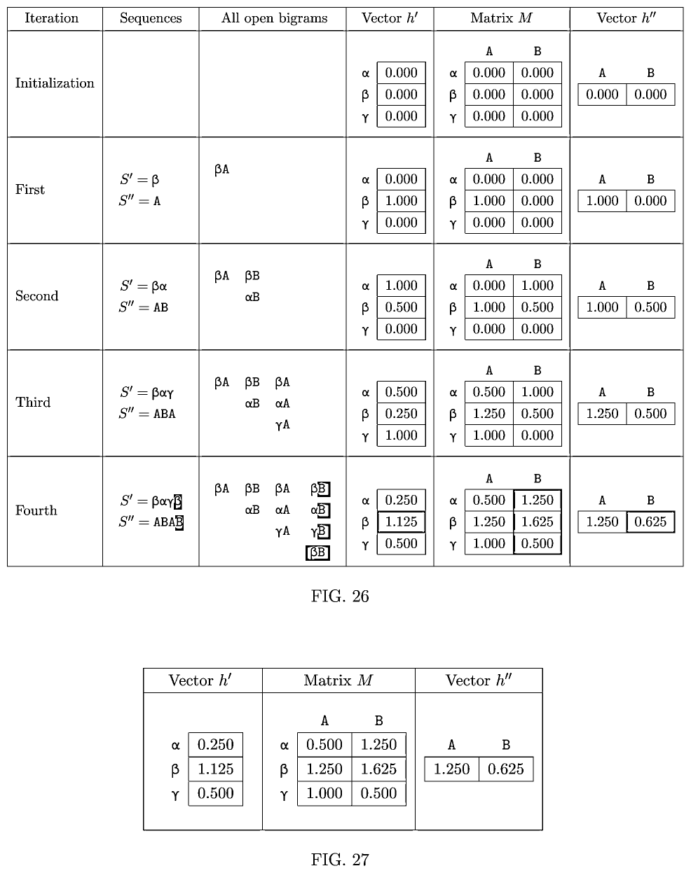

FIG. 26 gives an example that will be used to explain the encoding algorithm. The two input sequences in this case are S′=βαγβ and S″=ABAB. This is similar to the example in Section 2.9, but now there is also an exponential decay. This decay is controlled by the parameter z, which is equal to 2 in this case. The exponential decay affects how the vector h′ is computed. At the start of each iteration all elements of h′ are divided by two. Thus, the elements of h′ decay in half from one iteration to the next. The current character in S′ determines which element of h′ will be incremented by 1 (this contribution will decay in half by the next iteration). In other words, each element of h′ can be viewed as a leaky integrator. The vector h′ is still added to one column of the matrix. Which column? That is determined by the current character from the second sequence S″.

The exponential decay also affects the vector h″. In this case, however, the decay affects only what is added to this vector. In other words, the elements of h″ don't decay from one iteration to the next. What decays is the increment value, which is added to only one element. Note that for the vector h′ the increment value is implicitly set to 1 and it remains the same for all iterations. In this case the increment value is z, which is initially set to 1 and decays in half (divided by z) from one iteration to the next.

A side effect of the exponential decay is that the elements of the matrix are no longer integer numbers. In fact, all three components—h′, M, and h″—now have real values. Also, just like h″, the contents of the matrix don't decay during the encoding process. In other words, what is added to the matrix stays in the matrix. FIG. 27 shows the encoding SSM model for this example, which consists of the matrix M and the vectors h′ and h″. The vector h′ is used to compute the matrix, but it is not needed by the decoding algorithm; it is not used for aliasing detection either.

Note that in the exponential model the word histogram is no longer an accurate description for h′ or h″. A better word might be ‘history’, i.e., the history of each character in the corresponding input sequence. In any case, we will continue to use h′ and h″ to denote these two vectors. For lack of a better word sometimes we may refer to them as histograms. In should be noted, however, that these two vectors reduce to proper histograms only if z=1 (i.e., when there is no exponential decay or growth as in the previous sections). When z=1 the vector h′ is indeed the histogram of the characters in the first sequence S′ and the vector h″ is indeed the histogram of the second sequence S″. Once again, this is no longer the case when z≠1.

2.12 Decoding Example with Exponential Decay

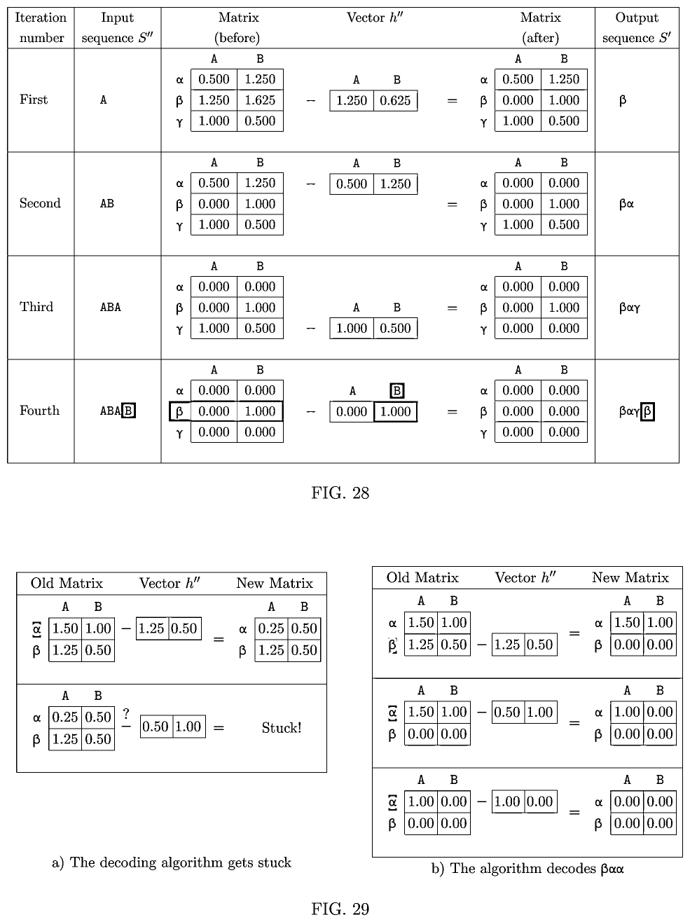

FIG. 28 gives an example that will be used to describe the decoding process. Each row of this figure corresponds to a separate decoding iteration. The goal of the algorithm is to decode the sequence S′ from the matrix M and the vector h″, given the sequence S″ at run time. During each iteration the goal is to find one row of the matrix from which to subtract the vector h″. This search is subject to the constraint that no matrix element could be negative after the subtraction. If a suitable row is identified, then the Greek letter associated with that row is added to the output sequence and the subtraction is performed. In addition, the element of the vector h″ that corresponds to the current character in S″ is decremented by 1. After this subtraction is performed all elements of h″ are multiplied by 2 and the algorithm proceeds to the next iteration.

The elements of M and h″ that are modified during the last iteration of the algorithm are highlighted in red and green in the last row of FIG. 28. At this moment the vector h″ can only be subtracted from the p row of the matrix (highlighted in red). Thus, the output character is p (also highlighted in red). The incoming character on S″ is B (highlighted in green) and thus a 1 is subtracted from the B-th element of h″. Since this is the last iteration, there is no need to multiply all elements of h″ by 2 (or, rather, the vector contains only zeros and that operation has no effect).

At the end of the decoding process both the matrix and the vector h″ should contain only zeros. If this is not the case, then the decoding process probably got stuck. The next section analyzes how the decodability properties of the exponential model depend on the sequence length.

### 2.13 Decoding Limitations of Exponential Matrices

This section shows that the same limit of T=3 holds for the deterministic decoding of exponential matrices as well. One difference in this case is that the mapping from sequence pairs to models is now one-to-one, i.e., due to the exponential decay there is no aliasing. This section also analyzes the decodability properties of the model for sequences of length up to 10.

FIG. 29 shows the decoding process for a matrix that was encoded from the sequences S′=βαα and S″=ABA. As with other decoding examples, this one assumes that the characters of the English sequence S″ are provided at run time. As can be seen from the figure, during the first iteration the algorithm has a choice. It can subtract the vector h″ from either the first row or from the second row of the matrix. If it picks the first row, then it gets stuck during the second iteration (i.e., it is no longer possible to subtract h″ from any row of the matrix without any of the matrix elements becoming negative). If it picks the second row, then it can successfully decode the Greek sequence. Thus, the decoding algorithm could get stuck for sequences of length 3. Therefore, the decoding process is not deterministic for T=3.

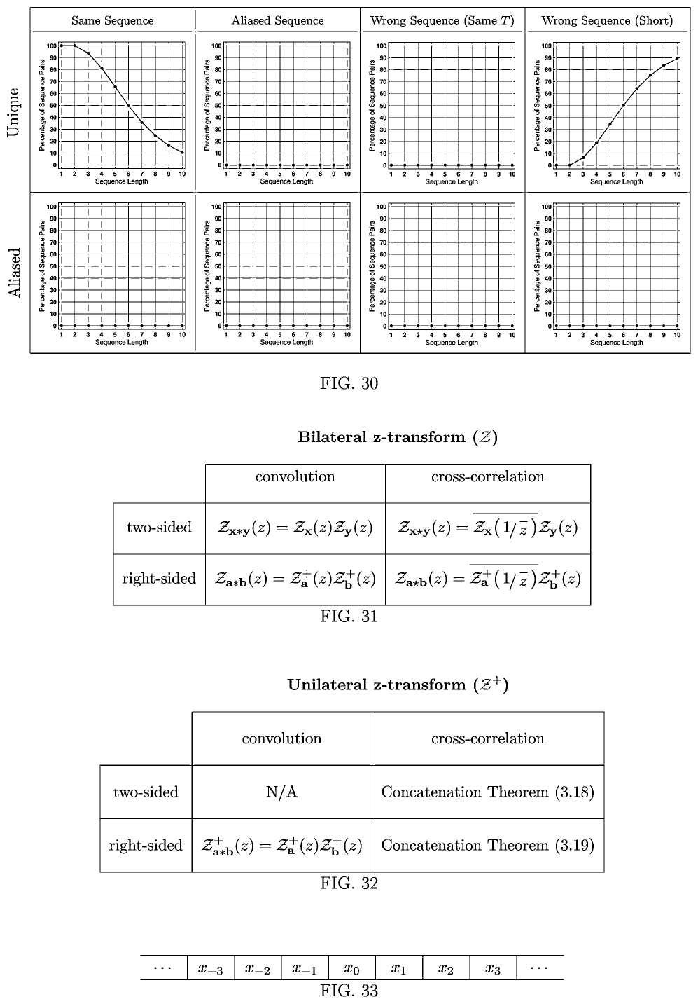

FIG. 30 shows the evaluation results for the decoding algorithm with exponential decay for sequences of length up to 10. These results are reported using the classification system described in FIG. 24. The exponential decay changes the properties of the model. In particular it eliminates aliasing, i.e., the mapping from a pair of sequences to an SSM model is now one-to-one. The decoding properties of the algorithm are also modified. Since there is no aliasing, it is not possible to decode an aliased sequence from the model (i.e., all plots in the bottom row of FIG. 30 are constant at 0%). The only two options in this case are to decode the correct sequence or to decode a wrong sequence that is shorter than the original sequence and then to get stuck. Thus, the algorithm either succeeds or it fails. Unfortunately, as the sequence length increases the percentage of failures increases dramatically, reaching almost 90% for T=10.

The example from FIG. 29 and the exhaustive enumeration results from FIG. 30 show that the SSM model for the exponential case is also not perfect. Even though aliasing is now eliminated, the decoding is not deterministic for sequences longer than two characters. These limitations prompted the search for an improved representation and its corresponding encoding and decoding algorithms, which are described in this disclosure.

### 2.14 Summary

This chapter provided a quick overview of the encoding and decoding algorithms for discrete sequences that were described in our previous document. Emphasis was added on evaluating the decodability properties of the model in each case. Section 2.10 showed that for sequences of length 3 the mapping from sequence pairs to models is aliased (i.e., many-to-one) and that the decoding process is no longer deterministic. Section 2.13 showed that the exponential version of the algorithms eliminates aliasing effects but the decodability limit is still equal to 3.

These results prompted the search for a new class of algorithms that could overcome this decodability limit and for a set of conditions under which deterministic decoding is always possible. This led to the development of the ZUV family of algorithms, which are described in the next couple of chapters. The algorithms described in this chapter can be viewed as special cases of the ZUV algorithms.

### 3 Discrete-Time Formulation

This chapter shows that the value of each element in a dual exponential SSM matrix is equal to the value of the unilateral z-transform, evaluated at a specific z, of the cross-correlation of the two right-sided sequences that correspond to the row channel and the column channel. This result is justified by the concatenation theorem and its corollaries, which are stated in Sections 3.4 and 3.5. Chapter 4 provides examples that complement the theory described here.

### 3.1 Infinite Sequences

A sequence is a collection of numbers that are arranged in a specific order. An infinite sequence is a sequence that has infinitely many numbers. In this chapter it is assumed that, by default, sequences consist of complex numbers. The cases in which the elements of the sequences are restricted to real numbers are explicitly indicated in the text.

There are two types of infinite sequences: right-sided sequences and two-sided sequences. A right-sided sequence is a collection of complex numbers that is indexed by nonnegative integers. A two-sided sequence is a collection of complex numbers that is indexed by the set of all integers, which consists of the positive integers, the negative integers, and zero.

Definition 3.1. A right-sided infinite sequence a=(a₀, a₁, a₂, . . . ) is a function that maps nonnegative integers to complex numbers. In other words, a; denotes the value of the function when its nonnegative integer argument is equal to i.
Definition 3.2. A two-sided sequence x=( . . . , x₋₂, x₋₁, x₀, x₁, x₂ . . . ) is a function that maps all integers (i.e., positive integers, negative integers, and zero) to complex numbers. In other words, xᵢ denotes the value of the function when its integer argument is equal to i.
Definition 3.3. A finite sequence u=(u₀, u₁, u₂, . . . , u_{T-1}) of length T is a function that maps each integer between 0 and T−1 to a complex number. In other words, u; denotes the value of the function when its argument is equal to i∈{0, 1, 2, . . . , T−1}.

### 3.2 The Z-Transform

This section introduces the z-transform of a sequence. If the sequence is right-sided, then only the unilateral z-transform can be obtained from it. If the sequence is two-sided, then both the unilateral z-transform and the bilateral z-transform can be derived. The formal definitions are given below.

Definition 3.4. Let a=(aa, a₁, a₂, . . . ) be a right-sided infinite sequence. The unilateral z-transform of the sequence a is a function, denoted by [symbol] ₐ⁺(z), that maps a complex scalar z to the value of the power series derived from a and evaluated at z⁻¹. More formally,

$${ℨ}_{a}^{+} ( 𝓏 ) = {\sum }_{n = 0}^{∞} {a}_{n} {𝓏}^{- n} . \qquad ( 3.1 )$$

The domain of [symbol] ₐ⁺ which is also called the region of convergence (ROC), consists of all complex numbers for which the series converges. More formally,

$$domain ( {ℨ}_{a}^{+} ) = \{ 𝓏 ∈ ℂ : \vert {\sum }_{n = 0}^{∞} {a}_{n} {𝓏}^{- n} \vert < ∞ \} . \qquad ( 3.2 )$$

Definition 3.5. Let y=( . . . , y₋₁, y₀, y₁, . . . ) be a two-sided infinite sequence. The bilateral z-transform of y is the function [symbol] _{y}(z) that maps a complex scalar z to the value of the bilateral power series derived from y and evaluated at z⁻¹. More formally,

$${ℨ}_{y} ( 𝓏 ) = {\sum }_{n = - ∞}^{∞} {y}_{n} {𝓏}^{- n} . \qquad ( 3.3 )$$

The domain of [symbol] _{y}, i.e., its region of convergence, consists of all complex scalars z for which the power series converges. More formally,

$$domain ( {ℨ}_{y} ) = \{ 𝓏 ∈ ℂ : \vert {\sum }_{n = - ∞}^{∞} {a}_{n} {𝓏}^{- n} \vert < ∞ \} . \qquad ( 3.4 )$$

### 3.3 The Cross-Correlation Theorem for the Z-Transform

Cross-correlation is an operation on a pair of sequences that is similar to convolution. Unlike convolution, however, cross-correlation is not a commutative operation. That is, the order of the two sequences is important for cross-correlation. Therefore, it makes sense to talk about the first and the second sequence for cross-correlation, but not for convolution. To distinguish between these two operations we will use * for convolution and * for cross-correlation.

### 3.3.1 Cross-Correlation: Definitions and Properties

Definition 3.6. Let x and y be two-sided infinite sequences. The discrete cross-correlation of x and y, which is denoted by x*y, is a two-sided infinite sequence in which the n-th element is defined using the following formula:

$${( x ★ y )}_{n} = {\sum }_{m = - ∞}^{∞} \overline{{x}_{m}} {y}_{m + n} , for each n ∈ ℤ = \{ … , - 2 , - 1 , 0 , 1 , 2 , … \} , \qquad ( 3.5 )$$

where x̅ₘ̅ denotes the complex conjugate of xₘ.
Definition 3.7. Let a and b be two right-sided infinite sequences. The discrete cross-correlation of a and b, which is also denoted by a*b, is a two-sided infinite sequence in which the n-th element is defined as follows:

$${( a ★ b )}_{n} = {\sum }_{m = max ( 0 , - n )}^{∞} \overline{{a}_{m}} {b}_{m + n} , for each n ∈ ℤ = \{ … , - 2 , - 1 , 0 , 1 , 2 , … \} . \qquad ( 3.6 )$$

Some problems require only the right tail of the cross-correlation sequence, e.g., calculating the unilateral z-transform of a*b. In these special cases n is a positive integer or zero, which implies that max(0, −n)=0. Therefore, the sum in formula (3.6) can start from 0, which leads to the following simplified expression for the elements of the cross-correlation sequence:

$${( a ★ b )}_{n} = {\sum }_{m = 0}^{∞} \overline{{a}_{m}} {b}_{m + n} , if n \geq 0. \qquad ( 3.7 )$$

Definition 3.8. Let a=(a₀, a₁, . . . , a_{T-1}) and b=(b₀, b₁, . . . , b_{T-1}) be two right-sided finite sequences of length T. Then, the discrete cross-correlation of a and b is a two-sided finite sequence of length 2T−1, i.e.,

$$( a ★ b ) = ( {( a ★ b )}_{- ( T - 1 )} , {( a ★ b )}_{- ( T - 2 )} , … , {( a ★ b )}_{- 1} , {( a ★ b )}_{0} , {( a ★ b )}_{1} , … , {( a ★ b )}_{T - 2} , {( a ★ b )}_{T - 1} ) . \qquad ( 3.8 )$$

Furthermore, the n-th element of this sequence is given by the following formula:

$${( a ★ b )}_{n} = {\sum }_{m = max ( 0 , - n )}^{min ( T - 1 , T - 1 - n )} \overline{{a}_{m}} {b}_{m + n} , for each n ∈ \{ - ( T - 1 ) , - ( T - 2 ) , … , - 1 , 0 , 1 , … , T - 2 , T - 1 \} . \qquad ( 3.9 )$$

If only the right tail of the cross-correlation sequence is needed, then (3.9) can be simplified as follows:

$${( a ★ b )}_{n} = {\sum }_{m = 0}^{T - 1 - n} \overline{{a}_{m}} {b}_{m + n} , if 0 \leq n \leq T - 1. \qquad ( 3.1 )$$

### Property 3.9. Additivity Property of Cross-Correlation.

Let u, v, x, and y be two-sided sequences such that the four cross-correlations u*x, u*y, v*x, and v*y are well-defined, i.e., each of the series that define their elements converges. More formally,

$$\vert {( u ★ x )}_{n} \vert = \vert {\sum }_{m = - ∞}^{∞} \overline{{u}_{m}} {x}_{m + n} \vert < ∞ , \qquad ( 3.11 )$$

$$\vert {( u ★ y )}_{n} \vert = \vert {\sum }_{m = - ∞}^{∞} \overline{{u}_{m}} {y}_{m + n} \vert < ∞ , \qquad ( 3.12 )$$

$$\vert {( v ★ x )}_{n} \vert = \vert {\sum }_{m = - ∞}^{∞} \overline{{v}_{m}} {x}_{m + n} \vert < ∞ , \qquad ( 3.13 )$$

$$\vert {( v ★ y )}_{n} \vert = \vert {\sum }_{m = - ∞}^{∞} \overline{{v}_{m}} {y}_{m + n} \vert < ∞ , \qquad ( 3.14 )$$

for each n∈ [symbol] ={ . . . , −2, −1, 0, 1, 2 . . . }.
Under these conditions, the discrete cross-correlation is additive in both arguments, i.e.,

$$( u + v ) ★ ( x + y ) = u ★ x + u ★ y + v ★ x + v ★ y . \qquad ( 3.15 )$$

### Property 3.10. Scalar Multiplication Property of Cross-Correlation.

Let x and y be two-sided sequences. Then,

$$( α x ) ★ y = \overline{α} ( x ★ y ) , \qquad ( 3.16 )$$

$$x ★ ( α y ) = α ( x ★ y ) , \qquad ( 3.17 )$$

for each α∈ [symbol] . Note that the complex scalar a is conjugated only in the first equation.

### Corollary 3.11. Combined Formula for Additivity and Scalar Multiplication.

Let u, v, x, and y be two-sided sequences. Also, let α, β, φ, and ψ be complex scalars. Then,

$$( α u + βv ) ★ ( φ x + ψ y ) = \overline{α} φ ( u ★ x ) + \overline{α} ψ ( u ★ y ) + \overline{β} φ ( v ★ x ) + \overline{β} ψ ( v ★ y ) . \qquad ( 3.18 )$$

Property 3.12. The cross-correlation of a pair of two-sided sequences is equal to the convolution of the elementwise complex conjugate of the reverse of the first sequence and the second sequence. More formally, let x and y be two-sided infinite complex sequences. Then,

$$x ★ y = \overline{\overleftarrow{x}} * y , \qquad ( 3.19 )$$

when [symbol] denotes the elementwise complex conjugate of the reverse of x, i.e.,

$$\overline{\overleftarrow{{x}_{i}}} = \overline{{x}_{- i}} , for each i ∈ ℤ = \{ … , - 2 , - 1 , 0 , 1 , 2 , … \} . \qquad ( 3.2 )$$

Property 3.13. Let x and y be a pair of two-sided sequences. The cross-correlation of x and y is equal to the cross-correlation of the reverse and conjugate of y and the reverse and conjugate of x. More formally,

$$x ★ y = \overline{\overleftarrow{y}} ★ \overline{\overleftarrow{x}} . \qquad ( 3.21 )$$

Lemma 3.14. Let a=(a₀, a₁, a₂, . . . ) and b=(b₀, b₁, b₂, . . . ) be two right-sided infinite sequences. Let x=( . . . , x₋₂, x₋₁, x₀, x₁, x₂, . . . ) be a two-sided sequence obtained by padding a with infinitely many zeros on the left, i.e.,

$${x}_{m} = \{ \begin{matrix}0 & if m < 0 , \\ {a}_{m} & if m \geq 0 .\end{matrix} \qquad ( 3.22 )$$

Similarly, let y=( . . . , y₋₂, y₋₁, y₀, y₁, y₂, . . . ) be a two-sided sequence obtained by padding b with infinitely many zeros on the left. More formally,

$${y}_{n} = \{ \begin{matrix}0 & if n < 0 , \\ {b}_{n} & if n \geq 0 .\end{matrix} \qquad ( 3.23 )$$

Then, the cross-correlation of a and b is equal to the cross-correlation of x and y, i.e.,

$${( a ★ b )}_{n} = {( x ★ y )}_{n} , for each n ∈ ℤ = \{ … , - 2 , - 1 , 0 , 1 , 2 , … \} . \qquad ( 3.24 )$$

### 3.3.2 The Cross-Correlation Theorem

The cross-correlation theorem, which is stated below, gives a formula for the bilateral z-transform of the cross-correlation of a pair of two-sided infinite sequences. It is similar to the convolution theorem, but because cross-correlation is not commutative there are some differences. In this case, the value of the z-transform of the cross-correlation at z can be obtained by multiplying the complex conjugate of the z-transform of the first sequence evaluated at the reciprocal of the complex conjugate of z by the z-transform of the second sequence evaluated at z.

Theorem 3.15. The Cross-Correlation Theorem for the Bilateral z-Transform (when the Sequences are Two-Sided).
Let x=( . . . , x₋₂, x₋₁, x₀, x₁, x₂, . . . ) and y=( . . . , y₋₂, y₋₁, y₀, y₁, y₂, . . . ) be a pair of two-sided infinite sequences and let z be a complex scalar such that the following two conditions are satisfied:
i) The bilateral z-transform of x is defined at the reciprocal of the complex conjugate of z, i.e., 1/z̅∈domain( [symbol] ₓ). The bilateral z-transform of y is defined at z, i.e., z∈domain( [symbol] _{y}). More formally,

$$\vert {ℨ}_{x} ( 1 / \overline{𝓏} ) \vert = \vert {\sum }_{m = - ∞}^{∞} {{x}_{m} ( 1 / \overline{𝓏} )}^{- m} \vert < ∞ , \qquad ( 3.25 )$$

$$\vert {ℨ}_{y} ( 𝓏 ) \vert = \vert {\sum }_{n = - ∞}^{∞} {y}_{n} {𝓏}^{- n} \vert < ∞ . \qquad ( 3.26 )$$

ii) Both series that define [symbol] ₓ(1/z̅) and [symbol] _{y}(z) converge absolutely. More formally,

$${\sum }_{m = - ∞}^{∞} \vert {{x}_{m} ( 1 / \overline{𝓏} )}^{- m} \vert < ∞ and {\sum }_{n = - ∞}^{∞} \vert {y}_{n} {𝓏}^{- n} \vert < ∞ . \qquad ( 3.27 )$$

Then, the value of the bilateral z-transform of the cross-correlation of x and y at z is equal to the product of the complex conjugate of the value of the bilateral z-transform of x at 1/z̅ and the value of the bilateral z-transform of y at z. More formally,

$${ℨ}_{x ★ y} ( 𝓏 ) = \overline{{ℨ}_{x} ( 1 / \overline{𝓏} )} {ℨ}_{y} ( 𝓏 ) . \qquad ( 3.2 8 )$$

If the two sequences are right-sided, then there is another version of the cross-correlation theorem. This version states that the value of the bilateral z-transform of the cross-correlation at z is equal to the product of the complex conjugate of the value of the unilateral z-transform of the first sequence evaluated at 1/z̅ and the value of the unilateral z-transform of the second sequence evaluated at z. This theorem is stated below.

Theorem 3.16. The cross-correlation theorem for the bilateral z-transform (when the sequences are right-sided). Let a=(a₀, a₁, a₂, . . . ) and b=(b₀, b₁, b₂, . . . ) be two right-sided infinite sequences and let z be a complex scalar such that the following two conditions are satisfied:
i) The unilateral z-transform of a is defined at the reciprocal of the complex conjugate of z, i.e., 1/z̅∈domain( [symbol] ₐ⁺). The unilateral z-transform of b is defined at z, i.e., z∈domain( [symbol] _{b}⁺). In other words,

$$\vert {ℨ}_{a}^{+} ( 1 / \overline{𝓏} ) \vert = \vert {\sum }_{m = 0}^{∞} {{a}_{m} ( 1 / \overline{𝓏} )}^{- m} \vert < ∞ , \qquad ( 3.2 9 )$$

$$\vert {ℨ}_{b}^{+} ( 𝓏 ) \vert = \vert {\sum }_{n = 0}^{∞} {b}_{n} {𝓏}^{- n} \vert < ∞ . \qquad ( 3.3 )$$

ii) At least one of the two series that define [symbol] ₐ⁺(1/z̅) and [symbol] _{b}⁺(z) converges absolutely. More formally,

[symbol]

$${\sum }_{m = 0}^{∞} \vert {{a}_{m} ( 1 / \overline{𝓏} )}^{- m} \vert < ∞ or {\sum }_{n = 0}^{∞} \vert {b}_{n} {𝓏}^{- n} \vert < ∞ . \qquad ( 3.31 )$$

Then, the value of the bilateral z-transform of the cross-correlation of a and b at z is equal to the product of the complex conjugate of the value of the unilateral z-transform of a at the reciprocal of the complex conjugate of z and the value of the unilateral z-transform of b at z. More formally,

$${ℨ}_{a ★ b} ( 𝓏 ) = \overline{{ℨ}_{a}^{+} ( 1 / \overline{𝓏} ) } {ℨ}_{b}^{+} ( 𝓏 ) . \qquad ( 3.32 )$$

Notice that in the second version of the theorem there is an asymmetry, i.e., in the formula

$${ℨ}_{a ★ b} ( 𝓏 ) = \overline{{ℨ}_{a}^{+} ( 1 / \overline{𝓏} )} {ℨ}_{b}^{+} ( 𝓏 ) \qquad ( 3.33 )$$

the bilateral z-transform is used in the left-hand side, but the unilateral z-transform is used in the right-hand side. This is due to the fact that the cross-correlation of two right-sided sequences is a two-sided sequence.

FIG. 31 summarizes the theorems for the bilateral z-transform. There are four different versions depending on the types of the sequences (two-sided or right-sided) and the type of operation performed on the pair of sequences (convolution or cross-correlation).

FIG. 32 summarizes the theorems for the unilateral z-transform. There is no version of the convolution theorem for the unilateral z-transform when the two sequences are two-sided. There are no versions of the cross-correlation theorem for the unilateral z-transform either. The next section, however, states two versions of the concatenation theorem, which make it possible to express [symbol] _{x*y}⁺(z) and [symbol] _{a*b}⁺(z) with a slightly different formula.

### 3.4 The Concatenation Theorem for the Z-Transform

This section states two versions of the concatenation theorem. The first version is for two-sided sequences. The second version is for right-sided sequences and its proof relies on the proof of the first theorem. The following lemma is used in the proof of the theorem.

Lemma 3.17. Let T be an integer, let x=( . . . , x_{T-1}, x_{T}, x_{T+1}, . . . ) be a two-sided sequence, and let y=( . . . , y_{T-1}, y_{T}, y_{T+1}, . . . ) be another two-sided sequence. Also, let z be a complex number such that the conditions of the cross-correlation theorem for the bilateral z-transform (Theorem 3.15) are satisfied, i.e.,
i) The bilateral z-transform of x is defined at the reciprocal of the complex conjugate of z, i.e., 1/z̅∈domain( [symbol] ₓ). The bilateral z-transform of y is defined at z, i.e., z∈domain( [symbol] _{y}). More formally,

$$\vert {ℨ}_{x} ( 1 / \overline{𝓏} ) \vert = \vert {\sum }_{m = - ∞}^{∞} {{x}_{m} ( 1 / \overline{𝓏} )}^{- m} \vert < ∞ , \qquad ( 3.34 )$$

$$\vert {ℨ}_{y} ( 𝓏 ) \vert = \vert {\sum }_{n = - ∞}^{∞} {y}_{n} {𝓏}^{- n} \vert < ∞ . \qquad ( 3.35 )$$

ii) Both series that define [symbol] ₓ(1/z̅) and [symbol] _{y}(z) converge absolutely. More formally,

$${\sum }_{m = - ∞}^{∞} \vert {{x}_{m} ( 1 / \overline{𝓏} )}^{- m} \vert < ∞ and {\sum }_{n = - ∞}^{∞} \vert {y}_{n} {𝓏}^{- n} \vert < ∞ . \qquad ( 3.36 )$$

Let x′ be a two-sided sequence that is obtained from x by replacing x_{T} and all elements that follow it with zeros. More formally, xₙ′=H(T−1−n)xₙ, for each n∈ [symbol] ={ . . . , −2, −1, 0, 1, 2, . . . }, where H(n) denotes the Heaviside function, which is defined as follows:

$$H ( n ) = \{ \begin{matrix}1 , if n \geq 0 , \\ 0 , if n < 0 .\end{matrix} \qquad ( 3.37 )$$

In other words,

$${x}_{n}^{′} = \{ \begin{matrix}{x}_{n} , & if n < T , \\ 0 , & if n \geq T .\end{matrix} \qquad ( 3.38 )$$

Similarly, let y′ be a two-sided sequence that is derived from y using the same procedure that was used to derive x′ from x, i.e., y′ₙ=H(T−1−n) yₙ for each n∈ [symbol] . In other words,

$${y}_{n}^{′} = \{ \begin{matrix}{y}_{n} , & if n < T , \\ 0 , & if n \geq T .\end{matrix} \qquad ( 3.39 )$$

Also, let x″ be a two-sided sequence that is obtained from x by replacing all elements up to and including X_{T-1} with zeros and keeping the remaining elements unchanged. More formally, xₙ″=H(n−T)xₙ for each n∈ [symbol] . In other words.

$${x}_{n}^{″} = \{ \begin{matrix}0 , & if n < T , \\ {x}_{n} , & if n \geq T .\end{matrix} \qquad ( 3.4 )$$

Similarly, let y″ be a two-sided sequence that is derived from y using the same procedure that was used to derive x″ from x, i.e., y″=H(n−T) yₙ for each n∈ [symbol] . That is,

$${y}_{n}^{″} = \{ \begin{matrix}0 , & if n < T , \\ {y}_{n} , & if n \geq T .\end{matrix} \qquad ( 3.41 )$$

$$Then ,$$

$${ℨ}_{x ★ y}^{+} ( 𝓏 ) = {ℨ}_{( {x}^{′} + {x}^{″} ) ★ ( {y}^{′} + {y}^{″} )}^{+} ( 𝓏 ) = {ℨ}_{{x}^{′} ★ {y}^{′}}^{+} ( 𝓏 ) + {ℨ}_{{x}^{′} ★ {y}^{″}}^{+} ( 𝓏 ) + {ℨ}_{{x}^{″} ★ {y}^{′}}^{+} ( 𝓏 ) + {ℨ}_{{x}^{″} ★ {y}^{″}}^{+} ( 𝓏 ) , \qquad ( 3.42 )$$

and each of the four terms in the right-hand side of (3.42) is well-defined and finite.
Theorem 3.18. Concatenation theorem for two-sided sequences. Let T be an integer, let x=( . . . , x_{T-1}, x_{T}, x_{T+1}, . . . ) be a two-sided sequence, and let y=( . . . , y_{T-1}, y_{T}, y_{T+1}, . . . ) be another two-sided sequence. Also, let z be a complex number such that the conditions of the cross-correlation theorem for the bilateral z-transform (Theorem 3.15) am satisfied, i.e.,
i) The bilateral z-transform of x is defined at the reciprocal of the complex conjugate of z, i.e., 1/z̅∈domain( [symbol] ₓ). The bilateral z-transform of y is defined at z, i.e., z∈domain( [symbol] _{y}). More formally,

$$\vert {ℨ}_{x} ( 1 / \overline{𝓏} ) \vert = \vert {\sum }_{m = - ∞}^{∞} {{x}_{m} ( 1 / \overline{𝓏} )}^{- m} \vert < ∞ , \qquad ( 3.43 )$$

$$\vert {ℨ}_{y} ( 𝓏 ) \vert = \vert {\sum }_{n = - ∞}^{∞} {y}_{n} {𝓏}^{- n} \vert < ∞ . \qquad ( 3.44 )$$

ii) Both series that define [symbol] ₓ(1/z̅) and [symbol] _{y}(z) converge absolutely. More formally,

$${\sum }_{m = - ∞}^{∞} \vert {{x}_{m} ( 1 / \overline{𝓏} )}^{- m} \vert < ∞ and {\sum }_{n = - ∞}^{∞} \vert {y}_{n} {𝓏}^{- n} \vert < ∞ . \qquad ( 3.45 )$$

Let x′ be a two-sided sequence that is obtained from x by replacing x_{T} and all elements that follow it with zeros. More formally, xₙ′=H(T−1−n) xₙ, for each n∈ [symbol] ={ . . . , −2, −1, 0, 1, 2, . . . }, where H(n) denotes the Heaviside function, which is defined as follows:

$$H ( n ) = \{ \begin{matrix}1 , & if n \geq 0 , \\ 0 , & if n < 0 .\end{matrix} \qquad ( 3.46 )$$

In other words,

$${x}_{n}^{′} = \{ \begin{matrix}{x}_{n} , & if n < T , \\ 0 , & if n \geq T .\end{matrix} \qquad ( 3.47 )$$

Similarly, let y′ be a two-sided sequence that is derived from y using the same procedure that was used to derive x′ from x, i.e, yₙ′=H(T−1−n) yₙ for each n∈ [symbol] . In other words,

$${y}_{n}^{′} = \{ \begin{matrix}{y}_{n} , & if n < T , \\ 0 , & if n \geq T .\end{matrix} \qquad ( 3.48 )$$

Also, let x″ be a two-sided sequence that is obtained from x by replacing all elements up to and including x_{T-1} with zeros and keeping the remaining elements unchanged. More formally, xₙ″=H(n−T)xₙ for each n∈ [symbol] . In other words,

$${x}_{n}^{″} = \{ \begin{matrix}0 , & if n < T , \\ {x}_{n} , & if n \geq T .\end{matrix} \qquad ( 3.49 )$$

Similarly, let y″ be a two-sided sequence that is derived from y using the same procedure that was used to derive x″ from x, i.e., yₙ″=H(n−T) y, for each n∈ [symbol] . That is,

$${y}_{n}^{″} = \{ \begin{matrix}0 , & if n < T , \\ {y}_{n} , & if n \geq T .\end{matrix} \qquad ( 3.5 )$$

Then, the value of the unilateral z-transform at z of the cross-correlation of x and y can be expressed as

$${ℨ}_{x ★ y}^{+} ( 𝓏 ) = {ℨ}_{{x}^{′} ★ {y}^{′}}^{+} ( 𝓏 ) + {ℨ}_{{x}^{″} ★ {y}^{″}}^{+} ( 𝓏 ) + \overline{{ℨ}_{{x}^{′}} ( 1 / \overline{𝓏} )} {ℨ}_{{y}^{″}} ( 𝓏 ) . \qquad ( 3.51 )$$

When the two input sequences are right-sided (i.e., causal), there is another version of the concatenation theorem, which is stated below.

Theorem 3.19. Concatenation theorem for right-sided sequences. Let T be a non-negative integer, let a=(a₀, a₁, a₂ . . . ) be a right-sided sequence and let b=(b₀, b₁, b₂, . . . ) be another right-sided sequence. Furthermore, let z be a complex number such that the following two conditions are satisfied:
i) The unilateral z-transform of a is defined at the reciprocal of the complex conjugate of z, i.e., 1/z̅∈domain( [symbol] ₐ⁺). The unilateral z-transform of b is defined at z, i.e., z∈domain( [symbol] _{b}⁺). More formally,

$$\vert {ℨ}_{a}^{+} ( 1 / \overline{𝓏} ) \vert = \vert {\sum }_{m = 0}^{∞} {{a}_{m} ( 1 / \overline{𝓏} )}^{- m} \vert < ∞ , \qquad ( 3.52 )$$

$$\vert {ℨ}_{b}^{+} ( 𝓏 ) \vert = \vert {\sum }_{n = 0}^{∞} {b}_{n} {𝓏}^{- n} \vert < ∞ . \qquad ( 3.53 )$$

ii) Both series that define [symbol] ₐ⁺(1/z̅) and [symbol] _{b}⁺(z) converge absolutely. More formally,

$${\sum }_{m = 0}^{∞} \vert {{a}_{m} ( 1 / \overline{𝓏} )}^{- m} \vert < ∞ and {\sum }_{n = 0}^{∞} \vert {b}_{n} {𝓏}^{- n} \vert < ∞ . \qquad ( 3.54 )$$

Let a′ be a right-sided sequence that is obtained from the sequence a by replacing a_{T} and all elements that follow it by zeros. More formally, aₙ′=H(T−1−n)aₙ for each n∈ [symbol] ⁺={0, 1, 2, . . . }, where H(n) denotes the Heaviside function, i.e.,

[symbol]

$$H ( n ) = \{ \begin{matrix}1 , & if n \geq 0 , \\ 0 , & if n < 0.\end{matrix} \qquad ( 3.55 )$$

In other words, the elements of the sequence a′ are defined as follows:

$${a}_{n}^{′} = \{ \begin{matrix}{a}_{n} , & if 0 \leq n < T , \\ 0 , & if n \geq T .\end{matrix} \qquad ( 3.56 )$$

Similarly, let b′ be a right-sided sequence that is derived from b using the same approach that was used to derive a′ from a, i.e., bₙ′=H(T−1−n)bₙ for each n∈ [symbol] ⁺={0, 1, 2, . . . }. That is, the elements of b′ are given by:

$${b}_{n}^{′} = \{ \begin{matrix}{b}_{n} , & if 0 \leq n < T , \\ 0 , & if n \geq T .\end{matrix} \qquad ( 3.57 )$$

Also, let a″ be a right-sided sequence that is obtained from a by replacing all elements up to and including a_{T-1} with zeros and keeping the remaining elements unchanged. More formally, aₙ″=H(n−T)aₙ for each n∈ [symbol] ⁺. In other words,

$${a}_{n}^{″} = \{ \begin{matrix}0 , & if 0 \leq n < T , \\ {a}_{n} , & if n \geq T .\end{matrix} \qquad ( 3.58 )$$

Similarly, let b″ be a right-sided sequence that is derived from the sequence b such that bₙ″=H(n−T)bₙ for each n∈ [symbol] ⁺. That is,

$${b}_{n}^{″} = \{ \begin{matrix}0 , & if 0 \leq n < T , \\ {b}_{n} , & if n \geq T .\end{matrix} \qquad ( 3.59 )$$

Then, the value of the unilateral z-transform at z of the cross-correlation of a and b can be expressed in the following form:

$${ℨ}_{a ★ b}^{+} ( 𝓏 ) = {ℨ}_{{a}^{′} ★ {b}^{′}}^{+} ( 𝓏 ) + {ℨ}_{{a}^{″} ★ {b}^{″}}^{+} ( 𝓏 ) + \overline{{ℨ}_{{a}^{′}}^{+} ( 1 / \overline{𝓏} )} {ℨ}_{{b}^{″}}^{+} , ( 𝓏 ) . \qquad ( 3.6 )$$

### 3.5 Special Cases of the Concatenation Theorem

This section states as corollaries several special cases of the concatenation theorem for right-sided sequences that have finite length. These corollaries are the mathematical foundation for both the encoding and the decoding algorithm.

Corollary 3.20. Let K be a positive integer and let T be another nonnegative integer such that 0≤T≤K. Let u=(u₀, u₁, . . . , u_{K-1}) and v=(v₀, v₁, . . . , v_{K-1}) be two finite sequences of length K. Let u′ be a finite sequence of length K that is obtained from u by replacing u_{T} and all elements that follow it with zeros, i.e., uₙ′, =H(T−1−n)uₙ for n∈{0, 1, 2, . . . , K−1} so that

$${u}_{n}^{′} = \{ \begin{matrix}{u}_{n} , & if 0 \leq n < T , \\ 0 , & if T \leq n < K .\end{matrix} \qquad ( 3.61 )$$

Similarly, let v′ be a finite sequence of length K that is obtained from v by replacing v_{T} and all elements that follow it with zeros, i.e., vₙ′=H(T−1−n)vₙ for n∈{0, 1, 2, . . . , K−1} so that

$${v}_{n}^{′} = \{ \begin{matrix}{v}_{n} , & if 0 \leq n < T , \\ 0 , & if T \leq n < K .\end{matrix} \qquad ( 3.62 )$$

Also, let u″ be a finite sequence of length K that is obtained from u by replacing all of its elements up to and including u_{T-1} with zeros, i.e., uₙ″=H(n−T)uₙ for each n∈{0, 1, 2, . . . , K−1}. In other words,

$${u}_{n}^{″} = \{ \begin{matrix}0 , & if 0 \leq n < T , \\ {u}_{n} , & if T \leq n < K .\end{matrix} \qquad ( 3.63 )$$

Similarly, let v″ be a finite sequence of length K that is obtained from v by replacing all of its elements up to and including v_{T-1} with zeros, i.e., vₙ″=H(n−T)vₙ for each n∈{0, 1, . . . , K−1} so that

$${v}_{n}^{″} = \{ \begin{matrix}0 , & if 0 \leq n < T , \\ {v}_{n} , & if T \leq n < K .\end{matrix} \qquad ( 3.64 )$$

$$Then ,$$

$${ℨ}_{u ★ v}^{+} ( 𝓏 ) = {ℨ}_{{u}^{′} ★ {v}^{′}}^{+} ( 𝓏 ) + {ℨ}_{{u}^{″} ★ {v}^{″}}^{+} ( 𝓏 ) + \overline{{ℨ}_{{u}^{′}}^{+} ( 1 / \overline{𝓏} )} {ℨ}_{{v}^{″}}^{+} ( 𝓏 ) . \qquad ( 3.65 )$$

Note that Corollary 3.20 does not have any convergence conditions, unlike some of the previous theorems, because the sequences u and v are finite. Thus, both series derived from these finite sequences converge and they also converge absolutely.

Corollary 3.21. Let T be a nonnegative integer. Also, let u=(u₀, u₁, u₂, . . . , u_{T}) and v=(v₀, v₁, v₂, . . . , v_{T}) be two finite sequences of length T+1. Furthermore, let u′=(u₀, u₁, . . . , u_{T-1}, 0) be a finite sequence formed by the first T elements of u followed by a single zero and let v′=(v₀, v₁, . . . , v_{T-1}, 0) be a finite sequence formed by the first T elements of v followed by a single zero as well. Then,

$${ℨ}_{u ★ v}^{+} ( 𝓏 ) = {ℨ}_{{u}^{′} ★ {v}^{′}}^{+} ( 𝓏 ) + ℨ \frac{+}{\overleftarrow{u}} ( 𝓏 ) {v}_{T} , \qquad ( 3.66 )$$

where [symbol] is a finite sequence of length T+1 that is obtained by reversing and conjugating the elements of u.
Corollary 3.22. Let u=(u₀, u₁, u₂, . . . , u_{T}) and v=(v₀, v₁, v₂, . . . , v_{T}) be two finite sequences of length T+1, where T is a nonnegative integer. Also, let u″=(0, u₁, u₂, . . . , u_{T-1}, u_{T}) be the finite sequence of length T+1 that is obtained from u by replacing its first element with zero. Similarly, let v″=(0, v₁, v₂, . . . , v_{T-1}, v_{T}) be a finite sequence of length T+1 that is obtained from v by replacing its first element with zero. Then,

$${ℨ}_{u ★ v}^{+} ( 𝓏 ) = \overline{{u}_{0}} {ℨ}_{v}^{+} ( 𝓏 ) + {ℨ}_{{u}^{″} ★ {v}^{″}}^{+} ( 𝓏 ) . \qquad ( 3.67 )$$

### 4 Discrete-Time Examples

This chapter provides examples that illustrate the z-transform theorems and the properties of exponential SSM matrices. Most of the examples in this chapter use right-sided finite sequences to illustrate the essence of a theorem or to visualize how an algorithm works. The theorems, however, are more general and apply to infinite sequences as well.

### 4.1 Types of Sequences

The z-transform theorems that were described previously use four different types of sequences. Some theorems are true for all four types. Others are valid for only a subset of them. The following examples illustrate the differences between these sequence types.

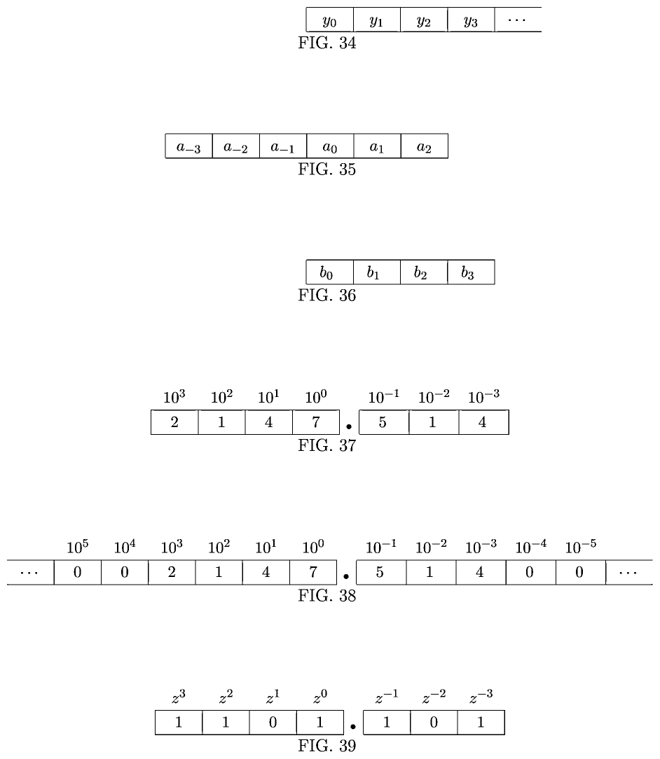

FIG. 33 gives an example of a two-sided infinite sequence x. The sequence extends infinitely in both directions and both positive and negative integers are used to index the elements of x. FIG. 34 gives an example of a right-sided infinite sequence y, which extends infinitely in only one direction. In this case there are no sequence elements with negative indices, i.e., only the positive integers and zero are used as indices. Right-sided sequences are often called causal sequences.

FIG. 35 shows an example of a two-sided finite sequence a that has only six elements. What makes this a two-sided sequence is the fact that the elements of a are indexed by both positive and negative integers. Finally, FIG. 36 visualizes the elements of the right-sided finite sequence b, which has a length of four. Because infinite sequences have infinitely many elements, it makes sense to talk about the length of a sequence only when we have a finite sequence.

### 4.2 Bilateral Z-Transform Example

### 4.2.1 Calculating the Z-Transform for One Specific Value of z

To illustrate the bilateral z-transform we will give an example using the decimal number system, which should be familiar to everyone. Every number in the decimal system can be viewed as a sequence of digits. For example, the number 2147.514 can be viewed as a two-sided finite sequence d=(d₋₃, d₋₂, d₋₁, d₀, d₁, d₂, d₃), the elements of which are: 2, 1, 4, 7, 5, 1, and 4. FIG. 37 shows one way to visualize this sequence in which each digit is placed in a separate box. The corresponding power of 10 is written above each box. The decimal point can be viewed as a separator between the nonnegative and the negative powers of 10.

The same number can also be represented with an infinite two-sided sequence as shown in FIG. 38. In this case, the left and the right tail of the sequence are padded with zeros. When we write decimal numbers, however, it is tacitly assumed that these zeros can be omitted.

The magnitude of this number in the decimal system is equal to the value of the bilateral z-transform of the two-sided digit sequence d, evaluated at z=10. In other words,

$$\begin{matrix}{ℨ}_{d} ( 10 ) = {\sum }_{n = - ∞}^{∞} {{d}_{n} ( 10 )}^{- n} = {\sum }_{n = - 3}^{3} {{d}_{n} ( 10 )}^{- n} \\ = {{d}_{- 3} ( 10 )}^{3} + {{d}_{- 2} ( 10 )}^{2} + {{d}_{- 1} ( 10 )}^{1} + {d}_{0} {( 10 )}^{0} + {{d}_{1} ( 10 )}^{- 1} + \\ {d}_{2} {( 10 )}^{- 2} + {{d}_{3} ( 10 )}^{- 3} \\ = 2 ( 10 \; 00 ) + 1 ( 100 ) + 4 ( 10 ) + 7 ( 1 ) + 5 ( 0.1 ) + 1 ( 0.01 ) + 4 ( 0.001 ) \\ = 2000 + 100 + 40 + 7 + 0.5 + 0.01 + 0.004 \\ = 2147.514\end{matrix} \qquad ( 4.1 )$$

The notation [symbol] {d} is typically used for the bilateral z-transform of the sequence d. This notation, however, is for the entire z-transform, i.e., for all possible values of z. In this case, however, we need the value of the z-transform at one specific z, e.g., z=10. This requires an extra set of brackets to specify that, i.e., [symbol] {d}(z), which makes the notation too cumbersome. Therefore, we will simplify the notation by putting the part in the curly brackets in a subscript. Thus, the value of the bilateral z-transform, evaluated at z, of the sequence d will be denoted with [symbol] _{d}(z) Similarly, the value of the unilateral z-transform at z of the sequence a will be denoted with [symbol] ₐ⁺(z).

This value is not the z-transform of the sequence d. Instead, this is the value of the z-transform of d evaluated at the specific point z=10. To get the entire z-transform we need to perform similar calculations for all possible values of z.

### 4.2.2 Calculating the Z-Transform for all Values of z

As another example, consider the number 1101.101, which can be represented as a finite two-sided sequence of digits as shown in FIG. 39. In other words, this number can be represented as the sequence b=(b₋₃, b₋₂, b₋₁, b₀, b₁, b₂, b₃) the elements of which are equal to: 1, 1, 0, 1, 1, 0, and 1. In this case, however, the value of z is not fixed to be just 10. Instead, the corresponding power of z is written above each digit in the figure.

To calculate the bilateral z-transform of b at a specific point we need to pick some z and plug it into the formula for the z-transform. For example, if we pick z=2 we get the following result:

$$\begin{matrix}{ℨ}_{b} ( 2 ) = {\sum }_{n = - ∞}^{∞} {{b}_{n} ( 2 )}^{- n} = {\sum }_{n = - 3}^{3} {{b}_{n} ( 2 )}^{- n} \\ = {{b}_{- 3} ( 2 )}^{3} + {{b}_{- 2} ( 2 )}^{2} + {{b}_{- 1} ( 2 )}^{1} + {{b}_{0} ( 2 )}^{0} + {{b}_{1} ( 2 )}^{- 1} + \\ {b}_{2} {( 2 )}^{- 2} + {{b}_{3} ( 2 )}^{- 3} \\ = 1 ( 8 ) + 1 ( 4 ) + 0 ( 2 ) + 1 ( 1 ) + 1 ( 0.5 ) + 0 ( 0.25 ) + 1 ( 0.125 ) \\ = 8 + 4 + 0 + 1 + 0.5 + 0 + 0.125 \\ = 13.625\end{matrix} \qquad ( 4.2 )$$

This result could be interpreted as the value of this number in the binary number system. If we pick z=10, then we get the value in the decimal number system:

$$( 4.3 )$$

$$\begin{matrix}{ℨ}_{b} ( 10 ) = {\sum }_{n = - ∞}^{∞} {{b}_{n} ( 10 )}^{- n} = {\sum }_{n = - 3}^{3} {{b}_{n} ( 10 )}^{- n} \\ = {{b}_{- 3} ( 10 )}^{3} + {{b}_{- 2} ( 10 )}^{2} + {{b}_{- 1} ( 10 )}^{1} + {{b}_{0} ( 10 )}^{0} + {{b}_{1} ( 10 )}^{- 1} + {{b}_{2} ( 10 )}^{- 2} + \\ {{b}_{3} ( 10 )}^{- 3} \\ = 1 ( 1000 ) + 1 ( 100 ) + 0 ( 10 ) + 1 ( 1 ) + 1 ( 0.1 ) + 0 ( 0.01 ) + 1 ( 0.001 ) \\ = 1000 + 100 + 0 + 1 + 0.1 + 0 + 0.001 \\ = 1101.101\end{matrix}$$

In fact, we could pick any other value of z and perform a similar calculation. For example, if we pick z=0.4 or z=−2.5, then we get:

$$( 4.4 )$$

$$\begin{matrix}{ℨ}_{b} ( 0.4 ) = {\sum }_{n = - ∞}^{∞} {b}_{n} {( 0.4 )}^{- n} = {\sum }_{n = - 3}^{3} {{b}_{n} ( 0.4 )}^{- n} \\ = {{b}_{- 3} ( 0.4 )}^{3} + {{b}_{- 2} ( 0.4 )}^{2} + {{b}_{- 1} ( 0.4 )}^{1} + {{b}_{0} ( 0.4 )}^{0} + {{b}_{1} ( 0.4 )}^{- 1} + \\ = {{b}_{2} ( 0.4 )}^{- 2} ++ {b}_{3} {( 0.4 )}^{- 3} \\ = 1 ( 0. 064 ) + 1 ( 0.16 ) + 0 ( 0.4 ) + 1 ( 1 ) + 1 ( 2.5 ) + 0 ( 6. 25 ) + 1 ( 15.625 ) \\ = 0.064 + 0.16 + 0 + 1 + 2.5 + 0 + 15.625 \\ = 19.349\end{matrix}$$

$$( 4.5 )$$

$$\begin{matrix}{ℨ}_{b} ( - 2.5 ) = {\sum }_{n = - ∞}^{∞} {{b}_{n} ( - 2.5 )}^{- n} {\sum }_{n = - 3}^{3} {{b}_{n} ( - 2.5 )}^{- n} \\ = {{b}_{- 3} ( - 2.5 )}^{3} + {{b}_{- 2} ( - 2.5 )}^{2} + {{b}_{- 1} ( - 2.5 )}^{1} + {{b}_{0} ( - 2.5 )}^{0} + \\ {{b}_{1} ( - 2.5 )}^{- 1} + {{b}_{2} ( - 2.5 )}^{- 2} + {{b}_{3} ( - 2.5 )}^{- 3} \\ = 1 ( - 15.625 ) + 1 ( 6.25 ) + 0 ( - 2.5 ) + 1 ( 1 ) + 1 ( - 0.4 ) + 0 ( 0. 16 ) + \\ 1 ( - 0.064 ) \\ = - 15. 625 + 6.25 + 0 + 1 - 0.4 + 0 - 0.064 \\ = - 8.8 \; 39\end{matrix}$$

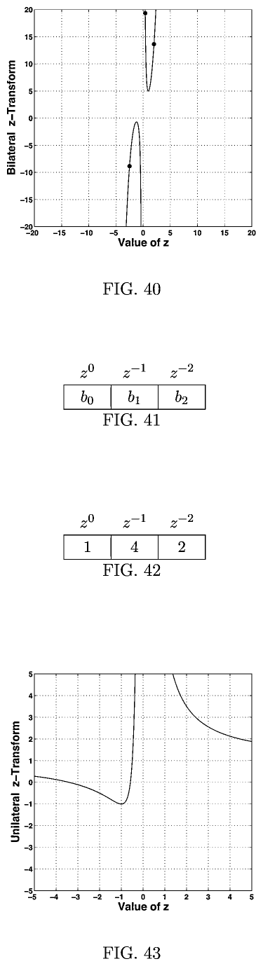

To plot the z-transform of this sequence we need to perform similar calculations for all possible values of z. FIG. 40 shows that for all real z in a small segment of the real line. The three blue circles in this plot show the value of the z-transform at z=−2.5, z=0.4, and z=2. The value for z=10 is not shown as it is too large for the chosen zoom level.

In general, z can be a complex number. Visualizing the transform in that case is not easy as it requires a four-dimensional plot.

In this example, the digit sequence was finite and for a finite sequence the value of the z-transform is always bounded. For infinite sequences, however, it is possible that for some values of z the value of the z-transform will diverge to either positive infinity or negative infinity. For example, the z-transform of the infinite digit sequence 0.333(3) evaluated at z=1 is equal to infinity because evaluating this value requires adding an infinite number of 3's.

### 4.3 Unilateral Z-Transform Example

The unilateral z-transform is similar to the bilateral z-transform, but in this case only the sequence elements at nonnegative indices are used in the calculations. Therefore, the unilateral z-transform is typically used with right-sided or causal sequences. If for some reason the sequence is two-sided, then its left tail is simply ignored.

Let b=(b₀, b₁, b₂) be a right-sided finite sequence of length three. The elements of this abstract sequence are shown in FIG. 41, along with the negative powers of z. The unilateral z-transform of b, denoted by [symbol] _{b}⁺(z), is given by the following formula:

$${ℨ}_{b}^{+} ( z ) = {b}_{0} {z}^{0} + {b}_{1} {z}^{- 1} + {b}_{2} {z}^{- 2} . \qquad ( 4.6 )$$

In other words, the unilateral z-transform of b is a function of z that maps the elements of b and the value of z to the value of [symbol] _{b}⁺(z).

To make this example more concrete, let b₀=1, b₁=4, and b₂=2, i.e., let b=(1, 4, 2). FIG. 42 shows the elements of this sequence along with their corresponding negative powers of z. The unilateral z-transform of this specific sequence is given by:

$${ℨ}_{b}^{+} ( z ) = 1 {z}^{0} + 4 {z}^{- 1} + 2 {z}^{- 2} . \qquad ( 4.7 )$$

Using this formula, the value of the transform can be calculated for any z. For example, for z=4, we have:

$$\begin{matrix}{ℨ}_{b}^{+} ( 4 ) = 1 {( 4 )}^{0} + 4 {( 4 )}^{- 1} + 2 {( 4 )}^{- 2} \\ = 1 ( 1 ) + 4 ( 0.25 ) + 2 ( 0.0625 ) \\ = 2.125\end{matrix} \qquad ( 4.8 )$$

If we perform similar calculations for all possible values of z, then we can plot the unilateral z-transform. This is shown in FIG. 43 for real values of z in the range [−5,5]. Note that the z-transform has a singularity at z=0.

So far in this example z was restricted to be a real number. In general, however, z can be a complex number. If we allow z to be complex, then the value of the z-transform can also be a complex number. Visualizing the z-transform in that case is a challenge as it requires a four-dimensional plot. In the most general case both z and the elements of b can be complex numbers. Visualizing the z-transform in this case would require a four-dimensional plot as well.

### 4.4 Convolution Examples

The discrete convolution of two infinite right-sided sequences a and b is defined as follows:

$${( a * b )}_{n} = {\sum }_{m = 0}^{n} {a}_{m} {b}_{n - m} \qquad ( 4.9 )$$

$$for each$$

$$n ∈ {ℤ}^{+} = \{ 0 , 1 , 2 , … \} .$$

The outcome of this operation is a sequence, which is called the convolution sequence. Sometimes the resulting sequence is also called the Cauchy product of a and b.

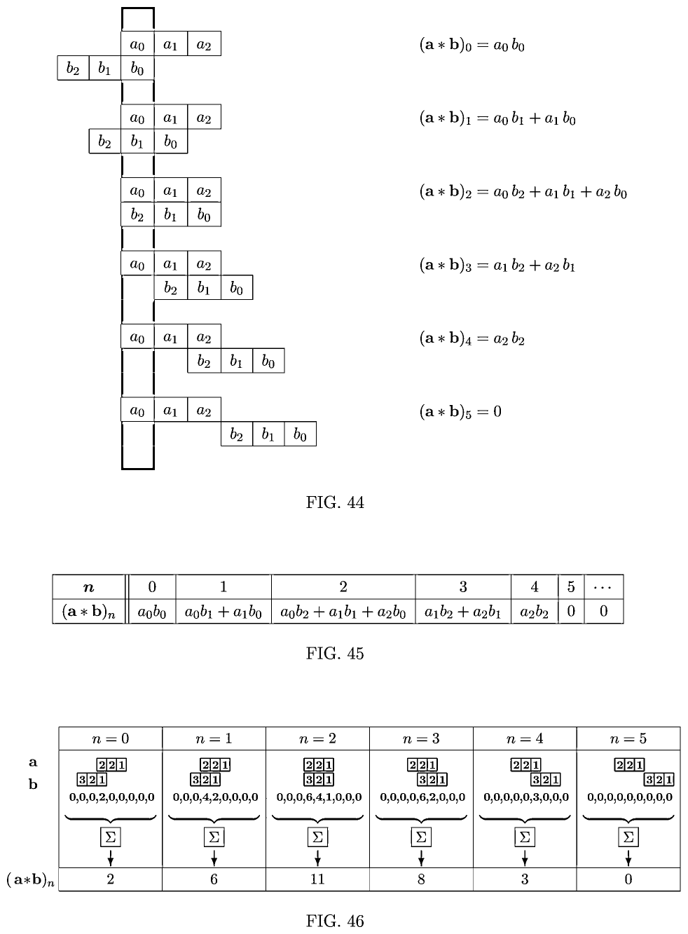

To illustrate this operation we will use two right-sided sequences of length three: a=(a₀, a₁, a₂) and b=(b₀, b₁, b₂). As with some of the earlier examples, we can write each sequence on a separate tape. In this visualization each tape has equally-sized boxes and each box contains only one element of the sequence that the tape represents. FIG. 44 uses this convention to illustrate how the convolution of a and b can be computed. The elements of the first sequence are written in order, i.e., a₀, a₁, and a₂. The elements of the second sequence are written in reversed order, i.e., b₂, b₁, and b₀. During all iterations the first tape is kept fixed such that a₀ is always at the origin, which is represented with a gray vertical line in the figure.

The convolution sequence, which is denoted by (a*b)=((a*b)₀, (a*b)₁, . . . ), is computed iteratively such that only one element of this sequence is computed during each iteration. Which element? That depends on the offset between the two tapes, where the offset is defined as the number of boxes in the horizontal direction that separate a₀ and b₀. For example, to compute the n-th element (a*b)ₙ of the convolution sequence the a-tape and the b-tape must be placed at an offset n relative to each other. Once this is done, the value of (a*b)ₙ can be computed by multiplying all vertically aligned elements from a and b and then adding all such pairwise products. If a sequence element is not aligned with an element from the other sequence, then that specific product is assumed to be zero.

For n=0 the two tapes are aligned such that a₀ is directly above b₀ (see the top part of FIG. 44). In this configuration no other elements of the two sequences overlap. Thus, the 0-th element of the resulting convolution sequence is: (a*b)₀=a₀b₀.

For n=1 the b-tape is shifted one position to the right, relative to the configuration for n=0. Now a₀ is directly above b₁ and a₁ is directly above b₀. By multiplying the elements that line up vertically and adding the two partial results we get: (a*b)₁=a₀b₁+a₁b₀. This is the value of the element at index 1 in the convolution sequence.

For n=2 the b-tape is shifted one position to the right, relative to the previous step. Now the three elements of a overlap with the three elements of b. Because the temporal order of b is reversed, however, the result is: (a*b)₂=a₀b₂+a₁b₁+a₂b₀. This is the element at index 2 in the resulting sequence.

Continuing in the same way we can compute all elements of the convolution sequence. Because both a and b are finite sequences, however, at some point the two tapes will no longer overlap. In our example this occurs when n=5. In this case the resulting product is assumed to be 0. Thus, (a*b)_{b}=0. The same is true for all n≥5, but these iterations are not shown in the figure.

FIG. 45 shows another way to visualize the elements of the convolution sequence that takes less space. In this figure the elements of the sequence are arranged horizontally instead of vertically. Also, the details of how they are computed are not shown. Once again, for n≥5 all elements are zero as the two tapes no longer overlap. If you expand the sum in formula (4.9) you should get the same result for each value of n. Try it!

To make this a bit more concrete, FIG. 46 gives a numerical example in which a=(2, 2, 1) and b=(1, 2, 3). This figure combines visualization techniques from the two previous figures in this section. In other words, each iteration is visualized in the same way as in FIG. 44, but now they are arranged horizontally as in FIG. 45. The product between two vertically aligned elements of a and b is indicated with a number that is written directly below them. That number is assumed to be zero if the two sequences don't overlap. After adding all pairwise products for each offset n, the resulting convolution sequence is: (a*b)=(2, 6, 11, 8, 3, 0, 0, . . . ).

### 4.4.1 The Unilateral Z-Transform of the Convolution Sequence

Let a=(a₀, a₁, a₂) and b=(b₀, b₁, b₂) be two right-sided sequences of length three. The convolution of a and b is a sequence, which is denoted by (a*b)=((a*b)₀, (a*b)₁ . . . ). FIG. 44 already showed how to compute each element of this sequence.

The unilateral z-transform of the convolution sequence can be computed from FIG. 44 or FIG. 45 by simply multiplying each element (a*b)ₙ of this sequence by its corresponding negative power of z, i.e., z⁻ⁿ, and then adding all of these products. That is,

$$( 4.1 )$$

$${ℨ}_{a * b}^{+} ( z ) = ( {a}_{0} {b}_{0} ) {z}^{0} + ( {a}_{0} {b}_{1} + {a}_{1} {b}_{0} ) {z}^{- 1} + ( {a}_{0} {b}_{2} + {a}_{1} {b}_{1} + {a}_{2} {b}_{0} ) {z}^{- 2} + ( {a}_{1} {b}_{2} + {a}_{2} {b}_{1} ) {z}^{- 3} + ( {a}_{2} {b}_{2} ) {z}^{- 4} .$$

If we perform the multiplications and arrange the resulting terms such that the first half of the rows are left justified while the second half are right justified, then we will get the following:

$$\begin{matrix}{ℨ}_{a * b}^{+} ( z ) = & {a}_{0} {b}_{0} {z}^{0} & & \\ & + {a}_{0} {b}_{1} {z}^{- 1} & + {a}_{1} {b}_{0} {z}^{- 1} & \\ & + {a}_{0} {b}_{2} {z}^{- 2} & + {a}_{1} {b}_{1} {z}^{- 2} & + {a}_{2} {b}_{0} {z}^{- 2} \\ & & + {a}_{1} {b}_{2} {z}^{- 3} & + {a}_{2} {b}_{1} {z}^{- 3} \\ & & & + {a}_{2} {b}_{2} {z}^{- 4} .\end{matrix} \qquad ( 4.11 )$$

By grouping the terms in each of the columns of this expression we get:

$$( 4.12 )$$

$$\begin{matrix}{ℨ}_{a * b}^{+} ( z ) = {a}_{0} ( {b}_{0} {z}^{0} + {b}_{1} {z}^{- 1} + {b}_{2} {z}^{- 2} ) + {a}_{1} ( {b}_{0} {z}^{- 1} + {b}_{1} {z}^{- 2} + {b}_{2} {z}^{- 3} ) + \\ {a}_{2} ( {b}_{0} {z}^{- 2} + {b}_{1} {z}^{- 3} + {b}_{2} {z}^{- 4} ) \\ = {a}_{0} {z}^{0} ( {b}_{0} {z}^{0} + {b}_{1} {z}^{- 1} + {b}_{2} {z}^{- 2} ) + {a}_{1} {z}^{- 1} ( {b}_{0} {z}^{0} + {b}_{1} {z}^{- 1} + {b}_{2} {z}^{- 2} ) + \\ {a}_{2} {z}^{- 2} ( {b}_{0} {z}^{0} + {b}_{1} {z}^{- 1} + {b}_{2} {z}^{- 2} ) \\ = ( {a}_{0} {z}^{0} + {a}_{1} {z}^{- 1} + {a}_{2} {z}^{- 2} ) ( {b}_{0} {z}^{0} + {b}_{1} {z}^{- 1} + {b}_{2} {z}^{- 2} ) \\ = {ℨ}_{a}^{+} ( z ) {ℨ}_{b}^{+} ( z ) .\end{matrix}$$

This result is the essence of the convolution theorem for the unilateral z-transform. This theorem is true even if a and b are infinite right-sided sequences.

### 4.4.2 The Bilateral Z-Transform of the Convolution Sequence

For the right-sided sequences a=(a₀, a₁, a₂) and b=(b₀, b₁, b₂) used in the previous example the bilateral z-transform of a*b is equivalent to the unilateral z-transform of a*b. In other words, [symbol] _{a*b}(z)= [symbol] _{a*b}⁺(z)= [symbol] ₐ⁺(z) [symbol] _{b}⁺(z). This is true for all right-sided sequences because the convolution of two right-sided sequences is itself a right-sided sequence.

For a pair of two-sided sequences x and y there is another version of the convolution theorem, which states that

$${ℨ}_{x * y} ( z ) = {ℨ}_{x} ( z ) {ℨ}_{y} ( z ) . \qquad ( 4.13 )$$

In other words, the value of the bilateral z-transform at z of the convolution of x and y is equal to the product of the bilateral z-transform of x at z and the bilateral z-transform of y at z.

### 4.5 Cross-Correlation Examples

The discrete cross-correlation of two infinite right-sided sequences a and b is a two-sided sequence, the elements of which are defined as follows:

$${( a ★ b )}_{n} = {\sum }_{m = max ( 0 , - n )}^{∞} \overline{{a}_{m}} {b}_{m + n} \qquad ( 4.14 )$$

$$for each$$

$$n ∈ ℤ = \{ … , - 2 , - 1 , 0 , 1 , 2 , … \} .$$

Alternatively, the formula for the n-th element of the cross-correlation sequence can be stated as:

$${( a ★ b )}_{n} = {\sum }_{m = - ∞}^{∞} \overline{{a}_{m}} {b}_{m + n} \qquad ( 4.15 )$$

$$for each$$

$$n ∈ ℤ = \{ … , - 2 , - 1 , 0 , 1 , 2 , … \} ,$$

assuming that the product a̅ₘ̅bₘ₊ₙ is equal to zero if either m<0 or m+n<0.

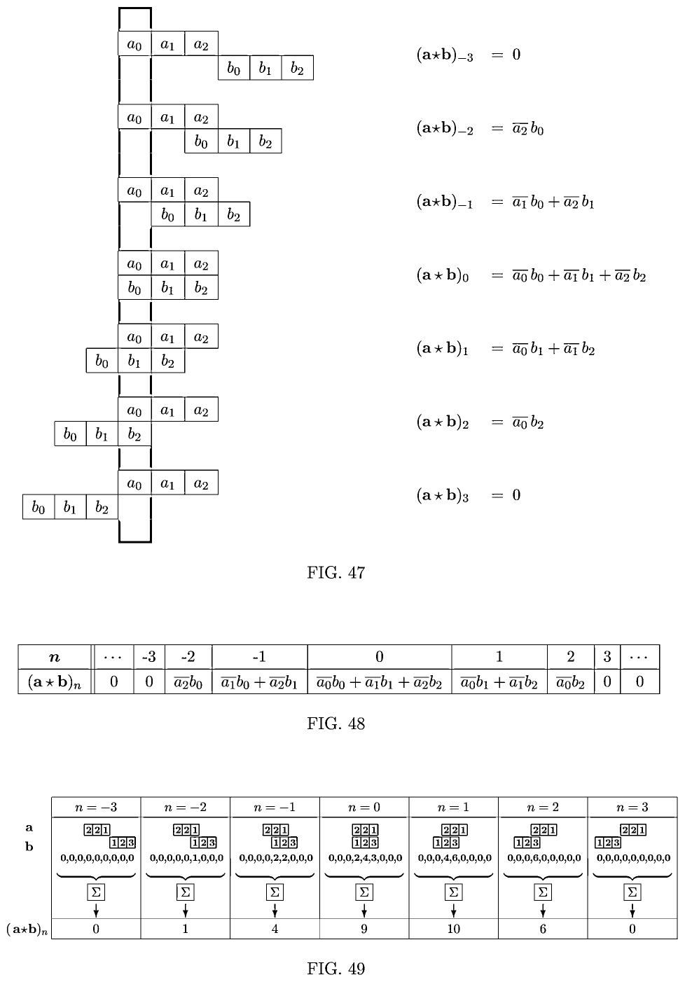

To illustrate this operation, FIG. 47 gives an example with two finite sequences of length three: a=(a₀, a₁, a₂) and b=(b₀, b₁, b₂). Once again, we can write the elements of each sequence on a separate tape. The first tape is fixed in place such that a₀ is always at the origin, which is represented in the figure with a vertical gray line. The second tape, the one for the sequence b, is shifted to the left by one position after each iteration. For each offset, n, we can calculate only one element of the cross-correlation sequence. As with convolution, the calculation involves pairwise multiplication of all elements of a that are vertically aligned with elements of b and then adding all such products. In this case, however, the elements of the first sequence must be conjugated before each multiplication. The elements of the resulting cross-correlation sequence are shown in FIG. 48.

From this example it should be clear that cross-correlation is similar to convolution, but also that there are some key differences. First, we don't need to reverse the second sequence. Its elements appear on the tape in their original order. Thus, the temporal order of both sequences is preserved by this operation. Second, we now need negative indices to index all elements of the cross-correlation sequence. In other words, the cross-correlation of two right-sided sequences is a two-sided sequence. This was not the case for convolution. Third, each element of the first sequence must be conjugated before it is multiplied by its corresponding element of the second sequence because this is how the operation is defined. If the first sequence is a real sequence, then the conjugation can be dropped as it has meaning only for complex numbers. For complex sequences, however, the conjugation is required. Finally, to distinguish between these two operations, we will use * for cross-correlation and * for convolution.

To make this example a bit more concrete, let's consider the case when a=(2, 2, 1) and b=(1, 2, 3). FIG. 49 shows the individual steps in calculating the sequence (a*b). This is similar to FIG. 47 but now each iteration is put in a separate box. The resulting cross-correlation sequence is (a*b)=( . . . , 0, 1, 4, 9, 10, 6, 0 . . . ). Note that the two tails contain infinitely many zeros. Also, note that unlike convolution, cross-correlation does not require that we reverse the order of the second sequence.

### 4.5.1 Cross-Correlation is not Commutative

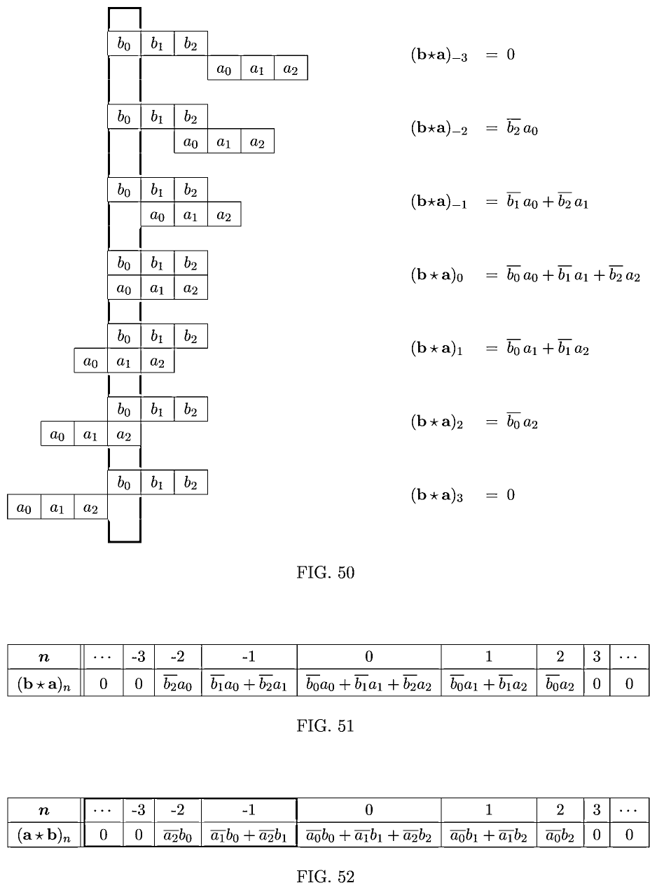

Unlike convolution, cross-correlation is not a commutative operation. In other words, swapping the order of the two sequences leads to a different result. To demonstrate this, FIG. 50 illustrates the computation of the elements (b*a)ₙ of the cross-correlation sequence for different values of n. This is similar to FIG. 47, but the order of the two sequences is now swapped: b is first and a is second. The resulting cross-correlation sequence is shown in FIG. 51. It is easy to see that the elements of this sequence are different from the elements of the sequence shown in FIG. 48. Therefore, a*b≠b*a. This result is true in general, not just for the two finite sequences used in this example. In other words, this result is true for infinite two-sided and infinite right-sided sequences as well.

### 4.5.2 The Bilateral Z-Transform of the Cross-Correlation Sequence

Let c be a two-sided sequence. The bilateral z-transform of c is defined as:

$${ℨ}_{c} ( z ) = {\sum }_{n = - ∞}^{∞} {c}_{n} {z}^{- n} . \qquad ( 4.16 )$$

This definition is true for any two-sided sequence c. In particular, if we set cₙ=(a*b)ₙ, then we can calculate the bilateral z-transform of the cross-correlation of a and b. In other words, because the cross-correlation of the sequence a and the sequence b is itself a sequence it is possible to compute the bilateral z-transform of that sequence as well. That is,

$${ℨ}_{a ★ b} ( z ) = {\sum }_{n = - ∞}^{∞} {( a ★ b )}_{n} {z}^{- n} = {\sum }_{n = - ∞}^{∞} ( {\sum }_{m = max ( 0 , - n )}^{∞} \overline{{a}_{m}} {b}_{m + n} ) {z}^{- n} . \qquad ( 4.17 )$$

As in the previous examples, let a=(a₀, a₁, a₂) and b=(o, b₁, b₂). Because in this case both a and b are finite right-sided sequences formula (4.17) simplifies to:

$${ℨ}_{a ★ b} ( z ) = {\sum }_{n = - 2}^{2} {( a ★ b )}_{n} {z}^{- n} = {\sum }_{n = - 2}^{2} ( {{\sum }_{m = max ( 0 , - n )}}^{min ( 2 , 2 - n )} \overline{{a}_{m}} {b}_{m + n} ) {z}^{- n} . \qquad ( 4.18 )$$

To compute the bilateral z-transform of a*b we can expand the double sum in (4.18). Alternatively, we can multiply each element (a*b)ₙ of this cross-correlation sequence by z⁻ⁿ and then add all products. In other words,

$$( 4.19 )$$

$${ℨ}_{a ★ b} ( z ) = {( a ★ b )}_{- 2} {z}^{2} + {( a ★ b )}_{- 1} {z}^{1} + {( a ★ b )}_{0} {z}^{0} + {( a ★ b )}_{1} {z}^{- 1} + {( a ★ b )}_{2} {z}^{- 2} .$$

Substituting the values for (a*b)ₙ from FIG. 48 into the previous equation we get:

$$( 4.2 )$$

$${ℨ}_{a ★ b} ( z ) = ( \overline{{a}_{2}} {b}_{0} ) {z}^{2} + ( \overline{{a}_{1}} {b}_{0} + \overline{{a}_{2}} {b}_{1} ) {z}^{1} + ( \overline{{a}_{0}} {b}_{0} + \overline{{a}_{1}} {b}_{1} + \overline{{a}_{2}} {b}_{2} ) {z}^{0} + ( \overline{{a}_{0}} {b}_{1} + \overline{{a}_{1}} {b}_{2} ) {z}^{- 1} + ( \overline{{a}_{0}} {b}_{2} ) {z}^{- 2} .$$

If we perform the multiplications in each row of (4.20) and arrange the terms in columns, such that they are grouped by their common elements of b, then the following pattern emerges:

$$\begin{matrix}{ℨ}_{a ★ b} ( z ) = & \overline{{a}_{2}} {b}_{0} {z}^{2} & & \\ & + \overline{{a}_{1}} {b}_{0} {z}^{1} & + \overline{{a}_{2}} {b}_{1} {z}^{1} & \\ & + \overline{{a}_{0}} {b}_{0} {z}^{0} & + \overline{{a}_{1}} {b}_{1} {z}^{0} & + \overline{{a}_{2}} {b}_{2} {z}^{0} \\ & & + \overline{{a}_{0}} {b}_{1} {z}^{- 1} & + \overline{{a}_{1}} {b}_{2} {z}^{- 1} \\ & & & + \overline{{a}_{0}} {b}_{2} {z}^{- 2} .\end{matrix} \qquad ( 4.21 )$$

If we “push up” the columns of the previous expression so that they lineup on top, then we get:

$$\begin{matrix}{ℨ}_{a ★ b} ( z ) = & \overline{{a}_{2}} {b}_{0} {z}^{2} & + \overline{{a}_{2}} {b}_{1} {z}^{1} & + \overline{{a}_{2}} {b}_{2} {z}^{0} \\ & + \overline{{a}_{1}} {b}_{0} {z}^{1} & + \overline{{a}_{1}} {b}_{1} {z}^{0} & + \overline{{a}_{1}} {b}_{2} {z}^{- 1} \\ & + \overline{{a}_{0}} {b}_{0} {z}^{0} & + \overline{{a}_{0}} {b}_{1} {z}^{- 1} & + \overline{{a}_{0}} {b}_{2} {z}^{- 2} .\end{matrix} \qquad ( 4.22 )$$

Swapping the order of the rows, such that the first becomes last and the last becomes first, leads to the following expression:

$$\begin{matrix}{ℨ}_{a ★ b} ( z ) = & \overline{{a}_{0}} {b}_{0} {z}^{0} & + \overline{{a}_{0}} {b}_{1} {z}^{- 1} & + \overline{{a}_{0}} {b}_{2} {z}^{- 2} \\ & + \overline{{a}_{1}} {b}_{0} {z}^{1} & + \overline{{a}_{1}} {b}_{1} {z}^{0} & + \overline{{a}_{1}} {b}_{2} {z}^{- 1} \\ & + \overline{{a}_{2}} {b}_{0} {z}^{2} & + \overline{{a}_{2}} {b}_{1} {z}^{1} & + \overline{{a}_{2}} {b}_{2} {z}^{0} .\end{matrix} \qquad ( 4.23 )$$

After factoring out the common terms and powers of z in each row we get:

$$( 4.24 )$$

$${ℨ}_{a ★ b} ( z ) = \overline{{a}_{0}} {z}^{0} ( {b}_{0} {z}^{0} + {b}_{1} {z}^{- 1} + {b}_{2} {z}^{- 2} ) + \overline{{a}_{1}} {z}^{1} ( {b}_{0} {z}^{0} + {b}_{1} {z}^{- 1} + {b}_{2} {z}^{- 2} ) + \overline{{a}_{2}} {z}^{2} ( {b}_{0} {z}^{0} + {b}_{1} {z}^{- 1} + {b}_{2} {z}^{- 2} ) .$$

Finally, this can be expressed as:

$$( 4.25 )$$

$$\begin{matrix}{ℨ}_{a ★ b} ( z ) = ( \overline{{a}_{0}} {z}^{0} + \overline{{a}_{1}} {z}^{1} + \overline{{a}_{2}} {z}^{2} ) ( {b}_{0} {z}^{0} + {b}_{1} {z}^{- 1} + {b}_{2} {z}^{- 2} ) \\ = \overline{( {{a}_{0} ( \overline{z} )}^{0} + {{a}_{1} ( \overline{z} )}^{1} + {{a}_{2} ( \overline{z} )}^{2} )} ( {b}_{0} {z}^{0} + {b}_{1} {z}^{- 1} + {b}_{2} {z}^{- 2} ) \\ = \overline{( {{a}_{0} ( 1 / \overline{z} )}^{0} + {{a}_{1} ( 1 / - \overline{z} )}^{- 1} + {{a}_{2} ( 1 / \overline{z} )}^{- 2} )} ( {b}_{0} {z}^{0} + {b}_{1} {z}^{- 1} + {b}_{2} {z}^{- 2} ) \\ = \overline{{ℨ}_{a}^{+} ( 1 / \overline{z} )} {ℨ}_{b}^{+} ( z ) .\end{matrix}$$

In other words, the value of the bilateral z-transform, evaluated at z, of the cross-correlation of a and b can be expressed as the product of the complex conjugate of the unilateral z-transform of a evaluated at 1/z̅ and the unilateral z-transform of b evaluated at z. Note the asymmetry in this equation: the left-hand side uses the bilateral z-transform, but the right-hand side uses the unilateral z-transform. This is the essence of the cross-correlation theorem for the bilateral z-transform when the two sequences are right-sided. The theorem is true even if a and b are infinite right-sided sequences.

### 4.5.3 The Unilateral Z-Transform of the Cross-Correlation Sequence

Similar to the previous section, let c be a complex sequence. The unilateral z-transform of c is defined as:

$${ℨ}_{c}^{+} ( z ) = {\sum }_{n = 0}^{∞} {c}_{n} {z}^{- n} . \qquad ( 4.26 )$$

Note that in this case the lower-bound of the sum starts from 0 and not from −∞ as it was the case with the bilateral z-transform. If c is a two-sided sequence, then the elements in its left tail are simply ignored for the purposes of calculating [symbol] _{c}⁺(z).

Let a and b be two right-sided sequences. The cross-correlation of a and b is a two-sided sequence denoted by (a*b)=( . . . , (a*b)₋₂, (a*b)₋₁, (a*b)₀, (a*b)₁, (a*b)₂, . . . ). If we set cₙ=(a*b)ₙ in formula (4.26), then we can calculate the unilateral z-transform of this cross-correlation sequence as follows:

$$( 4.27 )$$

$${ℨ}_{a ★ b}^{+} ( z ) = {\sum }_{n = 0}^{∞} {( a ★ b )}_{n} {z}^{- n} = {\sum }_{n = 0}^{∞} ( {\sum }_{m = max ( 0 , - n )}^{∞} \overline{{a}_{m}} {b}_{m + n} ) {z}^{- n} = {\sum }_{n = 0}^{∞} {\sum }_{m = 0}^{∞} \overline{{a}_{m}} {b}_{m + n} {z}^{- n} .$$

Once again, only the elements in the right tail of the cross-correlation sequence are needed to calculate [symbol] _{a*b}⁺(z). These elements are indexed by n, which is either positive or zero. Therefore, the inner sum in (4.27) can start from m=0, because max(0, −n)=0 when n≥0.

To give a concrete example, let a=(a₀, a₁, a₂) and b=(b₀, b₁, b₂). The cross-correlation sequence for a and b was already calculated and is shown in FIG. 48. For convenience, this result is replicated in FIG. 52. In this case, however, we don't need the entire sequence. We only need the elements that are indexed by nonnegative integers. In other words, the elements in the left tail of the cross-correlation sequence can be ignored. The ignored elements are highlighted in gray in FIG. 52.

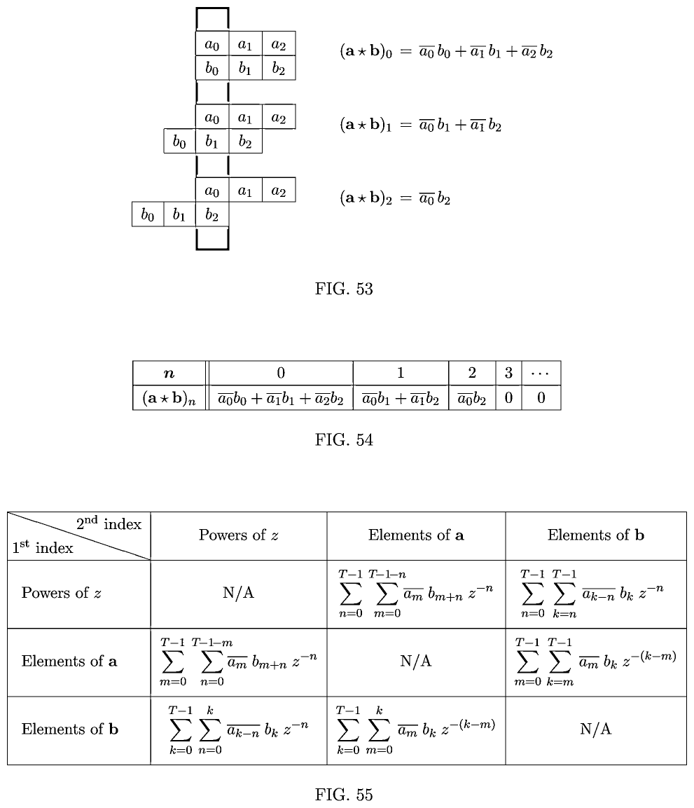

Alternatively, we could compute only the elements of the cross-correlation sequence for which n≥0. This is shown in FIG. 53, which is a subset of FIG. 47. This results in a smaller figure that shows only the elements that are needed to compute the unilateral z-transform. This shorthand format will be used in the following sections. Similarly, we can abbreviate FIG. 52 by removing the elements with negative indices as shown in FIG. 54.

From FIG. 54 we can easily calculate [symbol] _{a*b}⁺(z) by simply multiplying each element (a*b)ₙ of the sequence by its corresponding negative power of z and then adding all products. That is,

$$( 4.28 )$$

$${ℨ}_{a ★ b}^{+} ( z ) = {( a ★ b )}_{0} {z}^{0} + {( a ★ b )}_{1} {z}^{- 1} + {( a ★ b )}_{2} {z}^{- 2} = ( \overline{{a}_{0}} {b}_{0} + \overline{{a}_{1}} {b}_{1} + \overline{{a}_{2}} {b}_{2} ) {z}^{0} + ( \overline{{a}_{0}} {b}_{1} + \overline{{a}_{1}} {b}_{2} ) {z}^{- 1} + ( \overline{{a}_{0}} {b}_{2} ) {z}^{- 2} .$$

Unlike formulas (4.12), (4.13), and (4.25), the expression in (4.28) cannot be factored into a product of two z-transforms. However, as described in Section 4.6, this expression can be rewritten as the sum of three different terms, each of which can be computed incrementally as the two sequences unfold in time.

4.5.4 An Alternative Formula for [symbol] _{a*b}⁺(z) that Uses the Heaviside Function

This section states the formula for the unilateral z-transform of the cross-correlation of two sequences in an alternative form. To derive the new formula, we will start with formula (4.23) for the bilateral z-transform which is replicated below:

$$\begin{matrix}{ℨ}_{a ★ b} ( z ) = & \overline{{a}_{0}} {b}_{0} {z}^{0} & + \overline{{a}_{0}} {b}_{1} {z}^{- 1} & + \overline{{a}_{0}} {b}_{2} {z}^{- 2} \\ & + \overline{{a}_{1}} {b}_{0} {z}^{1} & + \overline{{a}_{1}} {b}_{1} {z}^{0} & + \overline{{a}_{1}} {b}_{2} {z}^{- 1} \\ & + \overline{{a}_{2}} {b}_{0} {z}^{2} & + \overline{{a}_{2}} {b}_{1} {z}^{1} & + \overline{{a}_{2}} {b}_{2} {z}^{0} .\end{matrix} \qquad ( 4.29 )$$

The terms of this formula are arranged in a grid pattern such that each row contains the same element of the sequence a and each column contains the same element of the sequence b. If we index the rows with j and the columns with k, then each of these terms will have the form ay a̅ⱼ̅bₖz^{-(k-j)}. Thus, [symbol] _{a*b}(z) can be expressed with the following double sum:

$${ℨ}_{a ★ b} ( z ) = {\sum }_{j = 0}^{2} {\sum }_{k = 0}^{2} \overline{{a}_{j}} {b}_{k} {z}^{- ( k - j )} . \qquad ( 4.3 )$$

Our goal is to derive a similar expression for the unilateral z-transform of a*b. To do this, we will start with formula (4.29) and highlight in gray all terms that don't appear in [symbol] _{a*b}⁺(z), i.e.,

All of these terms are in the lower-triangular part of the grid. Note that these same terms are also highlighted in gray in FIG. 52, but in that figure the multiplication with their corresponding power of z has not been performed yet. In other words, these terms have negative indices in the cross-correlation sequence and they are not needed for the calculation of the unilateral z-transform.

Note that in FIG. 52 the element at index −1 in the cross-correlation sequence is composed of two different terms, i.e., (a*b)₋₁=a̅₁̅b₀+a̅₂̅b₁. Both of these terms appear in formula (4.31), but they are now separated and each is multiplied by z¹. They are placed on the diagonal of this grid that is directly below the main diagonal. This rearrangement of terms will come up in several other formulas.

If we remove the highlighted terms from formula (4.31), then we get:

$$\begin{matrix}{ℨ}_{a ★ b}^{+} ( z ) = & \overline{{a}_{0}} {b}_{0} {z}^{0} & + \overline{{a}_{0}} {b}_{1} {z}^{- 1} & + \overline{{a}_{0}} {b}_{2} {z}^{- 2} \\ & & + \overline{{a}_{1}} {b}_{1} {z}^{0} & + \overline{{a}_{1}} {b}_{2} {z}^{- 1} \\ & & & + \overline{{a}_{2}} {b}_{2} {z}^{0} ,\end{matrix} \qquad ( 4.32 )$$

which is an expression for the value of the unilateral z-transform, evaluated at z, of the cross-correlation of a and b.

The formula for [symbol] _{a*b}⁺(z) can also be expressed in the following alternative form:

$${ℨ}_{a ★ b}^{+} ( z ) = {\sum }_{j = 0}^{2} {\sum }_{k = 0}^{2} H ( k - j ) \overline{{a}_{j}} {b}_{k} {z}^{- ( k - j )} . \qquad ( 4.33 )$$

In this formulation, H is the Heaviside function, which is defined as:

$$H ( n ) = \{ \begin{matrix}1 , & if n \geq 0 , \\ 0 , & otherwise\end{matrix} . \qquad ( 4.34 )$$

In other words, if the argument n of H(n) is greater than or equal to 0, then the function is equal to 1. If the argument is less than 0, then the function is equal to 0. This function is often called the unit step function and sometimes it is denoted with u(n).

It is worth spending some time to study formulas (4.29) and (4.30) and how they relate to formulas (4.32) and (4.33). From these it should be clear that the ‘+’ in [symbol] _{a*b}⁺(z) ignores the left tail of the cross-correlation sequence. It should also be clear that the same effect can be achieved with the Heaviside function. That is, the double sum in (4.33) enumerates all possible combinations of the j and k indices, but the H function multiplies some of them by 0 and others by 1. The ones that are multiplied by zero are the shaded elements in the lower-triangular part of (4.31), which come from the left tail of the cross-correlation sequence.

Formula (4.33) offers a compact way to express the value of the unilateral z-transform at z of a*b. From an algorithmic point of view, however, this expression is not computationally efficient. The reason for this is that the double sum in (4.33) explicitly enumerates all possible combinations of the two indices j and k. In other words, even though almost half of all terms are multiplied by the zeros generated by the Heaviside function they are still enumerated by the formula. Section 4.6 describes another way to calculate the same value that is much faster. Nevertheless, it is worth remembering formula (4.33) as it will be used in some sections below.

4.5.5 Six Different Formulas for Computing [symbol] _{a*b}⁺(z)

Let a=(a₀, a₁, . . . , a_{T-1}) and b=(b₀, b₁, . . . , b_{T-1}) be two complex sequences of length T. The formula for the unilateral z-transform of the cross-correlation of a and b can be expressed as a double sum in six different ways. More specifically, each formula contains elements of a, elements of b, and powers of z. Thus, there are three choices for the index of the outer sum and two choices for the index of the inner sum. In total, there are 3×2=6 possible combinations. Each combination leads to a formula, but all six formulas compute the same result, i.e., [symbol] _{a*b}⁺(z).

FIG. 55 arranges these six formulas in a table. The rows of this table correspond to the indices for the outer sum; the columns correspond to the indices for the inner sum. The indexing convention is: n for the powers of z, m for the elements of a, and k for the elements of b.

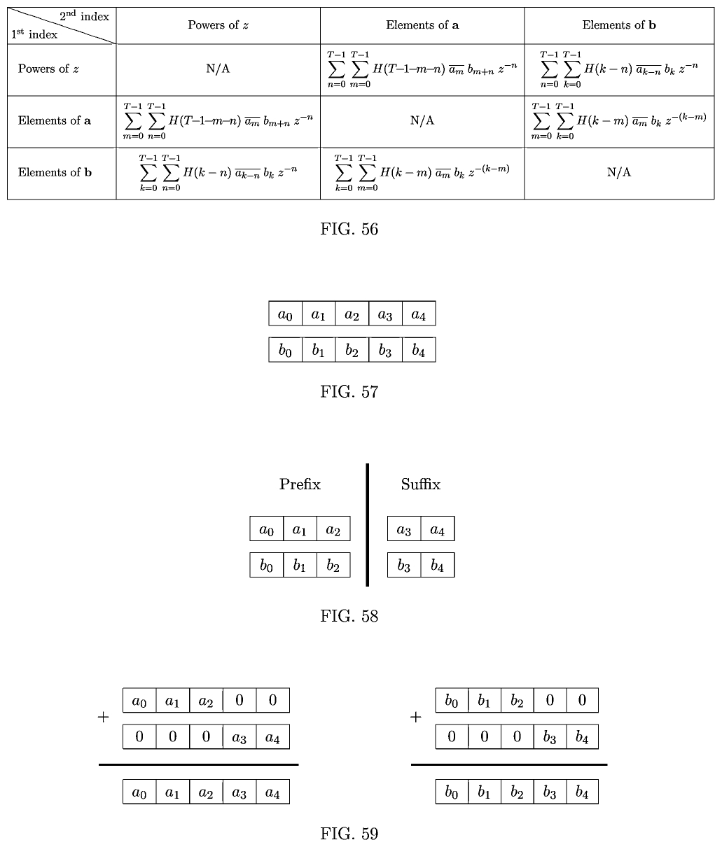

FIG. 56 shows how the six formulas can also be expressed using the Heaviside function. Each formula is equivalent to its corresponding formula that is located in the same cell of the table in FIG. 55. In this case, however, the indices for both sums always start from 0 and end at T−1. Therefore, the pruning of the terms is now accomplished by the Heaviside function, instead of the sum limits. The formulas located on the counter diagonals of FIG. 56 are identical, except that the two sums are swapped. Thus, there are only three unique formulas in this case.

### 4.6 Concatenation Theorem Example

To illustrate the concatenation theorem we will use two abstract right-sided sequences of length five: a=(a₀, a₁, a₂, a₃, a₄) and b=(b₀, b₁, b₂, b₃, b₄), which are shown in FIG. 57. The two sequences unfold in parallel over time, which is why their elements are aligned vertically in the figure. FIG. 58 shows the same sequences, but now each of them has been split into a prefix and a suffix part. We will use a′ and b′ to denote the two prefixes. Similarly, we will use a″ and b″ to denote the two suffixes.

Without loss of generality, we will represent both a′ and a″ with sequences of length five that are padded with the appropriate number of zeros. In other words, a′=(a₀, a₁, a₂, 0, 0) and a″=(0,0,0, a₃, a₄). Now the original sequence a can be represented as the elementwise sum of the “prefix” and the “suffix,” i.e., a=a′+a″. Similarly, the sequence b can be represented as b=b′+b″, where b′=(b₀, b₁, b₂, 0, 0) and b″=(0, 0, b₃, b₄). FIG. 59 illustrates this representation.

If we set a′=(a₀, a₁, a₂) and a″=(a₃, a₄), then it is also possible to represent a as the concatenation of a′ and a″, i.e., a=a′∥a″. A similar representation can also be used for b such that b=b′∥b″. The theorem was first proved in this way, which is why we called it the concatenation theorem. Mathematically speaking, however, the notation and the proof are simpler if the prefixes and the suffixes are padded with zeros.

The concatenation theorem states that the value of the unilateral z-transform at z of the cross-correlation of a and b can be expressed as the sum of three terms. The first of these terms is the unilateral z-transform of a′*b′evaluated at z. The second term is the unilateral z-transform of a″*b″ also evaluated at z. Finally, the third term is the product of the complex conjugate of the unilateral z-transform of a′ evaluated at 1/z̅ and the unilateral z-transform of b″ evaluated at z. Thus, [symbol] _{a*b}⁺(z) can be computed in three parts using only subsequences of the original sequences a and b. Furthermore, these subsequences respect the prefix-suffix boundary shown in FIG. 58.

In other words, the concatenation theorem states that:

$${ℨ}_{a ★ b}^{+} ( z ) = {ℨ}_{{a}^{′} ★ {b}^{′}}^{+} ( z ) + {ℨ}_{{a}^{″} ★ {b}^{″}}^{+} ( z ) + \overline{{ℨ}_{{a}^{′}}^{+} ( 1 / \overline{z} )} {ℨ}_{{b}^{″}}^{+} ( z ) . \qquad ( 4.35 )$$

The rest of this section starts with the left-hand side of this expression and shows that by rearranging and grouping its terms we can derive the right-hand side.

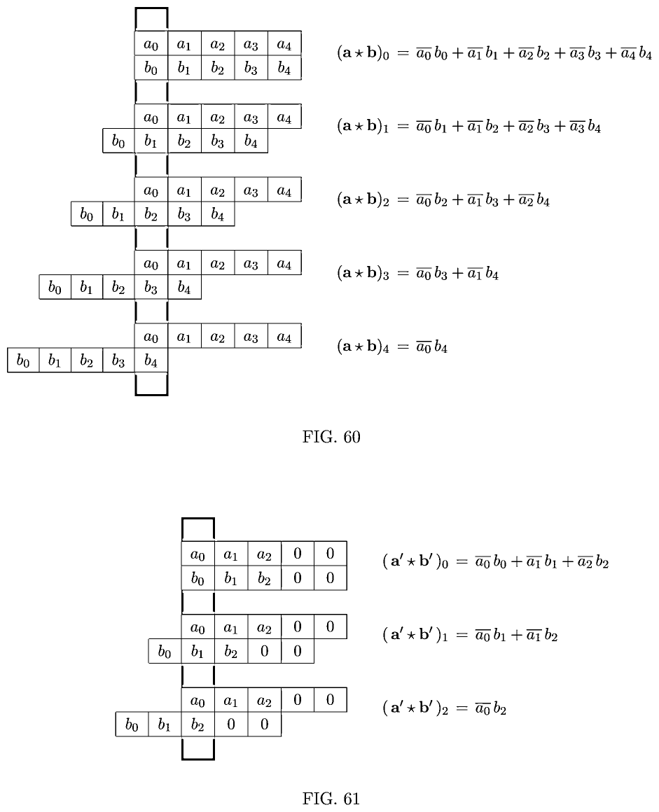

FIG. 60 illustrates the process of computing the elements (a*b). of the cross-correlation of a and b for nonnegative values of the offset n. By multiplying each element of this cross-correlation sequence by its corresponding negative power of z we can get the unilateral z-transform of a*b, which is equal to:

$$( 4.36 )$$

$${ℨ}_{a ★ b}^{+} ( z ) = ( \overline{{a}_{0}} {b}_{0} + \overline{{a}_{1}} {b}_{1} + \overline{{a}_{2}} {b}_{2} + \overline{{a}_{3}} {b}_{3} + \overline{{a}_{4}} {b}_{4} ) {z}^{0} + ( \overline{{a}_{0}} {b}_{1} + \overline{{a}_{1}} {b}_{2} + \overline{{a}_{2}} {b}_{3} + \overline{{a}_{3}} {b}_{4} ) {z}^{- 1} + ( \overline{{a}_{0}} {b}_{2} + \overline{{a}_{1}} {b}_{3} + \overline{{a}_{2}} {b}_{4} ) {z}^{- 2} + ( \overline{{a}_{0}} {b}_{3} + \overline{{a}_{1}} {b}_{4} ) {z}^{- 3} + ( \overline{{a}_{0}} {b}_{4} ) {z}^{- 4} .$$

If we perform the multiplications in (4.36) and arrange the resulting expression such that the terms from the first row are now placed along the main diagonal of an imaginary grid, the terms from the second row are placed on the superdiagonal, and so on until the only term from the last row is placed in the upper-right corner, then we will get the following:

$$( 4.37 )$$

$$\begin{matrix}{ℨ}_{a ★ b}^{+} ( z ) = & \overline{{a}_{0}} {b}_{0} {z}^{0} & + \overline{{a}_{0}} {b}_{1} {z}^{- 1} & + \overline{{a}_{0}} {b}_{2} {z}^{- 2} & + \overline{{a}_{0}} {b}_{3} {z}^{- 3} & + \overline{{a}_{0}} {b}_{4} {z}^{- 4} \\ & & + \overline{{a}_{1}} {b}_{1} {z}^{0} & + \overline{{a}_{1}} {b}_{2} {z}^{- 1} & + \overline{{a}_{1}} {b}_{3} {z}^{- 2} & + \overline{{a}_{1}} {b}_{4} {z}^{- 3} \\ & & & + \overline{{a}_{2}} {b}_{2} {z}^{0} & + \overline{{a}_{2}} {b}_{3} {z}^{- 1} & + \overline{{a}_{2}} {b}_{4} {z}^{- 2} \\ & & & & + \overline{{a}_{3}} {b}_{3} {z}^{0} & + \overline{{a}_{3}} {b}_{4} {z}^{- 1} \\ & & & & & + \overline{{a}_{4}} {b}_{4} {z}^{0} .\end{matrix}$$

The terms of the previous expression can be split into three groups as shown below:

$$\begin{matrix}\begin{matrix}{a * b}_{+}^{} = \\ \begin{matrix} \\ \end{matrix}\end{matrix} & {\underbrace{\begin{matrix}\overline{{a}_{0}} {b}_{0} {z}^{0} & + \overline{{a}_{0}} {b}_{1} {z}^{- 1} & + \overline{{a}_{0}} {b}_{2} {z}^{- 2} \\ & + \overline{{a}_{1}} {b}_{1} {z}^{0} & + \overline{{a}_{1}} {b}_{2} {z}^{- 1} \\ & & + \overline{{a}_{2}} {b}_{2} {z}^{0}\end{matrix}}}_{P}\end{matrix} \qquad ( 4.38 )$$

$$\begin{matrix}\underline{\vert \begin{matrix}+ \overline{{a}_{0}} {b}_{3} {z}^{- 3} & + \overline{{a}_{0}} {b}_{4} {z}^{- 4} \\ + \overline{{a}_{1}} {b}_{3} {z}^{- 2} & + \overline{{a}_{1}} {b}_{4} {z}^{- 3} \\ + \overline{{a}_{2}} {b}_{3} {z}^{- 1} & + \overline{{a}_{2}} {b}_{4} {z}^{- 2}\end{matrix} \} R} \\ {\underbrace{\begin{matrix}+ \overline{{a}_{3}} {b}_{3} {z}^{0} & + \overline{{a}_{3}} {b}_{4} {z}^{- 1} \\ & + \overline{{a}_{4}} {b}_{4} {z}^{0}\end{matrix}}}_{Q}\end{matrix}$$

Thus, the unilateral z-transform of a*b can be expressed as the sum of P, Q, and R, i.e.,

$${ℨ}_{a ★ b}^{+} ( z ) = P + 𝒬 + R . \qquad ( 4.39 )$$

The expression for P contains only elements of a′ and b′, i.e., elements from the prefixes of the two sequences. Furthermore, P can be expressed as the unilateral z-transform of the cross-correlation of a′ and b′ (see also FIG. 61). That is,

$$\begin{matrix}P = \overline{{a}_{0}} {b}_{0} {z}^{0} + \overline{{a}_{0}} {b}_{1} {z}^{- 1} + \overline{{a}_{0}} {b}_{2} {z}^{- 2} + \overline{{a}_{1}} {b}_{1} {z}^{0} + \overline{{a}_{1}} {b}_{2} {z}^{- 1} + \overline{{a}_{2}} {b}_{2} {z}^{0} \\ = ( \overline{{a}_{0}} {b}_{0} + \overline{{a}_{1}} {b}_{1} + \overline{{a}_{2}} {b}_{2} ) {z}^{0} + ( \overline{{a}_{0}} {b}_{1} + \overline{{a}_{1}} {b}_{2} ) {z}^{- 1} + ( \overline{{a}_{0}} {b}_{2} ) {z}^{- 2} \\ = {ℨ}_{{a}^{′} ★ {b}^{′}}^{+} ( z ) .\end{matrix} \qquad ( 4.4 )$$

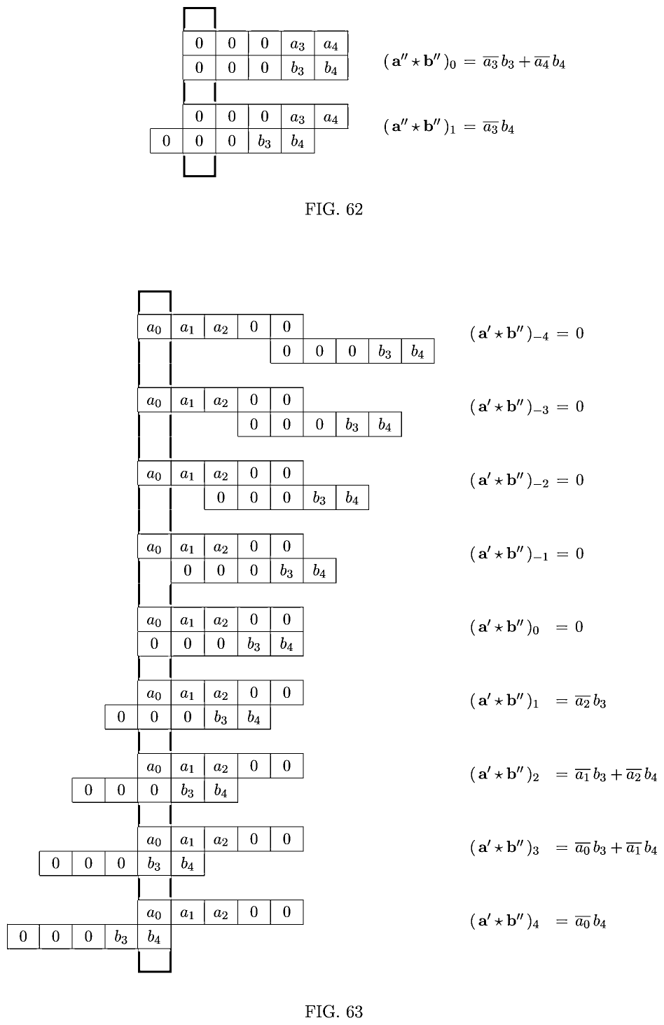

Similarly, Q contains only elements from the suffixes of the two sequences and can be expressed as the unilateral z-transform of a″ *b″ (see also FIG. 62). In other words,

$$\begin{matrix}𝒬 = \overline{{a}_{3}} {b}_{3} {z}^{0} + \overline{{a}_{3}} {b}_{4} {z}^{- 1} + \overline{{a}_{4}} {b}_{4} {z}^{0} \\ = ( \overline{{a}_{3}} {b}_{3} + \overline{{a}_{4}} {b}_{4} ) {z}^{0} + \overline{{a}_{3}} {b}_{4} {z}^{- 1} \\ = {ℨ}_{{a}^{″} ★ {b}^{″}}^{+} ( z ) .\end{matrix} \qquad ( 4.41 )$$

The expression for R contains terms from both a′ and b″. In other words, this is the only expression that does not respect the prefix-suffix boundary shown in FIG. 58. Nevertheless, R can be expressed as the product of two unilateral z-transforms, each of which respects this boundary. That is,

$$\begin{matrix}R = \overline{{a}_{0}} {b}_{3} {z}^{- 3} + \overline{{a}_{0}} {b}_{4} {z}^{- 4} + \overline{{a}_{1}} {b}_{3} {z}^{- 2} + \overline{{a}_{1}} {b}_{4} {z}^{- 3} + \overline{{a}_{2}} {b}_{3} {z}^{- 1} + \overline{{a}_{2}} {b}_{4} {z}^{- 2} \\ = \overline{{a}_{0}} {z}^{0} ( {b}_{3} {z}^{- 3} + {b}_{4} {z}^{- 4} ) + \overline{{a}_{1}} {z}^{1} ( {b}_{3} {z}^{- 3} + {b}_{4} {z}^{- 4} ) + \overline{{a}_{2}} {z}^{2} ( {b}_{3} {z}^{- 3} + {b}_{4} {z}^{- 4} ) \\ = ( \overline{{a}_{0}} {z}^{0} + \overline{{a}_{1}} {z}^{1} + \overline{{a}_{2}} {z}^{2} ) ( {b}_{3} {z}^{- 3} + {b}_{4} {z}^{- 4} ) \\ = \overline{( {{a}_{0} ( \overline{z} )}^{0} + {{a}_{1} ( \overline{z} )}^{1} + {{a}_{2} ( \overline{z} )}^{2} )} ( {b}_{3} {z}^{- 3} + {b}_{4} {z}^{- 4} ) \\ = \begin{matrix}\overline{( {{a}_{0} ( 1 / \overline{z} )}^{0} + {{a}_{1} ( 1 / z - )}^{- 1} + {{a}_{2} ( 1 / z - )}^{- 2} )} \\ ( 0 {z}^{0} + 0 {z}^{- 1} + 0 {z}^{- 2} + {b}_{3} {z}^{- 3} + {b}_{4} {z}^{- 4} )\end{matrix} \\ = \overline{{ℨ}_{{a}^{′}}^{+} ( 1 / \overline{z} )} {ℨ}_{{b}^{″}}^{+} ( z ) .\end{matrix} \qquad ( 4.42 )$$

By adding the expressions for P, Q, and R we can get the equation for the concatenation theorem:

$${ℨ}_{a ★ b}^{+} ( z ) = P + 𝒬 + R = {ℨ}_{{a}^{′} ★ {b}^{′}}^{+} ( z ) + {ℨ}_{{a}^{″} ★ {b}^{″}}^{+} ( z ) + \overline{{ℨ}_{{a}^{′}}^{+} ( 1 / \overline{z} )} {ℨ}_{{b}^{″}}^{+} ( z ) . \qquad ( 4.43 )$$

To summarize, the concatenation theorem splits the computation of the unilateral z-transform of the cross-correlation of a and b into three expressions. The first of these expressions depends only on the elements of a′ and b′. Thus, it does not depend on the suffix of a and the suffix of b. The second expression depends only on the elements of a″ and b″. Thus, it does not depend on the prefix of a and the prefix of b. The third expression depends only on a′ and b″. In other words, it depends on the prefix of the first sequence and on the suffix of the second sequence. Luckily, this third expression can be expressed as the product of the unilateral z-transform of a′ evaluated at 1/z̅ and the unilateral z-transform of b″ evaluated at z.

Finally, it is worth stating explicitly something that should be clear from the previous discussion, but may be lost in all of the details. The concatenation theorem is not about a fast way of computing the cross-correlation of two sequences. It is about a fast way of computing the unilateral z-transform, evaluated at one specific z, of the cross-correlation of two sequences. There is a difference between these two. For example, to compute the cross-correlation sequence we need to compute each and every one of its elements. To compute the unilateral z-transform at z of the cross-correlation sequence, however, we don't need to compute the individual elements of this sequence explicitly. Instead, each element is computed implicitly as a set of product terms, the sum of which is equal to that element. This sum, however, is never performed. Instead, the product terms for each element of the cross-correlation sequence are multiplied by their corresponding power of z and are then added in a specific order with the product terms for the other elements of the cross-correlation sequence to the large sum that constitutes the unilateral z-transform. At the end, the result for [symbol] _{a*b}⁺(z) is still the same, but the freedom to add these terms in this specific order allows for a fast incremental computation. The encoding algorithm uses this property, which gives it its nice computational complexity. The algorithm is illustrated in one of the following sections.

### 4.6.1 Alternative Derivation

This subsection gives an alternative derivation of the concatenation theorem using the same two sequences, a=(a₀, a₁, a₂, a₃, a₄) and b=(b₀, b₁, b₂, b₃, b₄), as in the previous example. Once again, the sequence a is split into a prefix a′ and a suffix a″ such that a=a′+a″. The sequence b is split in a similar way such that b=b′+b″. This representation is illustrated in FIG. 59.

Because both a and b can be expressed as the sum of two sequences, the cross-correlation of a and b can be expressed as follows:

$$a ★ b = ( {a}^{′} + {a}^{″} ) ★ ( {b}^{′} + {b}^{″} ) . \qquad ( 4.44 )$$

Using the properties of cross-correlation this can be further expanded as:

$$a ★ b = {a}^{′} ★ {b}^{′} + {a}^{′} ★ {b}^{″} + {a}^{″} ★ {b}^{′} + {a}^{″} ★ {b}^{″} . \qquad ( 4.45 )$$

Furthermore, because the z-transform is a linear operation we can take the unilateral z-transform of both sides of the previous equation to obtain:

$${ℨ}_{a ★ b}^{+} ( z ) = {ℨ}_{{a}^{′} ★ {b}^{′}}^{+} ( z ) + {ℨ}_{{a}^{′} ★ {b}^{″}}^{+} ( z ) + {ℨ}_{{a}^{″} ★ {b}^{′}}^{+} ( z ) + {ℨ}_{{a}^{″} ★ {b}^{″}}^{+} ( z ) . \qquad ( 4.46 )$$

This expression has four terms. The first term, [symbol] _{a*b}⁺(z), is equal to P as derived above in equation (4.40). Because this term appears in the formula for the concatenation theorem we don't need to modify it any further. Similarly, the fourth term, [symbol] _{a″*b″}(z), is equal to Q, which was derived in equation (4.41), and thus it also does not need to be modified any further.

The second term in (4.46) is [symbol] _{a′*b′}⁺(z). This term is equal to the expression for R that was derived in (4.42). To see why this is the case, consider FIG. 63, which shows the individual steps in the calculation of the cross-correlation of a′ and b″. This figure shows both tails of the cross-correlation sequence. Due to the specific form of a′ and b″, however, when n<0 the elements (a′*b″)ₙ are all zeros. In other words, because the non-zero elements of a′ and b″ don't overlap for n<0 the left tail of the cross-correlation sequence contains only zeros. Thus, in this special case, it follows that the unilateral z-transform of a′*b″ is equal to the bilateral z-transform of a′*b″. In other words, for these two sequences, the following is true:

$${ℨ}_{{a}^{′} ★ {b}^{′}}^{+} ( z ) = {ℨ}_{{a}^{′} ★ {b}^{″}} ( z ) . \qquad ( 4.47 )$$

Using the cross-correlation theorem for the bilateral z-transform, i.e., formula (4.25), this derivation can be continued in the following way:

$${ℨ}_{{a}^{′} ★ {b}^{″}}^{+} ( z ) = {ℨ}_{{a}^{′} ★ {b}^{″}} ( z ) = \overline{{ℨ}_{{a}^{′}}^{+} ( 1 / \overline{z} )} {ℨ}_{{b}^{″}}^{+} ( z ) . \qquad ( 4.48 )$$

As expected, the right-hand side of this expression is equal to the expression for R.

The third term in equation (4.46) is [symbol] _{a″*b′}⁺(z). This term, however, is equal to 0 and thus it can be dropped. FIG. 64 illustrates why this is the case. Essentially, the non-zero elements of a″ and b′ don't overlap during any iteration of this calculation. They don't overlap at the beginning when n=0. They also don't overlap for n≥0 since the sequence b′ is always shifted to the left. Thus, the elements (a″*b′)ₙ of this cross-correlation sequence are all zero for n≥0.

By combining all of these results we can express formula (4.46) in the following way:

$${ℨ}_{a ★ b}^{+} ( z ) = {\underbrace{{ℨ}_{{a}^{′} ★ {b}^{′}}^{+} ( z )}}_{P} + {\underbrace{{ℨ}_{{a}^{′} ★ {b}^{″}}^{+} ( z )}}_{R} + {\underbrace{{ℨ}_{{a}^{″} ★ {b}^{′}}^{+} ( z )}}_{0} + {\underbrace{{ℨ}_{{a}^{″} ★ {b}^{″}}^{+} ( z )}}_{𝒬} . \qquad ( 4.46 )$$

If we express R in terms of (4.48) and drop the third term because it is equal to zero, then we will get:

$${ℨ}_{a ⋆ b}^{+} ( 𝓏 ) = {ℨ}_{{a}^{′} ⋆ {b}^{′}}^{+} ( 𝓏 ) + {ℨ}_{{a}^{″} ⋆ {b}^{″}}^{+} ( 𝓏 ) + \overline{{ℨ}_{{a}^{′}}^{+} ( 1 / \overline{𝓏} )} {ℨ}_{{b}^{″}}^{+} ( 𝓏 ) , \qquad ( 4.5 )$$

which is the familiar expression for the concatenation theorem.

### 4.7 Two Special Cases of the Concatenation Theorem

This section illustrates two special cases of the concatenation theorem. In the first case the two sequences a and b are split such that the two suffixes are both of length 1. In the second case the sequences are split such that the two prefixes are of length 1. The reason why these two special cases are interesting is because they lay the mathematical foundations for the encoding and the decoding algorithm, respectively.

### 4.7.1 When Both Suffixes are of Length One

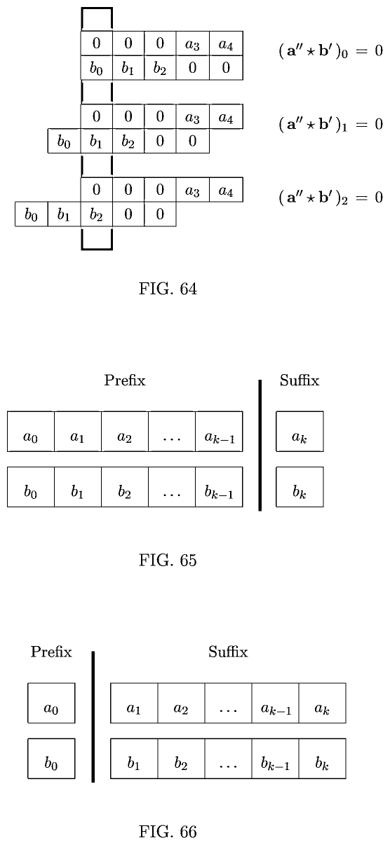

The two sequences in this example are a=(a₀, a₁, a₂, . . . , aₖ₋₁, aₖ) and b=(b₀, b₁, b₂, . . . , bₖ₋₁, bₖ). Both sequences are of length k+1. In this special case the sequences are split as shown in FIG. 65, which uses a thick vertical line to represent the prefix-suffix boundary. In other words, the sequences are split such that the two prefixes are of length k and the two suffixes are of length 1.

Once again, it is mathematically more convenient if we represent both the prefix and the suffix with sequences of length k+1 that are padded with the appropriate number of zeros. In this case, a′=(a₀, a₁, a₂, . . . , aₖ₋₁, 0) and a″=(0, 0, 0, . . . , 0, aₖ). The sequence a can be obtained from the elementwise sum of a′ and a″, i.e., a=a′+a″. Similarly, the sequence b is the sum of b′ and b″, where b′=(b₀, b₁, b₂, . . . , bₖ₋₁, 0) and b″=(0, 0, 0, . . . , 0, bₖ).

The concatenation theorem applied to these sequences states that:

$${ℨ}_{a ⋆ b}^{+} ( 𝓏 ) = {ℨ}_{{a}^{′} ⋆ {b}^{′}}^{+} ( 𝓏 ) + {ℨ}_{{a}^{″} ⋆ {b}^{″}}^{+} ( 𝓏 ) + \overline{{ℨ}_{{a}^{′}}^{+} ( 1 / \overline{𝓏} )} {ℨ}_{{b}^{″}}^{+} ( 𝓏 ) . \qquad ( 4.51 )$$

Because in this special case a″=(0, 0, 0, . . . , 0, aₖ) and b″=(0, 0, 0, . . . , 0, bₖ) it is easy to see that [symbol] _{a″*b″}⁺(z)=a̅ₖ̅bₖz⁰=a̅ₖ̅bₖ. It is also easy to see that [symbol] _{b″}⁺(z)=bₖz^{-k}. By substituting these values into equation (4.51) the formula simplifies to:

$${ℨ}_{a ⋆ b}^{+} ( 𝓏 ) = {ℨ}_{{a}^{′} ⋆ {b}^{′}}^{+} ( 𝓏 ) + \overline{{a}_{k}} {b}_{k} + \overline{{ℨ}_{{a}^{′}}^{+} ( 1 / \overline{𝓏} )} {b}_{k} {𝓏}^{- k} . \qquad ( 4.52 )$$

By factoring out the common term bₖ, the previous expression can be further simplified to:

$${ℨ}_{a ⋆ b}^{+} ( 𝓏 ) = {ℨ}_{{a}^{′} ⋆ {b}^{′}}^{+} ( 𝓏 ) + ( \overline{{a}_{k}} + {𝓏}^{- k} \overline{{ℨ}_{{a}^{′}}^{+} ( 1 / \overline{𝓏} )} ) {b}_{k} . \qquad ( 4.53 )$$

Furthermore, the term in the brackets simplifies to the value of the unilateral z-transform of the reversed and conjugated sequence a (the entire sequence a, not just the prefix a′), evaluated at z. In other words,

$$\begin{matrix}\overline{{a}_{k}} + {𝓏}^{- k} \overline{{ℨ}_{{a}^{′}}^{+} ( 1 / \overline{𝓏} )} & = \overline{{a}_{k}} + {𝓏}^{- k} \overline{( {a}_{0} {( 1 / \overline{𝓏} )}^{0} + {{a}_{1} ( 1 / \overline{𝓏} )}^{- 1} + … + {{a}_{k - 1} ( 1 / \overline{𝓏} )}^{- ( k - 1 )} + 0 {( 1 / \overline{𝓏} )}^{- k} )} \\ & = \overline{{a}_{k}} + {𝓏}^{- k} ( {\overline{{a}_{0}} ( 1 / 𝓏 )}^{0} + {\overline{{a}_{1}} ( 1 / \overline{𝓏} )}^{- 1} + … + {\overline{{a}_{k - 1}} ( 1 / 𝓏 )}^{- ( k - 1 )} + 0 ) \\ & = \overline{{a}_{k}} + {𝓏}^{- k} ( \overline{{a}_{0}} {z}^{0} + \overline{{a}_{1}} {z}^{1} + … + \overline{{a}_{k - 1}} {z}^{k - 1} ) \\ & = \overline{{a}_{k}} + \overline{{a}_{0}} {𝓏}^{- k} + \overline{{a}_{1}} {𝓏}^{- ( k - 1 )} + … + \overline{{a}_{k - 1}} {𝓏}^{- 1} \\ & = \overline{{a}_{k}} {z}^{0} + \overline{{a}_{k - 1}} {𝓏}^{- 1} + … + \overline{{a}_{1}} {𝓏}^{- ( k - 1 )} + \overline{{a}_{0}} {𝓏}^{- k} \\ & = {ℨ}_{\overline{\overleftarrow{a}}}^{+} - ( 𝓏 ) .\end{matrix} \qquad ( 4.54 )$$

Thus, in this special case, the concatenation theorem simplifies to:

$${ℨ}_{a ⋆ b}^{+} ( 𝓏 ) = {ℨ}_{{a}^{′} ⋆ {b}^{′}}^{+} ( 𝓏 ) + {ℨ}_{\overline{\overleftarrow{a}}}^{+} ( 𝓏 ) {b}_{k} \qquad ( 4.55 )$$

This expression justifies the encoding algorithm. In this notation [symbol] _{a*b}⁺(z) can be interpreted as the value of an element of the SSM matrix at the end of some iteration. This matrix element corresponds to the row associated with a and the column associated with b. Similarly, [symbol] (z) is the value of the same matrix element at the beginning of the iteration. Finally, [symbol] (z) can be interpreted as the value of the element of the vector h′ that corresponds to the a-channel. For binary sequences, the value of bₖ determines if the addition should be performed during this iteration. For example, if bₖ=1, then the a-th element of h′ is added to the corresponding matrix element. On the other hand, if bₖ=0, then the matrix element remains the same. It is worth emphasizing, once again, that this formula is for just one matrix element.

### 4.7.2 When Both Prefixes are of Length One

To explain the second special case we will use the same two sequences as in the previous example. In this case, however, the sequences a and b are split such that the two prefixes are of length 1 and the two suffixes are of length k. This split is shown in FIG. 66.

Using the convention from the previous section, the two prefixes a′ and b′ can be represented with sequences that contain one element followed by k zeros, i.e., a′=(a₀, 0, 0, . . . , 0) and b′=(b₀, 0, 0, . . . , 0). Similarly, the two suffixes a″ and b″ can be represented with sequences that have one leading zero followed by k elements, i.e., a″=(0, a₁, a₂, . . . , aₖ₋₁, aₖ) and b″=(0, b₁, b₂, . . . , bₖ₋₁, bₖ). As before, the sequence a can be represented as the elementwise sum of a′ and a″, i.e., a=a′+a″. Similarly, b=b′+b″.

Applying the concatenation theorem to the sequences in this special case we get:

$${ℨ}_{a ⋆ b}^{+} ( 𝓏 ) = {ℨ}_{{a}^{′} ⋆ {b}^{′}}^{+} ( 𝓏 ) + {ℨ}_{{a}^{″} ⋆ {b}^{″}}^{+} ( 𝓏 ) + \overline{{ℨ}_{{a}^{′}}^{+} ( 1 / \overline{𝓏} )} {ℨ}_{{b}^{″}}^{+} ( 𝓏 ) . \qquad ( 4.56 )$$

Because both a′ and b′ contain only one non-zero element we can simplify this formula b noting that [symbol] _{a′*b′}⁺(z)=a̅₀̅b₀z⁰=a̅₀̅b₀. It is also easy to see

$$\overline{{ℨ}_{{a}^{′}}^{+} ( 1 / \overline{𝓏} )} = \overline{{a}_{0} {( 1 / \overline{𝓏} )}^{0}} = \overline{{a}_{0}} .$$

If we plug these values into (4.56), then we will get the following simpler formula:

$${ℨ}_{a ⋆ b}^{+} ( 𝓏 ) = \overline{{a}_{0}} {b}_{0} + {ℨ}_{{a}^{″} ⋆ {b}^{″}}^{+} ( 𝓏 ) + \overline{{a}_{0}} {ℨ}_{{b}^{″}}^{+} ( 𝓏 ) . \qquad ( 4.57 )$$

By factoring out the common term a̅₀̅, this can also be expressed as:

$${ℨ}_{a ⋆ b}^{+} ( 𝓏 ) = \overline{{a}_{0}} ( {b}_{0} + {ℨ}_{{b}^{″}}^{+} ( 𝓏 ) ) + {ℨ}_{{a}^{″} ⋆ {b}^{″}}^{+} ( 𝓏 ) . \qquad ( 4.58 )$$

Using the properties of the z-transform, the expression in the brackets can be simplified to [symbol] _{b}⁺(z), i.e., the unilateral z-transform of the entire sequence b, not just the suffix b″. That is,

$$\begin{matrix}{b}_{0} + {ℨ}_{{b}^{″}}^{+} ( 𝓏 ) & = {b}_{0} + ( 0 {𝓏}^{0} + {b}_{1} {𝓏}^{- 1} + {b}_{2} {𝓏}^{- 2} + ⋯ + {b}_{k - 1} {𝓏}^{- ( k - 1 )} + {b}_{k} {𝓏}^{- k} ) \\ & = {b}_{0} {𝓏}^{0} + {b}_{1} {𝓏}^{- 1} + {b}_{2} {𝓏}^{- 2} + ⋯ + {b}_{k - 1} {𝓏}^{- ( k - 1 )} + {b}_{k} {𝓏}^{- k} \\ & = {ℨ}_{b}^{+} ( 𝓏 )\end{matrix} . \qquad ( 4.59 )$$

Using this result, the concatenation theorem simplifies to:

$${ℨ}_{a ⋆ b}^{+} ( 𝓏 ) = \overline{{a}_{0}} {ℨ}_{b}^{+} ( 𝓏 ) + {ℨ}_{{a}^{″} ⋆ {b}^{″}}^{+} ( 𝓏 ) . \qquad ( 4.6 )$$

By rearranging the terms, the formula can also be stated in this form:

$${ℨ}_{{a}^{″} ⋆ {b}^{″}}^{+} ( 𝓏 ) = {ℨ}_{a ⋆ b}^{+} ( 𝓏 ) - \overline{{a}_{0}} {ℨ}_{b}^{+} ( 𝓏 ) \qquad ( 4.61 )$$

This expression is the mathematical justification for the decoding algorithm. The term [symbol] _{a*b}⁺(z) can be interpreted as the value of an element of the SSM matrix after the first decoding iteration. The term [symbol] _{a*b}⁺(z) can be interpreted as the value of the same matrix element before the decoding starts. The term [symbol] _{b}⁺(z) can be interpreted as the value of the b-th element of the vector h″, i.e., the one that corresponds to the b-channel. For binary sequences, the value of a̅₀̅ determines if the subtraction will be performed during this iteration. If a̅₀̅=1, then the value of the b-th element of h″ is subtracted from the matrix element. On the other hand, if a̅₀̅=0, then nothing is subtracted.

### 4.8 Three Different Ways to Calculate the Same Sum

By definition, the value of the unilateral z-transform, evaluated at z, of the cross-correlation of two right-sided sequences a and b is a sum. Each term of this sum is equal to the product between an element of the sequence (a*b) and a corresponding negative power of z. Each element of the cross-correlation sequence, however, is also expressible as a sum. Thus, the z-transform expression can be viewed as a sum of sums. If all terms of this expression are expanded, then certain regularities emerge that make it possible to compute the value of [symbol] _{a*b}⁺(z) in three different ways.

To give a concrete example, let a=(a₀, a₁, a₂, a₃, a₄) and b=(b₀, b₁, b₂, b₃, b₄) be two right-sided sequences. The value of the unilateral z-transform, evaluated at z, of the cross-correlation of a and b is given by formula (4.37), which is replicated below:

$$\begin{matrix}{ℨ}_{a ⋆ b}^{+} ( 𝓏 ) & = \overline{{a}_{0}} {b}_{0} {𝓏}^{0} & + \overline{{a}_{0}} {b}_{1} {𝓏}^{- 1} & + \overline{{a}_{0}} {b}_{2} {𝓏}^{- 2} & + \overline{{a}_{0}} {b}_{3} {𝓏}^{- 3} & + \overline{{a}_{0}} {b}_{4} {𝓏}^{- 4} \\ & & + \overline{{a}_{1}} {b}_{1} {𝓏}^{0} & + \overline{{a}_{1}} {b}_{2} {𝓏}^{- 1} & + \overline{{a}_{1}} {b}_{3} {𝓏}^{- 2} & + \overline{{a}_{1}} {b}_{4} {𝓏}^{- 3} \\ & & & + \overline{{a}_{2}} {b}_{2} {𝓏}^{0} & + \overline{{a}_{2}} {b}_{3} {𝓏}^{- 1} & + \overline{{a}_{2}} {b}_{4} {𝓏}^{- 2} \\ & & & & + \overline{{a}_{3}} {b}_{3} {𝓏}^{0} & + \overline{{a}_{3}} {b}_{4} {𝓏}^{- 1} \\ & & & & & + \overline{{a}_{4}} {b}_{4} {𝓏}^{0}\end{matrix} . \qquad ( 4.62 )$$

This formula expresses the value of [symbol] _{a*b}⁺(z) as a sum and arranges the individual terms of this sum in a specific grid pattern. Each term of this sum has the following form: a̅ⱼ̅bₖ z^{-(k-j)}. In other words, each term is the product of three things: 1) the complex conjugate of an element from the sequence a; 2) an element of the sequence b; and 3) a negative power of z. This suggests that the terms in the large sum in (4.62) can be grouped in three different ways depending on which of the three variables is factored out. These three cases correspond to factoring out z^{-(k-j)}, bₖ, and a̅ⱼ̅, respectively. Each of these is briefly discussed below.

4.8.1 Summing Along the Diagonals

[symbol]

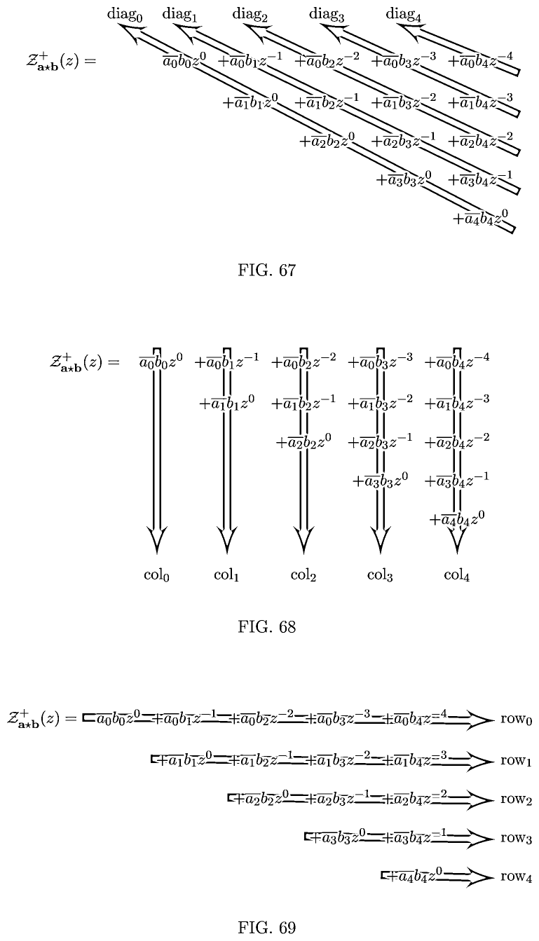

The first method of computing [symbol] _{a*b}⁺(z) starts by adding the terms in each diagonal of formula (4.62) and then adds all partial results. FIG. 67 illustrates this process and uses arrows to indicate the way in which the terms are grouped. As can be seen from the figure, all terms along the main diagonal contain z⁰. The terms along the first upper off-diagonal contain z⁻¹, and so on. In other words, this method groups the terms by their common power of z.

The five diagonal sums in this example can be expressed in the following form:

$${diag}_{0} = ( \overline{{a}_{0}} {b}_{0} + \overline{{a}_{1}} {b}_{1} + \overline{{a}_{2}} {b}_{2} + \overline{{a}_{3}} {b}_{3} + \overline{{a}_{4}} {b}_{4} ) {𝓏}^{0} , \qquad ( 4.63 )$$

$${diag}_{1} = ( \overline{{a}_{0}} {b}_{1} + \overline{{a}_{1}} {b}_{2} + \overline{{a}_{2}} {b}_{3} + \overline{{a}_{3}} {b}_{4} ) {𝓏}^{- 1} , \qquad ( 4.64 )$$

$${diag}_{2} = ( \overline{{a}_{0}} {b}_{2} + \overline{{a}_{1}} {b}_{3} + \overline{{a}_{2}} {b}_{4} ) {𝓏}^{- 2} , \qquad ( 4.65 )$$

$${diag}_{3} = ( \overline{{a}_{0}} {b}_{3} + \overline{{a}_{1}} {b}_{4} ) {𝓏}^{- 3} , \qquad ( 4.66 )$$

$${diag}_{4} = ( \overline{{a}_{0}} {b}_{4} ) {𝓏}^{- 4} . \qquad ( 4.67 )$$

The terms in the parentheses are equal to the elements of the cross-correlation sequence (a*b). Thus, the sum in (4.62) can be expressed as:

$$( 4.68 )$$

$$\begin{matrix}{ℨ}_{a ★ b}^{+} ( z ) = {diag}_{0} + {diag}_{1} + {diag}_{2} + {diag}_{3} + {diag}_{4} \\ = {( a ★ b )}_{0} {z}^{0} + {( a ★ b )}_{1} {z}^{- 1} + {( a ★ b )}_{2} {z}^{- 2} + {( a ★ b )}_{3} {z}^{- 3} + {( a ★ b )}_{4} {z}^{- 4} \\ = {\sum }_{n = 0}^{4} {( a ★ b )}_{n} {z}^{- n} ,\end{matrix}$$

which is equal to the value of the unilateral z-transform of a*b, evaluated at z.

### 4.8.2 Summing Along the Columns

The second method calculates the same value, [symbol] _{a*b}⁺(z), but it groups the terms of formula (4.62) based on their common element from the sequence b. As shown in FIG. 68 this groups the terms by columns, where the grouping is indicated with vertical arrows. That is, the only term in the 0-th column contains b₀; the two terms in the 1-st column both contain b₁; and so on. Adding the values of all column sums results in [symbol] _{a*b}⁺(z).

### 4.8.3 Summing Along the Rows

The third way of calculating the sum factors out the common element a̅ⱼ̅ from the first sequence. As shown in FIG. 69 this has the effect of grouping the elements by rows. In other words, all terms in row 0 contain a̅₀̅; all terms in row 1 contain a̅₁̅; and so on. Adding the values of all row sums results in [symbol] _{a*b}⁺(z), which is the same value that was computed by the previous two methods.

### 4.8.4 Summary

All three of these methods produce the same result, namely the value of the unilateral z-transform, evaluated at z, of the cross-correlation of a and b. This should not be surprising as all three methods add the same terms, they just add them in different order. The first method is the traditional method of computing [symbol] _{a*b}⁺(z). It groups the terms of formula (4.62) along the diagonals and then adds all diagonal sums. If the elements of the cross-correlation sequence (a*b) are known, then this should be the preferred way to calculate [symbol] _{a*b}⁺(z). However, if the cross-correlation sequence is not known in advance, then one of the other two methods should be used as they can be implemented to run faster by reusing partial results from the previous iterations, which is not possible with this method.

The second method groups the terms by columns and then adds the values of all column sums. This method can be further optimized as the value of the next column sum can be efficiently computed using the value of the previous column sum. This is the method that the encoding algorithm uses.

The third method is used by the decoding algorithm. Instead of computing the value of [symbol] _{a*b}⁺(z), however, the decoding algorithm starts with this value and subtracts the values of the row sums from it, one by one. Computational efficiency can be achieved in this case as well, because it is possible to quickly calculate the value of row k+1 given the value of row k.

### 5 ZUV Algorithms

This chapter extends the encoding and decoding algorithms to work with exponentially weighted sequences. These extensions were designed to overcome the decoding limitations described in Chapter 2. The modified algorithms can decode the matrices for sequence pairs of arbitrary length.

The names of the new algorithms start with the prefix ZUV. The three letters in this prefix correspond to three parameters of the algorithms that have the following meaning: z is the point at which all unilateral z-transforms in the formulas are evaluated; u is a parameter that determines the rate of exponential decay (or growth) of the elements of the first sequence; and v is another parameter that determines the rate of exponential decay (or growth) of the elements of the second sequence.

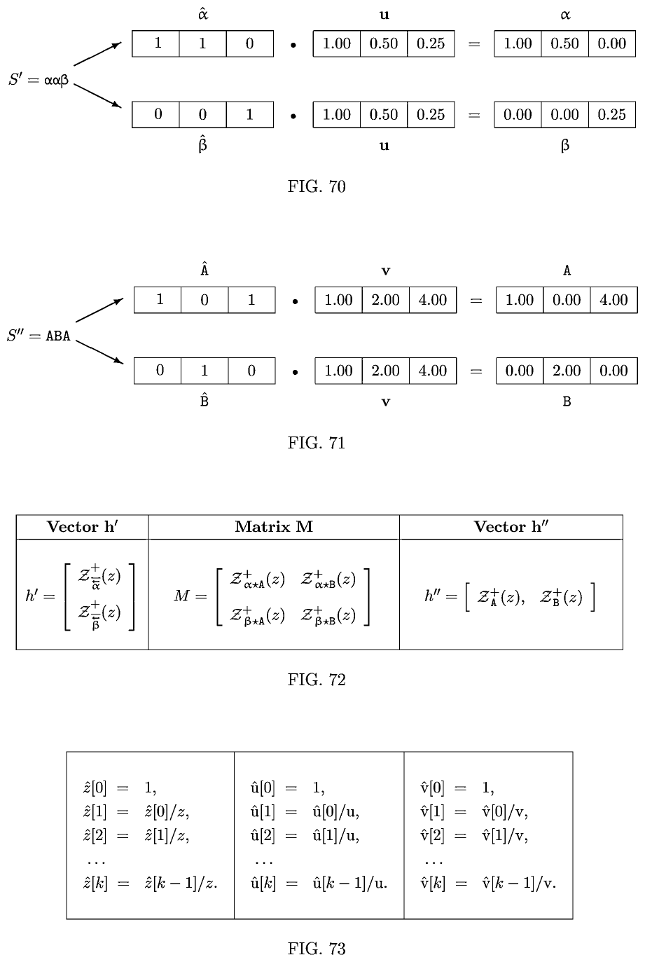

In all previously described encoding algorithms the input character sequences S′ and S″ were represented with a collection of binary sequences. The ZUV encoding algorithm also works with a pair of character sequences, each of which is represented with a set of binary sequences. Before these binary sequences are processed, however, the encoding algorithm scales each of them using exponentially decaying (or exponentially growing) weights. The resulting scaled sequences are no longer binary. The parameter u controls the exponential weights for the sequences that jointly represent S′. FIG. 70 gives an example with the character sequence S′=ααβ. Similarly, the parameter v controls the exponential weights for the sequences that correspond to S″. FIG. 71 illustrates this process using the character sequence S″=ABA. The ZUV decoding algorithm performs the same mapping of S″, which is provided at run time.

In FIG. 70, the sequence S′ is first mapped to two binary sequences \hat{α}=(1, 1, 0) and =(0, 0, 1). A value of 1 in \hat{α} indicates that the character α occurs at that position in S′. Similarly, the 1 in \hat{β} indicates the location of the only β in S′. Each element of \hat{α} is then multiplied by its corresponding element of u=(u⁰, u⁻¹, u⁻²). In this example u=2 so u=(1.0, 0.5, 0.25). The same multiplication is performed between \hat{β} and u. If the sequences are mapped to vectors, then the value of α would be equal to the element-by-element product of \hat{α} and u. Similarly, the value of β would be equal to the element-by-element product between \hat{β} and u. The sequences α and β are no longer binary. Note, however, that they contain zeros in the same places in which the binary sequences \hat{α} and \hat{β} contain zeros. Thus, only the ones in the binary sequences are scaled by the exponential weights.

The mapping shown in FIG. 71 is similar to the one shown in FIG. 70. However, in FIG. 71 this case, S″ is first mapped to two binary sequences Â=(1, 0, 1) and \hat{B}=(0, 1, 0). Both of these are then multiplied by the elements of v=(v⁰, v⁻¹, v⁻²). In this case v=0.5 so v=(1, 2, 4). Thus, A can be viewed as the element-by-element product between Â and v. Similarly, B can be viewed as the element-by-element product between \hat{B} and v. This mapping of S″ is performed during encoding and also during decoding.

FIG. 72 shows the formulas for the three components that are computed by the ZUV encoding algorithm for S′=ααβ and S″=ABA. These are expressed in the same way as in previous examples. What is different is that the underlying sequences α, β, A, and B are now exponentially weighted instead of binary. Another difference is that the encoding results now depend on the values of z, u and v, instead of just z.

To explain the mathematical justifications behind the ZUV algorithms we will extend the derivations from Chapter 4 to exponentially weighted sequences. Using this previous methodology, we will focus on only one element of the matrix. Without loss of generality, we will pick the element in the a-th row and b-th column. This element, which will be denoted with M_{a,b}, is equal to the value of the unilateral z-transform at z of the cross-correlation of the sequences a and b. In other words, M_{a,b}= [symbol] _{a*b}⁺(z). So far this is similar to the derivations in Chapter 4. The difference is that the sequences a and b are now exponentially weighted as described below.

Let â=(a₀, a₁, . . . , a_{T-1}) and \hat{b}=(b₀, b₁, . . . , b_{T-1}) be two binary sequences, i.e., each of their elements is equal to either 0 or 1. Also, let a be an exponentially weighted version of â. In other words, a=(a₀u⁰, a₁u⁻¹, . . . , a_{T-1}u^{-(T-1)}), where u is a parameter that determines the rate of decay (or growth) of the weight assigned to each element of â. If the sequences are represented with vectors, then a will be equal to the element-by-element product of â and u, where u=(u⁰, u⁻¹, . . . , u^{-(T-1)}). Note that the sequence a is no longer binary. Similarly, let b be an exponentially weighted version of \hat{b}. That is, b=(b₀v⁰, b₁v⁻¹, . . . , b_{T-1}v^{-(T-1)}), where v is a parameter that determines the rate of decay (or growth) of the weight for each element of \hat{b}. If the sequences are treated as vectors, then b will be equal to the element-by-element product of \hat{b} and v, where v=(v⁰, v⁻¹, . . . , v^{-(T-1)}). The sequence b is not binary either.

Using the methodology described in Chapter 4, we can express the value of the unilateral z-transform at z of the cross-correlation of the exponentially weighted sequences a and b as follows:

$${ℨ}_{a ★ b}^{+} ( z ) = \overline{{a}_{0} {u}^{0}} {b}_{0} {v}^{0} {z}^{0} + \overline{{a}_{0} {u}^{0}} {b}_{1} {v}^{- 1} {z}^{- 1} + \overline{{a}_{0} {u}^{0}} {b}_{2} {v}^{- 2} {z}^{- 2} + \overline{{a}_{0} {u}^{0}} {b}_{3} {v}^{- 3} {z}^{- 3} + \overline{{a}_{0} {u}^{0}} {b}_{4} {v}^{- 4} {z}^{- 4} + \overline{{a}_{1} {u}^{- 1}} {b}_{1} {v}^{- 1} {z}^{0} + \overline{{a}_{1} {u}^{- 1}} {b}_{2} {v}^{- 2} {z}^{- 1} + \overline{{a}_{1} {u}^{- 1}} {b}_{3} {v}^{- 3} {z}^{- 2} + \overline{{a}_{1} {u}^{- 1}} {b}_{4} {v}^{- 4} {z}^{- 3} + \overline{{a}_{2} {u}^{- 2}} {b}_{2} {v}^{- 2} {z}^{0} + \overline{{a}_{2} {u}^{- 2}} {b}_{3} {v}^{- 3} {z}^{- 1} + \overline{{a}_{2} {u}^{- 2}} {b}_{4} {v}^{- 4} {z}^{- 2} + \overline{{a}_{3} {u}^{- 3}} {b}_{3} {v}^{- 3} {z}^{0} + \overline{{a}_{3} {u}^{- 3}} {b}_{4} {v}^{- 4} {z}^{- 1} + \overline{{a}_{4} {u}^{- 4}} {b}_{4} {v}^{- 4} {z}^{0} . \qquad ( 5.1 )$$

All terms in this sum have the following form:

$$\overline{{a}_{j} {u}^{- j}} {b}_{k} {v}^{- k} {z}^{- ( k - j )} . \qquad ( 5.2 )$$

This pattern suggests that the terms of formula (5.1) can be grouped in three different ways, i.e., there are three different ways to compute this sum (see Section 4.8). First, the terms can be grouped by their common power of z. This corresponds to adding the terms along each diagonal and then adding all partial sums, which is the traditional way to compute [symbol] _{a*b}⁺(z). Second, the terms can be grouped based on their common bₖv^{-k} factor. In this case, computing the overall sum is done by adding the terms in each column and then adding all column sums. Because each column sum can be computed very quickly using the value of the previous column sum this leads to a nice optimization that is used by the ZUV encoding algorithm. Finally, the terms of (5.1) can be grouped by their common a̅ⱼ̅u̅^̅{̅-̅j̅}̅ factor. This corresponds to adding the terms in each row and then adding all row sums. This process can also be optimized because each row sum can be efficiently computed from the value of the previous row sum. The ZUV decoding algorithm uses this optimization, but it subtracts the row sums from the overall sum instead of trying to compute this sum.

For the sake of convenience, the encoding formulas are shown below:

$${h}_{a}^{′} [ k ] = \overline{{a}_{k}} + \frac{1}{z} {h}_{a}^{′} [ k - 1 ] , \qquad ( 5.3 )$$

$${M}_{a , b} [ k ] = {M}_{a , b} [ k - 1 ] + {h}_{a}^{′} [ k ] {b}_{k} , \qquad ( 5.4 )$$

$${h}_{b}^{″} [ k ] = {h}_{b}^{″} [ k - 1 ] + {b}_{k} {z}^{- k} . \qquad ( 5.5 )$$

The decoding formulas from are also shown below:

$${M}_{a , b} [ k + 1 ] = {M}_{a , b} [ k ] - \overline{{a}_{k}} {h}_{b}^{″} [ k ] , \qquad ( 5.6 )$$

$${h}_{b}^{″} [ k + 1 ] = ( {h}_{b}^{″} [ k ] - {b}_{k} ) z . \qquad ( 5.7 )$$

The ZUV algorithms use these same formulas, but replace all instances of aₖ and bₖ with aₖu^{-k} and bₖv^{-k}, respectively. The derivations and optimizations are discussed in the next two sections.

### 5.1 ZUV Encoding Algorithm

This section describes the ZUV encoding algorithm. This is done in three steps. First, the update formulas are derived for individual elements of the matrix and the two vectors. Next, the algorithm is described. Finally, four numerical examples of encoding are given for different values of the parameters z, u, and v.

Let a=(a₀u⁰, a₁u⁻¹, . . . , a_{T-1} u^{-(T-1)}) and b=(b₀v⁰, b₁v⁻¹, . . . , b_{T-1} v^{-(T-1)}) be two exponentially weighted sequences of length T. Let a′=(a₀u⁰, a₁u⁻¹, . . . , a_{T-2} u^{-(T-2)}, 0) and a″=(0, 0, . . . , 0, a_{T-1} u^{-(T-1)}) be two other sequences of length T such that the sequence a can be obtained from the elementwise sum of a′ and a″, i.e., a=a′+a″. Also, the sequence b can be represented as the sum of b′ and b″, where b′=(b₀v⁰, b₁v⁻¹, . . . , b_{T-2}v^{-(T-2)}, 0) and b″=(0, 0, . . . , 0, b_{T-1} v^{-(T-1)}). The concatenation theorem for the unilateral z-transform applied to the sequences a and b states that

$${ℨ}_{a ★ b}^{+} ( z ) = {ℨ}_{{a}^{′} ★ {b}^{′}}^{+} ( z ) + {ℨ}_{{a}^{″} ★ {b}^{″}}^{+} ( z ) + \overline{{ℨ}_{a ′}^{+} ( 1 / \overline{z} )} {ℨ}_{{b}^{″}}^{+} ( z ) . \qquad ( 5.8 )$$

Because in this special case both a″ and b″ contain only one element that is not explicitly set to zero, the previous formula can be simplified as shown in Section 4.7.1, i.e.,

$${ℨ}_{a ★ b}^{+} ( z ) = {ℨ}_{a ★ {b}^{′}}^{+} ( z ) + ℨ \frac{+}{\overleftarrow{a}} ( z ) {b}_{k} {v}^{- k} , \qquad ( 5.9 )$$

where it is assumed that k=T−1. The individual terms of this expression can be interpreted as follows:

$${\underbrace{{ℨ}_{a ★ b}^{+} ( z )}}_{{M}_{a , b} [ k ]} = {\underbrace{{ℨ}_{{a}^{′} ★ {b}^{′}}^{+} ( z )}}_{{M}_{a , b} [ k - 1 ]} + {\underbrace{ℨ \frac{+}{\overleftarrow{a}} ( z )}}_{{h}_{a}^{′} [ k ]} {\underbrace{{b}_{k} {v}^{- k}}}_{{b}_{k} {v}^{- k}} . \qquad ( 5.1 )$$

In other words, [symbol] _{a*b}⁺(z) is the value of the matrix element M_{a,b} in the a-th row and b-th column after the k-th iteration. Similarly, [symbol] _{a*b′}⁺(z) is the value of the same matrix element after the (k−1)-st iteration. The term [symbol] is the value of the a-th element of the vector h′ during the k-th iteration. Finally, bₖv^{-k} is the k-th element of the exponentially weighted sequence b. A similar reasoning can be used to extend this formula to any k between 0 and T−1. This turns this formula into an iterative update formula.

Formula (5.10) requires the value of v^{-k}. To avoid computing this value from scratch during each iteration we will use a helper variable \hat{v}. This variable is initially set to 1. It is updated using the following recurrence: \hat{v}[k]=\hat{v}[k−1]/v, where v is one of the parameters of the ZUV algorithm. Thus, \hat{v}[k]=v^{-k}. Using this helper variable we can rewrite the bottom row of (5.10) as follows:

$${M}_{a , b} [ k ] = {M}_{a , b} [ k - 1 ] + {b}_{k} {h}_{a}^{′} [ k ] \hat{v} [ k ] . \qquad ( 5.11 )$$

Formula (5.11) uses the value of hₐ′[k], i.e., the value of the a-th element of the vector h′ during the k-th iteration. This value can be computed with the following iterative formula

$${h}_{a}^{′} [ k ] = \overline{{a}_{k} {u}^{- k}} + \frac{1}{z} {h}_{a}^{′} [ k - 1 ] , \qquad ( 5.12 )$$

which is derived from formula (5.3). In other words, to compute the new value of hₐ′ at iteration k, this formula uses the old value of hₐ′ at iteration k−1, and divides it by z. It also adds the conjugate of the k-th element of a, i.e., a̅ₖ̅u̅^̅{̅-̅k̅}̅. This iterative procedure computes [symbol] , i.e., the unilateral z-transform at z of the reversed and conjugated sequence a. All of this is done in place and there is no need to buffer the sequence.

Formula (5.12) needs the value of u^{-k}, which must be computed for each iteration. To avoid doing extra work, we will introduce another helper variable û such that û[k]=u^{-k}. Initially this variable is set to 1 and it is updated as follows: û[k]=û[k−1]/u. Substituting û[k] into (5.12) we get the following update formula:

$${h}_{a}^{′} [ k ] = \overline{{a}_{k} \hat{u} [ k ]} + \frac{1}{z} {h}_{a}^{′} [ k - 1 ] . \qquad ( 5.13 )$$

The ZUV encoding algorithm also needs to compute the vector h″. Adapting formula (5.5) to the exponentially weighted sequence b we get

$${h}_{b}^{″} [ k ] = {h}_{b}^{″} [ k - 1 ] + ( {b}_{k} {v}^{- k} ) {z}^{- k} . \qquad ( 5.14 )$$

Using the helper variables \hat{v}[k]=v^{-k} and \hat{z}[k]=z^{-k}, this expression can be rewritten as

$${h}_{b}^{″} [ k ] = {h}_{b}^{″} [ k - 1 ] + {b}_{k} \hat{v} [ k ] \hat{z} [ k ] . \qquad ( 5.15 )$$

To get a better understanding of how formula (5.15) works, recall that if the sequence length is T=k+1 the value of h_{b}″[k] is equal to [symbol] _{b″}⁺(z). In other words, the b-th element of the vector h″ is equal to the value of the unilateral z-transform at z of the exponentially weighted sequence b. Since b=(b₀v⁰, b₁v⁻¹, . . . , bₖv^{-k}) we can express h_{b}″[k] as follows:

$${h}_{b}^{″} [ k ] = ( {b}_{0} {v}^{0} ) {z}^{0} + ( {b}_{1} {v}^{- 1} ) {z}^{- 1} + … + ( {b}_{k - 1} {v}^{- ( k - 1 )} ) {z}^{- ( k - 1 )} + ( {b}_{k} {v}^{- k} ) {z}^{- k} . \qquad ( 5.16 )$$

Using the helper variables \hat{v} and \hat{z}, we can rewrite this formula as follows:

$${h}_{b}^{″} [ k ] = {\underbrace{{b}_{0} \hat{v} [ 0 ] \hat{z} [ 0 ] + {b}_{1} \hat{v} [ 1 ] \hat{z} [ 1 ] + … + {b}_{k - 1} \hat{v} [ k - 1 ] \hat{z} [ k - 1 ]}}_{{h}_{b}^{″} [ k - 1 ]} + {b}_{k} \hat{v} [ k ] \hat{z} [ k ] . \qquad ( 5.17 )$$

Because the sum of all terms except the last one is equal to h_{b}″[k−1], it should be easy to see why this is equivalent to (5.15).

To summarize, the ZUV encoding algorithm uses the following iterative formulas:

$${h}_{a}^{′} [ k ] = \overline{{a}_{k} \hat{u} [ k ]} + \frac{1}{z} {h}_{a}^{′} [ k - 1 ] , \qquad ( 5.18 )$$

$${M}_{a , b} [ k ] = {M}_{a , b} [ k - 1 ] + {b}_{k} {h}_{a}^{′} [ k ] \hat{v} [ k ] , \qquad ( 5.19 )$$

$${h}_{b}^{″} [ k ] = {h}_{b}^{″} [ k - 1 ] + {b}_{k} \hat{v} [ k ] \hat{z} [ k ] . \qquad ( 5.2 )$$

The algorithm also uses three helper variables: \hat{z}, û, and \hat{v} such that \hat{z}[k]=z^{-k}, û[k]=u^{-k}, and \hat{v}[k]=v^{-k}. These are also computed iteratively.

The ZUV encoding algorithm has five input arguments. The first two are the two input sequences S′ and S″. It is assumed that these are integer sequences, such that each integer maps to a character from the corresponding alphabet. Also, it is assumed that the sizes of the two alphabets are M′ and M″, respectively. The other three input arguments are z, u, and v. Their meaning was described above. In this implementation these three arguments are assumed to be real numbers.

The algorithm starts by initializing the matrix M, which is of size M′ by M″, with zeros. It zeros the vector h′, which is a vector of size M′. It initializes the vector h″, a vector of size M″, with zeros as well. The three helper variables \hat{z}, û, and \hat{v} are initialized to 1.

The main loop of the algorithm goes from 1 to T, where T is the length of the two input sequences. If the sequence length is unknown, then the algorithm can read the sequences one character at a time until a timeout occurs or until a terminating character is reached.

The algorithm has two independent inner loops. The first inner loop divides the values of all elements of the vector h′ by z. This implements the division by z in formula (5.18). Because this algorithm works with real numbers, the conjugation in this formula can be dropped. Also, the multiplication by aₖ may not be performed explicitly since aₖ is binary (see the discussion below).

The incoming characters from both sequences are assumed to be integers. Formula (5.20) updates the value of one element of the h″ vector The multiplication by bₖ can be implicit in this algorithm because bₖ is either 0 or 1. In other words, the formulas described in this section use the exponentially weighted sequence b, but the underlying sequence \hat{b}=(b₀, b₁, . . . , bₖ) is binary. If bₖ=1 then the multiplication by bₖ can be skipped as anything multiplied by 1 is equal to itself. On the other hand, if bₖ=0, then the entire product is equal to zero so there is no need to perform the multiplication either. The algorithm can use the mutual exclusivity between the binary sequences that correspond to each element of the vector h″. For example, in the representation for the sequence S″=ABA shown in FIG. 71 there is only one binary number equal to 1 per iteration in Â and \hat{B}. In other words, Âₖ+\hat{B}ₖ=1 for all k, where the addition is regular addition and not boolean addition. Thus, even though the algorithm uses the variable name b, this corresponds to the binary sequence that contains the 1 in the current iteration and not to the sequence that corresponds the b-th element of h″. Similar optimizations can be made in the calculation of the vector h′ and the matrix.

The second inner loop of the algorithm updates the matrix by implementing formula (5.19). The value of hᵢ′ can be scaled by the current value of \hat{v} before the product is added to the corresponding element of the matrix. The value of hᵢ′, however, is not modified. The multiplication by bₖ can be implicit here as well.

The helper variables \hat{z}, û, and \hat{v} can be updated by dividing each variable by the corresponding parameter z, u, or v. In other words, each update implements an exponential decay (or growth). FIG. 73 visualizes the recurrences for computing these helper variables. Note that each depends only on the value of the same variable during the previous iteration.

At the end of all iterations the algorithm returns the computed value of the matrix M, the vector h′, and the vector h″.

The computational complexity of this algorithm is O(TM′). This is the same complexity as with all previous encoding algorithms that are not performed on a parallel machine. In other words, the outer loop is executed T times and each of the two inner loops, which are independent of each other, is executed M′ times.

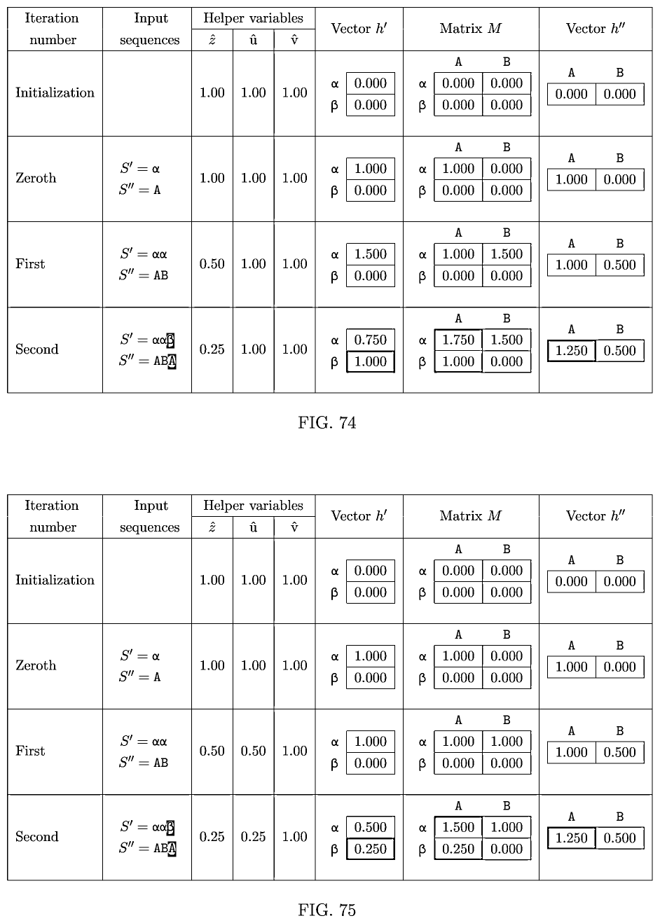

FIGS. 74-77 give four numerical examples of ZUV encoding for different values of the arguments z, u, and v. The four sets of values are: 1) z=2, u=1, and v=1; 2) z=2, u=2, and v=1; 3) z=1, u=2, and v=0.5; and 4) z=2, u=4, and v=0.5. The character sequences S′=ααβ and S″=ABA are used in all four examples. Note that even though the input sequences are the same, the encoded values of the matrix and the two vectors are completely different depending on the interplay between the values of z, u, and v. The values of the helper variables \hat{z}, û, and \hat{v} for each iteration are also shown in these figures.

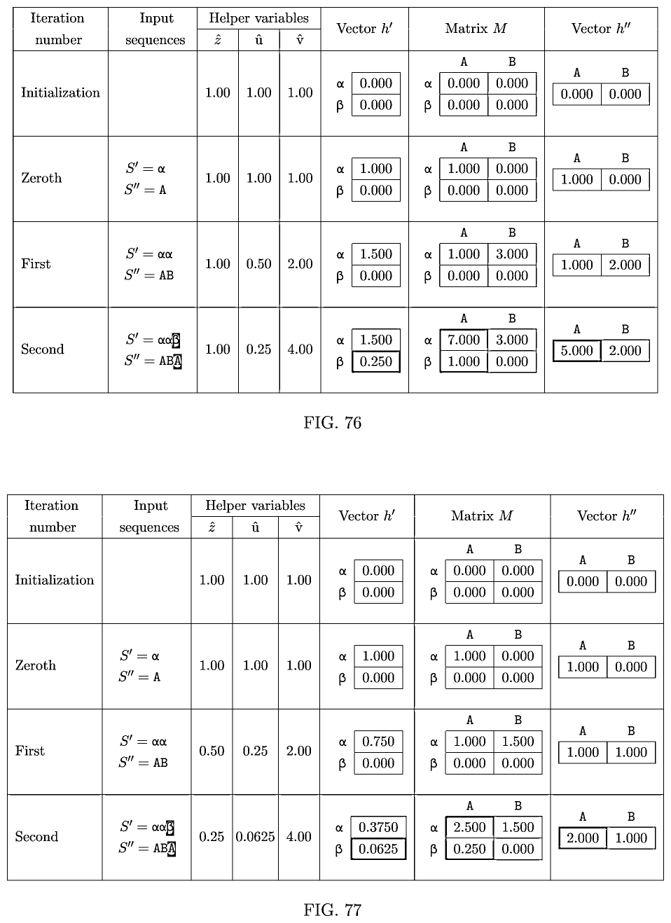

When studying these figures, recall that the contents of h′ decay with each iteration and that the rate of decay is controlled by z. Alternatively, if z=1, then the values in h′ don't decay (see FIG. 76). The value of 6 is added to one element of h′ during each iteration. Thus, the magnitude of what is added to h′ is controlled by u. Also, recall that h′ is scaled by \hat{v}[k] before it is added to one column of the matrix. That is why in FIG. 76 the value in the upper-left corner of the matrix is 7, even though there are no such large numbers in h′. Finally, the values in h″ don't decay during each iteration. What is added to an element of h″, however, is the product of \hat{v} and \hat{z}.

### 5.2 ZUV Decoding Algorithm

The decoding algorithm is justified by another special case of the concatenation theorem. In this case the prefixes of the two sequences are one character long. Let a=(a₀u⁰, a₁u⁻¹, . . . , a_{T-1} u^{-(T-1)}) and b=(b₀v⁰, b₁v⁻¹, . . . , b_{T-1}v^{-(T-1)}) be two exponentially weighted sequences of length T. Furthermore, let a′=(a₀u⁰, 0, 0, . . . , 0) and a″=(0, a₁u⁻¹, a₂u⁻², . . . , a_{T-1} u^{-(T-1)}) be two other sequences of length T such that the sequence a is equal to the element-by-element sum of these two sequences, i.e., a=a′+a″. Similarly, let b=b′+b″, where b′=(b₀v⁰, 0, 0, . . . , 0) and b″=(0, b₁v⁻¹, b₂v⁻², . . . , b_{T-1} v^{-(T-1)}) are two exponentially weighted sequences. The concatenation theorem, applied to these sequences, states that

$${ℨ}_{a ★ b}^{+} ( z ) = {ℨ}_{{a}^{′} ★ {b}^{′}}^{+} ( z ) + {ℨ}_{{a}^{″} ★ {b}^{″}}^{+} ( z ) + \overline{{ℨ}_{{a}^{′}}^{+} ( 1 / \overline{z} )} {ℨ}_{{b}^{″}}^{+} ( z ) . \qquad ( 5.21 )$$

As described in Section 4.7.2, for this special case, in which a′ and b′ have only one element that is not explicitly set to zero, the formula can be simplified to the following form

$${ℨ}_{{a}^{″} ★ {b}^{″}}^{+} ( z ) = {ℨ}_{a ★ b}^{+} ( z ) - \overline{{a}_{0} {u}^{0}} {ℨ}_{b}^{+} ( z ) , \qquad ( 5.22 )$$

where a₀u⁰ is the zeroth element of the sequence a.

The individual terms of formula (5.22) can be interpreted as follows:

$${\underbrace{{ℨ}_{{a}^{″} ★ {b}^{″}}^{+} ( z )}}_{{M}_{a , b} [ 1 ]} = {\underbrace{{ℨ}_{a ★ b}^{+} ( z )}}_{{M}_{a , b} [ 0 ]} - {\underbrace{\overline{{a}_{0} {u}^{0}}}}_{\overline{{a}_{0} {u}^{0}}} {\underbrace{{ℨ}_{b}^{+} ( z )}}_{{h}_{b}^{″} [ 0 ]} . \qquad ( 5.23 )$$

In this case, M_{a,b}[0] is the value of the matrix element in row a and column b at the start of decoding. This is the same value that the encoding algorithm computed at the end of encoding. M_{a,b}[1] is the value of the same matrix element at the start of the next iteration. The term [symbol] _{b}⁺(z) can be interpreted as the value of the b-th element of the vector h″ at the start of decoding. For an arbitrary iteration, formula (5.23) can be stated as:

$${M}_{a , b} [ k + 1 ] = {M}_{a , b} [ k ] - \overline{{a}_{k} {u}^{- k}} {h}_{b}^{″} [ k ] . \qquad ( 5.24 )$$

The decoding algorithm also needs to update the vector h″. Adapting formula (5.7) to exponentially weighted sequences we get

$${h}_{b}^{″} [ k + 1 ] = ( {h}_{b}^{″} [ k ] - {b}_{k} {v}^{- k} ) z . \qquad ( 5.25 )$$

To optimize the computation of the negative powers of u and v, we will use two helper variables û and \hat{v} such that û[k]=u^{-k} and \hat{v}[k]=v^{-k}. These variables are initially set to 1. During each iteration u is updated as follows: û[k+1]=û[k]/u. Similarly, v is updated using the following recurrence: \hat{v}[k+1]=\hat{v}[k]/v. Using these helper variables, the update formulas for the ZUV decoding algorithm can be stated as follows:

$${M}_{a , b} [ k + 1 ] = {M}_{a , b} [ k ] - \overline{{a}_{k} \hat{u} [ k ]} {h}_{b}^{″} [ k ] , \qquad ( 5.26 )$$

$${h}_{b}^{″} [ k + 1 ] = ( {h}_{b}^{″} [ k ] - {b}_{k} \hat{v} [ k ] ) z . \qquad ( 5.27 )$$

Note that the update of the matrix element is first, followed by the update of the vector h″.

The ZUV decoding algorithm has six input arguments. The first three arguments are the matrix M, the vector h″, and the character sequence S″. The other three arguments are the parameters z, u, and v, which were described above and after which the algorithm is named. All three arguments can be real numbers.

The algorithm can use two helper variables u and v to compute the negative powers of u and v. Both of these can be initially set to 1. Their values can updated at the end of each iteration.

The main loop of the algorithm performs T iterations, where T is the length of the second sequence S″. To find the next character to decode, the algorithm iterates over all M′ rows of the matrix. For each row it also iterates over all M″ columns. In each of these iterations, the algorithm checks whether the elements of the vector h″, scaled by the current value of u, can be subtracted from their corresponding elements of the matrix without any of the matrix elements becoming negative. This condition must be true for all elements in the row. In other words, a single row element has veto power, which is suggested by the variable with the same name. If all elements in some row satisfy this condition, then the algorithm decodes the character that corresponds to this row. If no rows satisfy this condition, then the algorithms breaks out of its main loop and returns the partial sequence that has been decoded up to this point. If the elements of h″ are all zeros while the algorithm is searching for the next character to decode, the algorithm exits as well. In a way, this approach implicitly checks if T or the length of S″ is longer than the length of the sequences that were used to encode the matrix. If that were the case, then the vector h″ would be depleted before the last iteration and would contain only zeros.

Next, the algorithm performs the subtraction in formula (5.26). More specifically, it multiplies h″ by u and subtracts the resulting vector from the selected row of the matrix. This can be done in a loop that iterates over all elements of the row. Just in case, the algorithm may check if the new value of each row element is still positive. Finally, the algorithm appends the index of the decoded row to the output sequence S′. This process is repeated T times.

The incoming character from the second character sequence S″ can be stored in the variable b. Once again, it is assumed that the characters are uniquely mapped to the integers from 1 to M″. The value of the b-th element of the h″ is reduced by v, as described by formula (5.27). The second part of this update, i.e., the multiplication by z that completes the left shift, is performed for all elements of h″. That is, formula (5.27) can be implemented by the algorithm in two parts; first the subtraction and then multiplication by z. Once again, because bₖ is binary, the multiplication by bₖ can be implicit. The same is true for the multiplication by aₖ in formula (5.26). This optimization can also be used during encoding and was explained in Section 5.1.

The algorithm also checks whether the element of h″ from which v was subtracted becomes negative. If yes, then the algorithm exits and returns what was decoded up to that point. This condition should not be triggered if the same S″ is used for decoding as the one that was used during encoding.

After the last iteration the algorithm returns the decoded sequence S′. Note that the output sequence is not exponentially weighted. It is just a character sequence that is mapped to an integer sequence.

The computational complexity of this algorithm is O(TM′M″). If the search for the next character to decode is implemented to run in parallel, then the complexity can be reduced to O(TM″).

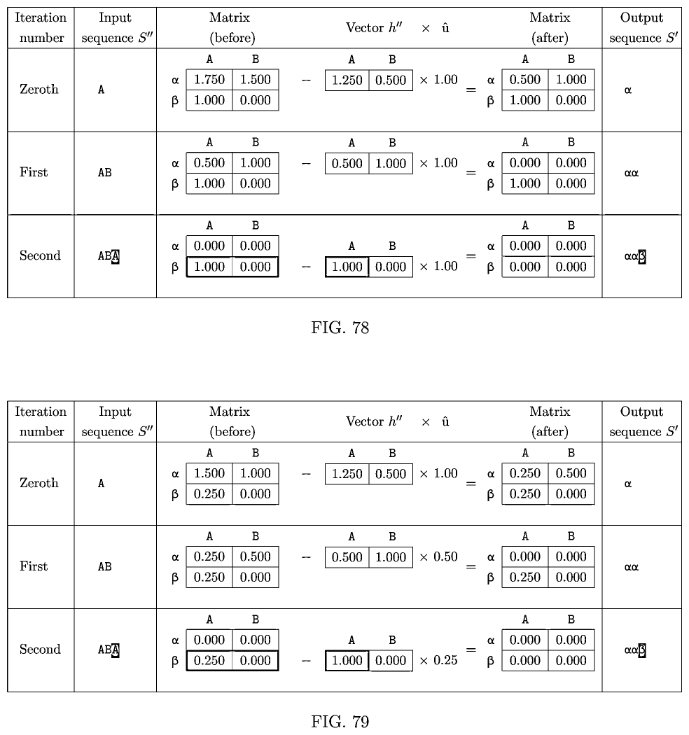

FIGS. 78-81 give four examples of ZUV decoding. In all four examples the matrix was encoded from the pair of sequences S′=ααβ and S″=ABA. The values of the arguments z, u, and v, however, are different in each example. Also, these figures are slightly different from previous decoding examples because h″ must be multiplied by u before it is subtracted from a row of the matrix. This multiplication is now indicated in the figures. Note, however, that the value of h″ is not affected by this; only what is subtracted from the matrix depends on u, i.e., this is how formula (5.26) works. The incoming character on S″ still selects one element of h″, but now the current value of v is subtracted from that element instead of 1. All elements of h″ are still multiplied by z at the end of each iteration as indicated in formula (5.27).

FIG. 78 shows an example in which z=2 and both u and v are equal to 1. Thus, this special case reduces to the traditional exponential decoding that depends only on z. Therefore, both û and \hat{v} are equal to 1 during all iterations and thus they don't affect the decoding process.

FIG. 79 gives another example in which z=2, u=2, and v=1. Since v=1, this is a special case of ZUV that can be called ZU. Because \hat{v} is equal to 1 for all iterations, what is subtracted from the elements of h″ is always equal to 1.

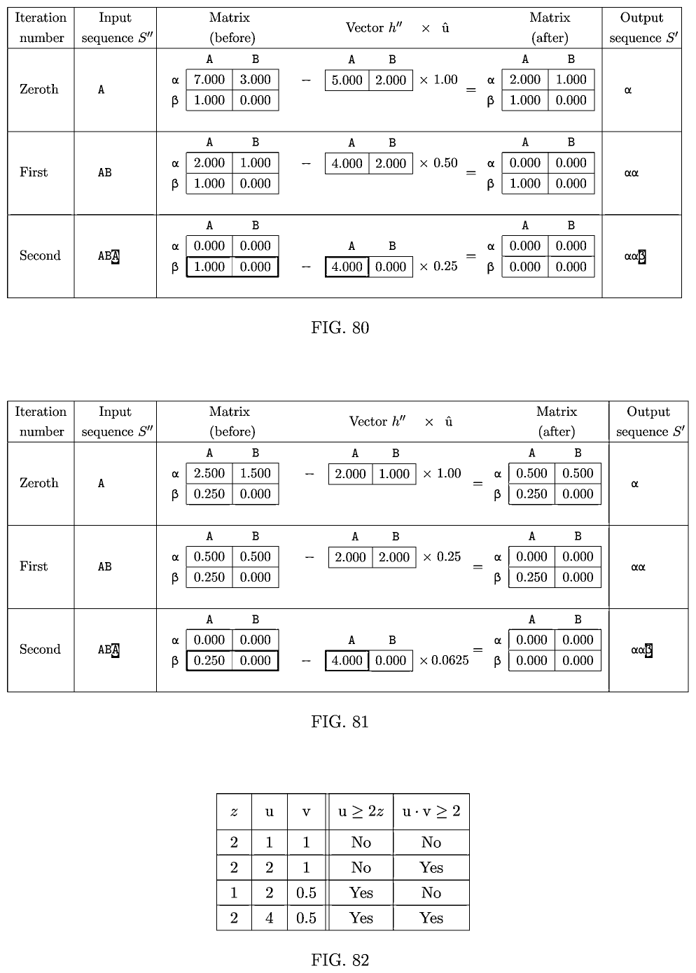

FIG. 80 gives another example with z=1, u=2, and v=0.5. Because v<1, the value of \hat{v} grows exponentially from 1 to 2 to 4. These values correspond to what is subtracted from the selected element of h″ during each iteration. Once again, this selection depends on the incoming character on S″.

Finally, FIG. 81 shows an example with z=2, u=4, and v=0.5. Now all parameters are different from 1. Thus, this example shows the richest from of interaction between the three parameters and how they affect the decoding process.

### 5.3 ZUV Evaluation

Two sufficient conditions for deterministic ZUV decoding were derived. They depend only on the values of the parameters z, u, and v. These conditions are:

$$u \cdot v \geq 2 or u \geq 2 z . \qquad ( 5.28 )$$

Because these two conditions are independent of each other, there are four possible cases depending on which one of them is satisfied or not satisfied. These four cases are listed in FIG. 82.

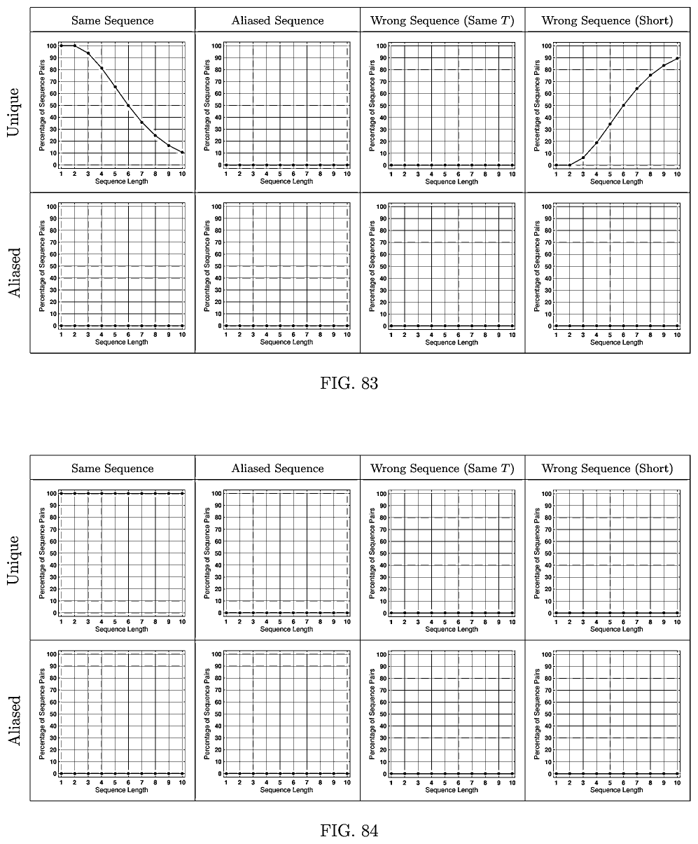

FIGS. 83-86 evaluate the decodability properties of the ZUV model for each of these four cases. These results were computed using a Python script.

FIG. 83 shows the only case in which both conditions are not satisfied. In this case both u=1 and v=1 and this reduces to the exponential case with z=2 that was analyzed in Section 2.13. In other words, this is a degenerate case of ZUV in which the input sequences are not exponentially weighted.

FIGS. 84-86 show the results for the parameter values specified in the last three rows of FIG. 82. These figures confirm that when one or both sufficient conditions are met the ZUV decoding process is deterministic. In all three cases the upper-left plot in each figure is at 100% and the remaining seven plots are at 0%.

6 Encoding and Decoding Algorithms for Sequences with Gaps

The algorithms described so far assumed that their input sequences are like words, i.e., that they contain no spaces. This chapter modifies the algorithms so that they can work with input sequences that are more like sentences, or strings, which may contain spaces. We will refer to these spaces as gaps. In the examples the gaps will be denoted with the underscore character, i.e., ‘_’. The algorithms introduced in this chapter are special cases of the ZUV algorithms when u=v=1. The ZUV algorithms for sequences with gaps are described in Chapter 7.

A gap can be modeled in several ways. One way is to treat the gap as yet another letter in the alphabet. In this case the algorithms do not have to be modified. The drawback of this approach is that the dimensions of the matrix have to be increased, i.e., both M′ and M″ have to be incremented by one, which requires additional storage for the matrices and also increases the amount of computation. This chapter models the gaps in a different way that keeps the alphabet size the same (much like the space symbol is not part of the English alphabet). As a result of this the matrix size remains the same, but the algorithms have to be modified. Understanding these changes and their effects on the encoding and decoding process could provide some valuable insights for understanding the continuous-time algorithms described in Chapter 8.

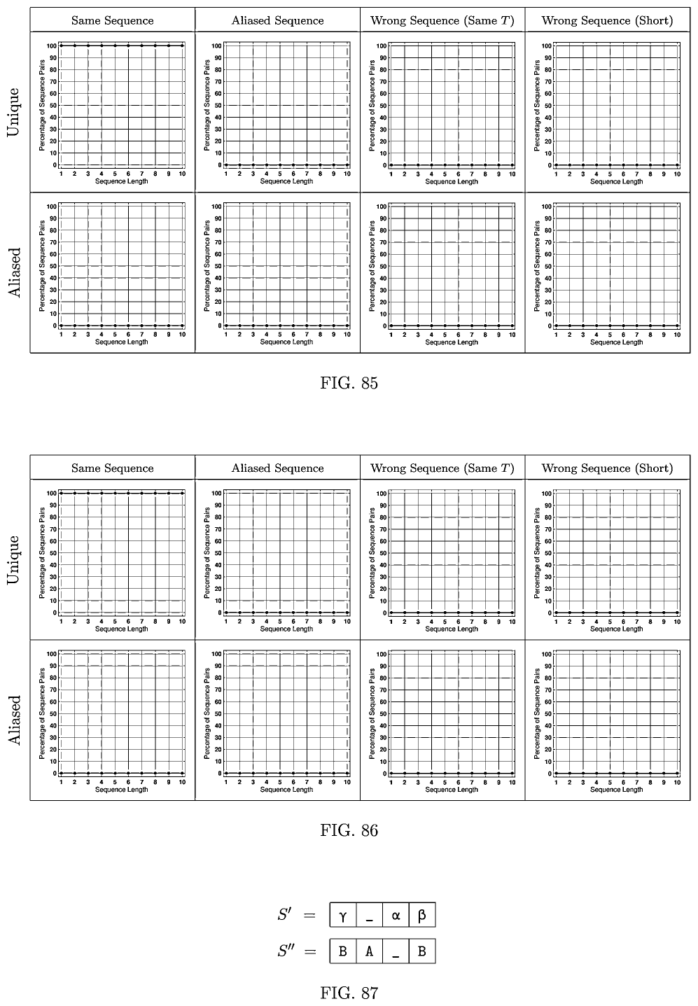

FIG. 87 shows the two sequences that will be used in the examples below. Both sequences are of length four and each contains one gap. The first sequence is S′=γ_αβ and it contains three unique characters, i.e., M′=3. The second sequence is S″=BA_B and it contains only two unique characters, i.e., M″=2.

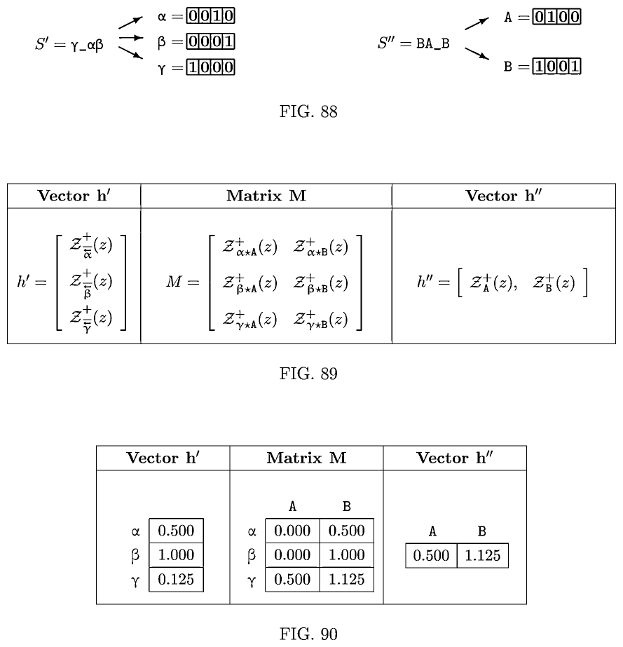

Character sequences can be represented with a collection of binary sequences. FIG. 88 shows this mapping for the two sequences in this example. The first sequence S′, which is spelled with Greek letters, is represented with three binary sequences: α, β, and γ. These have the same names as the characters in S′, but each is now a binary sequence of length 4. In other words, α=(α₀, α₁, α₂, α₃)=(0, 0, 1, 0), β=(β₀, β₁, β₂, β₃)=(0, 0, 0, 1), and γ=(γ₀, γ₁, γ₂, γ₃)=(1, 0, 0, 0). In each binary sequence a value of 1 indicates that the corresponding character occurs at that index in the character sequence; a value of 0 indicates that this character is not present at that index. The gap in S′ is at index 1 and it is represented with a 0 in all three binary sequences, i.e., α₁=β₁=γ₁=0. Similarly, the second character sequence S″=BA_B is represented with two binary sequences: A=(0, 1, 0, 0) and B=(1, 0, 0, 1). The gap in this case is at index 2 and it is represented with a zero at that position in both binary sequences, i.e., A₂=B₂=0.

FIG. 89 shows the three components that are computed by the encoding algorithm for this example: the vector h′, the matrix M, and the vector h″. In this figure each of their elements is expressed in an abstract form, i.e., in terms of the value of the z-transform of a specific sequence or the value of the z-transform of the cross-correlation of a pair of sequences.

FIG. 90 gives the concrete numerical values for these three components for the sequences shown in FIG. 88. These were computed using the encoding algorithm for sequences with gaps, which is described next.

6.1 The Encoding Algorithm (with Gaps)

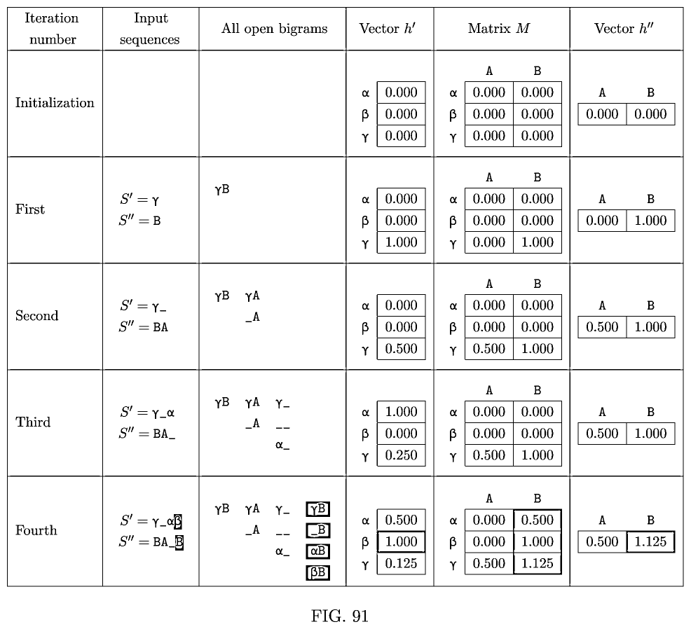

FIG. 91 illustrates how the encoding algorithm works for the two sequences shown in FIG. 87. This figure is similar to previous encoding examples. The new aspect is that now one or both sequences can have gaps in them, where the gaps are indicated with underscores. A gap in the first sequence, S′, means that no element of h′ will be incremented by 1 during that iteration (see the second iteration in the figure). A gap in S″, on the other hand, means that the matrix will not be updated during that iteration, i.e., h′ will not be added to any column of the matrix (see the third iteration in this example). A gap in S″ also suppresses the update of the vector h″ as shown in the third iteration. In this example, z is equal to 2.

The algorithm is similar to the previous encoding algorithms, but this one can handle sequences with gaps, while the previous ones cannot. The new modifications here are two if statements. The first one checks the incoming character on the sequence S′. If it is a gap, then the update of the vector h′ is skipped. The exponential decay of h′, however, is still performed at each iteration. The second if statement checks whether the incoming character on the sequence S″ is a gap, and if that is the case the updates of the vector h″ and the matrix M are skipped. The update of the helper variable z, however, is performed during all iterations. In other words, a gap in S″ will suppress the update of h″, but the magnitude of z, which will be added to h″ during the next iteration, will be properly updated.

6.2 The Decoding Algorithm (with Gaps)

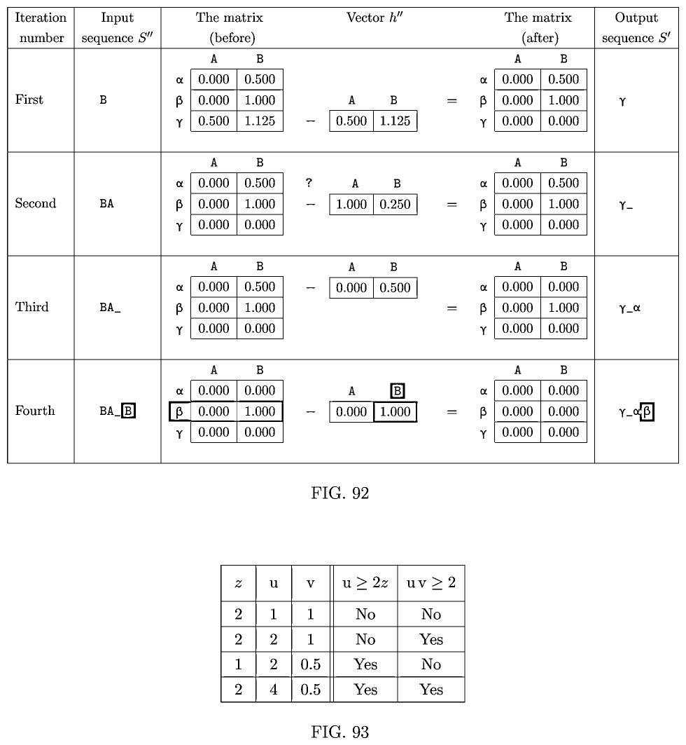

FIG. 92 gives a step-by-step example of the decoding algorithm. Each row of the figure corresponds to one decoding iteration. As with other decoding examples, the goal is to subtract the vector h″ from one row of the matrix without any matrix elements becoming negative. In all previous algorithms, however, if this subtraction was not possible from any row, then the decoding process was declared to be stuck and the short wrong sequence decoded so far was returned. This algorithm, on the other hand, outputs a gap for the current iteration and continues the decoding process. This is illustrated in the second iteration, when the vector h″ is too large to be subtracted from any row of the matrix. The output at that iteration is a gap (i.e., an underscore character) and the matrix remains the same. Note, however, that h″ is updated during that iteration, i.e., the A-th element is decremented by 1 and then both elements are multiplied by z=2.

Another feature of the algorithm is demonstrated on the third row of FIG. 92. Now the incoming character on the sequence S″ is a gap. In this case, the subtraction of 1 from h″ is suppressed. Both elements of h″, however, are still multiplied by 2 before the next iteration.

The decoding algorithm is similar in structure to other decoding algorithms. The new things here are two if statements. The first one checks if the candidate character for decoding is a gap. If it is, then the vector h″ is not subtracted from any row of the matrix during this iteration. The second if statement checks whether the incoming character on the sequence S″ is a gap. If that is the case, then no element of h″ is decremented during this iteration. The location of the gaps in S″, however, does not affect the multiplication of all elements of h″ by z, which is always performed in the main loop.

The matrix row from which to subtract h″ is selected similarly to the other decoding algorithms. However, this algorithm is modified to return a null if no suitable row can be identified. This null character is treated as a gap, which is appended it to the output sequence. Another modification checks if the vector h″ contains only zeros. This case is also treated as a gap by the main algorithm. This condition is added in order to handle sequences that end with gaps more uniformly. Thus, if for some reason h″ is depleted and contains only zeros, the algorithm will output only gaps until the length of the output sequence reaches T. An alternative implementation is also possible in which the algorithm terminates immediately and returns the sequence decoded so far.

The computational complexity of this version of the decoding algorithm is O(TM′M″). In other words, the main loop runs for T iterations and during each one of them it calls the helper function, which runs in O(M′M″) time. The extra check during the search for the next decoded character does not affect the overall complexity because summing the elements of h″ takes only O(M″) time. If this search is implemented to run in parallel, then the overall complexity of the algorithm can be reduced to O(TM″).

7 ZUV Algorithms for Sequences with Gaps

This chapter describes modifications to the ZUV algorithms, which were introduced in Chapter 5, that enable them to work with sequences with gaps. These modifications are similar to the modifications that were added to the algorithms described in Chapter 6. In this case, however, gaps are introduced in input sequences that are exponentially weighted.

7.1 ZUV Encoding Algorithm (with Gaps)

The ZUV encoding algorithm with gaps is similar to the original version. The difference is that the encoding algorithm now checks if the current character in either of the two sequences is empty, i.e., if it is a gap. If this is the case for the character from the sequence S′, then the update for the vector h′ is skipped. If the character from the sequence S″ is empty, then both the update of the vector h″ and the update of the matrix M are skipped.

7.2 ZUV Decoding Algorithm (with Gaps)

The ZUV decoding algorithm with gaps is similar to the non-gap version. In this version, however, the character that is decoded during the current iteration can be a gap. Also, the incoming character from the second sequence can be a gap as well. The corresponding updates of the matrix M and the vector h″ are skipped in these cases.

7.3 Evaluation Results for ZUV with Gaps

This section describes an evaluation of the ZUV decoding algorithm that focuses on the case when the sequences may contain gaps.

FIG. 93 summarizes the four different experimental conditions. The first three columns of the figure show the parameter values for z, u, and v. The last two columns show whether that particular set of parameters satisfy the two sufficient conditions that were derived in Chapter 5. FIGS. 94-97 show the evaluation results. Each of these four figures corresponds to one row of FIG. 93. The meaning of the eight plots in each figure was explained in Section 2.10.4.

FIG. 94 shows the results for z=2, u=1, and v=1. In this case both u≥2z and uv≥2 are not satisfied. As could be expected, the results show that as the sequence length increases the decoding performance degenerates.

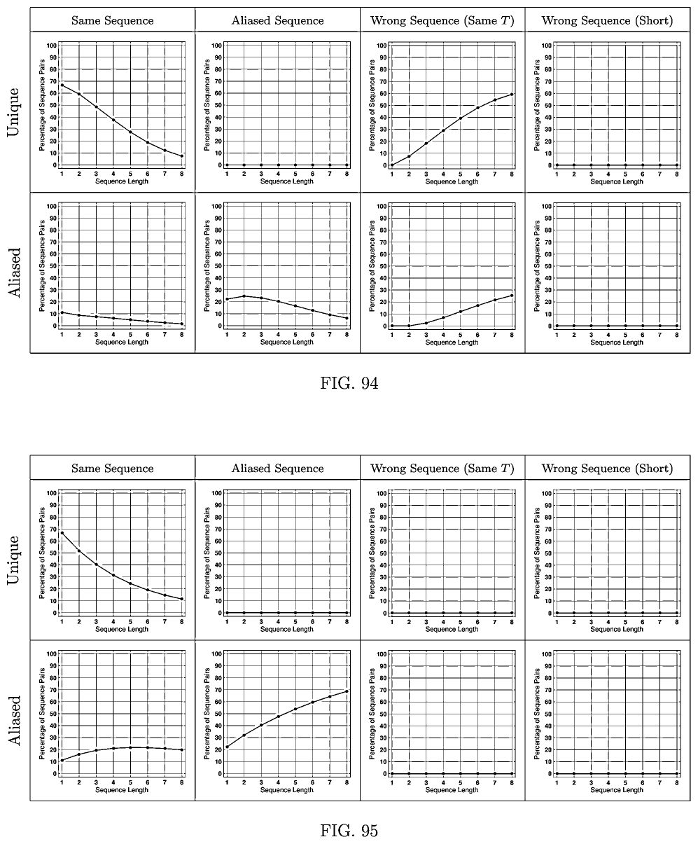

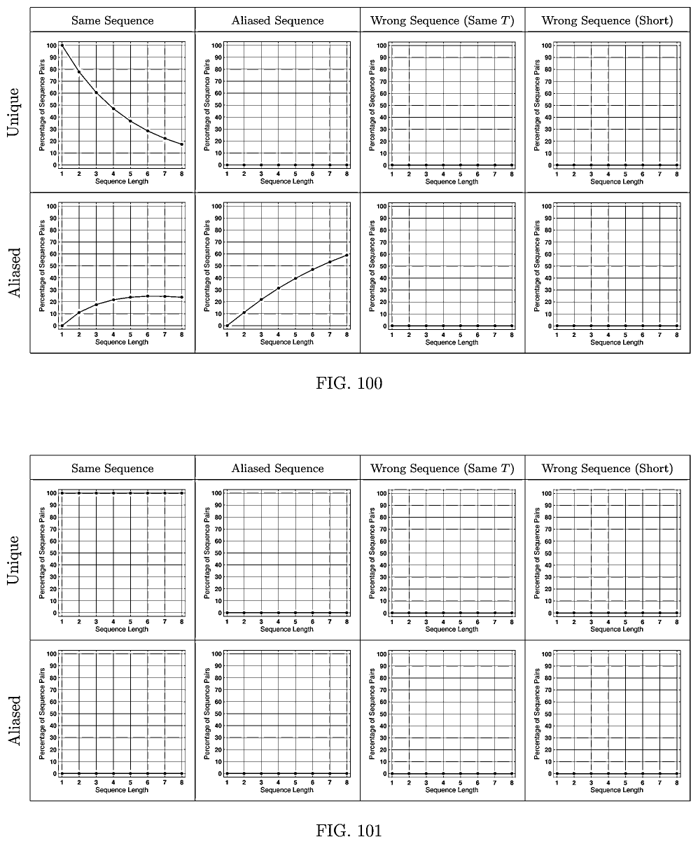

FIG. 95 shows another set of results for z=2, u=2, and v=1. In this case u≥2z is not satisfied, but uv≥2 is satisfied. If the decoding results without gaps would extend to decoding with gaps we would expect the decoding performance to be perfect. However, this is not the case. There are two reasons for that. First, there is no filtering of S″ sequences that end with gaps, which leads to aliasing. Second, the condition uv≥2 is not a sufficient condition for the case with gaps. This result is proven with the counter example in FIG. 100 (which has the S″ filter, and thus decouples the two types of aliasing).

FIG. 96 shows the next set of results for z=1, u=2, and v=0.5. The condition u≥2z is satisfied in this case. This condition is sufficient even in the case with gaps (see Section 7.6 below). The reason why the decoding is not perfect is that there is no filtering of S″ sequences that end with one or more gaps, which introduces aliasing. If this filter is applied, then all aliasing disappears and the decoding is perfect (see FIG. 101).

FIG. 97 shows the results for z=2, u=4, and v=0.5. In this case, both u≥2z and uv≥2 hold. Because the second condition is no longer sufficient for the case with gaps, the results are similar to those in FIG. 96. In fact, the last three columns of plots in both figures are identical. However, there is a difference in the first column. The fraction of aliased/same sequence pairs is greater in FIG. 97. This is due to h″ aliasing because in this case zv=1. This does not affect the decoding process because this aliasing is disambiguated when the S″ sequence is provided at run time.

In FIG. 96 and FIG. 97 an asymptotic fraction of the sequence pairs is encoded as aliased and decoded as aliased. This is due to the suffix in S″ that could end with gap(s).

7.4 Another Evaluation with Suffix Filtering of S″

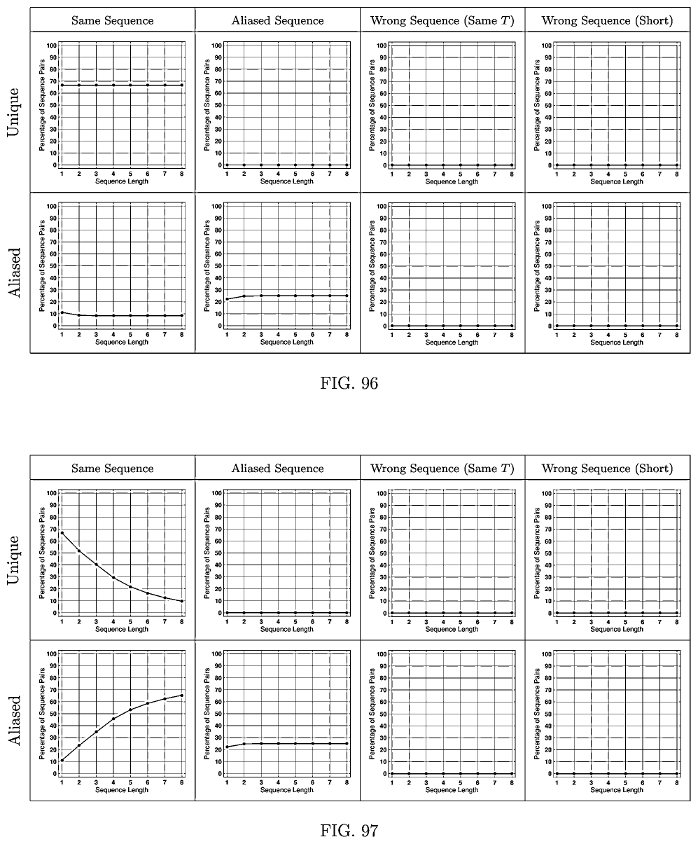

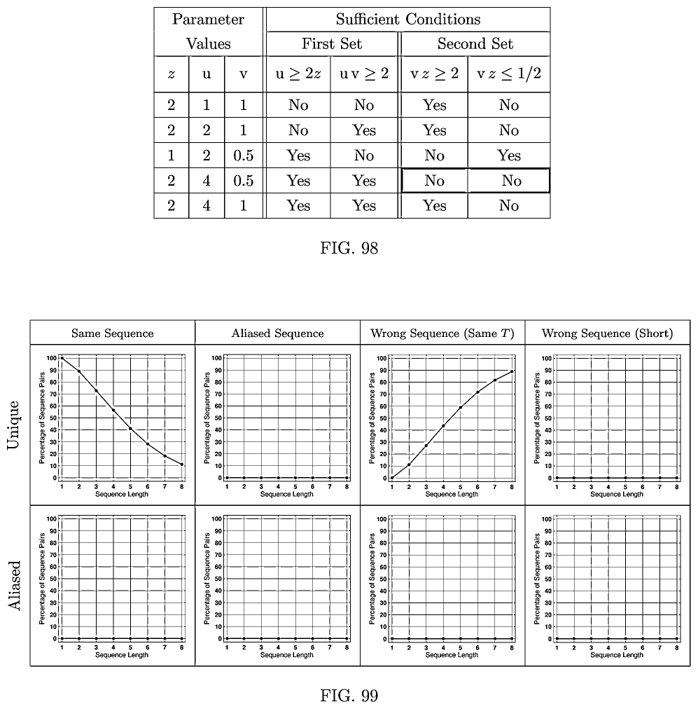

This section repeats the exhaustive enumeration analysis from the previous section, but now the sequence S″ cannot end with a gap (or several gaps in a row). FIG. 98 is an extended version of FIG. 93 in which two additional conditions are added: vz≥2 and vz≤½. These conditions control the aliasing of h″ (i.e., if one of them is satisfied, then there is no h″ aliasing). The five rows of this figure correspond to FIGS. 99-103.

FIG. 99 shows the evaluation results for z=2, u=1, and v=1. The values of these parameters correspond to the first row of FIG. 98. In this case, both u≥2z and uv≥2 are not satisfied. Because u=1 and v=1, this is a degenerate case that does not apply exponential weighting to the sequences.

FIG. 100 shows the results for the second row of FIG. 98 in which z=2, u=2, and v=1. In this case uv≥2 is satisfied, but, as mentioned above, this condition is no longer sufficient for the case with gaps. The aliasing that remains is due to aliasing of the matrix and not due to trailing gaps in S″.

FIG. 101 shows the third evaluation in which z=1, u=2, and v=0.5. In this case the condition u≥2z is satisfied. As shown below in Section 7.6 this is a sufficient condition for perfect ZUV decoding with gaps and suffix filtering of S″. This is confirmed by these results in which the plot in the upper-left is at 100% and all other plots are at 0%.

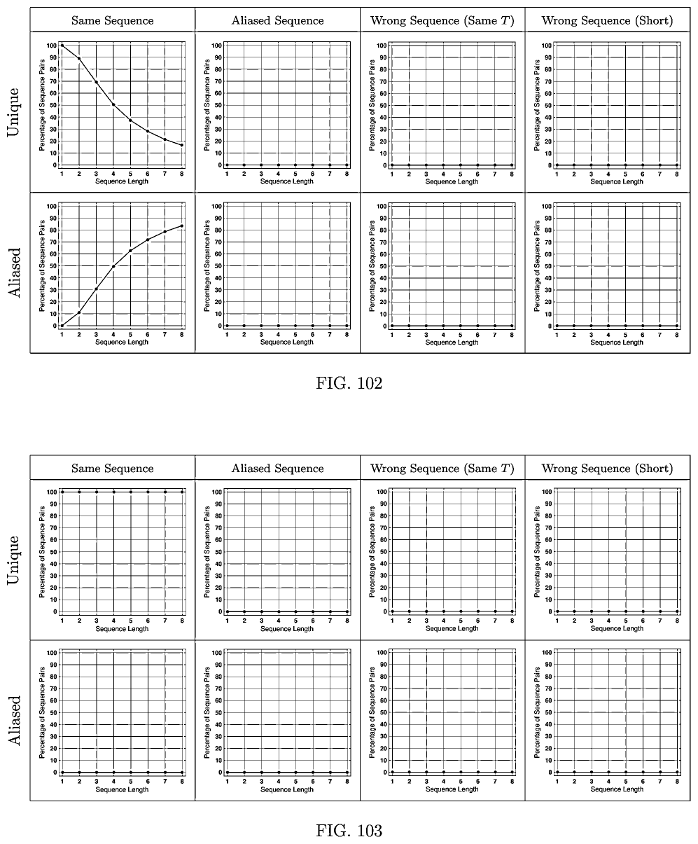

FIG. 102 shows the results of the fourth evaluation in which z=2, u=4, and v=0.5. Because the condition u≥2z is satisfied we would expect this figure to look the same as FIG. 101. This is not the case, however, because both vz≥2 and vz≤½ are not satisfied, which leads to h″ aliasing. Because the two plots in the first column of FIG. 102 sum up to 100%, this aliasing does not affect the decoding outcomes. In other words, the S″ sequence, which is provided at run time during decoding resolves the h″ aliasing and leads to perfect decoding.

To verify that this is indeed the reason for the aliasing reported in FIG. 102, we ran another set of experiments in which z=2, u=4, and v=1 (see the fifth row of FIG. 98). The results for this case are given in FIG. 103, which shows perfect decoding and confirms the previous conclusions. In this case, we have u≥2z and vz≥2. The first condition ensures perfect decoding and the second condition eliminates h″ aliasing.

All results in this chapter are for M′=2 and M″=2. It is sufficient to perform this analysis only for 2×2 matrices, because if a ZUV model is aliased for 2×2, then it will also be aliased for larger matrices, given that the values of z, u, and v remain the same. To summarize, if u≥2z and either vz≥2 of vz≤½, then the ZUV decoding will be perfect, provided that the S″ sequence does not end with a gap.

### 7.5 Example for T=3

This section gives an example with sequences of length three that shows how a condition for unambiguous decoding of the ZUV model can be derived. This example uses a pair of binary channels a and b, instead of using character sequences. These channels can be viewed as representations of sequences drawn from alphabets that consist of only one character. That is, zeros in a and b correspond to gaps and ones correspond to characters. This example covers only the initial iteration and shows that the first element of a is decoded correctly. The parameters z, u, and v are assumed to be non-zero real numbers.

Let T=3 and let a, b∈{0, 1}^{T} be two binary sequences of length T. In this case the ZUV decoding algorithm performs three iterations. For each iteration, the value of ^{d}h_{b}″ is given by the following equations:

$${}^{d} {h}_{b}^{″} [ 0 ] = {b}_{0} {v}^{0} {z}^{0} + {b}_{1} {v}^{- 1} {z}^{- 1} + {b}_{2} {v}^{- 2} {z}^{- 2} , \qquad ( 7.1 )$$

$${}^{d} {h}_{b}^{″} [ 1 ] = {b}_{1} {v}^{- 1} {z}^{0} + {b}_{2} {v}^{- 2} {z}^{- 1} , \qquad ( 7.2 )$$

$${}^{d} {h}_{b}^{″} [ 2 ] = {b}_{2} {v}^{- 2} {z}^{0} . \qquad ( 7.3 )$$

The value of ^{d}M_{a,b}[0] can be expressed as follows:

$$\begin{matrix}{}^{d} {M}_{a , b} [ 0 ] = {\sum }_{i = 0}^{2} {a}_{i} {u}^{- i} {}^{} {}^{d} {h}_{b}^{″} [ i ] \\ = {a}_{0} {u}^{0 d} {h}_{b}^{″} [ 0 ] + {a}_{1} {u}^{- 1 d} {h}_{b}^{″} [ 1 ] + {a}_{2} {u}^{- 2 d} {h}_{b}^{″} [ 2 ]\end{matrix} \qquad ( 7.4 )$$

If a₀=1, then the decoding constraint ^{d}M_{a,b}[0]−^{d}h_{b}″[0]≥0 is satisfied because ^{d}h_{b}″[0] is already a part of the sum in equation 7.4. Thus, we only need to focus on the case when a₀=0 and derive the conditions for which the constraint ^{d}M_{a,b}[0]−^{d}h_{b}″[0]≥0 is satisfied. That is, we need to find values for z, u, and v such that if a₀=0, then the following inequality must hold:

$${}^{d} {M}_{a , b} [ 0 ] < {}^{d} {h}_{b}^{″} [ 0 ] . \qquad ( 7.5 )$$

Expanding the value of ^{d}M_{a,b}[0] as specified by (7.4) transforms (7.5) into the following inequality:

$${a}_{0} {u}^{0} {}^{d} {h}_{b}^{″} [ 0 ] + {a}_{1} {u}^{- 1 d} {h}_{b}^{″} [ 1 ] + {a}_{2} {u}^{- 2 d} {h}_{b}^{″} [ 2 ] - {}^{d} {h}_{b}^{″} [ 0 ] < 0. \qquad ( 7.6 )$$

Subsequently, we can plug a₀=0 and expand ^{d}h_{b}″[0], ^{d}h_{b}″[1], and ^{d}h_{b}″[2] as follows:

$${a}_{1} {u}^{- 1} ( {b}_{1} {v}^{- 1} {z}^{0} + {b}_{2} {v}^{- 2} {z}^{- 1} ) + {a}_{2} {u}^{- 2} ( {b}_{2} {v}^{- 2} {z}^{0} ) - ( {b}_{0} {v}^{0} {z}^{0} + {b}_{1} {v}^{- 1} {z}^{- 1} + {b}_{2} {v}^{- 2} {z}^{- 2} ) < 0. \qquad ( 7.7 )$$

The terms in the previous inequality are grouped by the elements of a. Instead, we can regroup them by the elements of b as shown below:

$$- {b}_{0} {v}^{0} {z}^{0} + {b}_{1} {v}^{- 1} ( {a}_{1} {u}^{- 1} {z}^{0} - {z}^{- 1} ) + {b}_{2} {v}^{- 2} ( {a}_{1} {u}^{- 1} {z}^{- 1} + {a}_{2} {u}^{- 2} {z}^{0} - {z}^{- 2} ) < 0 . \qquad ( 7.8 )$$

Furthermore, in each of the terms it is possible to rearrange the powers of u, v, and z so that the inequality is expressed using integer powers of (uv) and u/z:

$$- {b}_{0} {u}^{0} {{v}^{0} ( \frac{u}{z} )}^{0} + {b}_{1} {u}^{- 1} {v}^{- 1} ( {{a}_{1} ( \frac{u}{z} )}^{0} - {( \frac{u}{z} )}^{1} ) + {b}_{2} {v}^{- 2} {u}^{- 2} ( {{a}_{1} ( \frac{u}{z} )}^{1} + {{a}_{2} ( \frac{u}{z} )}^{0} - {( \frac{u}{z} )}^{2} ) < 0. \qquad ( 7.9 )$$

Multiplying both sides by −1, leads to the following alternative form:

$$\\ \begin{matrix}{{b}_{0} ( uv )}^{0} {( \frac{u}{𝓏} )}^{0} + {{b}_{1} ( uv )}^{- 1} ( {( \frac{u}{𝓏} )}^{1} - {{a}_{1} ( \frac{u}{𝓏} )}^{0} ) + {{b}_{2} ( uv )}^{- 2} ( {( \frac{u}{𝓏} )}^{2} - {{a}_{1} ( \frac{u}{𝓏} )}^{1} - {{a}_{2} ( \frac{u}{𝓏} )}^{0} ) > 0. & ( 7.1 )\end{matrix}$$

This inequality is easier to express if we let w=uv and x=u/z:

$$\\ \begin{matrix}{b}_{0} {w}^{{}_{} 0} ( {x}^{{}_{} 0} ) + {b}_{1} {w}^{{}_{} - 1} ( {x}^{{}_{} 1} - {a}_{1} {x}^{{}_{} 0} ) + {b}_{2} {w}^{{}_{} - 2} ( {x}^{{}_{} 2} - {a}_{1} {x}^{{}_{} 1} - {a}_{2} {x}^{{}_{} 0} ) > 0. & ( 7.11 )\end{matrix}$$

Furthermore, because a₁, a₂ ∈{0, 1}, a lower bound can be derived for the left-hand side of inequality (7.11). That is, if we set all a's to 1, then we get:

$$\\ \begin{matrix}{b}_{0} {w}^{{}_{} 0} ( {x}^{{}_{} 0} ) + {b}_{1} {w}^{{}_{} - 1} ( {x}^{{}_{} - 1} - {x}^{{}_{} 0} ) + {b}_{2} {w}^{{}_{} - 2} ( {x}^{{}_{} 2} - {x}^{{}_{} 1} - {x}^{{}_{} 0} ) > 0. & ( 7.12 )\end{matrix}$$

Therefore, a sufficient condition for (7.11) to be satisfied is for the left-hand side of the previous inequality to be positive.

Assuming that at least one of b₀, b₁, or b₂ is nonzero, which is required to have a nonzero M_{a,b}, the previous inequality holds if each of the three expressions in the parentheses is positive. In other words, a sufficient condition for (7.12) to hold is that the following system of inequalities holds:

$$\\ \begin{matrix}{x}^{{}_{} 0} > 0 , & ( 7.13 )\end{matrix}$$

$$\\ \begin{matrix}{x}^{{}_{} 1} - {x}^{{}_{} 0} > 0 , & ( 7.14 )\end{matrix}$$

$$\\ \begin{matrix}{x}^{{}_{} 2} - {x}^{{}_{} 1} - {x}^{{}_{} 0} > 0. & ( 7.15 )\end{matrix}$$

All three inequalities are satisfied if x>1.618. This constant, however, depends on the sequence length. If we let T→∞, then we can use an argument based on the formula for the sum of a geometric progression to prove that the larger system of inequalities is satisfied for each x≥2. In other words, a₀ will be correctly decoded provided that uv>0 and u/z≥2 and at least one bᵢ is 1 for i∈{0, 1, . . . , T−1}. This argument is generalized below to decoding of all elements of a using mathematical induction.

7.6 Decoding Theorems for the ZUV Model with Gaps

The following theorem proves that ū/z≥2 is a sufficient condition for the correct decoding of the first element a₀ of the binary sequence a, given the matrix element M_{a,b} and the vector element h_{b}″. The proof examines two cases depending on the value of a₀. If a₀=1, then the proof shows that M_{a,b}−h_{b}″≥0. On the other hand, if a₀=0, then the proof shows that M_{a,b}−h_{b}″<0. This is accomplished by using the formulas for the sum of a geometric progression to derive an upper bound for that difference.

The rest of this section uses [symbol] _{a*b}^{(u,v)}(z) to denote the value of the matrix element M_{a,b} and [symbol] _{b}^{(v)}(z) to denote the value of the vector element h_{b}″. This notation captures all parameters that affect these values, which makes it more convenient to use in the proofs. The superscripts capture the exponential weighting applied to the elements of the binary sequences a and b. This notation also uses [symbol] instead of [symbol] to denote the unilateral z-transform. More formally,

$$\\ \begin{matrix}{ℨ}^{+} {}_{a ★ b}^{{}_{} ( u , v )} ( 𝓏 ) = {M}_{a , b} = {\sum }_{n = 0}^{T - 1} {\sum }_{m = 0}^{T - 1 - n} \overline{{a}_{m} {u}^{{}_{} - m}} {b}_{m + n} {v}^{{}_{} - ( m + n )} {𝓏}^{{}_{} - n} , & ( 7.16 )\end{matrix}$$

$$\\ \begin{matrix}{ℨ}^{+} {}_{b}^{{}_{} ( v )} ( 𝓏 ) = {h}_{b}^{{}_{} ″} = {\sum }_{k = 0}^{T - 1} {b}_{k} {v}^{{}_{} - k} {𝓏}^{{}_{} - k} . & ( 7.17 )\end{matrix}$$

Theorem 7.1. Sufficient conditions for decoding of the first element of a binary sequence. Let a and b be two binary sequences of length T, i.e.,

$$\\ \begin{matrix}a = ( {a}_{0} , {a}_{1} , {a}_{2} , … , {a}_{T - 1} ) ∈ {\{ 0 , 1 \}}^{T} , & ( 7.18 )\end{matrix}$$

$$\\ \begin{matrix}b = ( {b}_{0} , {b}_{1} , {b}_{2} , … , {b}_{T - 1} ) ∈ {\{ 0 , 1 \}}^{T} . & ( 7.19 )\end{matrix}$$

Let the sequence b have at least one non-zero element, i.e., bᵢ=1 for at least one i∈{0, 1, . . . , T−1}. Also, let z, u, and v be three non-zero complex numbers such that ū/z≥2 and vz>0.
Let M_{a,b}= [symbol] _{a*b}^{(u,v)}(z) and let h_{b}″= [symbol] _{b}^{(v)}(z). Then, the value of a₀∈{0, 1} can be determined from the sign of the difference M_{a,b}−h_{b}″ as follows:

$$\\ \begin{matrix}{a}_{0} = \{ \begin{matrix}1 , & if {M}_{a , b} \geq {h}_{b}^{{}_{} ″} , \\ 0 , & if {M}_{a , b} < {h}_{b}^{{}_{} ″} .\end{matrix} & ( 7.2 )\end{matrix}$$

In other words, if M_{a,b}−h_{b}″ is non-negative, then a₀=1. On the other hand, if M_{a,b}−h_{b}″ is negative, then a₀=0.

Theorem 7.1 implies that the ZUV decoding algorithm always decodes the first element of S′ correctly whenever ū/z≥2 and vz>0. This is true even if the S″ sequence given to the algorithm at run time is not identical to the S″ sequence used for encoding.

The following theorem generalizes Theorem 7.1 to all elements of the binary sequence a.

### Theorem 7.2. Sufficient Conditions for Decoding of all Elements of a Binary Sequence.

Let T be a positive integer, i.e., T∈ [symbol] ={1, 2, . . . }. Let a=(a₀, a₁, a₂, . . . , a_{T-1})∈{0, 1}^{T} and let b=(b₀, b₁, b₂, . . . , b_{T-1})∈{0, 1}^{T} be two binary sequences of length T. Let the last element of b be equal to 1, i.e., b_{T-1}=1. Also, let z, u, and v be three non-zero complex numbers such that ū≥2z and vz>0.
Then, the element of a at index t can be decoded as follows:

$$\\ \begin{matrix}{a}_{t} = \{ \begin{matrix}1 , & if {ℨ}^{+} {}^{{}_{} ( u , v )} \{ a [ t , T - 1 ] ★ b [ t , T - 1 ] \} ( 𝓏 ) \geq {ℨ}^{+} {}^{{}_{} ( v )} \{ b [ t , T - 1 ] \} ( 𝓏 ) , \\ 0 , & if {ℨ}^{+} {}^{{}_{} ( u , v )} \{ a [ t , T - 1 ] ★ b [ t , T - 1 ] \} ( 𝓏 ) < {ℨ}^{+} {}^{{}_{} ( v )} \{ b [ t , T - 1 ] \} ( 𝓏 ) ,\end{matrix} & ( 7.21 )\end{matrix}$$

for all t∈{0, 1, 2, . . . , T−1}. In this formula a[t, T−1] and b[t, T−1] denote the suffixes of a and b that start from aₜ and bₜ, i.e.,

$$\\ \begin{matrix}a [ t , T - 1 ] = ( {a}_{t} , {a}_{t + 1} , {a}_{t + 2} , … , {a}_{T - 1} ) , & ( 7.22 )\end{matrix}$$

$$\\ \begin{matrix}b [ t , T - 1 ] = ( {b}_{t} , {b}_{t + 1} , {b}_{t + 2} , … , {b}_{T - 1} ) . & ( 7.23 )\end{matrix}$$

Note that the unilateral z-transform formulas in (7.21) map to the values of M_{a,b} and h_{b}″ at iteration t during decoding. That is, formula (7.21) is similar to (7.20), but it covers the general case. In other words, it covers not just the case when t=0, but also the cases when t=1, 2, . . . , T−1.

In general, the problem of decoding a given b, h_{b}″, M_{a,b}, z, u, and v is ill-posed. There may be many solutions. However, under the conditions of Theorem 7.2 the decoding problem is well-posed, i.e., there is a unique solution and the decoding of a is perfect.

The next theorem generalizes Theorem 7.2 to a complete ZUV matrix. It states that if ū≥2z, vz>0, and the last character of S″ is not a gap, then the decoding is perfect, given that S″ is provided at run time. That is, under these conditions, there is an unique decoding path and there is no need for additional constraints, e.g., row constraints, because each element is always in agreement with all other elements in the same matrix row.

Theorem 7.3. Let R and C be two positive integers. Let Γ′={φ₁, φ₂, . . . , φ_{R}} be an alphabet of size R. Let Γ″={γ₁, ψ₂, . . . , ψ_{C}} be an alphabet of size C. Let T be a positive integer. Let S′ be a sequence of length T that is drawn from Γ′ such that each element in S′ may be a gap, which is denoted with ϵ. More formally,

$$\\ \begin{matrix}{S}^{{}_{} ′} ∈ {\{ {Γ}^{{}_{} ′} ⋃ ε \}}^{T} . & ( 7.24 )\end{matrix}$$

Let S″ be a sequence of length T drawn from Γ″ such that each element of S″ may be a gap, except for the last element, which is not a gap. More formally,

$$\\ \begin{matrix}{S}^{{}_{} ″} ∈ {\{ {Γ}^{{}_{} ″} ⋃ ε \}}^{T - 1} \times {Γ}^{{}_{} ″} . & ( 7.25 )\end{matrix}$$

Let u, v, and z be three non-zero complex numbers such that the following two conditions are satisfied:

$$\\ \begin{matrix}( i ) & \\ \overline{u} / 𝓏 \geq 2 , & ( 7.26 )\end{matrix}$$

$$\\ \begin{matrix}( ii ) & \\ v 𝓏 > 0. & ( 7.27 )\end{matrix}$$

Let M be an SSM matrix and let h″ be its corresponding vector computed by the ZUV encoding algorithm. Let Ŝ′ be a sequence computed by the ZUV decoding algorithm from M, h″, and S″. Then, Ŝ′=S′.

### 7.7 Distributed ZUV Encoding

This section states distributed versions of the ZUV algorithms. The encoding version is distributed by the elements of the matrix. The decoding version is distributed by the rows of the matrix.

The distributed ZUV encoding algorithm encodes just one matrix element, which is denoted with m to distinguish it from the entire matrix M. To encode the whole matrix one needs to run a separate instance of this algorithm for each matrix element. This distributed encoding possibility was mentioned several times. In fact, all encoding formulas were derived for a channel pair, where the two channels were called a and b. In this implementation the binary channel pair is (s′, s″). Note that these are labeled with small letters to distinguish them from S′ and S″, which denote character sequences. The complexity of this algorithm is O(T), where T is the length of both s′ and s″.

### 7.8 Distributed ZUV Decoding

The computation in the ZUV decoding algorithm can be distributed by rows. To decode the entire matrix the distributed ZUV decoding algorithm decodes each row in parallel.

The algorithm has 6 inputs. The first input is m, which is an array that holds the values of the matrix elements in one row of the matrix. The second argument is the vector h″. The third argument is S″, which is the English sequence represented as a set of binary channels. Note that it is 2D in this case and the indexing is S_{j,t}″, where j is one of the M″ channels and t is the current index into all channels. The last three arguments are z, u, and v, which control the exponential decay as usual. In this case, however, these can be arrays, not just numbers. Thus, this algorithm makes it possible to handle the case in which each element of the matrix has a different z, u, and v.

### 8 Continuous-Time Formulation for Spike Trains

This chapter derives the mathematical expressions for the SSM representation when the inputs are spike trains instead of discrete sequences. In this continuous-time formulation the temporal distances between the spikes are represented with real numbers and not with integers as in the discrete case. The proofs are analogous to the proofs in the discrete-time case, but now the formulas use functions instead of sequences.

### 8.1 The Continuous Cross-Correlation

The continuous cross-correlation has similar properties to the discrete cross-correlation, but it works with functions of time instead of discrete sequences. This section defines this operation and states some of its basic properties.

Definition 8.1. Let f(t) and g(t) be two complex functions that have one real argument t. The continuous cross-correlation of f and g, which is denoted by (f*g), is defined as:

$$\\ \begin{matrix}( f ★ g ) ( t ) = {\int }_{- ∞}^{∞} \overline{f ( τ )} g ( τ + t ) d τ , & ( 8.1 )\end{matrix}$$

where f̅(̅τ̅)̅ denotes the complex conjugate of the value of the function f at τ.

The result of the continuous cross-correlation is a function, which is called the cross-correlation function (CCF). Formula (8.1) gives the value of the CCF at only one point. To get all values of this function we need to evaluate this formula for all real t.

If both f and g are real-valued functions, then the definition simplifies to:

$$\\ \begin{matrix}( f ★ g ) ( t ) = {\int }_{- ∞}^{∞} f ( τ ) g ( τ + t ) d τ . & ( 8.2 )\end{matrix}$$

In other words, the value of the function f no longer has to be conjugated. In fact, only f needs to be a real function; g can still be a complex-valued function.

Property 8.2. The continuous cross-correlation is additive in both of its arguments. That is,

$$\\ \begin{matrix}( x + y ) ★ ( u + v ) = x ★ u + x ★ v + y ★ u + y ★ v , & ( 8.3 )\end{matrix}$$

where x, y, u, and v are complex functions with one real argument. This expression is true if the four cross-correlations in the right-hand side are well defined, i.e., given that the following four inequalities hold:

$$\vert {\int }_{- ∞}^{∞} \overline{x ( τ )} u ( τ + t ) d τ \vert < ∞ and \vert {\int }_{- ∞}^{∞} \overline{x ( τ )} v ( τ + t ) d τ \vert < ∞ , \qquad ( 8.4 )$$

$$\vert {\int }_{- ∞}^{∞} \overline{y ( τ )} u ( τ + t ) d τ \vert < ∞ and \vert {\int }_{- ∞}^{∞} \overline{y ( τ )} v ( τ + t ) d τ \vert < ∞ . \qquad ( 8.5 )$$

### 8.2 The Laplace Transform

This section defines the Laplace transform and states some of its properties that are relevant to the topic of this chapter.

Definition 8.3. Let f(t) be a complex function of a real argument. Then, the Laplace transform of the function f(t) is defined as follows:

$${ℒ}_{f} ( s ) = {\int }_{0 -}^{∞} f ( t ) {e}^{- s t} dt , \qquad ( 8.6 )$$

where s is a complex number.

If f(t)=0 for t<0, then the Laplace transform can also be defined as follows:

$${ℒ}_{f} ( s ) = {\int }_{0}^{∞} f ( t ) {e}^{- s t} dt . \qquad ( 8.7 )$$

The notation [symbol] {f(t)} or [symbol] {f} is typically used to denote the Laplace transform of the function f(t). This notation, however, is for the entire transform, which includes all values of s. In many of our formulas, however, we need only one value of the transform at one specific s, e.g., s=1. To specify that we can add an extra set of parentheses, i.e., [symbol] {f}(s). We will use this notation in some of the formulas, but it is somewhat cumbersome. More often we will use a simpler notation in which the curly brackets are omitted and the function name is used as a subscript of [symbol] . That is, [symbol] _{f}(s) will be used to denote the value of the Laplace transform of the function f(t), where the transform is evaluated at s.

Definition 8.4. The bilateral Laplace transform of the function f(t) is defined as follows:

$${ℬ}_{f} ( s ) = {\int }_{- ∞}^{∞} f ( t ) {e}^{- s t} dt , \qquad ( 8.8 )$$

where s is a complex number. The lower limit of the integral is now −∞ instead of 0⁻.

In the discrete case we used two different symbols, [symbol] and [symbol] ⁺, to denote the bilateral and the unilateral z-transform of a sequence. In the continuous case the accepted notation is to use [symbol] for the unilateral Laplace transform, which is typically called simply the Laplace transform. The bilateral Laplace transform is rarely used, but in order to distinguish between the two the symbol [symbol] is often used. In other words, to complete the analogy with the discrete case, [symbol] corresponds to [symbol] ⁺ and [symbol] corresponds to [symbol] .

Property 8.5. The Laplace transform is a linear operation. In other words,

$${ℒ}_{f + g} ( s ) = {ℒ}_{f} ( s ) + {ℒ}_{g} ( s ) , \qquad ( 8.9 )$$

provided that [symbol] _{f}(s) and [symbol] _{g}(s) are well defined. Also, if c is a complex scalar, then

$${ℒ}_{c f} ( s ) = c {ℒ}_{f} ( s ) . \qquad ( 8.1 )$$

Property 8.6. The Laplace transform of the Heaviside function H(t) is equal to 1/s. That is,

$${ℒ}_{H} ( s ) = \frac{1}{s} , \qquad ( 8.11 )$$

where H(t) is defined using the following formula:

$$H ( t ) = \{ \begin{matrix}0 , & if t < 0 , \\ 1 , & if t \geq 0 .\end{matrix} \qquad ( 8.12 )$$

### Theorem 8.7. The Right-Shift Theorem for the Laplace Transform.

Let f(t) be a bounded Laplace-transformable function and let a be a nonnegative real number. That is domain ( [symbol] _{f})≠∅ and |f(t)|≤M for each t∈ [symbol] . Furthermore, let g(t) be the function obtained by shifting f by a to the right and setting g(t) to zero for all t<a, i.e.,

$$g ( t ) = \{ \begin{matrix}0 , & if t < a , \\ f ( t - a ) , & if t \geq a .\end{matrix} \qquad ( 8.13 )$$

Then, for each s in the domain of the Laplace transform of f, the value of the Laplace transform of g at s can be obtained by multiplying the value of the Laplace transform of f at s by e^{-as}. More formally,

$${ℒ}_{g} ( s ) = {e}^{- a s} {ℒ}_{f} ( s ) , for each s ∈ domain ( {ℒ}_{f} ) . \qquad ( 8.14 )$$

### Theorem 8.8. The Left-Shift Theorem for the Laplace Transform.

Let f(t) be a Laplace-transformable function and let a be a nonnegative real number, i.e., a≥0. Also, let g(t) be the function obtained by shifting f by a to the left. That is, for each t∈

[symbol]

$$g ( t ) = f ( t + a ) . \qquad ( 8.15 )$$

Then, for each s in the domain of the Laplace transform of f the value of the Laplace transform of g at s can be computed using the following formula:

$${ℒ}_{g} ( s ) = {e}^{a s} ( {ℒ}_{f} ( s ) - {\int }_{0 -}^{{a}^{-}} f ( t ) {e}^{- s t} dt ) , for each s ∈ domain ( {ℒ}_{f} ) . \qquad ( 8.16 )$$

### 8.3 Dirac's Delta

The delta function, which is also often called Dirac's delta, is the standard way to model an impulse. Dirac's delta is usually modeled as the limit of a sequence of template functions of decreasing width and increasing height. The following definition introduces one such sequence.

Definition 8.9. The model δ for approximating Dirac's delta is defined as the following sequence of functions (δ₁(t), δ₂(t), . . . , δₙ(t), . . . ), where δₙ(t) denotes the following template function:

$${δ}_{n} ( t ) = \{ \begin{matrix}0 , & if t < - \frac{1}{2 n} , \\ n , & if - \frac{1}{2 n} \leq t \leq \frac{1}{2 n} , \\ 0 , & if t > \frac{1}{2 n} .\end{matrix} \qquad ( 8.17 )$$

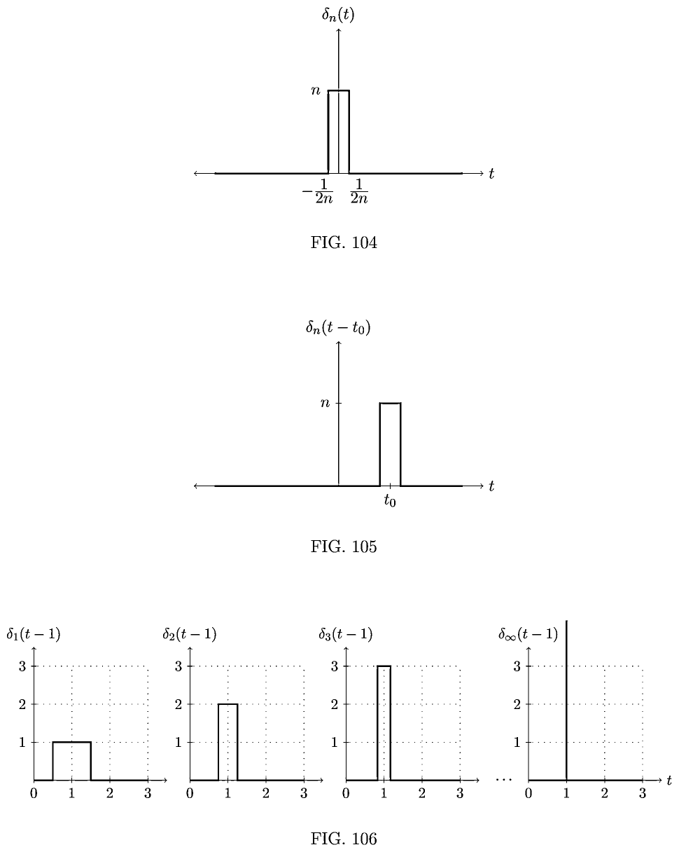

FIG. 104 shows a plot of δₙ(t). In this model, the nonzero part of the template function has a value of n. The width of the curve is 1/n, centered around the vertical axis. The area under the curve is equal to 1. Note that δₙ is an even function, i.e., δₙ(t)=δₙ(−t).

Definition 8.10. The Laplace transform of δ is defined as the function obtained by taking the limit of the sequence of Laplace transforms of each function in the model sequence for δ as defined in Definition 8.9. That is,

$${ℒ}_{δ} ( s ) = {lim}_{n \to ∞} {ℒ}_{{δ}_{n}} ( s ) = {lim}_{n \to ∞} {\int }_{0 -}^{∞} {δ}_{n} ( t ) {e}^{- s t} dt . \qquad ( 8.18 )$$

Property 8.11. The Laplace transform of Dirac's delta is equal to 1 for any s, i.e., [symbol] {δ(t)}(s)=1.

Note that Property 8.11 is true only if the lower limit of the integral is 0⁻, which is how the Laplace transform is defined. If that limit is set to 0, then only the right half of the template δₙ will be included in the region of integration and the result will be ½ instead of 1, i.e.,

$${lim}_{n \to ∞} {\int }_{0}^{∞} {δ}_{n} ( t ) {e}^{- s t} d t = {lim}_{n \to ∞} {\int }_{0}^{\frac{1}{2 n}} n {e}^{- s t} d t = {lim}_{n \to ∞} n ( \frac{{e}^{- \frac{s}{2 n}}}{- s} - \frac{1}{- s} ) = {lim}_{n \to ∞} \frac{{e}^{- \frac{s}{2 n}} - 1}{- s {n}^{- 1}} = {lim}_{n \to ∞} \frac{\frac{1}{2} {e}^{- \frac{s}{2 n}}}{} = \frac{1}{2} {lim}_{n \to ∞} ( {e}^{- \frac{s}{2 n}} ) = \frac{1}{2} . \qquad ( 8.19 )$$

The formulation described so far can be used to model a single spike and only if this spike is at time t=0. To model a spike at t=t₀ we can shift the template function δₙ(t) by t₀ to the right, i.e., we can use δₙ(t−t₀). This shifted template function, which is shown in FIG. 105, can be used to model the shifted Dirac's delta. The formal definition is given below.

Definition 8.12. The model δ(t−t₀) for approximating a shifted Dirac's delta is defined as the sequence of functions (δ₁(t−t₀), δ₂(t−t₀), . . . , δₙ(t−t₀), . . . ), where t₀ is the offset and δₙ(t−t₀) denotes the following shifted template function:

$${δ}_{n} ( t - {t}_{0} ) = \{ \begin{matrix}0 , & if t < {t}_{0} - \frac{1}{2 n} , \\ n , & if {t}_{0} - \frac{1}{2 n} \leq t \leq {t}_{0} + \frac{1}{2 n} , \\ 0 , & if t > {t}_{0} + \frac{1}{2 n} .\end{matrix} \qquad ( 8.2 )$$

FIG. 106 illustrates the shape of δₙ(t−t₀) for different values of n. The shift t₀ is equal to 1 in this case. In the limit when n→→ the curve is visualized as an idealized impulse.

Definition 8.13. The Laplace transform of δ shifted by to is defined as the function obtained by taking the limit of the sequence of Laplace transforms of each function in the model sequence for shifted δ as defined in Definition 8.12. More formally,

$$ℒ \{ δ ( t - {t}_{0} ) \} ( s ) = {lim}_{n \to ∞} ℒ \{ {δ}_{n} ( t - {t}_{0} ) \} ( s ) = {lim}_{n \to ∞} {\int }_{0 -}^{∞} {δ}_{n} ( t - {t}_{0} ) {e}^{- st} dt \qquad ( 8.21 )$$

Property 8.14. The Laplace transform of a shifted Dirac's delta is equal to:

$$ℒ \{ δ ( t - {t}_{0} ) \} ( s ) = \{ \begin{matrix}{e}^{- {st}_{0}} , & if {t}_{0} \geq 0 , \\ 0 , & if {t}_{0} < 0 .\end{matrix} \qquad ( 8.22 )$$

Theorem 8.15. Let f(t) be a complex function of a real argument and let t₀∈R be a real number such that the limit L of f(t) as t→t₀ exists and is finite, i.e.,

$$ℒ = {lim}_{t \to {t}_{0}} f ( t ) , \qquad ( 8.23 )$$

$$such that$$

$$\vert ℒ \vert < ∞ .$$

### Then,

$${lim}_{n \to ∞} {\int }_{- ∞}^{∞} {δ}_{n} ( t - {t}_{0} ) f ( t ) dt = {lim}_{t \to {t}_{0}} f ( t ) , \qquad ( 8.24 )$$

provided that the limit in the left-hand side of (8.24) is well defined.
Theorem 8.16. Let f t be a complex function of a real argument that is continuous at t₀∈ [symbol] , i.e.,

$${lim}_{t \to {t}_{0}} f ( t ) = f ( {t}_{0} ) .$$

### Then,

$${lim}_{n \to ∞} {\int }_{- ∞}^{∞} {δ}_{n} ( t - {t}_{0} ) f ( t ) dt = f ( {t}_{0} ) . \qquad ( 8.25 )$$

### 8.4 Modeling Spikes and Spike Trains

A spike is an event that has a limited temporal extent. We will model a spike that occurs at time to with a shifted Dirac's delta. The model for approximating the shifted Dirac's delta was defined in Section 8.3 as a sequence of progressively narrowing and peaking template functions δₙ(t−t₀) as n→∞, where each shifted template function is defined as:

$${δ}_{n} ( t - {t}_{0} ) = \{ \begin{matrix}0 , & if t < {t}_{0} - \frac{1}{2 n} , \\ n , & \begin{matrix}if {t}_{0} - \frac{1}{2 n} \leq t \leq {t}_{0} + \frac{1}{2 n} , \\ \end{matrix} \\ 0 , & if t > {t}_{0} + \frac{1}{2 n} .\end{matrix} \qquad ( 8.26 )$$

A spike train is a collection of spikes that are generated on the same channel. We will use the notation b=(b₁, b₂, . . . , b_{K}) to denote a spike train b that has K spikes that occur at times b₁, b₂, . . . , b_{K}. This notation assumes that the spike times are sorted in increasing order and that there are no duplicates in this list. We will model the spike train b as a sequence of functions b⁽ⁿ⁾(t), where each function is obtained by summing K shifted template functions δₙ(t−bₖ). The following definition states this more formally.

Definition 8.17. The model for a spike train b=(b₁, b₂, . . . , b_{K}), where b₁, b₂, . . . , b_{K} specify the times of individual spikes, is the sequence of functions (b⁽¹⁾(t), b⁽²⁾(t), . . . , b⁽ⁿ⁾(t), . . . ), where

$${b}^{( n )} ( t ) = {\sum }_{k = 1}^{K} {δ}_{n} ( t - {b}_{k} ) , \qquad ( 8.27 )$$

$$for each$$

$$n ∈ ℕ = \{ 1 , 2 , … \} .$$

By analogy we can define the spike train a=(a₁, a₂, . . . , a_{J}) that contains J spikes that occur at times a₁, a₂, . . . , a_{J} as the sum of J shifted template functions, where the shifts are equal to the times at which the spikes occur. In other words,

$${a}^{( m )} ( t ) = {\sum }_{j = 1}^{J} {δ}_{m} ( t - {a}_{j} ) , \qquad ( 8.28 )$$

$$for each$$

$$m ∈ ℕ = \{ 1 , 2 , … \} .$$

In this case the number of spikes is J and the shifted template function is δₘ, which is defined as

$${δ}_{m} ( t - {t}_{0} ) = \{ \begin{matrix}0 , & if t < {t}_{0} - \frac{1}{2 m} , \\ m , & if {t}_{0} - \frac{1}{2 m} \leq t \leq {t}_{0} + \frac{1}{2 m} , \\ 0 , & if t > {t}_{0} + \frac{1}{2 m} .\end{matrix} \qquad ( 8.29 )$$

Note that this chapter uses 1-based indexing for the spikes in the spike train, while the previous chapters used 0-based indexing for the elements of a sequence. Another difference is that in the discrete case there is a one-to-one correspondence between the index of an element and its temporal location in the sequence. In the continuous case the index of the spike does not correspond to the time at which the spike occurs. It is just an index into a list of times that don't occur at regular intervals and there is no formula for converting from spike indices to spike times. In other words, aⱼ is the time at which the j-th spike occurred on channel a and j is just the index of that spike in the list of times that specify the spike train a=(a₁, a₂, . . . , a_{J}).

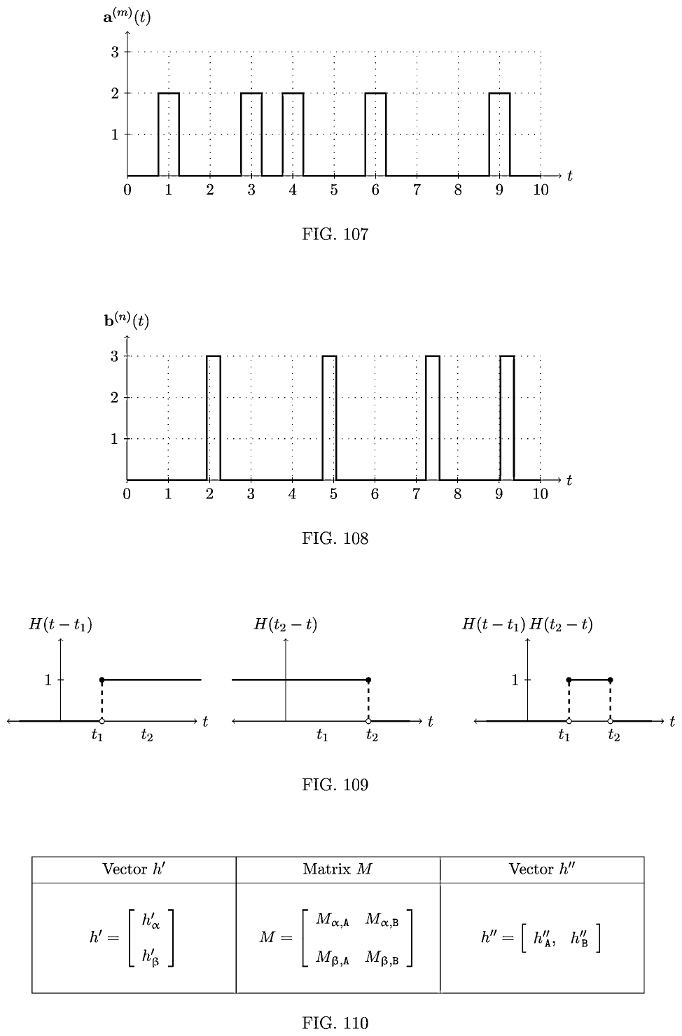

FIG. 107 gives an example with the spike train a=(a₁, a₂, a₃, a₄, a₅) that has five spikes. Each of these spikes is modeled with a shifted template function δₘ(t−aⱼ) where m=2. FIG. 108 gives another example with the spike train b=(b₁, b₂, b₃, b₄), in which each spike is modeled with a shifted template function δₙ(t−bₖ). In this case n is equal to 3, which makes the templates more narrow and more peaked than the templates used in FIG. 107.

### 8.5 Operations on Spike Trains

This section defines some operations on spike trains and pairs of spike trains. These operations are used and extended in later sections.

### 8.5.1 The Laplace Transform of a Spike Train

As described above, a spike train can be approximated with a sum of shifted template functions. For example, the spike train a=(a₁, a₂, . . . , a_{J}), which has J spikes that occur at times a₁, a₂, . . . , a_{J}, can be approximated with the function a^{(m)}=(a₁, a₂, . . . , a_{J}) in which each spike is modeled with the shifted template function δₘ(t−aⱼ) that was defined in formula (8.29). For each m<<∞ the template δₘ has a nonzero width and a^{(m)} can be treated just like any regular function. In particular, the Laplace transform of a^{(m)} can be evaluated using the standard formula. As m approaches infinity, however, the Laplace transform of the spike train is defined as shown below.

Definition 8.18. The Laplace transform of a spike train a=(a₁, a₂, . . . , a_{J}), where a₁, a₂, . . . , a_{J} specify the times of the spikes, is a function obtained by taking the limit of the sequence of Laplace transforms of functions in the model for the spike train a. More formally,

$${ℒ}_{a} ( s ) = {lim}_{m \to ∞} {ℒ}_{a ( m )} ( s ) = {lim}_{m \to ∞} {\int }_{0 -}^{∞} {a}^{( m )} ( t ) {e}^{- st} dt . \qquad ( 8.3 )$$

In other words, the Laplace transform of the spike train a can be obtained from its approximation a^{(m)}, in which shifted templates δₘ of height m and width 1/m are used to model the spikes, and then taking the limit as m→∞. This derivation is shown below:

$$\begin{matrix}{ℒ}_{a} ( s ) = {lim}_{m \to ∞} {\int }_{0 -}^{∞} {a}^{( m )} ( t ) {e}^{- st} dt \\ = {lim}_{m \to ∞} {\int }_{0 -}^{∞} {\sum }_{j = 1}^{J} {δ}_{m} ( t - {a}_{j} ) {e}^{- st} dt \\ = {\sum }_{j = 1}^{J} {lim}_{m \to ∞} {\int }_{0 -}^{∞} {δ}_{m} ( t - {a}_{j} ) {e}^{- st} dt \\ = {\sum }_{j = 1}^{J} {lim}_{m \to ∞} {\int }_{- ∞}^{∞} H ( t - {0}^{-} ) {δ}_{m} ( t - {a}_{j} ) {e}^{- st} dt \\ = {\sum }_{j = 1}^{J} H ( {a}_{j} - 0 ) ( {lim}_{m \to ∞} {\int }_{- ∞}^{∞} {δ}_{m} ( t - {a}_{j} ) {e}^{- st} dt ) ( Theorem 8.16 ) \\ = {\sum }_{j = 1}^{J} H ( {a}_{j} ) {e}^{- {sa}_{j}} .\end{matrix} \qquad ( 8.31 )$$

The Heaviside function is used to change the lower bound of the integral from 0⁻ to −∞ in the fourth line of formula (8.31). The value of the integral remains the same because everything in the interval −∞ to 0⁻ will be multiplied by 0, i.e., H(t−0⁻)=0 for t<0⁻. Note that 0⁻ is used in both the integral and H to prevent cutting the δ-templates in half if aⱼ=0.

To summarize, the value of the Laplace transform at s of the spike train a=(a₁, a₂, . . . , a_{J}), which has J spikes, is equal to:

$${ℒ}_{a} ( s ) = {\sum }_{j = 1}^{J} H ( {a}_{j} ) {e}^{- {sa}_{j}} . \qquad ( 8.32 )$$

If we assume that aⱼ≥0 for all j=1, 2, . . . , J (i.e., if we assume that the spike train is causal), then the Heaviside function always evaluates to 1 and the previous expression simplifies to:

$${ℒ}_{a} ( s ) = {\sum }_{j = 1}^{J} {e}^{- {sa}_{j}} . \qquad ( 8.33 )$$

That is, [symbol] ₐ(s) is equal to the sum of J exponentials of the form e^{-sa}^{ⱼ}, where the complex variable s is the argument of the transform and aⱼ is the time at which the j-th spike occurred.

By analogy, the value of the Laplace transform at s of the spike train b=(b₁, b₂, . . . , b_{K}), which has K spikes, is equal to:

$${ℒ}_{b} ( s ) = {\sum }_{k = 1}^{K} H ( {b}_{k} ) {e}^{- {sb}_{k}} . \qquad ( 8.34 )$$

Once again, if bₖ≥0 for all k=1, 2, . . . , K, then the Heaviside function is equal to 1 and this formula simplifies to:

$${ℒ}_{b} ( s ) = {\sum }_{k = 1}^{K} {e}^{- {sb}_{k}} . \qquad ( 8.35 )$$

### 8.5.2 The Cross-Correlation of Two Spike Trains

This section gives a mathematical formulation for the cross-correlation of two different spike trains. Let a=(a₁, a₂, . . . , a_{J}) be the first spike train, which consists of J spikes that occur at times a₁, a₂, . . . , a_{J}. Similarly, let b=(b₁, b₂, . . . , b_{K}) be the second spike train, which has K spikes that occur at times b₁, b₂, . . . , b_{K}.

The spikes on the first spike train will be modeled with the template function δₘ, which is defined as:

$${δ}_{m} ( t ) = \{ \begin{matrix}0 , & if t < - \frac{1}{2 m} , \\ m , & if - \frac{1}{2 m} \leq t \leq \frac{1}{2 m} , \\ 0 , & if t > \frac{1}{2 m} .\end{matrix} \qquad ( 8.36 )$$

The spikes on the second spike train will be modeled with a different template function, δₙ, which is defined as:

$${δ}_{n} ( t ) = \{ \begin{matrix}0 , & if t < - \frac{1}{2 n} , \\ n , & if - \frac{1}{2 n} \leq t \leq \frac{1}{2 n} , \\ 0 , & if t > \frac{1}{2 n} .\end{matrix} \qquad ( 8.37 )$$

In this case, n determines the height of the template for the second spike train, which may be different from the height m of the template for the first spike train.

As described above, the notation a^{(m)}=(a₁, a₂, . . . , a_{J}) will be used to denote the approximation for the spike train a that is modeled with the template δₘ. The value of a^{(m)}(t) is given by:

$${a}^{( m )} ( t ) = {\sum }_{j = 1}^{J} {δ}_{m} ( t - {a}_{j} ) . \qquad ( 8.38 )$$

Similarly, the notation b⁽ⁿ⁾=(b₁, b₂, . . . , b_{K}) denotes an approximation for the spike train b that uses the template δₙ. This approximation can be represented as follows:

$${b}^{( n )} ( t ) = {\sum }_{k = 1}^{K} {δ}_{n} ( t - {b}_{k} ) . \qquad ( 8.39 )$$

The cross-correlation of a^{(m)} and b⁽ⁿ⁾ is formally defined below. Note that in (8.40) the conjugation in a̅^̅{̅(̅m̅)̅}̅(̅τ̅)̅ can be dropped because δₘ is real and conjugation only affects complex numbers.

Definition 8.19. A model for the cross-correlation of two spike trains a=(a₁, a₂, . . . , a_{J}) and b=(b₁, b₂, . . . , b_{K}) is formed by functions (a^{(m)}*b⁽ⁿ⁾)(t), where m, n∈ [symbol] ={1, 2, . . . } such that

$$( {a}^{( m )} ⋆ {b}^{( n )} ) ( t ) = {\int }_{- ∞}^{∞} \overline{{a}^{( m )} ( τ )} {b}^{( n )} ( τ + t ) d τ = {\int }_{- ∞}^{∞} {a}^{( m )} ( τ ) {b}^{( n )} ( τ + t ) d τ . \qquad ( 8.4 )$$

For any m<<∞ and any n<<∞ the templates δₘ and δₙ have some temporal extent and the integral in (8.40) can be evaluated for a specific value of t in the usual way. For the cross-correlation of idealized spike trains, however, a different approach is needed that can be applied when there are two limits, i.e., when m→∞ and n→∞. In this case, both δₘ and δₙ tend to the delta function δ, but they do this independently of each other. This is addressed more formally in the next section in the context of the Laplace transform.

### 8.5.3 The Laplace Transform of the Cross-Correlation of Two Spike Trains

The Laplace transform of the cross-correlation of two spike trains a and b is defined using iterated limits of the Laplace transform of the cross-correlation of a^{(m)} and b⁽ⁿ⁾ as the width of the template δₘ and the width of the template δₙ tend to zero. A formal definition is stated below.

Definition 8.20. The Laplace transform of the cross-correlation of two spike trains that am given by a=(a₁, a₂, . . . , a_{J}) and b=(b₁, b₂, . . . , b_{K}) is a function obtained by taking the iterated limit over Laplace transforms of the cross-correlation functions in the model for the cross-correlation of a^{(m)} and b⁽ⁿ⁾ as m and n approach infinity. More formally,

$${ℒ}_{a ⋆ b} ( s ) = {lim}_{m \to ∞} {lim}_{n \to ∞} {ℒ}_{{a}^{( m )} * {b}^{( n )}} ( s ) = {lim}_{m \to ∞} {lim}_{n \to ∞} {\int }_{0 -}^{∞} ( {a}^{( m )} ⋆ {b}^{( n )} ) ( t ) {e}^{- st} dt . \qquad ( 8.41 )$$

Using this definition we can derive a closed-form formula for the value of the Laplace transform of the cross-correlation of two causal spike trains, evaluated at s. This derivation is shown below.

$$\begin{matrix}{ℒ}_{a ⋆ b} ( s ) & = {lim}_{m \to ∞} {lim}_{n \to ∞} {\int }_{0 -}^{∞} ( {a}^{( m )} ⋆ {b}^{( n )} ) ( t ) {e}^{- st} dt \\ & = {lim}_{m \to ∞} {lim}_{n \to ∞} {\int }_{0 -}^{∞} {\int }_{- ∞}^{∞} \overline{{a}^{( m )} ( τ )} {b}^{( n )} ( τ + t ) {e}^{- st} d τ dt \\ & = {lim}_{m \to ∞} {lim}_{n \to ∞} {\int }_{0 -}^{∞} {\int }_{- ∞}^{∞} ( {\sum }_{j = 1}^{J} {δ}_{m} ( τ - {a}_{j} ) ) ( {\sum }_{k = 1}^{K} {δ}_{n} ( τ + t - {b}_{k} ) ) {e}^{- st} d τ dt \\ & = {lim}_{m \to ∞} {lim}_{n \to ∞} {\int }_{- ∞}^{∞} {\sum }_{j = 1}^{J} {δ}_{m} ( τ - {a}_{j} ) {\int }_{0 -}^{∞} {\sum }_{k = 1}^{K} {δ}_{n} ( τ + t - {b}_{k} ) {e}^{- st} dt d τ \\ & = {lim}_{m \to ∞} {\int }_{- ∞}^{∞} {\sum }_{j = 1}^{J} {δ}_{m} ( τ - {a}_{j} ) ( {lim}_{n \to ∞} {\int }_{0 -}^{∞} {\sum }_{k = 1}^{K} {δ}_{n} ( τ + t - {b}_{k} ) {e}^{- st} dt ) dτ \\ & = {lim}_{m \to ∞} {\int }_{- ∞}^{∞} {\sum }_{j = 1}^{J} {δ}_{m} ( τ - {a}_{j} ) ( {lim}_{n \to ∞} {\int }_{- ∞}^{∞} {\sum }_{k = 1}^{K} H ( t - {0}^{-} ) {δ}_{n} ( τ + t - {b}_{k} ) {e}^{- st} dt ) d τ \\ & = {\sum }_{j = 1}^{J} {\sum }_{k = 1}^{K} {lim}_{m \to ∞} {\int }_{- ∞}^{∞} {δ}_{m} ( τ - {a}_{j} ) {\underbrace{( {lim}_{n \to ∞} {\int }_{- ∞}^{∞} H ( t - {0}^{-} ) {δ}_{n} ( t - ( {b}_{k} - τ ) ) {e}^{- st} dt )}}_{{f}_{k} ( τ )} d τ \\ & = {\sum }_{j = 1}^{J} {\sum }_{k = 1}^{K} {lim}_{m \to ∞} {\int }_{- ∞}^{∞} {δ}_{m} ( τ - {a}_{j} ) {f}_{k} ( τ ) d τ .\end{matrix} \qquad ( 8.42 )$$

Note that the conjugation in a̅^̅{̅(̅m̅)̅}̅(̅τ̅)̅ can be dropped early on because δₘ is real. Also, note that this derivation uses Fubini's theorem to swap the order of the two integrals in the fourth line of this formula. This is possible because the template functions vanish outside a finite interval and the exponential functions are bounded on these intervals. Finally, as described in Section 8.5.1, the Heaviside function is used to change the lower limit of one of the integrals from 0⁻ to −∞ without affecting the result.

The last step in formula (8.42) used the following substitution:

$${f}_{k} ( τ ) = {lim}_{n \to ∞} {\int }_{- ∞}^{∞} H ( t - {0}^{-} ) {δ}_{n} ( t - ( {b}_{k} - τ ) ) {e}^{- st} dt . \qquad ( 8.43 )$$

We will show that for each t₀∈ [symbol] the limit of fₖ(τ) as τ→t₀ exists and is finite. This is done by deriving a closed-form expression for its value. Using the variable substitution \hat{τ}=bₖ−τ we can express this limit as follows:

$$\begin{matrix}{lim}_{τ \to {t}_{0}} {f}_{k} ( τ ) & = & {lim}_{τ \to {t}_{0}} {lim}_{n \to ∞} {\int }_{- ∞}^{∞} H ( t - {0}^{-} ) {δ}_{n} ( t - ( {b}_{k} - τ ) ) {e}^{- st} dt & ( 8.44 ) \\ & = & {lim}_{\hat{τ} \to ( {b}_{k} - {t}_{0} )} {lim}_{n \to ∞} {\int }_{- ∞}^{∞} H ( t - {0}^{-} ) {δ}_{n} ( t - \hat{τ} ) {e}^{- st} dt & \\ & = & H ( ( {b}_{k} - {t}_{0} ) - 0 ) ( {lim}_{\hat{τ} \to ( {b}_{k} - {t}_{0} )} {\underbrace{( {lim}_{n \to ∞} {\int }_{- ∞}^{∞} {δ}_{n} ( t - \hat{τ} ) {e}^{- st} dt )}}_{{e}^{- s \hat{τ}}} ) & ( Theorem 8.16 ) \\ & = & H ( {b}_{k} - {t}_{0} ) ( {lim}_{\hat{τ} \to ( {b}_{k} - {t}_{0} )} {e}^{- s \hat{τ}} ) & \\ & = & H ( {b}_{k} - {t}_{0} ) {e}^{- s ( {b}_{k} - {t}_{0} )} . & \end{matrix}$$

Finally, we can substitute the result from (8.44) into (8.42) and then use Theorem 8.15 to express the value of [symbol] _{a*b}(s) in closed form as follows:

$$\begin{matrix}{ℒ}_{a ⋆ b} ( s ) = {\sum }_{j = 1}^{J} {\sum }_{k = 1}^{K} {lim}_{m \to ∞} {\int }_{- ∞}^{∞} {δ}_{m} ( τ - {a}_{j} ) {f}_{k} ( τ ) d τ & ( Theorem 8.15 ) \\ = {\sum }_{j = 1}^{J} {\sum }_{k = 1}^{K} {lim}_{τ \to {a}_{j}} {f}_{k} ( τ ) & ( Formula ( 8.44 ) ) \\ = {\sum }_{j = 1}^{J} {\sum }_{k = 1}^{K} H ( {b}_{k} - {a}_{j} ) {e}^{- s ( {b}_{k} - {a}_{j} )} . & \end{matrix} \qquad ( 8.45 )$$

Thus, the formula for the Laplace transform of the cross-correlation of two causal spike trains is:

$${ℒ}_{a ⋆ b} ( s ) = {\sum }_{j = 1}^{J} {\sum }_{k = 1}^{K} H ( {b}_{k} - {a}_{j} ) {e}^{- s ( {b}_{k} - {a}_{j} )} . \qquad ( 8.46 )$$

This formula filters spike pairs for which the spike in the second train precedes the spike in the first train. This filtering is done using the Heaviside function, which acts as an open bigram filter. Because the value of H(bₖ−aⱼ) can be only 0 or 1, this expression reduces to a sum of exponentials. Each exponential in this sum is of the form e^{-(b}^{ₖ}^{-a}^{ⱼ}⁾, where (bₖ−aⱼ) is an interval between two spikes on two different channels and s is the argument of the Laplace transform.

There is a clear analog between formula (8.46) and the formula for discrete sequences. The main difference is that here the iterations are over spikes, where aⱼ and bₖ are the times at which they occur, while in the discrete case the iterations are over sequence elements that are assumed to occur at fixed time intervals. Another difference is that here the number of spikes on the a and b channels don't have to be the same. In the sequence domain, however, the two sequences are usually assumed to have the same number of elements.

There is an interesting relationship between the argument s of the Laplace transform and the argument z of the unilateral z-transform. If we assume that the time is discretized, then the two are related as follows: s=ln z. Under these conditions, formula (8.46) produces identical results to the formula for the discrete case.

To better understand what formula (8.46) does we will take a closer look at two special cases. In the first special case s=ln 2. This is analogous to encoding with z=2 in the discrete case. In this case the formula simplifies to:

$$\begin{matrix}{ℒ}_{a ⋆ b} ( ln 2 ) & = {\sum }_{j = 1}^{J} {\sum }_{k = 1}^{K} H ( {b}_{k} - {a}_{j} ) {e}^{- ( ln 2 ) ( {b}_{k} - {a}_{j} )} \\ & = {\sum }_{j = 1}^{J} {\sum }_{k = 1}^{K} H ( {b}_{k} - {a}_{j} ) {2}^{- ( {b}_{k} - {a}_{j} )}\end{matrix} . \qquad ( 8.47 )$$

In the second special case, s=ln 1=0, which corresponds to encoding with z=1 in the discrete case. Now, formula (8.46) simplifies to:

$${ℒ}_{a ⋆ b} ( 0 ) = {\sum }_{j = 1}^{J} {\sum }_{k = 1}^{K} H ( {b}_{k} - {a}_{j} ) . \qquad ( 8.48 )$$

The inner sum in this expression adds the number of spikes in the spike train b that occur after the j-th spike in the spike train a. The outer sum adds up these results for all j. Another way to interpret (8.48 is as follows:

$${ℒ}_{a ★ b} ( 0 ) = {\sum }_{j = 1}^{J} {\sum }_{k = 1}^{K} H ( {b}_{k} - {a}_{j} ) \cdot 1. \qquad ( 8.49 )$$

In other words, the decaying exponential in this case reduces to a constant function f(t)=1.

It is worth mentioning that while formula (8.46) is the most compact way to state the value of the Laplace transform of the cross-correlation of a and b, this formula may obscure the elegance of the encoding algorithm that is described below. This expression computes the value of one matrix element, but does it very inefficiently. The reason is that the double sum iterates over all spikes on the first channel a and over all spikes on the second channel b. The encoding algorithm computes the same result but it does not need to enumerate all possible pairs of spikes. Instead, it performs the computation incrementally using a single pass through both spike trains. This results in a fast and elegant algorithm.

The interplay between the Heaviside function and the indices j and k is not easy to decouple. Nevertheless, the Heaviside function simplifies the expressions so that they are easier to manipulate. Some of the following sections will use this formula. But, once again, from an algorithmic point of view a direct implementation of formula (8.46) is not advisable.

### 8.6 Operations on Truncated Spike Trains

In some cases it is necessary to work with only a subsection of some spike train. We will use the notation b[t₁, t₂] to denote a truncated spike train that is derived from the spike train b by keeping only the spikes that occur in the temporal interval [t₁, t₂] and removing all other spikes. For example, if b=(b₁, b₂, . . . , b_{K}), then b[t₁, t₂]=(bₚ, bₚ₊₁, . . . , b_{q}), where p=min{k: bₖ≥t₁} and q=max{k: bₖ≤t₂}. This notation can be extended to open intervals as well. Note that the truncated spike train may have fewer spikes, but the remaining spikes are not shifted in time.

### 8.6.1 Modeling Truncated Spike Trains

A truncated spike train is defined similarly to a regular spike train (see Definition 8.17), but now the train is truncated using two Heaviside step functions. The first function cuts all spikes that occur before time t₁. The second function cuts all spikes that occur after time t₂. The following definition states this more formally.

Definition 8.21. Let b=(b₁, b₂, . . . , b_{K}) be a spike train that contains K spikes and let t₁ and t₂ be two real numbers such that t₁≤t₂. The model for the truncated spike train b[t₁, t₂] is the sequence of functions (b_{[t}_{₁}_{, t}_{₂}_{]}⁽¹⁾(t), b_{[t}_{₁}_{, t}_{₂}_{]}⁽²⁾(t), . . . , b_{[t}_{₁}_{,t}_{₂}_{]}⁽ⁿ⁾(t), . . . ), where

$${b}_{[ {t}_{1} , {t}_{2} ]}^{( n )} ( t ) = H ( t - {t}_{1}^{-} ) H ( {t}_{2}^{+} - t ) {b}^{( n )} ( t ) . \qquad ( 8.5 )$$

To ensure that spikes that occur exactly at t₁ or exactly at t₂ are included in the truncated train, the definition uses left and right limits for these two boundaries. That is, it uses t₁⁻ as the left boundary and t₂⁺ as the right boundary in the Heaviside functions. Because b⁽ⁿ⁾(t) is modeled as a sum of shifted template functions δₙ, this is needed to include the entire region where δₙ(t−τ) is non-zero into the region of integration as n approaches infinity even if τ=t₁ or τ=t₂.

To understand how the truncation process works, it is useful to study the interaction of two Heaviside step functions. FIG. 109 shows three different plots. The first one is for H(t−t₁), i.e., a Heaviside function shifted to the right by t₁. The second plot is for H(t₂−t). In this case the direction of the step is inverted and the cutoff point is at t₂. The third plot shows the product of the previous two. In this case the resulting function is equal to 1 only in the interval [t₁, t₂], which is closed on both sides. Any spike train that is multiplied by this function will be truncated and only the spikes that occur in the interval [t₁, t₂] will be preserved. It is worth emphasizing again that after the multiplication the remaining spikes are not shifted in time.

Using the properties of the limit, formula (8.50) can also be stated in the following alternative form:

$$\begin{matrix}{b}_{[ {t}_{1} , {t}_{2} ]}^{( n )} ( t ) = H ( t - {t}_{1}^{-} ) H ( {t}_{2}^{+} - t ) {b}^{( n )} ( t ) \\ = {lim}_{{Δ}_{1} \to {0}^{+}} {lim}_{{Δ}_{2} \to {0}^{+}} H ( t - ( {t}_{1} - {Δ}_{1} ) ) H ( ( {t}_{2} + {Δ}_{2} ) - t ) {b}^{( n )} ( t ) .\end{matrix} \qquad ( 8.51 )$$

Furthermore, by combining Definition 8.21 and Definition 8.17, which defines the value of b⁽ⁿ⁾(t), we get:

$${b}_{[ {t}_{1} , {t}_{2} ]}^{( n )} ( t ) = {\sum }_{k = 1}^{K} H ( t - {t}_{1}^{-} ) H ( {t}_{2}^{+} - t ) {δ}_{n} ( t - {b}_{k} ) . \qquad ( 8.52 )$$

For open-ended intervals this formula can be adjusted as follows:

$${b}_{{[ {t}_{1} , {t}_{2} )}^{}}^{( n )} ( t ) = {\sum }_{k = 1}^{K} H ( t - {t}_{1}^{-} ) H ( {t}_{2}^{-} - t ) {δ}_{n} ( t - {b}_{k} ) , \qquad ( 8.53 )$$

$${b}_{( {t}_{1} , {t}_{2} ]}^{( n )} ( t ) = {\sum }_{k = 1}^{K} H ( t - {t}_{1}^{+} ) H ( {t}_{2}^{+} - t ) {δ}_{n} ( t - {b}_{k} ) , \qquad ( 8.54 )$$

$${b}_{( {t}_{1} , {t}_{2} )}^{( n )} ( t ) = {\sum }_{k = 1}^{K} H ( t - {t}_{1}^{+} ) H ( {t}_{2}^{-} - t ) {δ}_{n} ( t - {b}_{k} ) . \qquad ( 8.55 )$$

Note that the superscript pluses and minuses in these formulas are useful only when each formula is embedded in the limit of an integral as n→∞. Also, note that in these cases

$${lim}_{n \to ∞}$$

is the innermost limit. This is illustrated in the following sections.

### 8.6.2 The Laplace Transform of a Truncated Spike Train

This operation on truncated trains is defined similarly to the Laplace transform of regular spike trains (see Definition 8.18). In this case, however, the Laplace integral is extended with two Heaviside functions that perform the truncation.

Definition 8.22. Let b=(b₁, b₂, . . . , b_{K}) be a spike train that has K spikes and let t₁ and t₂ be two real numbers such that t₁≤t₂. The Laplace transform of the truncated spike train b[t₁, t₂] is a function that is obtained by taking the limit of the sequence of Laplace transforms of functions in the model or the truncated spike train. In other words,

$$ℒ \{ b [ {t}_{1} , {t}_{2} ] \} ( s ) = {lim}_{n \to ∞} ℒ \{ {b}_{[ {t}_{1} , {t}_{2} ]}^{( n )} \} ( s ) . \qquad ( 8.56 )$$

If we combine Definition 8.22 and Definition 8.21 we can expand (8.56) and derive an explicit formula for the Laplace transform of the truncated spike train b[t₁, t₂]. This derivation, which uses some properties of the Heaviside function, is shown below.

$$\begin{matrix}ℒ \{ b [ {t}_{1} , {t}_{2} ] \} ( s ) = {lim}_{n \to ∞} ℒ \{ {b}_{[ {t}_{1} , {t}_{2} ]}^{( n )} \} ( s ) \\ = {lim}_{n \to ∞} {\int }_{0 -}^{∞} H ( t - {t}_{1}^{-} ) H ( {t}_{2}^{+} - t ) {b}^{( n )} ( t ) {e}^{- st} dt \\ = {lim}_{n \to ∞} {\int }_{{0}^{-}}^{∞} H ( t - {t}_{1}^{-} ) H ( {t}_{2}^{+} - t ) ( {\sum }_{k = 1}^{K} {δ}_{n} ( t - {b}_{k} ) ) {e}^{- st} dt \\ = {\sum }_{k = 1}^{K} {lim}_{n \to ∞} {\int }_{{0}^{-}}^{∞} H ( t - {t}_{1}^{-} ) H ( {t}_{2}^{+} - t ) {δ}_{n} ( t - {b}_{k} ) {e}^{- st} dt \\ = {\sum }_{k = 1}^{K} {lim}_{n \to ∞} {\int }_{- ∞}^{∞} H ( t - {t}_{1}^{-} ) H ( {t}_{2}^{+} - t ) H ( t - {0}^{-} ) {δ}_{n} ( t - {b}_{k} ) {e}^{- st} dt \\ = {\sum }_{k = 1}^{K} H ( {b}_{k} - {t}_{1} ) H ( {t}_{2} - {b}_{k} ) H ( {b}_{k} - 0 ) ( {lim}_{n \to ∞} {\int }_{- ∞}^{∞} {δ}_{n} ( t - {b}_{k} ) {e}^{- st} dt ) \\ = {\sum }_{k = 1}^{K} H ( {b}_{k} - {t}_{1} ) H ( {t}_{2} - {b}_{k} ) H ( {b}_{k} ) {e}^{- s {b}_{k}} .\end{matrix} \qquad \begin{matrix}( Theorem 8.16 ) \\ ( 8.57 )\end{matrix}$$

This derivation is similar to (8.31). The main difference is that, because the train is truncated, there are now three Heaviside functions instead of just one. Finally, we can apply Theorem 8.16 because e^{-st} is a continuous function.

Next, we will derive three special cases of formula (8.57). The first special case computes the Laplace transform of the truncated spike train b[0, t]. In this case it is assumed that the original spike train b=(b₁, b₂, . . . , b_{K}) is causal (i.e., bₖ≥0 for all k) and that t₁=0 and t₂=t. That is, only the tail of the spike train is cut after time t. Under these conditions H(bₖ−t₁)=H(bₖ−0)=1 and H(bₖ)=1. Thus, formula (8.57) simplifies as follows:

$$\begin{matrix}ℒ \{ b [ 0 , t ] \} ( s ) = {\sum }_{k = 1}^{K} {\underbrace{H ( {b}_{k} - 0 )}}_{1} H ( t - {b}_{k} ) {\underbrace{H ( {b}_{k} )}}_{1} {e}^{- s {b}_{k}} \\ = {\sum }_{k = 1}^{K} H ( t - {b}_{k} ) {e}^{- s {b}_{k}} .\end{matrix} \qquad ( 8.58 )$$

The second special case computes the Laplace transform of the truncated spike train b[t, T]. In this case it is assumed that the original spike train b=(b₁, b₂, . . . , b_{K}) is causal and that bₖ≤T for all k={1, 2, . . . , K}, i.e., all spikes occur no later than time T. Under these assumptions formula (8.57) simplifies as follows:

$$\begin{matrix}ℒ \{ b [ t , T ] \} ( s ) = {\sum }_{k = 1}^{K} H ( {b}_{k} - t ) {\underbrace{H ( T - {b}_{k} )}}_{1} {\underbrace{H ( {b}_{k} )}}_{1} {e}^{- s {b}_{k}} \\ = {\sum }_{k = 1}^{K} H ( {b}_{k} - t ) {e}^{- s {b}_{k}} .\end{matrix} \qquad ( 8.59 )$$

The third special case is similar to the second case, but now both sides of (8.59) are multiplied by e^{st}. This leads to the following expression:

$$\begin{matrix}{e}^{st} ℒ \{ b [ t , T ] \} ( s ) = {e}^{s t} ( {\sum }_{k = 1}^{K} H ( {b}_{k} - t ) {e}^{- s {b}_{k}} ) \\ = {\sum }_{k = 1}^{K} H ( {b}_{k} - t ) {e}^{- s ( {b}_{k} - t )} .\end{matrix} \qquad ( 8.6 )$$

This formula can be viewed as a special case of the left-shift theorem, i.e., Theorem 8.8, when the shifted function is a spike train. To see this, we can represent b[t, T] as the following difference:

$$b [ t , T ] = b [ 0 , T ] - b [ 0 , t ) . \qquad ( 8.61 )$$

Taking the Laplace transform of both sides we get:

$$ℒ \{ b [ t , T ] \} ( s ) = ℒ \{ b [ 0 , T ] \} ( s ) - ℒ \{ b [ 0 , t ) \} ( s ) . \qquad ( 8.62 )$$

Finally, we can multiply both sides by e^{st} and use the fact that [symbol] {b[0, T]}(s)= [symbol] _{b}(s) to derive:

$${e}^{s t} ℒ \{ b [ t , T ] \} ( s ) = {e}^{s t} ( {ℒ}_{b} ( s ) - ℒ \{ b [ 0 , t ) \} ( s ) ) . \qquad ( 8.63 )$$

Note that the right-hand side is similar to the right-hand side of (8.16). Thus, the left-hand side can be viewed as the Laplace transform of the truncated spike train b[t, T] that has been shifted to the left by t. In other words, assuming that the integration variable for the Laplace transform is τ, we get:

$${e}^{s t} ℒ \{ b [ t , T ] \} ( s ) = {e}^{s t} ( {lim}_{n \to ∞} ℒ \{ {b}_{[ t , T ]}^{( n )} ( τ ) \} ( s ) ) = {lim}_{n \to ∞} ℒ \{ {b}_{[ t , T ]}^{( n )} ( τ + t ) \} ( s ) . \qquad ( 8.64 )$$

### 8.6.3 The Laplace Transform of the Cross-Correlation of Two Truncated Spike Trains

The definition for the cross-correlation of two truncated spike trains is similar to Definition 8.19, but uses the truncation notation. Once again, the conjugation can be dropped because the truncated spike trains are also modeled with shifted template functions, which are real-valued. The following definition states this more formally.

### Definition 8.23. Model for the Cross-Correlation of Two Truncated Spike Trains.

Let a=(a₁, a₂, . . . , a_{J}) be a spike train that contains J spikes and let b=(b₁, b₂, . . . , b_{K}) be another spike train that contains K spikes. Also, let a[t₁, t₂] and b[τ₁, τ₂] be two truncated spike trains that are derived from the original spike trains a and b. A model for the cross-correlation of the two truncated spike trains is formed by the functions (a_{[t}_{₁}_{,t}_{₂}_{]}^{(m)}*b_{[τ}_{₁}_{, τ}_{₂}_{]}⁽ⁿ⁾)(t), where m, n∈ [symbol] ={1, 2, . . . } such that

$$( {a}_{[ {t}_{1} , {t}_{2} ]}^{( m )} {★ b}_{[ {τ}_{1} , {τ}_{2} ]}^{( n )} ) ( t ) = {\int }_{- ∞}^{∞} \overline{{a}_{[ {t}_{1} , {t}_{2} ]}^{( m )} ( τ )} {b}_{[ {τ}_{1} , {τ}_{2} ]}^{( n )} ( τ + t ) d τ = {\int }_{- ∞}^{∞} {a}_{{[ {t}_{1} , {t}_{2} ]}^{}}^{( m )} ( τ ) {b}_{[ {τ}_{1} , {τ}_{2} ]}^{( n )} ( τ + t ) d τ . \qquad ( 8.65 )$$

The next definition, which is similar to Definition 8.20, formalizes the Laplace transform of the cross-correlation of two truncated spike trains.

Definition 8.24. The Laplace transform of the cross-correlation of two truncated spike trains. Let a=(a₁, a₂, . . . , a_{J}) and b=(b₁, b₂, . . . , b_{K}) be two spike trains and let t₁, t₂, τ₁, and τ₂ be four real numbers such that t₁≤t₂ and τ₁≤τ₂. Then, the Laplace transform of the cross-correlation of a[t₁, t₂] and b[τ₁, τ₂] (i.e., two truncated spike trains) is a function obtained by taking the iterated limit of Laplace transforms of the cross-correlation of a_{[t}_{₁}_{,t}_{₂}_{]}^{(m)} and b_{[τ}_{₁}_{, τ}_{₂}_{]}⁽ⁿ⁾ as m and n tend to infinity. In other words,

$$ℒ \{ a [ {t}_{1} , {t}_{2} ] ★b [ {τ}_{1} , {τ}_{2} ] \} ( s ) = {lim}_{m \to ∞} {lim}_{n \to ∞} {\int }_{0 -}^{∞} ( {a}_{{[ {t}_{1} , {t}_{2} ]}^{}}^{( m )} {★b}_{[ {τ}_{1} , {τ}_{2} ]}^{( n )} ) ( t ) {e}^{- s t} dt . \qquad ( 8.66 )$$

This definition is used below to derive a closed-form formula for the Laplace transform the cross-correlation of two truncated spike trains. To reduce the length of the formulas, however, we will introduce two shortcut functions Fₘ and Gₙ that are defined as follows:

$${F}_{m} ( {t}_{1} , {t}_{2} , {a}_{j} , t ) = H ( t - {t}_{1}^{-} ) H ( {t}_{2}^{+} - t ) {δ}_{m} ( t - {a}_{j} ) , \qquad ( 8.67 )$$

$${G}_{n} ( {τ}_{1} , {τ}_{2} , {b}_{k} , t ) = H ( t - {τ}_{1}^{-} ) H ( {τ}_{2}^{+} - t ) {δ}_{n} ( t - {b}_{k} ) . \qquad ( 8.68 )$$

Using the functions Fₘ and Gₙ, the first step of this derivation is to express the Laplace transform of the cross-correlation of a[t₁,t₂] and b[τ₁, τ₂], which will be denoted with L, as follows:

$$\begin{matrix}L = ℒ \{ a [ {t}_{1} , {t}_{2} ] ★ b [ {τ}_{1} , {τ}_{2} ] \} ( s ) \\ = {lim}_{m \to ∞} {lim}_{n \to ∞} {\int }_{0 -}^{∞} ( {a}_{[ {t}_{1} , {t}_{2} ]}^{( m )} {★ b}_{[ {τ}_{1} , {τ}_{2} ]}^{( n )} ) ( t ) {e}^{- s t} dt \\ = {lim}_{m \to ∞} {lim}_{n \to ∞} {\int }_{0 -}^{∞} {\int }_{- ∞}^{∞} {a}_{[ {t}_{1} , {t}_{2} ]}^{( m )} ( τ ) {b}_{[ {τ}_{1} , {τ}_{2} ]}^{( n )} ( τ + t ) {e}^{- s t} d τ d t \\ = {lim}_{m \to ∞} {lim}_{n \to ∞} {\int }_{0 -}^{∞} {\int }_{- ∞}^{∞} ( {\sum }_{j = 1}^{J} {F}_{m} ( {t}_{1} , {t}_{2} , {a}_{j} , τ ) ) ( {\sum }_{k = 1}^{K} {G}_{n} ( {τ}_{1} , {τ}_{2} , {b}_{k} , τ + t ) ) {e}^{- s t} d τ dt \\ = {lim}_{m \to ∞} {lim}_{n \to ∞} {\int }_{- ∞}^{∞} {\sum }_{j = 1}^{J} {F}_{m} ( {t}_{1} , {t}_{2} , {a}_{j} , τ ) {\int }_{0 -}^{∞} {\sum }_{k = 1}^{K} {G}_{n} ( {τ}_{1} , {τ}_{2} , {b}_{k} , τ + t ) {e}^{- s t} dtd τ \\ = {\sum }_{j = 1}^{J} {\sum }_{k = 1}^{K} {lim}_{m \to ∞} {lim}_{n \to ∞} {\int }_{- ∞}^{∞} {F}_{m} ( {t}_{1} , {t}_{2} , {a}_{j} , τ ) {\int }_{0 -}^{∞} {G}_{n} ( {τ}_{1} , {τ}_{2} , {b}_{k} , τ + t ) {e}^{- s t} dtd τ \\ = {\sum }_{j = 1}^{J} {\sum }_{k = 1}^{K} {lim}_{m \to ∞} {\int }_{- ∞}^{∞} {F}_{m} ( {t}_{1} , {t}_{2} , {a}_{j} , τ ) ( {lim}_{n \to ∞} {\int }_{0 -}^{∞} {G}_{n} ( {τ}_{1} , {τ}_{2} , {b}_{k} , τ + t ) {e}^{- s t} d t ) d τ \\ = {\sum }_{j = 1}^{J} {\sum }_{k = 1}^{K} {lim}_{m \to ∞} {\int }_{- ∞}^{∞} H ( τ - {t}_{1}^{-} ) H ( {t}_{2}^{+} - τ ) {δ}_{m} ( τ - {a}_{j} ) {\underbrace{( {lim}_{n \to ∞} {\int }_{0 -}^{∞} {G}_{n} ( {τ}_{1} , {τ}_{2} , {b}_{k} , τ + t ) {e}^{- s t} d t )}}_{{g}_{k} ( τ )} d τ \\ = {\sum }_{j = 1}^{J} {\sum }_{k = 1}^{K} {lim}_{m \to ∞} {\int }_{- ∞}^{∞} H ( τ - {t}_{1}^{-} ) H ( {t}_{2}^{+} - τ ) {δ}_{m} ( τ - {a}_{j} ) {g}_{k} ( τ ) d τ .\end{matrix} \qquad ( 8.69 )$$

The last step above used the shorthand notation gₖ(τ), which can be expressed as:

$$\begin{matrix}{g}_{k} ( τ ) = {lim}_{n \to ∞} {\int }_{0 -}^{∞} {G}_{n} ( {τ}_{1} , {τ}_{2} , {b}_{k} , τ + t ) {e}^{- s t} d t \\ = {lim}_{n \to ∞} {\int }_{- ∞}^{∞} H ( t - {0}^{-} ) {G}_{n} ( {τ}_{1} , {τ}_{2} , {b}_{k} , τ + t ) {e}^{- s t} d t \\ = {lim}_{n \to ∞} {\int }_{- ∞}^{∞} H ( t - {0}^{-} ) H ( ( τ + t ) - {τ}_{1}^{-} ) H ( {τ}_{2}^{+} - ( τ + t ) ) {δ}_{n} ( ( τ + t ) - {b}_{k} ) {e}^{- s t} d t \\ = {lim}_{n \to ∞} {\int }_{- ∞}^{∞} H ( t - ( {τ}_{1}^{-} - τ ) ) H ( ( {τ}_{2}^{+} - τ ) - t ) H ( t - {0}^{-} ) {δ}_{n} ( t - ( {b}_{k} - τ ) ) {e}^{- s t} dt .\end{matrix} \qquad ( 8.7 )$$

The value of

$${lim}_{τ \to {a}_{j}} {g}_{k} ( τ )$$

exists and is finite. The value of this limit in closed form is:

$${lim}_{τ \to {a}_{j}} {g}_{k} ( τ ) = H ( {b}_{k} - {τ}_{1} ) H ( {τ}_{2} - {b}_{k} ) H ( {b}_{k} - {a}_{j} ) {e}^{- s ( {b}_{k} - {a}_{j} )} . \qquad ( 8.71 )$$

Now we can derive the final formula:

$$\begin{matrix}ℒ \{ a [ {t}_{1} , {t}_{2} ] ★ b [ {τ}_{1} , {τ}_{2} ] \} ( s ) = {\sum }_{j = 1}^{J} {\sum }_{k = 1}^{K} {lim}_{m \to ∞} {\int }_{- ∞}^{∞} H ( τ - {t}_{1}^{-} ) H ( {t}_{2}^{+} - τ ) {δ}_{m} ( τ - {a}_{j} ) {g}_{k} ( τ ) d τ \\ = {\sum }_{j = 1}^{J} {\sum }_{k = 1}^{K} H ( {a}_{j} - {t}_{1} ) H ( {t}_{2} - {a}_{j} ) ( {lim}_{τ \to {a}_{j}} {g}_{k} ( τ ) ) \\ = {\sum }_{j = 1}^{J} {\sum }_{k = 1}^{K} H ( {a}_{j} K - {t}_{1} ) H ( {t}_{2} - {a}_{j} ) H ( {b}_{k} - {τ}_{1} ) H ( {τ}_{2} - {b}_{k} ) H ( {b}_{k} - {a}_{j} ) {e}^{- s ( {b}_{k} - {a}_{j} )} .\end{matrix} \qquad ( 8.72 )$$

Next, we will derive two special cases of formula (8.72) that will be used in the following sections. In the first case it is assumed that t₁=τ₁=0 and t₂=τ₂=t and that aⱼ≥0 and bₖ≥0 for all j and for all k. In other words, the two original spike trains a and b are causal and they are truncated at the same ending time t. Under these conditions formula (8.72) simplifies as follows:

$$\begin{matrix}ℒ \{ a [ 0 , t ] ★ b [ 0 , t ] \} ( s ) = {\sum }_{j = 1}^{J} {\sum }_{k = 1}^{K} {\underbrace{H ( {a}_{j} - 0 )}}_{1} H ( t - {a}_{j} ) {\underbrace{H ( {b}_{k} - 0 )}}_{1} H ( t - {b}_{k} ) H ( {b}_{k} - {a}_{j} ) {e}^{- s ( {b}_{k} - {a}_{j} )} \\ = {\sum }_{j = 1}^{J} {\sum }_{k = 1}^{K} H ( t - {a}_{j} ) H ( t - {b}_{k} ) H ( {b}_{k} - {a}_{j} ) {e}^{- s ( {b}_{k} - {a}_{j} )} .\end{matrix} \qquad ( 8.73 )$$

In the second special case it is assumed that t₁=τ₁=t and t₂=τ₂=T and that all spikes in the original trains a=(a₁, a₂, . . . , a_{J}) and b=(b₁, b₂, . . . , b_{K}) occur no later than time T, i.e., aⱼ≤T and bₖ≤T for all j and for all k. Under these conditions formula (8.72) simplifies as shown below:

$$\begin{matrix}ℒ \{ a [ t , T ] ★ b [ t , T ] \} ( s ) = {\sum }_{j = 1}^{J} {\sum }_{k = 1}^{K} H ( {a}_{j} - t ) {\underbrace{H ( T - {a}_{j} )}}_{1} H ( {b}_{k} - t ) {\underbrace{H ( T - {b}_{k} )}}_{1} H ( {b}_{k} - {a}_{j} ) {e}^{- s ( {b}_{k} - {a}_{j} )} \\ = {\sum }_{j = 1}^{J} {\sum }_{k = 1}^{K} H ( {a}_{j} - t ) H ( {b}_{k} - t ) H ( {b}_{k} - {a}_{j} ) {e}^{- s ( {b}_{k} - {a}_{j} )} .\end{matrix} \qquad ( 8.74 )$$

### 8.6.4 Modeling Reversed Spike Trains

In some cases the spikes in a spike train need to be temporally reversed. This section defines this operation and also the Laplace transform of a reversed spike train.

Definition 8.25. Reversed spike train. Let a=(a₁, a₂, . . . , a_{J}) be a causal spike train that contains J spikes that fall between 0 and T, i.e., 0≤aⱼ≤T for each j. The reversed spike train \vec{a} is obtained from a by reversing the times of the spikes on [0, T]. The model for [symbol] is a sequence of functions ( [symbol] ⁽¹⁾(t), [symbol] ⁽²⁾(t), . . . , [symbol] ^{(m)}(t), . . . ), where each function [symbol] ^{(m)}(t) is obtained by reversing a^{(m)}(t) on [0, T]. In other words,

$${\overleftarrow{a}}^{( m )} ( t ) = {a}^{( m )} ( T - t ) = {\sum }_{j = 1}^{J} {δ}_{m} ( T - t - {a}_{j} ) , \qquad ( 8.75 )$$

for each m∈ [symbol] ⁺={1, 2, . . . }. The time of the n-th spike in [symbol] is given the following formula:

$$\begin{matrix}{( \overleftarrow{a} )}_{n} = T - {a}_{J + 1 - n} , & for each n ∈ \{ 1 , 2 , … , J \}\end{matrix} . \qquad ( 8.76 )$$

The reversed spike train can also be expressed as follows:

$$\overleftarrow{a} = ( {( \overleftarrow{a} )}_{1} , {( ← \overleftarrow{a} )}_{2} , … , {( ← \overleftarrow{a} )}_{J} ) = ( T - {a}_{J} , T - {a}_{J - 1} , … , T - {a}_{2} , T - {a}_{1} ) . \qquad ( 8.77 )$$

It should be noted that the notation [symbol] is useful only if the interval for reversal is specified. By default, it will be assumed that this interval is [0, T], i.e., [symbol] = [symbol] [0, T]. If that is not the case, then the interval must be explicitly provided. Also, if the right bound of the interval is not equal to T, then the original spike train must be truncated before it can be reversed (see below).

Property 8.26. The Laplace transform of a reversed spike train. Let T be a non-negative real number and let a=(a₁, a₂, . . . , a_{J}) be a causal spike train such that 0≤a₃≤T for each j∈{1, 2, . . . , J}. Let [symbol] be the reversed spike train, as defined by Definition 8.25. Then, for each s∈ [symbol] , the Laplace transform of the reversed spike train can be expressed as follows:

$$ℒ \{ \overleftarrow{a} [ 0 , T ] \} ( s ) = {e}^{- s T} {ℒ}_{a} ( - s ) . \qquad ( 8.78 )$$

Definition 8.27. Truncated and reversed spike train. Let a=(a₁, a₂, . . . , a_{J}) be a causal spike train and let t≥0 be a real number. The truncated and reversed spike train [symbol] [0, t] is obtained from a by reversing the times of the spikes on the interval [0, t]. The model for [symbol] is a sequence of functions ( [symbol] _{[0, t]}⁽¹⁾(τ), [symbol] _{[0, t]}⁽²⁾(τ), . . . , [symbol] _{[0, t]}^{(m)}(τ), . . . ), where each function [symbol] _{[0, t]}^{(m)}(τ) is obtained by truncating and reversing a^{(m)}(τ) on [0, t]. More formally,

$${\overleftarrow{a}}_{{[ 0 , t ]}^{}}^{( m )} ( τ ) = {\sum }_{j = 1}^{J} H ( τ - {0}^{-} ) H ( {t}^{+} - τ ) {δ}_{m} ( ( t - τ ) - {a}_{j} ) , \qquad ( 8.79 )$$

for each m∈ [symbol] ⁺={1, 2, . . . }.

### Property 8.28. The Laplace Transform of a Truncated and Reversed Spike Train.

Let a=(a₁, a₂, . . . , a_{J}) be a causal spike train that has J spikes. Also, let t be a non-negative real number. Then, the Laplace transform of the truncated and reversed spike train [symbol] [0, t] can be expressed as follows:

$$ℒ \{ \overleftarrow{a} [ 0 , t ] \} ( s ) = {e}^{- st} ℒ \{ a [ 0 , t ] \} ( - s ) . \qquad ( 8.8 )$$

### 8.7 The Concatenation Theorem for Spike Trains

This section derives the concatenation theorem for spike trains. The derivation uses the result from Section 8.5.3, which derived the Laplace transform of the cross-correlation of two spike trains with finite number of spikes. This section also states several corollaries of the concatenation theorem for pairs of spike trains that meet certain conditions.

Theorem 8.29. The concatenation theorem for spike trains. Let a=(a₁, a₂, . . . , a_{J}) and b=(b₁, b₂, . . . , b_{K}) be two spike trains that unfold simultaneously over time. The first spike train consists of J spikes that occur at times a₁, a₂, . . . , a_{J}. It is assumed that the spike times are sorted in increasing order and that them are no duplicates in this list. It is also assumed that the spike train a is causal, i.e., aⱼ≥0 for all j. The second spike train has K spikes that occur at times b₁, b₂, . . . , b_{K}. Once again, it is assumed that b is causal, i.e., bₖ≥0 for all k, and that the list of spike times does not contain duplicates and is sorted in increasing order.
Let C be a nonnegative real constant that specifies the time at which the two spike trains am cut into two parts. Let a′ denote the prefix of a that contains the spikes in a up to and including time C. Let a″ denote the suffix of a that includes all remaining spikes that am not in a′, i.e., a″ contains each spike in a that occurs strictly after time C. Similarly, let b′ be the prefix of b that contains the spikes in b that occur strictly before time C. Let b″ be the suffix of b that includes all remaining spikes in b that are not present in b′. In other words, b″ contains each spike in b that occurs after time C, including spikes at C.
More formally, the spike train a is split into two spike trains a′ and a″ that are defined as:

$${a}^{′} = ( {a}_{1} , {a}_{2} , … , {a}_{p} ) , \qquad ( 8.81 )$$

$${a}^{″} = ( {a}_{p + 1} , {a}_{p + 2} , … , {a}_{J} ) , \qquad ( 8.82 )$$

$$where$$

$$p = max \{ j : {a}_{j} \leq C \} . \qquad ( 8.83 )$$

Note that by combining the list of spike times in a′ and a″ we can get back the original list of spike times in a, i.e., a=a′∥a″, where ∥ denotes concatenation.
Similarly, the spike train b is split into two spike trains b′ and b″ as follows:

$${b}^{′} = ( {b}_{1} , {b}_{2} , … , {b}_{q} ) , \qquad ( 8.84 )$$

$${b}^{″} = ( {b}_{q + 1} , {b}_{q + 2} , … , {b}_{K} ) , \qquad ( 8.85 )$$

$$where$$

$$p = max \{ k : {b}_{k} < C \} . \qquad ( 8.86 )$$

Once again, concatenating the lists of spike times for b′ and b″ results in the original spike train b, i.e., b=b′∥b″.
Note that formula (8.83) uses ≤, while formula (8.86) uses <. Essentially, a′ includes all spikes in a that fall in the closed interval [0, C] and a″ includes all spikes in a that fall in (C, ∞). On the other hand, b′ includes all spikes in b that fall in the interval [0, C) and b″ includes all spikes in b that fall in [C, ∞). More formally,

$${a}^{′} ← a [ 0 , C ] , {a}^{″} ← a ( C , ∞ ) , \qquad ( 8.87 )$$

$${b}^{′} ← b [ 0 , C ) , {b}^{″} ← b ( C , ∞ ) .$$

Then, the concatenation theorem for spike trains states that:

$${ℒ}_{a ★ b} ( s ) = {ℒ}_{{a}^{′} ★ {b}^{′}} ( s ) + {ℒ}_{{a}^{″} ★ {b}^{″}} ( s ) + \overline{{ℒ}_{{a}^{′}} ( - \overline{s} )} {ℒ}_{{b}^{″}} ( s ) . \qquad ( 8.88 )$$

In other words, the value of the Laplace transform of the cross-correlation of the spike trains a and b is equal to the value of the Laplace transform of a′*b′ plus the value of the Laplace transform of a″*b″ plus the conjugated value of the Laplace transform of a′ multiplied by the value of the Laplace transform of b″. In this expression all transforms ae evaluated at s, except the Laplace transform of a′, which is evaluated at −s̅.

It is worth mentioning that the splitting of the two trains as stated in the theorem is designed to reduce the number of special cases that have to be considered if there are spikes that occur exactly at time C, which is the time of the split. This split reduces these cases from 4 to 1.

The concatenation theorem is stated with conjugations to keep the final formula similar to the formula for the discrete case. Another reason is that conjugations are needed in Chapter 9, which extends the theory to weighted spike trains. In this chapter, however, each spike is modeled with a shifted template function that always returns a real number. Therefore, the conjugations can be dropped. Thus, another way to state the concatenation theorem for spike trains is:

$${ℒ}_{a ★ b} ( s ) = {ℒ}_{{a}^{′} ★ {b}^{′}} ( s ) + {ℒ}_{{a}^{″} ★ {b}^{″}} ( s ) + {ℒ}_{{a}^{′}} ( - s ) {ℒ}_{{b}^{″}} ( s ) . \qquad ( 8.89 )$$

Note that the concatenation theorem implicitly binds two types of abstractions. The first abstraction is a list that contains the spike times for some spike train. Lists can be concatenated and truncated. The second abstraction is a sequence of functions that models a spike train, where each spike is modeled with a shifted template function δₙ(t−t₀). Instead of concatenation, this abstraction allows for addition, which can be used to combine models. A spike train modeled in this way can also be truncated, but this requires the use of left- or right-limits with the Heaviside functions to correctly handle spikes that fall on one or both of the truncation boundaries. Without these limits the two types of abstractions lead to different results under some conditions. The list abstraction is used in the algorithms that are described later. The second abstraction is used to derive the theory and its mathematical formulas, which use the properties of the Laplace transform.

### 8.7.1 Special Cases of the Concatenation Theorem for Spike Trains

This section states two special cases of the concatenation theorem for spike trains as corollaries of Theorem 8.29.

The first corollary is a special case of the concatenation theorem when the two spike trains are split such that the suffix a″ is empty and the suffix b″ contains just one spike.

Corollary 8.30. When the suffix b″ contains just one spike and the suffix a″ is empty. Let a=(a₁, a₂, . . . , a_{J}) and b=(b₁, b₂, . . . , b_{K}) be two different spike trains such that aⱼ<b_{K} for j=1, 2, . . . , J and b_{K}=T. In other words, all spikes on a occur strictly before the last spike on b, which is at time T. Let a be divided into two spike trains a′ and a″ such that a′=a=(a₁, a₂, . . . , a_{J}) and a″=( ). That is, the prefix a′ contains all spikes from the original train and the suffix a″ is empty and contains no spikes. Also, let b be divided into two spike trains b′ and b″ where b′=(b₁, b₂, . . . , b_{K-1}) and b″=(b_{K}). That is, the suffix b″ contains just one spike, which is the last spike on b. Furthermore, it is assumed that all spike trains are causal, i.e., aⱼ≥0 and bₖ≥0 for all j=1, 2, . . . , J and all k=1, 2, . . . , K. Then,

$${ℒ}_{a ★ b} ( s ) = {ℒ}_{{a}^{′} ★ {b}^{′}} ( s ) + {ℒ}_{\overleftarrow{a}} ( s ) , \qquad ( 8.9 )$$

where [symbol] denotes the spike train obtained by reversing the spikes in a in the interval [0, T](see Definition 8.25). More formally, the time of the n-th spike in [symbol] is given by

$${( \overleftarrow{a} )}_{n} = T - {a}_{J + 1 - n} , \qquad ( 8.91 )$$

$$for$$

$$n = 1 , 2 , … , J ,$$

and the reversed spike train [symbol] is given by

$$\overleftarrow{a} = ( T - {a}_{J} , T - {a}_{J - 1} , … , T - {a}_{2} , T - {a}_{1} ) . \qquad ( 8.92 )$$

The second corollary is a special case of the concatenation theorem when the two spike trains are split such that the prefix a′ contains just one spike and the prefix b′ is empty.

Corollary 8.31. When the prefix a′ contains just one spike and the prefix b′ is empty. Let a=(a₁, a₂, . . . , a_{J}) and b=(b₁, b₂, . . . , b_{K}) be two spike trains. Also, let the train a be split into two non-overlapping spike trains a′ and a″ such that a′=(a₁) and a″=(a₂, a₃, . . . , a_{J}). In other words, the prefix a′ contains only the first spike from the original train a and the suffix a″ contains all remaining spikes form a. Furthermore, let b be split into b′ and b″ such that b′=( ) and b″=b=(b₁, b₂, . . . , b_{K}). That is, the prefix b′ is empty and the suffix b″ is equal to the original train b. In addition, the first spike on a occurs before all spikes on b such that a₁<bₖ for k=1, 2, . . . , K. Also, aⱼ≥0 and bₖ≥0 for all j and k. Then,

$${ℒ}_{a ★ b} ( s ) = {e}^{s {a}_{1}} {ℒ}_{b} ( s ) + {ℒ}_{{a}^{″} ★ {b}^{″}} ( s ) . \qquad ( 8.93 )$$

### 8.7.2 Special Cases of the Concatenation Theorem for Truncated Spike Trains

The concatenation theorem also applies to truncated spike trains. The next two corollaries form the mathematical basis for the algorithms that are described later in this chapter.

Corollary 8.32. Let x=(x₁, x₂, . . . , x_{J}) and y=(y₁, y₂, . . . , y_{K}) be two causal spike trains, i.e., xⱼ≥0 for each j∈{1, 2, . . . , J} and yₖ≥0 for each k∈{1, 2, . . . , K}. Then, for any integer n∈{2, 3, . . . , K} the following equation holds:

$$( 8.94 )$$

$$ℒ \{ x [ 0 , {y}_{n} ] ★ y [ 0 , {y}_{n} ] \} ( s ) = ℒ \{ x [ 0 , {y}_{n - 1} ] ★ y [ 0 , {y}_{n - 1} ] \} ( s ) + ℒ \{ \overleftarrow{x} [ 0 , {y}_{n} ] \} ( s ) ,$$

where [symbol] [0, yₙ] denotes the spike train obtained by reversing the truncated spike train x[0, yₙ] in the interval [0, yₙ]. In other words,

$$\overleftarrow{x} [ 0 , {y}_{n} ] = ( {y}_{n} - {x}_{p} , {y}_{n} - {x}_{p - 1} , … , {y}_{n} - {x}_{1} ) , \qquad ( 8.95 )$$

where p=max{xⱼ≤yₙ}.
Furthermore, for the special case when n=1, the following equation holds:

$$ℒ \{ x [ 0 , {y}_{1} ] ★ y [ 0 , {y}_{1} ] \} ( s ) = ℒ \{ \overleftarrow{x} [ 0 , {y}_{1} ] \} ( s ) . \qquad ( 8.96 )$$

Corollary 8.33. Let x=(x₁, x₂, . . . , x_{J}) and y=(y₁, y₂, . . . , y_{K}) be two causal spike trains such that their spikes occur no later than time T, i.e., 0≤xⱼ≤T and 0≤yₖ≤T for all j∈{1, 2, . . . , J} and for all k∈{1, 2, . . . , K}. Then, for any integer m∈{1, 2, . . . , J−1} the following formula holds:

$$( 8.97 )$$

$$ℒ \{ x [ {x}_{m} , T ] ★ y [ {x}_{m} , T ] \} ( s ) = {e}^{s {x}_{m}} ℒ \{ y [ {x}_{m} , T ] \} ( s ) + ℒ \{ x [ {x}_{m + 1} , T ] ★ y [ {x}_{m + 1} , T ] \} ( s ) .$$

Furthermore, in the special case when m=J, it has the following form:

$$ℒ \{ x [ {x}_{J} , T ] ★ y [ {x}_{J} , T ] \} ( s ) = {e}^{s {x}_{J}} ℒ \{ y [ {x}_{J} , T ] \} ( s ) . \qquad ( 8.98 )$$

The following two properties show that, under some conditions, the Laplace transform of the cross-correlation of two truncated spike trains is identical to the Laplace transform of the cross-correlation of two slightly different truncated spike trains. These properties were used to prove Corollary 8.32 and Corollary 8.33, which were stated above.

Property 8.34. Let x=(x₁, x₂, . . . , x_{J}) and y=(y₁, y₂, . . . , y_{K}) be two causal spike trains. Also, let x[0, t] and y[0, τ] be two truncated spike trains such that τ<t. Then,

$$ℒ \{ x [ 0 , t ] ⋆ y [ 0 , τ ] \} ( s ) = ℒ \{ x [ 0 , τ ] ⋆ y [ 0 , τ ] \} ( s ) . \qquad ( 8.99 )$$

In other words, the Laplace transform of the cross-correlation of x[0, t] and y[0, τ] is equal to the Laplace transform of the cross-correlation of x[0, τ] and y[0, τ]. Yet another way to say this is that the spikes from x that fall in the temporal interval (τ, t] don't contribute to the overall result.
Property 8.35. Let x=(x₁, X₂, . . . , x_{J}) and y=(y₁, y₂, . . . , y_{K}) be two causal spike trains such that all of their spikes occur no later than time T, i.e., 0≤xⱼ≤T and 0≤yₖ≤T for all j and for all k. Also, let x[t, T] and y[τ, T] be two truncated spike trains such that τ<t. Then,

$$ℒ \{ x [ t , T ] ⋆ y [ τ , T ] \} ( s ) = ℒ \{ x [ t , T ] ⋆ y [ t , T ] \} ( s ) . \qquad ( 8.1 )$$

In other words, the Laplace transform of the cross-correlation of x[t, T] and y[τ, T] is equal to the Laplace transform of the cross-correlation of x[t, T] and y[t, T]. Another way to state this is that the spikes from y that fall in the temporal interval [τ, t) don't affect the result.

### 8.8 The SSM Model

The SSM model consists of three components: a matrix M, a vector h′, and a vector h″. In general, the matrix is of size M′×M″, h′ is a column vector of size M′, and h″ is a row vector of size M″. To make this more concrete, we will assume that M′=M″=2. FIG. 110 shows the notation for the three components in that case.

In this example the model is computed from four causal spike trains that are denoted with α, β, A, and B. Each element of the matrix is computed from two spike trains where the first train is denoted with a Greek letter and the second train is denoted with an English letter. The first element of h′ is computed from the spike train α and its second element is computed from the spike train β. Similarly, the first element of h″ is computed from the spike train A and its second element is computed from the spike train B.

### 8.8.1 The Model at the End of Encoding

At the end of encoding each element of the matrix is equal to the value of the Laplace transform of the cross-correlation of the corresponding pair of spike trains. Each element of the vector h′ is equal to the value of the Laplace transform of the corresponding spike train, which is denoted with a Greek letter, after this spike train has been reversed in the interval [0, T]. Finally, each element of the vector h″ is equal to the value of the Laplace transform of the corresponding spike train that is denoted with an English letter. FIG. 111 shows the values of the three components in terms of the Laplace transform. Note that all transforms are evaluated at s, which is a parameter of the encoding algorithm.

Using the formulas derived in the previous sections, the values of the three components of the SSM model at the end of encoding can also be stated as:

$${h}^{′} = [ \begin{matrix}{\sum }_{j = 1}^{\vert α \vert} {e}^{- s ( T - {α}_{j} )} \\ {\sum }_{j = 1}^{\vert β \vert} {e}^{- s ( T - {β}_{j} )}\end{matrix} ] , \qquad ( 8.101 )$$

$${h}^{″} = [ {\sum }_{k = 1}^{\vert A \vert} {e}^{- {sA}_{k}} , {\sum }_{k = 1}^{\vert B \vert} {e}^{- s {B}_{k}} ] , \qquad ( 8.102 )$$

$$M = [ \begin{matrix}{\sum }_{j = 1}^{\vert α \vert} {\sum }_{k = 1}^{\vert A \vert} H ( {A}_{k} - {α}_{j} ) {e}^{- s ( {A}_{k} - {α}_{j} )} & {\sum }_{j = 1}^{\vert α \vert} {\sum }_{k = 1}^{\vert B \vert} H ( {B}_{k} - {α}_{j} ) {e}^{- s ( {B}_{k} - {α}_{j} )} \\ {\sum }_{j = 1}^{\vert β \vert} {\sum }_{k = 1}^{\vert A \vert} H ( {A}_{k} - {β}_{j} ) {e}^{- s ( {A}_{k} - {β}_{j} )} & {\sum }_{j = 1}^{\vert β \vert} {\sum }_{k = 1}^{\vert B \vert} H ( {B}_{k} - {β}_{j} ) {e}^{- s ( {B}_{k} - {β}_{j} )}\end{matrix} ] . \qquad ( 8.103 )$$

### 8.8.2 The Model at a Specific Time During Encoding

In some cases it is useful to know the value of a specific element of the model at a specific time during the encoding process. The previous formulas do not provide this information because they express the value of each element at the end of encoding. FIG. 112 summarizes the notation that will be used to express the state of the model during encoding.

Because the encoding algorithm does a single pass through all spike trains, going forward in time, the new formulas are given in terms of truncated spike trains. The truncation interval is [0, t] for all spike trains. To distinguish that these values are not the final values, but only the values during the encoding process, we will use a superscript e on the left, i.e., ^{e}h′, ^{e}h″, and ^{e}M. Because t may vary, the expression for each element is now a function of time.

Using the Laplace transform notation, the state of the model at time t during encoding can be expressed as follows:

$${e}^{} {h}^{′} ( t ) = [ \begin{matrix}ℒ \{ \overleftarrow{α} [ 0 , t ] \} ( s ) \\ ℒ \{ \overleftarrow{β} [ 0 , t ] \} ( s )\end{matrix} ] = [ \begin{matrix}{e}^{- st} ℒ \{ α [ 0 , t ] \} ( - s ) \\ {e}^{- st} ℒ \{ β [ 0 , t ] \} ( - s )\end{matrix} ] , \qquad ( 8.104 )$$

$${e}^{} {h}^{″} ( t ) = [ ℒ \{ A [ 0 , t ] \} ( s ) , ℒ \{ B [ 0 , t ] \} ( s ) ] , \qquad ( 8.105 )$$

$${}^{e} M ( t ) = [ \begin{matrix}ℒ \{ α [ 0 , t ] ⋆ A [ 0 , t ] \} ( s ) & ℒ \{ α [ 0 , t ] ⋆ B [ 0 , t ] \} ( s ) \\ ℒ \{ β [ 0 , t ] ⋆ A [ 0 , t ] \} ( s ) & ℒ \{ β [ 0 , t ] ⋆ B [ 0 , t ] \} ( s )\end{matrix} ] . \qquad ( 8.106 )$$

Using the Heaviside function each of these formulas can be stated in an alternative form:

$${e}^{} {h}^{′} ( t ) = [ \begin{matrix}{\sum }_{j = 1}^{\vert α \vert} H ( t - {α}_{j} ) {e}^{- s ( t - {α}_{j} )} \\ {\sum }_{j = 1}^{\vert β \vert} H ( t - {β}_{j} ) {e}^{- s ( t - {β}_{j} )}\end{matrix} ] , \qquad ( 8.107 )$$

$${e}^{} {h}^{″} ( t ) = [ {\sum }_{k = 1}^{\vert A \vert} H ( t - {A}_{k} ) {e}^{- {sA}_{k}} , {\sum }_{k = 1}^{\vert B \vert} H ( t - {B}_{k} ) {e}^{- {sB}_{k}} ] , \qquad ( 8.108 )$$

$${}^{e} M ( t ) = [ \begin{matrix}\begin{matrix}{\sum }_{j = 1}^{\vert α \vert} {\sum }_{k = 1}^{\vert A \vert} H ( t - {α}_{j} ) H ( t - {A}_{k} ) \\ H ( {A}_{k} - {α}_{j} ) {e}^{- s ( {A}_{k} - {α}_{j} )}\end{matrix} & \begin{matrix}{\sum }_{j = 1}^{\vert α \vert} {\sum }_{k = 1}^{\vert B \vert} H ( t - {α}_{j} ) H ( t - {B}_{k} ) \\ H ( {B}_{k} - {α}_{j} ) {e}^{- s ( {B}_{k} - {α}_{j} )}\end{matrix} \\ \begin{matrix}{\sum }_{j = 1}^{\vert β \vert} {\sum }_{k = 1}^{\vert A \vert} H ( t - {β}_{j} ) H ( t - {A}_{k} ) \\ H ( {A}_{k} - {β}_{j} ) {e}^{- s ( {A}_{k} - {β}_{j} )}\end{matrix} & \begin{matrix}{\sum }_{j = 1}^{\vert β \vert} {\sum }_{k = 1}^{\vert B \vert} H ( t - {β}_{j} ) H ( t - {B}_{k} ) \\ H ( {B}_{k} - {β}_{j} ) {e}^{- s ( {B}_{k} - {β}_{j} )}\end{matrix}\end{matrix} ] . \qquad ( 8.109 )$$

It is worth pointing out that all formulas in this section give the correct values for the elements at time t, but this is not how these elements are computed by the encoding algorithm. The algorithm uses iterative versions of these formulas, which are derived in Section 8.10.

### 8.8.3 The Model at a Specific Time During Decoding

The decoding process starts with the matrix M and the vector h″ and gradually depletes both of them down to zero. The initial values of M and h″, which are the same as their final values at the end of the encoding process, are shown in FIG. 111. This section states explicit formulas for the elements of the model at time t during the decoding process. To distinguish these formulas from the encoding formulas, we will use the small letter d in a superscript on the left, i.e., ^{d}h″ and ^{d}M. This notation is summarized in FIG. 113. Note that the vector h′ is not used during the decoding process.

The first set of formulas expresses the elements of h″ in terms of the Laplace transform of the corresponding truncated spike train and the elements of M in terms of the Laplace transform of the cross-correlation of two truncated spike trains. It is assumed that the spikes in all spike trains occur no later than time T. Thus, the truncation interval is [t, T] for all spike trains. More formally,

$${}^{d} M ( t ) = [ \begin{matrix}ℒ \{ α [ t , T ] ⋆ A [ t , T ] \} ( s ) & ℒ \{ α [ t , T ] ⋆ B [ t , T ] \} ( s ) \\ ℒ \{ β [ t , T ] ⋆ A [ t , T ] \} ( s ) & ℒ \{ β [ t , T ] ⋆ B [ t , T ] \} ( s )\end{matrix} ] , \qquad ( 8.11 )$$

$${d}^{} {h}^{″} ( t ) = [ {e}^{st} ℒ \{ A [ t , T ] \} ( s ) , {e}^{st} ℒ \{ B [ t , T ] \} ( s ) ] . \qquad ( 8.111 )$$

These formulas can also be stated in the following alternative form see (8.74 and (8.60)):

$${}^{d} M ( t ) = [ \begin{matrix}\begin{matrix}{\sum }_{j = 1}^{\vert α \vert} {\sum }_{k = 1}^{\vert A \vert} H ( {α}_{j} - t ) H ( {A}_{k} - t ) \\ H ( {A}_{k} - {α}_{j} ) {e}^{- s ( {A}_{k} - {α}_{j} )}\end{matrix} & \begin{matrix}{\sum }_{j = 1}^{\vert α \vert} {\sum }_{k = 1}^{\vert B \vert} H ( {α}_{j} - t ) H ( {B}_{k} - t ) \\ H ( {B}_{k} - {α}_{j} ) {e}^{- s ( {B}_{k} - {α}_{j} )}\end{matrix} \\ \begin{matrix}{\sum }_{j = 1}^{\vert β \vert} {\sum }_{k = 1}^{\vert A \vert} H ( {β}_{j} - t ) H ( {A}_{k} - t ) \\ H ( {A}_{k} - {β}_{j} ) {e}^{- s ( {A}_{k} - {β}_{j} )}\end{matrix} & \begin{matrix}{\sum }_{j = 1}^{\vert β \vert} {\sum }_{k = 1}^{\vert B \vert} H ( {β}_{j} - t ) H ( {B}_{k} - t ) \\ H ( {B}_{k} - {β}_{j} ) {e}^{- s ( {B}_{k} - {β}_{j} )}\end{matrix}\end{matrix} ] , \qquad ( 8.112 )$$

$${d}^{} {h}^{″} ( t ) = [ {\sum }_{k = 1}^{\vert A \vert} H ( {A}_{k} - t ) {e}^{- s ( {A}_{k} - t )} , {\sum }_{k = 1}^{\vert B \vert} H ( {B}_{k} - t ) {e}^{- s ( {B}_{k} - t )} ] . \qquad ( 8.113 )$$

### 8.8.4 The Formulas for an Abstract Element

For the sake of completeness, we will also state the formulas for an abstract element of the matrix and the two vectors. Using our convention, the matrix element will be called M_{a,b}, where a stands for any Greek letter and b stands for any English letter. Its corresponding elements in the two vectors will be denoted with hₐ′ and h_{b}″. Without loss of generality it will be assumed that the spike train a=(a₁, a₂, . . . , a_{J}) contains J spikes and the spike train b=(b₁, b₂, . . . , b_{K}) contains K spikes. Two sets of formulas are given below. The first set uses notation that is based on the Heaviside function. The second set uses the Laplace transform notation.

At the end of encoding (i.e., at time T):

$${h}_{a}^{′} = {}^{e} {h}_{a}^{′} ( T ) = {\sum }_{j = 1}^{J} {e}^{- s ( T - {a}_{j} )} , \qquad ( 8.114 )$$

$${h}_{b}^{″} = {}^{e} {h}_{b}^{″} ( T ) = {\sum }_{k = 1}^{K} {e}^{- {sb}_{k}} , \qquad ( 8.115 )$$

$${M}_{a , b} = {}^{e} {M}_{a , b} ( T ) = {\sum }_{j = 1}^{J} {\sum }_{k = 1}^{K} H ( {b}_{k} - {a}_{j} ) {e}^{- s ( {b}_{k} - {a}_{j} )} . \qquad ( 8.116 )$$

At time t during encoding:

$${}^{e} {h}_{a}^{′} ( t ) = {\sum }_{j = 1}^{J} H ( t - {a}_{j} ) {e}^{- s ( t - {a}_{j} )} , \qquad ( 8.117 )$$

$${}^{e} {h}_{b}^{″} ( t ) = {\sum }_{k = 1}^{K} H ( t - {b}_{k} ) {e}^{- {sb}_{k}} , \qquad ( 8.118 )$$

$${}^{e} {M}_{a , b} ( t ) = {\sum }_{j = 1}^{J} {\sum }_{k = 1}^{K} H ( t - {a}_{j} ) H ( t - {b}_{k} ) H ( {b}_{k} - {a}_{j} ) {e}^{- s ( {b}_{k} - {a}_{j} )} . \qquad ( 8.119 )$$

At time t during decoding:

$${}^{d} {M}_{a , b} ( t ) = {\sum }_{j = 1}^{J} {\sum }_{k = 1}^{K} H ( {a}_{j} - t ) H ( {b}_{k} - t ) H ( {b}_{k} - {a}_{j} ) {e}^{- s ( {b}_{k} - {a}_{j} )} , \qquad ( 8.12 )$$

$${}^{d} {h}_{b}^{″} ( t ) = {\sum }_{k = 1}^{K} H ( {b}_{k} - t ) {e}^{- s ( {b}_{k} - t )} . \qquad ( 8.121 )$$

The encoding and decoding formulas for an abstract element can also be stated using the Laplace transform notation. These formulas are given below and also shown in FIG. 114.

At the end of encoding (i.e., at time T):

$${h}_{a}^{′} = {}^{e} {h}_{a}^{′} ( T ) = ℒ \{ \overleftarrow{a} [ 0 , T ] \} ( s ) = {e}^{- sT} ℒ \{ a \} ( - s ) , \qquad ( 8.122 )$$

$${h}_{b}^{″} = {}^{e} {h}_{b}^{″} ( T ) = ℒ \{ b \} ( s ) , \qquad ( 8.123 )$$

$${M}_{a , b} = {}^{e} {M}_{a , b} ( T ) = ℒ \{ a ⋆ b \} ( s ) . \qquad ( 8.124 )$$

At time t during encoding:

$${}^{e} {h}_{a}^{′} ( t ) = ℒ \{ \overleftarrow{a} [ 0 , t ] \} ( s ) = {e}^{- st} ℒ \{ a [ 0 , t ] \} ( - s ) , \qquad ( 8.125 )$$

$${}^{e} {h}_{b}^{″} ( t ) = ℒ \{ b [ 0 , t ] \} ( s ) , \qquad ( 8.126 )$$

$${}^{e} {M}_{a , b} ( t ) = ℒ \{ a [ 0 , t ] ⋆ b [ 0 , t ] \} ( s ) . \qquad ( 8.127 )$$

At time t during decoding:

$${}^{d} {M}_{a , b} ( t ) = ℒ \{ a [ t , T ] ⋆ b [ t , T ] \} ( s ) , \qquad ( 8.128 )$$

$${}^{d} {h}_{b}^{″} ( t ) = {e}^{st} ℒ \{ b [ t , T ] \} ( s ) . \qquad ( 8.129 )$$

### 8.9 Duality of the Matrix Representation

This section shows that the values stored in the matrix at the end of encoding can be interpreted in two different ways. The first interpretation suggests how the matrix can be encoded. The second interpretation suggests how the matrix can be decoded.

To motivate the discussion we will start by repeating the formulas for the value of hₐ′ during encoding and the value of h_{b}″ during decoding:

$${}^{e} {h}_{a}^{′} ( t ) = {\sum }_{j = 1}^{J} H ( t - {a}_{j} ) {e}^{- s ( t - {a}_{j} )} , \qquad ( 8.13 )$$

$${}^{d} {h}_{b}^{″} ( t ) = {\sum }_{k = 1}^{K} H ( {b}_{k} - t ) {e}^{- s ( {b}_{k} - t )} . \qquad ( 8.131 )$$

Also, recall that, at the end of encoding the value of the matrix element in row a and column b is given by the following formula:

$${M}_{a , b} = {\sum }_{j = 1}^{J} {\sum }_{k = 1}^{K} H ( {b}_{k} - {a}_{j} ) {e}^{- s ( {b}_{k} - {a}_{j} )} . \qquad ( 8.132 )$$

### 8.9.1 Encoding View of the Matrix

Because each spike train contains a finite number of spikes, we can swap the order of the two sums in (8.132) to get the following result:

$$ \begin{matrix}{M}_{a , b} = {\sum }_{j = 1}^{J} {\sum }_{k = 1}^{K} H ( {b}_{k} - {a}_{j} ) {e}^{- s ( {b}_{k} - {a}_{j} )} \\ = {\sum }_{k = 1}^{K} {\underbrace{( {\sum }_{j = 1}^{J} H ( {b}_{k} - {a}_{j} ) {e}^{- s ( {b}_{k} - {a}_{j} )} )}}_{{}^{e} {h}_{a}^{′} ( {b}_{k} )} \\ = {\sum }_{k = 1}^{K} {}^{e} {h}_{a}^{′} ( {b}_{k} ) .\end{matrix} \qquad ( 8.133 )$$

In other words, the element M_{a,b} of the matrix can be computed by adding the values of hₐ′ at the times of the spikes on channel b.

This expression generalizes to all elements of the matrix. For example, the elements of a 2×2 matrix that is encoded from the spike trains a, A, A, and B can be expressed as follows:

$$M = [ \begin{matrix}{\sum }_{k = 1}^{\vert A \vert} {}^{e} {h}_{α}^{′} ( {A}_{k} ) & {\sum }_{k = 1}^{\vert B \vert} {}^{e} {h}_{α}^{′} ( {B}_{k} ) \\ {\sum }_{k = 1}^{\vert A \vert} {}^{e} {h}_{β}^{′} ( {A}_{k} ) & {\sum }_{k = 1}^{\vert B \vert} {}^{e} {h}_{β}^{′} ( {B}_{k} )\end{matrix} ] . \qquad ( 8.134 )$$

### 8.9.2 Decoding View of the Matrix

Formula (8.132) can also be factored in another way that leads to the decoding view of the matrix. This derivation is shown below:

$$\begin{matrix}{M}_{a , b} = {\sum }_{j = 1}^{J} {\sum }_{k = 1}^{K} H ( {b}_{k} - {a}_{j} ) {e}^{- s ( {b}_{k} - {a}_{j} )} \\ = {\sum }_{j = 1}^{J} {\underbrace{( {\sum }_{k = 1}^{K} H ( {b}_{k} - {a}_{j} ) {e}^{- s ( {b}_{k} - {a}_{j} )} )}}_{{}^{d} {h}_{b}^{″} ( {a}_{j} )} \\ = {\sum }_{j = 1}^{J} {}^{d} {h}_{b}^{″} ( {a}_{j} ) .\end{matrix} \qquad ( 8.135 )$$

In other words, the value of the element M_{a,b} can also be computed by adding the values of ^{d}h_{b}″ at the times of the spikes on channel a. Because ^{d}h_{b}″ is computed during decoding, however, this is not how the matrix can be computed. Instead, this suggests how the matrix can be decoded. That is, if the value of ^{d}h_{b}″ is subtracted from the value of M_{a,b} at the times of the spikes in a, then the matrix can be depleted down to zero.

Once again, this view of the matrix requires knowing the spike times on channel a. In general, these times are not available during decoding as that spike train is not provided. Thus, any decoding algorithm will have to infer these times.

The expression in (8.135) generalizes to all elements of the matrix. For example, the elements of a 2×2 matrix can be expressed using the following formula:

$$M = [ \begin{matrix}{\sum }_{j = 1}^{\vert α \vert} {}^{d} {h}_{A}^{″} ( {α}_{j} ) & {\sum }_{j = 1}^{\vert α \vert} {}^{d} {h}_{B}^{″} ( {α}_{j} ) \\ {\sum }_{j = 1}^{\vert β \vert} {}^{d} {h}_{A}^{″} ( {β}_{j} ) & {\sum }_{j = 1}^{\vert β \vert} {}^{d} {h}_{B}^{″} ( {β}_{j} )\end{matrix} ] . \qquad ( 8.136 )$$

### 8.10 Derivation of the Iterative Encoding Formulas

This section derives the iterative formulas that are used by the encoding algorithm, which is described in Section 8.11. These formulas are for the a-th element of the vector h′, the b-th element of the vector h″, and the element in the a-th row and b-th column of the matrix M. By analogy, these formulas can be extended to cover all elements of the three components of the SSM model.

8.10.1 Computing the a-th Element of the Vector h′

We would like to derive an iterative formula for computing the value of hₐ′ at the time of the m-th spike on channel a in terms of its value at the time of the (m−1)-st spike on a. That is, we would like to express ^{e}hₐ′(aₘ) in terms of ^{e}hₐ′(aₘ₋₁). To do this we will start by splitting the truncated spike train a[0, aₘ] into two segments:

$$a [ 0 , {a}_{m} ] = a [ 0 , {a}_{m - 1} ] + a ( {a}_{m - 1} , {a}_{m} ] , \qquad ( 8.137 )$$

where the second segment contains just one spike at time t=aₘ. Recall that, at time t during encoding the value of the a-th element of the vector h′ is given by formula (8.125), which is replicated below:

$${}^{e} {h}_{a}^{′} ( t ) = ℒ \{ \overleftarrow{a} [ 0 , t ] \} ( s ) = {e}^{- s t} ℒ \{ a [ 0 , t ] \} ( - s ) . \qquad ( 8.138 )$$

This formula is valid for any time t. In particular, if we set t=aₘ and use (8.137), then the value of ^{e}hₐ′(aₘ) can be expressed in the following way:

$$\begin{matrix}{}^{e} {h}_{a}^{′} ( {a}_{m} ) = ℒ \{ \overleftarrow{a} [ 0 , {a}_{m} ] \} ( s ) \\ = {e}^{- s {a}_{m}} ℒ \{ a [ 0 , {a}_{m} ] \} ( - s ) \\ = {e}^{- s {a}_{m}} ℒ \{ a [ 0 , {a}_{m - 1} ] \} ( - s ) + {e}^{- s {a}_{m}} ℒ \{ {\underbrace{a ( {a}_{m - 1} , {a}_{m} ]}}_{δ ( t - {a}_{m} )} \} ( - s ) \\ = {e}^{- s ( {a}_{m} - {a}_{m - 1} )} {\underbrace{{e}^{- s {a}_{m - 1}} ℒ \{ a [ 0 , {a}_{m - 1} ] \} ( - s )}}_{{}^{e} {h}_{a}^{′} ( {a}_{m} - 1 )} + {\underbrace{{e}^{- s {a}_{m}} {e}^{- ( - s {a}_{m} )}}}_{1} \\ = {}^{e} {h}_{a}^{′} ( {a}_{m - 1} ) {e}^{- s ( {a}_{m} - {a}_{m - 1} )} + 1.\end{matrix} \qquad ( 8.139 )$$

A similar approach can be used to express the value of ^{e}hₐ′ at t=bₙ, i.e., at the time of the n-th spike on channel b. Let p be the index of the last spike on a that occurs no later than the time of the n-th spike on b, i.e., p=max{j: aⱼ≤bₙ}. Then, a[0, bₙ] can be expressed as:

$$a [ 0 , {b}_{n} ] = a [ 0 , {a}_{p} ] + a ( {a}_{p} , {b}_{n} ] , \qquad ( 8.14 )$$

where a(aₚ, bₙ] is empty. Therefore,

$$\begin{matrix}{}^{e} {h}_{a}^{′} ( {b}_{n} ) = ℒ \{ \overleftarrow{a} [ 0 , {b}_{n} ] \} ( s ) = {e}^{- s {b}_{n}} ℒ \{ a [ 0 , {b}_{n} ] \} ( - s ) \\ = {e}^{- s {b}_{n}} ℒ \{ a [ 0 , {a}_{p} ] \} ( - s ) + {e}^{- s {b}_{n}} {\underbrace{ℒ \{ a ( {a}_{p} , {b}_{n} ] \} ( - s )}}_{0} \\ = {e}^{- s ( {b}_{n} - {a}_{p} )} {\underbrace{{e}^{- {sa}_{p}} ℒ \{ a [ 0 , {a}_{p} ] \} ( - s )}}_{{}^{e} {h}_{a}^{′} ( {a}_{p} )} \\ = {}^{e} {h}_{a}^{′} ( {a}_{p} ) {e}^{- s ( {b}_{n} - {a}_{p} )} .\end{matrix} \qquad ( 8.141 )$$

To summarize, the two formulas for updating ^{e}hₐ′ during the encoding process are:

$${}^{e} {h}_{a}^{′} ( {a}_{m} ) = {}^{e} {h}_{a}^{′} ( {a}_{m - 1} ) {e}^{- s ( {a}_{m} - {a}_{m - 1} )} + 1 , \qquad ( 8.142 )$$

$${}^{e} {h}_{a}^{′} ( {b}_{n} ) = {}^{e} {h}_{a}^{′} ( {a}_{p} ) {e}^{- s ( {b}_{n} - {a}_{p} )} . \qquad ( 8.143 )$$

Note that these formulas are used at different times. The first one is used at the times of the spikes on channel a. The second one is used at the spike times on channel b. Because this is somewhat cumbersome, Section 8.10.4 combines these two into a single formula by using a combined timeline that includes the spike times from both channels.
8.10.2 Computing the b-th Element of the Vector h″

Let b=(b₁, b₂, . . . , bₙ₋₁, bₙ, . . . , b_{K}) be a causal spike train that has K spikes. At time t during encoding the value of ^{e}h_{b}″ is given by formula (8.126), which is replicated below:

$${}^{e} {h}_{b}^{″} ( t ) = ℒ \{ b [ 0 , t ] \} ( s ) . \qquad ( 8.144 )$$

To derive an iterative formula for computing the value of ^{e}h_{b}″(bₙ) in terms of ^{e}h_{b}″(bₙ₋₁) we will start by representing the truncated spike train b[0, bₙ] as follows:

$$b [ 0 , {b}_{n} ] = b [ 0 , {b}_{n - 1} ] + b ( {b}_{n - 1} , {b}_{n} ] . \qquad ( 8.145 )$$

The additivity of the Laplace transform implies that the Laplace transform of b[0, bₙ] is equal to the sum of the Laplace transform of b[0, bₙ₋₁] and the Laplace transform of b(bₙ₋₁, bₙ]. Furthermore, b(bₙ₋₁, bₙ] contains just one spike at t=bₙ and reduces to the delta function shifted by bₙ. Using these properties and formula (8.144), we can derive the following expression:

$$\begin{matrix}{}^{e} {h}_{b}^{″} ( {b}_{n} ) = ℒ \{ b [ 0 , {b}_{n} ] \} ( s ) \\ = {\underbrace{ℒ \{ b [ 0 , {b}_{n - 1} ] \} ( s )}}_{{}^{e} {h}_{b}^{″} ( {b}_{n} - 1 )} + ℒ \{ {\underbrace{b ( {b}_{n - 1} , {b}_{n} ]}}_{δ ( t - {b}_{n} )} \} ( s ) \\ = {}^{e} {h}_{b}^{″} ( {b}_{n - 1} ) + ℒ \{ δ ( t - {b}_{n} ) \} ( s ) \\ = {}^{e} {h}_{b}^{″} ( {b}_{n - 1} ) + {e}^{- s {b}_{n}} .\end{matrix} \qquad ( 8.146 )$$

To summarize, the value of ^{e}h_{b}″ is updated only at the times of the spikes on channel b and the iterative update formula is:

$${}^{e} {h}_{b}^{″} ( {b}_{n} ) = {}^{e} {h}_{b}^{″} ( {b}_{n - 1} ) + {e}^{- s {b}_{n}} . \qquad ( 8.147 )$$

In other words, during encoding the value of the b-th element of the vector h″ at the time of the n-th spike on channel b is equal to the value of the same element at the time of the (n−1)-st spike plus e^{-sb}^{ₙ}, where s is the argument of the Laplace transform and bₙ is the time of the n-th spike.
8.10.3 Computing the Matrix Element in the a-th Row and b-th Column

Let a=(a₁, a₂, . . . , a_{J}) and b=(b₁, b₂, . . . , b_{K}) be two causal spike trains. The value of the matrix element M_{a,b} at time t of the encoding process is given by formula (8.127), which is replicated below:

$${}^{e} {M}_{a , b} ( t ) = ℒ \{ a [ 0 , t ] ★ b [ 0 , t ] \} ( s ) . \qquad ( 8.148 )$$

In other words, at time t this element is equal to the value of the Laplace transform at s of the cross-correlation of the spike train a and the spike train b, both of which are truncated at time t.

This formula is valid for any time t. In particular, if we set t=bₙ₋₁, i.e., the time of the (n−1)-st spike on channel b, then we will get the following expression:

$${}^{e} {M}_{a , b} ( {b}_{n - 1} ) = ℒ \{ a [ 0 , {b}_{n - 1} ] ★ b [ 0 , {b}_{n - 1} ] \} ( s ) . \qquad ( 8.149 )$$

Similarly, if we evaluate the same formula at the time of the n-th spike on channel b, i.e., at t=bₙ, then we will get:

$${}^{e} {M}_{a , b} ( {b}_{n} ) = ℒ \{ a [ 0 , {b}_{n} ] ★ b [ 0 , {b}_{n} ] \} ( s ) . \qquad ( 8.15 )$$

Corollary 8.32 implies that formula (8.150) can be expressed as follows:

$${\underbrace{ℒ \{ a [ 0 , {b}_{n} ] ★ b [ 0 , {b}_{n} ] \} ( s )}}_{{}^{e} {M}_{a , b} ( {b}_{n} )} = {\underbrace{ℒ \{ a [ 0 , {b}_{n - 1} ] ★ b [ 0 , {b}_{n - 1} ] \} ( s )}}_{{}^{e} {M}_{a , b} ( {b}_{n - 1} )} + {\underbrace{ℒ \{ \overleftarrow{a} [ 0 , {b}_{n} ] \} ( s )}}_{{}^{e} {h}_{a}^{′} ( {b}_{n} )} . \qquad ( 8.151 )$$

The last term in the right-hand side is equal to the value of the a-th element of h′ at the time of the n-th spike on b, i.e., at time t=bₙ (see formula (8.141)).

In the special case when t=b₁ (i.e., the time of the first spike on b), formula (8.151) reduces to:

$${\underbrace{ℒ \{ a [ 0 , {b}_{1} ] ★ b [ 0 , {b}_{1} ] \} ( s )}}_{{}^{e} {M}_{a , b} ( {b}_{1} )} = {\underbrace{ℒ \{ \overleftarrow{a} [ 0 , {b}_{1} ] \} ( s )}}_{{}^{e} {h}_{a}^{′} ( {b}_{1} )} , \qquad ( 8.152 )$$

which also follows from Corollary 8.32.

To summarize, during encoding, the value of the matrix element M_{a,b} is updated at the times of the spikes on channel b and it is computed using the following iterative formula:

$${}^{e} {M}_{a , b} ( {b}_{n} ) = {}^{e} {M}_{a , b} ( {b}_{n - 1} ) + {}^{e} {h}_{a}^{′} ( {b}_{n} ) . \qquad ( 8.153 )$$

In other words, the value of ^{e}M_{a,b} at the time of the n-th spike on b is equal to the value of that same element at the time of the (n−1)-st spike on b plus the value of the a-th element of the vector h′ at the time of the n-th spike on b.

### 8.10.4 The Iterative Encoding Formulas for a Common Timeline

The previous sections derived iterative formulas for ^{e}hₐ′, ^{e}h_{b}″, and ^{e}M_{a,b}. This section rewrites these formulas and states them for a common timeline that includes all spikes on a and all spikes on b. The resulting formulas form the mathematical foundation for the encoding algorithm that is described in Section 8.11.

Let a=(a₁, a₂, . . . , a_{J}) and let b=(b₁, b₂, . . . , b_{K}) be two causal spike trains from which the values of ^{e}hₐ′, ^{e}h_{b}″, and ^{e}M_{a,b} are computed. Also, let c=(c₁, c₂, . . . , c_{J+K}) be a list of spike times that combines all spikes from a and all spikes from b such that the resulting list c is sorted in increasing order.

It is possible to construct the array c from the elements of a and b. Because both a and b are initially sorted, the merging of the two spike trains can be accomplished in O(J+K) time. By definition, the original lists a and b contain no duplicates. It is possible, however, that an element of a may be equal to an element of b (e.g., two simultaneous spikes on two different channels). In that case the precedence is given to the spike from a, i.e., it will be listed before the spike from b in the list c.

In addition to the array c, it is also possible to generate another array â, which is a binary array of length J+K. The purpose of this array is to indicate the channel from which the spike in c came from. If âᵢ=1, then the i-th spike in the combined timeline came from a. On the other hand, if âᵢ=0, then the i-th spike came from b. In other words, for each i∈{1, 2, . . . , J+K} the value of the i-th element of â is defined as:

$${\hat{a}}_{i} = \{ \begin{matrix}1 , & if {c}_{i} comes from a , \\ 0 , & if {c}_{i} comes from b .\end{matrix} \qquad ( 8.154 )$$

The encoding algorithm, which is described in the next section, computes both â and c implicitly. The array a is replaced with one boolean variable that is called spikeOnA, which keeps the origin of the most recent spike, i.e., spikeOnA=âᵢ. The array c is not constructed either. Instead, the algorithm keeps only the two most recent elements in the variables t and tₚᵣₑᵥ.

To make the formulas more amenable to an algorithmic implementation, we will use i as an index for the elements of c. We will also use square brackets instead of round brackets, e.g., ^{e}hₐ′[i] instead of ^{e}hₐ′(cᵢ). We will use the value of âᵢ to check if an element of c comes from a or b.

At the start of the encoding process all variables are initialized to zero. In other words,

$${}^{e} {h}_{a}^{′} [ 0 ] = 0 , \qquad ( 8.155 )$$

$${}^{e} {h}_{b}^{″} [ 0 ] = 0 , \qquad ( 8.156 )$$

$${}^{e} {M}_{a , b} [ 0 ] = 0 , \qquad ( 8.157 )$$

Note that in this case the 0-th iteration counter is used to capture the initial conditions. This index is not used for actual spikes because the first spike in any spike train has an index of 1.

If a spike from a and a spike from b coincide, then a and c must be constructed to ensure that the spike from a has a lower index than the spike from b in the common timeline. In other words, in addition to (8.154) the values in â and c also satisfy the following two conditions:

$$\begin{matrix} & 1 ) & {c}_{i} \leq {c}_{i + 1} , & for each i ∈ \{ 1 , 2 , … , J + K - 1 \}\end{matrix} , \qquad ( 8.158 )$$

$$\begin{matrix} & 2 ) & if {c}_{i} = {c}_{i + 1} , & then {\hat{a}}_{i} = 1 and {\hat{a}}_{i + 1} = 0.\end{matrix} \qquad ( 8.159 )$$

If the pair (c, â) satisfies conditions (8.158) and (8.159), then the iterative formula for updating ^{e}hₐ′ can be stated as follows:

$${}^{e} {h}_{a}^{′} [ i ] = {}^{e} {h}_{a}^{′} [ i - 1 ] {e}^{- s ( {c}_{i} - {c}_{i - 1} )} + \{ \begin{matrix}1 , & if {\hat{a}}_{i} = 1 , \\ 0 , & otherwise ,\end{matrix} \qquad ( 8.16 )$$

for each i∈{1, 2, . . . , J+K}. This formula combines (8.142) and (8.143).

The update formula for the value of ^{e}M_{a,b} is based on (8.153). It follows a similar logic:

$${}^{e} {M}_{a , b} [ i ] = {}^{e} {M}_{a , b} [ i - 1 ] + \{ \begin{matrix}0 , & if {\hat{a}}_{i} = 1 , \\ {}^{e} {h}_{a}^{′} [ i ] , & otherwise ,\end{matrix} \qquad ( 8.161 )$$

for each i∈{1, 2, . . . , J+K}. Note that there is an implicit order dependency between formula (8.160) and formula (8.161). That is, the value of ^{e}hₐ′ must be computed first before it is used to update the value of ^{e}M_{a,b}.

The iterative update formula for the value of ^{e}h_{b}″ is based on (8.147). It can be stated as:

$${}^{e} {h}_{b}^{″} [ i ] = {}^{e} {h}_{b}^{″} [ i - 1 ] + \{ \begin{matrix}0 , & if {\hat{a}}_{i} = 1 , \\ {e}^{- s {c}_{i}} , & otherwise ,\end{matrix} \qquad ( 8.162 )$$

for each i∈{1, 2, . . . , J+K}.

FIG. 115 summarizes the encoding formulas for a common timeline, assuming that conditions (8.158) and (8.159) are satisfied. The formulas in the first column are applied when the current spike is on channel a (i.e., âᵢ=1). The formulas in the second column are applied when the current spike is on channel b (i.e., âᵢ=0).

If aⱼ=bₖ, i.e., if two spikes on different channels coincide, then precedence is given to the spike from a (see the formulas in the first column of FIG. 115). This is followed in the next iteration by the formulas in the second column. Note that in this case cᵢ=cᵢ₋₁ and e^{-s(c}^{ᵢ}^{-c}^{_{i−1}}⁾=e⁰=1. Thus, the value of hₐ′ will not change during the second iteration (i.e., the one that processes the spike on b), but its previous value will be subtracted from the value of M_{a,b}.

FIG. 116 shows how the iterative update formulas can be mapped to the formulas for the state of the SSM model at a specific iteration. The formulas in this figure describe how what is computed up to a given iteration of the algorithm maps to the theoretical model. In two of these formulas the truncation interval for the spike train b is open on the right. As explained below, this approach handles coincident spikes properly.

The formulas in the previous subsections were stated as functions of time and applied to only one spike train, in which, by definition, there are no coincidences. When the formulas are restated for a common timeline, however, it is possible to have ambiguities (e.g., ^{e}h_{b}″(aⱼ)≠^{e}h_{b}″(bₖ) even though aⱼ=bₖ). Then, ^{e}h_{b}″(t) is no longer a proper mathematical function because a function can have only one value for each point in its domain. The square bracket notation resolves this issue by assigning different values of the iteration counter to the two coincident spikes, i.e., it performs two iterations for each pair of coincident spikes. FIG. 116 captures this by explicitly formulating the state of the model at each iteration. It uses round truncation brackets in two of the formulas to resolve the ambiguities.

### 8.11 The Encoding Algorithm

Given two spike trains a and b and a value for the parameter s, the algorithm returns the value of the matrix element M_{a,b} and the values of hₐ″ and h_{b}″. To encode the entire matrix, the algorithm can be run in parallel, i.e., one instance of the algorithm for each matrix element. This is possible because each element can be computed independently of all other elements.

If aⱼ=bₖ for some j and some k, then the algorithm gives preference to the spike from a, but then performs another iteration to process the spike from b. During this second iteration hₐ″ does not change because t=tₚᵣₑᵥ due to the coincidence of the two spikes.

The computational complexity is O(J+K), where J is the number of spikes on channel a and K is the number of spikes on channel b.

### 8.12 Derivation of the Iterative Decoding Verification Formulas

This section derives iterative formulas for decreasing the value of the matrix element M_{a,b} and the vector element h_{b}″ down to zero. These formulas rely on knowing the times of the spikes on a, which are not available at run time. The goal of a proper decoding algorithm would be to estimate these values. Assuming that these estimates are correct, the formulas given here can be used to ensure that both the matrix element and the vector element will be depleted down to zero. In other words, this section states the formulas for verifying the solution obtained by a decoding algorithm.

8.12.1 Updating the Matrix Element in the a-th Row and b-th Column

Let a=(a₁, a₂, . . . , a_{J}) and b=(b₁, b₂, . . . , b_{K}) be two causal spike trains such that all of their spikes occur before time T. The value of the matrix element M_{a,b} at time t during the decoding process is given by formula (8.128), which is replicated below:

$${}^{d} {M}_{a , b} ( t ) = ℒ \{ a [ t , T ] ★ b [ t , T ] \} ( s ) . \qquad ( 8.163 )$$

If we evaluate this expression at the time of the m-th spike on channel a (i.e., at time t=aₘ), then we will get the following formula:

$${}^{d} {M}_{a , b} ( {a}_{m} ) = ℒ \{ a [ {a}_{m} , T ] ★ b [ {a}_{m} , T ] \} ( s ) . \qquad ( 8.164 )$$

Similarly, if we evaluate the same expression at the time of the (m+1)-st spike on a, then we will get another similar formula:

$${}^{d} {M}_{a , b} ( {a}_{m + 1} ) = ℒ \{ a [ {a}_{m + 1} , T ] ★ b [ {a}_{m + 1} , T ] \} ( s ) . \qquad ( 8.165 )$$

Using Corollary 8.33 we can express (8.164) as follows:

$${\underbrace{ℒ \{ a [ {a}_{m} , T ] ★ b [ {a}_{m} , T ] \} ( s )}}_{{}^{d} {M}_{a , b} ( {a}_{m} )} = {\underbrace{{e}^{s {a}_{m}} ℒ \{ b [ {a}_{m} , T ] \} ( s )}}_{{}^{d} {h}_{b}^{″} ( {a}_{m} )} + {\underbrace{ℒ \{ a [ {a}_{m + 1} , T ] ★ b [ {a}_{m + 1} , T ] \} ( s )}}_{{}^{d} {M}_{a , b} ( {a}_{m + 1} )} , \qquad ( 8.166 )$$

where the first term in the right-hand side is equal to the value of the b-th element of h″ at the time of the m-th spike on a. At the very last iteration, i.e., at time t=a_{J}, the following holds:

$$\underbrace{ℒ \{ a [ {a}_{J} , T ] ⋆ b [ {a}_{J} , T ] \} ( s )}_{{d}_{{M}_{a , b} ( {a}_{J} )}} = \underbrace{{e}^{s {a}_{J}} ℒ \{ b [ {a}_{J} , T ] \} ( s )}_{{}^{d} {h}_{b}^{″} ( {a}_{J} )} , \qquad ( 8.167 )$$

which also follows from Corollary 8.33.

After rearranging the three terms in (8.166), we get the following iterative formula for updating ^{d}M_{a,b} from aₘ to aₘ₊₁:

$${d}^{} {M}_{a , b} ( {a}_{m + 1} ) {=}^{d} {M}_{a , b} ( {a}_{m} ) {-}^{d} {h}_{b}^{″} ( {a}_{m} ) . \qquad ( 8.168 )$$

8.12.2 Updating the b-th Element of the Vector h″

During decoding the value of the b-th element of the vector h″ is given by formula (8.129), which is replicated below:

$${d}^{} {h}_{b}^{″} ( t ) = {e}^{st} ℒ \{ b [ t , T ] \} ( s ) . \qquad ( 8.169 )$$

This formula is valid for any time t, but we would like to derive its iterative version. That is, we would like to express the value of ^{d}h_{b}″ at the time of the (n+1)-st spike on channel b in terms of its value at the time of the n-th spike on b.

This can be done by expressing the truncated spike train b[bₙ, T] as follows:

$$b [ {b}_{n} , T ] = b [ {b}_{n} , {b}_{n + 1} ) + b [ {b}_{n + 1} , T ] . \qquad ( 8.17 )$$

By setting t to bₙ and by combining (8.169) and (8.170), we get:

$$\begin{matrix}{d}^{} {h}_{b}^{″} ( {b}_{n} ) = {e}^{{sb}_{n}} ℒ \{ b [ {b}_{n} , T ] \} ( s ) \\ = {e}^{{sb}_{n}} ℒ \{ \underbrace{b [ {b}_{n} , {b}_{n + 1} )}_{δ ( t - {b}_{n} )} \} ( s ) + {e}^{s {b}_{n}} ℒ \{ b [ {b}_{n + 1} , T ] \} ( s ) \\ = \underbrace{{e}^{s {b}_{n}} {e}^{- s {b}_{n}}}_{1} + {e}^{s ( {b}_{n} - {b}_{n + 1} )} \underbrace{{e}^{s {b}_{n + 1}} ℒ \{ b [ {b}_{n + 1} , T ] \} ( s )}_{{}^{d} {h}_{b}^{″} ( {b}_{n + 1} )}\end{matrix} \qquad ( 8.171 )$$

$$= 1 + {d}^{} {h}_{b}^{″} ( {b}_{n + 1} ) {e}^{s ( {b}_{n} - {b}_{n + 1} )} .$$

After rearranging the terms we can express ^{d}h_{b}″(bₙ₊₁) using ^{d}h_{b}″(bₙ) as follows:

$${d}^{} {h}_{b}^{″} ( {b}_{n + 1} ) = {[}^{d} {h}_{b}^{″} ( {b}_{n} ) - 1 ] {e}^{s ( {b}_{n + 1} - {b}_{n} )} . \qquad ( 8.172 )$$

A similar approach can be used to derive the formula for ^{d}h_{b}″ at the time of the m-th spike on channel a. In this case, the idea is to set t=bₚ in (8.169) and to express b[bₚ, T] as:

$$b [ {b}_{p} , T ] = b [ {b}_{p} , {a}_{m} ) + b [ {a}_{m} , T ] , \qquad ( 8.173 )$$

where p=max{k: bₖ<aₘ}. In other words, p is the index of the last spike on channel b that occurs strictly before the m-th spike on channel a. Using the properties of the Laplace transform we can express ^{d}h_{b}″(bₚ) in terms of ^{d}h_{b}″(aₘ) as follows:

$$\begin{matrix}{d}^{} {h}_{b}^{″} ( {b}_{p} ) = {e}^{{sb}_{p}} ℒ \{ b [ {b}_{p} , T ] \} ( s ) \\ = {e}^{{sb}_{p}} ℒ \{ \underbrace{b [ {b}_{p} , {a}_{m} )}_{δ ( t - {b}_{p} )} \} ( s ) + {e}^{s {b}_{p}} ℒ \{ b [ {a}_{m} , T ] \} ( s ) \\ = {e}^{s {b}_{p}} ℒ \{ δ ( t - {b}_{p} ) \} ( s ) + {e}^{s ( {b}_{p - {a}_{m} )}} \underbrace{{e}^{{sa}_{m}} ℒ \{ b [ {a}_{m} , T ] \} ( s )}_{{}^{d} {h}_{b}^{″} ( {a}_{m} )}\end{matrix} \qquad ( 8.174 )$$

$$\begin{matrix}= \underbrace{{e}^{s {b}_{p}} {e}^{- {sb}_{p}}}_{1} {+}^{d} {h}_{b}^{″} ( {a}_{m} ) {e}^{s ( {b}_{p} - {a}_{m} )} \\ = 1 + {d}^{} {h}_{b}^{″} ( {a}_{m} ) {e}^{s ( {b}_{p} - {a}_{m} )} .\end{matrix}$$

By rearranging the terms in the previous expression we get the following formula:

$${d}^{} {h}_{b}^{″} ( {a}_{m} ) = {[}^{d} {h}_{b}^{″} ( {b}_{p} ) - 1 ] {e}^{s ( {a}_{m} - {b}_{p} )} . \qquad ( 8.175 )$$

To summarize, the iterative decoding verification formulas for the b-th element of h″ are:

$${d}^{} {h}_{b}^{″} ( {b}_{n + 1} ) = {[}^{d} {h}_{b}^{″} ( {b}_{n} ) - 1 ] {e}^{s ( {b}_{n + 1} - {b}_{n} )} , \qquad ( 8.176 )$$

$${d}^{} {h}_{b}^{″} ( {a}_{m} ) = {[}^{d} {h}_{b}^{″} ( {b}_{p} ) - 1 ] {e}^{s ( {a}_{m} - {b}_{p} )} . \qquad ( 8.177 )$$

The first formula is used to update this element at the time of the spikes on channel b. The second one is used at the spike times on channel a. Section 8.12.3 combines both of these into a single iterative update formula for a common timeline, which is denoted by c. Note that formula (8.177) is not strictly iterative because bₚ is temporally before aₘ but it may also be temporally before aₘ₋₁ and other spikes on a. This formula will be modified in the next section to make it truly iterative (i.e., updated at the current spike time using the result from the previous spike time in the common timeline). This modification also resolves the ambiguities that arise when spikes from a and b coincide.

The initial value of ^{d}h_{b}″ can be computed from formula (8.169) by setting t to zero, i.e.,

$${d}^{} {h}_{b}^{″} ( 0 ) = \underbrace{{e}^{0}}_{1} ℒ \{ b [ 0 , T ] \} ( s ) = ℒ \{ b [ 0 , T ] \} ( s ) {=}^{e} {h}_{b}^{″} ( T ) . \qquad ( 8.178 )$$

That is, the initial value of h_{b}″ during decoding is equal to the final value of h_{b}″ during encoding.

A small technical detail has to be addressed during the very first iteration. Let t₁ be the time of the first spike on either channel a or channel b, i.e., tᵢ=min(a₁, b₁). Then, by definition, the truncated spike train b[0, t₁) contains no spikes. Thus, the Laplace transform of b[0, T] reduces to the Laplace transform of b[t₁, T], i.e.,

$$( 8.179 )$$

$$ℒ \{ b [ 0 , T ] \} ( s ) = \underbrace{L \{ b [ 0 , {t}_{1} ) \} ( s )}_{0} + ℒ \{ b [ {t}_{1} , T ] \} ( s ) = ℒ \{ b [ {t}_{1} , T ] \} ( s ) .$$

Therefore, setting t=t₁ in equation 8.169 leads to the following formula:

$${d}^{} {h}_{b}^{″} ( {t}_{1} ) = {e}^{s {t}_{1}} ℒ \{ b [ {t}_{1} , T ] \} ( s ) = {e}^{s {t}_{1}} ℒ \{ b [ 0 , T ] \} ( s ) = {e}^{s {t}_{1} d} {h}_{b}^{″} ( 0 ) . \qquad ( 8.18 )$$

In other words, the initial value ^{d}h_{b}″(0), which is equal to ^{e}h_{b}″(T), is multiplied by e^{st}^{₁}. Thus, in the first iteration the value of ^{d}h_{b}″ is computed using formula (8.180). In all subsequent iterations ^{d}h_{b}″ is updated using formula (8.176) or formula (8.177).

### 8.12.3 The Iterative Decoding Verification Formulas for a Common Timeline

This section states the decoding verification formulas for a common timeline. Let a=(a₁, a₂, . . . , a_{J}) be the list of spikes that we want to verify. Let b=(b₁, b₂, . . . , b_{K}) be the list of spikes on channel b that are available at run time. Finally, let c=(c₁, c₂, . . . , c_{J+K}) be another list that is derived from a and b by combining and sorting the spike times of these two lists in increasing order.

At the start of this process ^{d}h_{b}″ and ^{d}M_{a,b} are equal to the values computed at the end of encoding, i.e., at the (J+K)-th encoding iteration. In other words, the initial conditions are:

$${d}^{} {h}_{b}^{″} [ 0 ] {=}^{e} {h}_{b}^{″} [ J + K ] , \qquad ( 8.181 )$$

$${d}^{} {M}_{a , b} [ 0 ] = e {M}_{a , b} [ J + K ] . \qquad ( 8.182 )$$

Once again, the 0-th iteration counter is used to capture the initial conditions. Also, in keeping with the previous convention, we will use i instead of cᵢ and the square bracket notation, e.g, ^{d}h_{b}″(cᵢ)=^{d}h_{b}″[i].

As in the encoding case, it is assumed that c and â are ordered in a way that ensures correct processing of coincident spikes (i.e., in the common timeline, spikes from a are processed before their coincident counterparts from b). More formally, (c, â) must satisfy the following two conditions, which are identical to (8.158) and (8.159):

$$1 ) {c}_{i} \leq {c}_{i + 1} , for each i ∈ \{ 1 , 2 , … , J + K - 1 \} , \qquad ( 8.183 )$$

$$2 ) if {c}_{i} = {c}_{i + 1} , then {\hat{a}}_{i} = 1 and {\hat{a}}_{i + 1} = 0. \qquad ( 8.184 )$$

where â is a binary indicator array defined by (8.154).

Combining formulas (8.171) and (8.174) leads to the following formula for ^{d}h_{b}″[i]:

$${}^{d} {h}_{b}^{″} [ i ] = {}^{d} {h}_{b}^{″} [ i + 1 ] {e}^{s ( {c}_{i} - {c}_{i + 1} )} + \{ \begin{matrix}0 , if {\hat{a}}_{i + 1} = 1 , \\ 1 , otherwise .\end{matrix} \qquad ( 8.185 )$$

This expression, however, works backward in time. To get the iterative update formula we need to rearrange the terms as follows:

$${}^{d} {h}_{b}^{″} [ i + 1 ] = {}^{d} {h}_{b}^{″} [ i ] {e}^{s ( {c}_{i + 1} - {c}_{i} )} - \{ \begin{matrix}0 , if {\hat{a}}_{i + 1} = 1 , \\ 1 , otherwise ,\end{matrix} \qquad ( 8.186 )$$

where i∈{0, 1, 2, . . . , J+K−1}. This formula combines formulas (8.176) and (8.177). It states that the value of ^{d}h_{b}″ is multiplied by the exponential e^{s(c}^{ᵢ₊₁}^{-c}^{ᵢ}⁾ during all iterations. If the current spike came from channel b, then there is also a subtraction. As in the encoding case, if a spike from a coincides with a spike from b, then there would be two consecutive updates, but precedence will be given to the spike from a. Note that during the second update cᵢ₊₁=cᵢ and the multiplication by e^{s(c}^{ᵢ₊₁}^{-c}^{ᵢ}⁾ has no effect, only the subtraction of 1 is performed.

The value of the matrix element is updated as follows:

$${d}^{} {M}_{a , b} [ i + 1 ] = {d}^{} {M}_{a , b} [ i ] - \{ \begin{matrix}{}^{d} {h}_{b}^{″} [ i + 1 ] , if {\hat{a}}_{i + 1} = 1 , \\ 0 , otherwise ,\end{matrix} \qquad ( 8.187 )$$

for each i∈{0, 1, 2, . . . , J+K−1}. This formula performs the updates specified by formula (8.168). Note that the updates are performed only at the spike times from a, otherwise the matrix element remains unchanged.

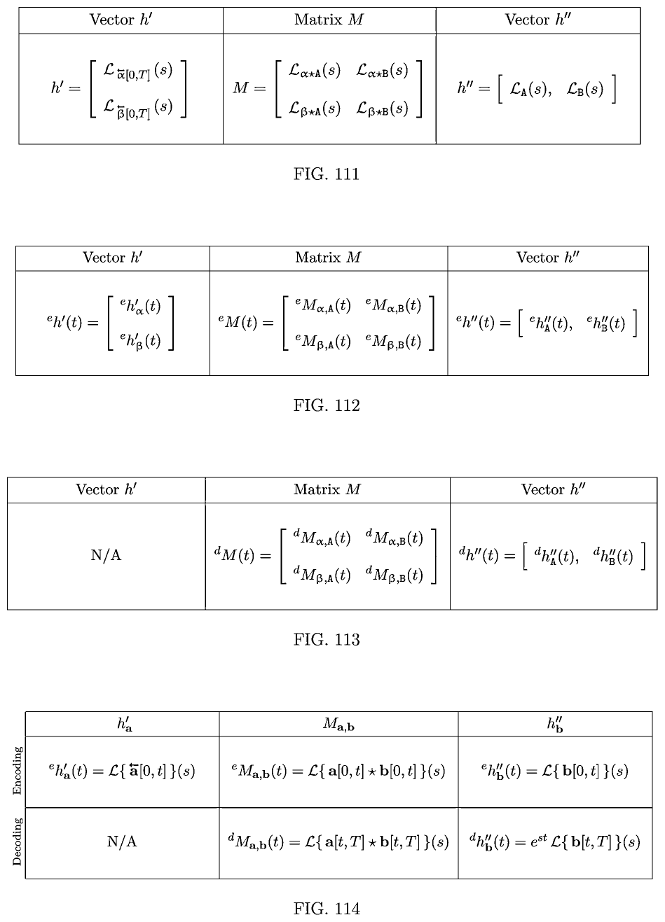

FIG. 117 summarizes the decoding verification formulas, assuming that conditions (8.183) and (8.184) are true. That is, the formulas in the left column of the figure have priority over the formulas in the right column when a spike from a and a spike from b coincide. The verification is successful if after the last iteration both ^{d}h_{b}″ and ^{d}M_{a,b} are equal to zero, i.e., ^{d}M_{a,b}[J+K]=0 and ^{d}h_{b}″[J+K]=0.

Processing the first spike in c requires special attention. As described in formula (8.180), the value of h_{b}″ is updated as follows in this case:

$${}^{d} {h}_{b}^{″} [ 1 ] = {}^{d} {h}_{b}^{″} [ 0 ] {e}^{s {c}_{1}} . \qquad ( 8.188 )$$

The algorithm handles this case implicitly by augmenting this formula as follows:

$${}^{d} {h}_{b}^{″} [ 1 ] = {}^{d} {h}_{b}^{″} [ 0 ] {e}^{s ( {c}_{1} - {c}_{0} )} , \qquad ( 8.189 )$$

where c₀=0. In other words, it augments the array c with an implicit 0-th spike at time t=0.

Similarly to the encoding case, the verification algorithm does not construct the array c explicitly. Instead, it uses the variables t and tₚᵣₑᵥ to keep only its two most recent elements. The algorithm does not construct the array â either. Instead, it uses the boolean variable spikeOnA to track only its most recent element, i.e., spikeOnA is equal to âᵢ₊₁. This ensures that coincident spikes are processed in the correct order.

FIG. 118 shows the mapping of the update formulas to the state of the SSM model at the time of the (i+1)-st verification iteration. This mapping is stated using the Laplace transform notation for truncated spike trains. As in the encoding case, some of the formulas use round truncation brackets to resolve ambiguities due to coincident spikes on a and b.

8.12.4 Deriving the Update Formulas for ^{d}h_{b}″ from the Model

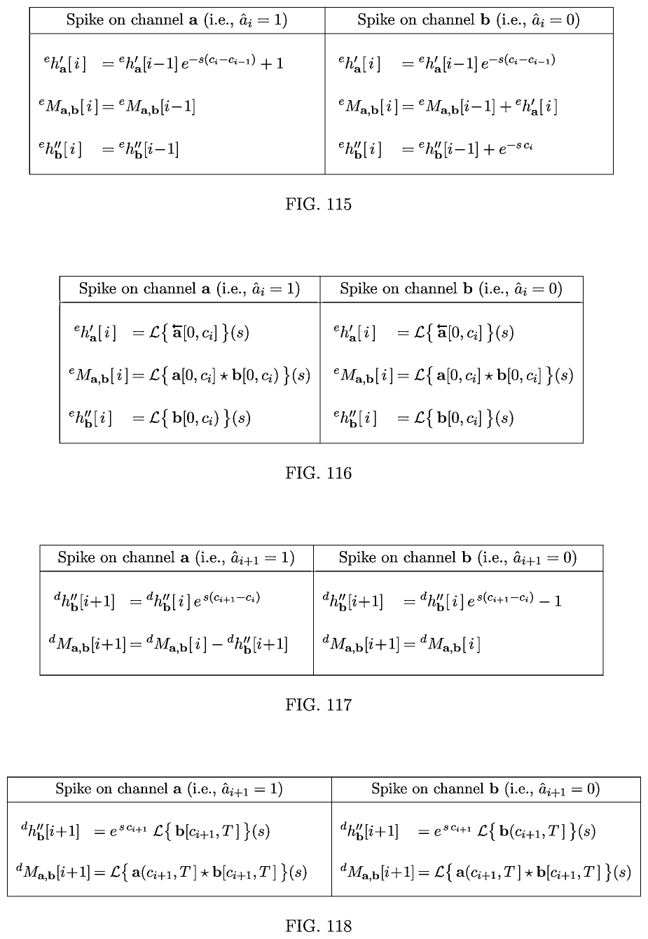

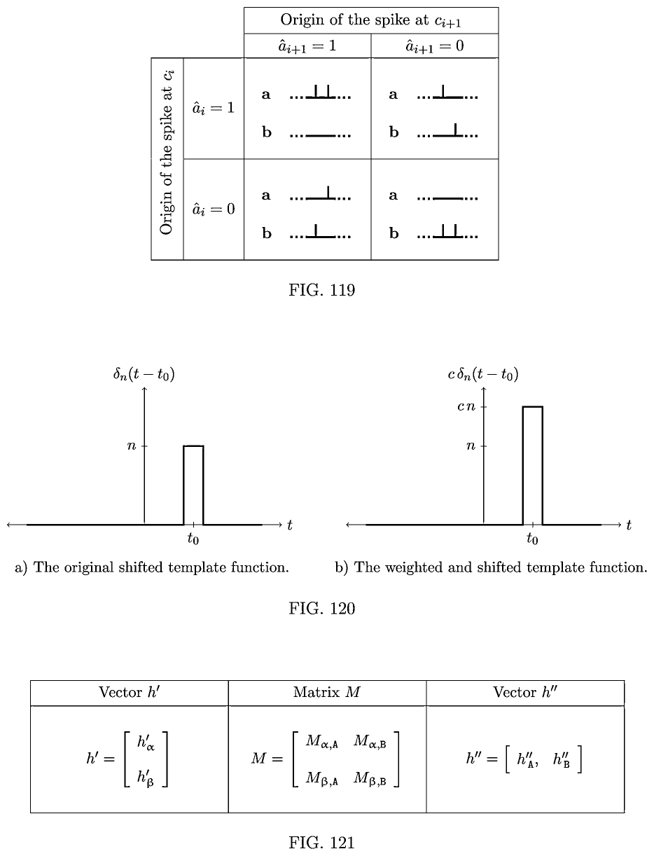

This section derives the common-timeline version of the iterative decoding verification formulas for ^{d}h_{b}″ shown in FIG. 117. The formulas are derived from the formulas for the state of the SSM model shown in FIG. 118. The derivation examines four special cases and shows that they reduce to two update formulas. FIG. 119 visualizes these four cases, which depend on the origin of the two most recent spikes (i.e., aa, ab, ba, or bb). As shown below, the two update formulas depend only on the origin of the most recent spike (i.e., a or b).

The formulas in FIG. 118 imply that the value of ^{d}h_{b}″ after the i-th iteration and after the (i+1)-st iteration can be expressed as follows:

$${}^{d} {h}_{b}^{″} [ i ] = \{ \begin{matrix}{e}^{s {c}_{i}} ℒ \{ b [ {c}_{i} , T ] \} ( s ) , if {\hat{a}}_{i} = 1 , \\ {e}^{s {c}_{i}} ℒ \{ b [ {c}_{i} , T ] \} ( s ) , if {\hat{a}}_{i} = 0 ,\end{matrix} \qquad ( 8.19 )$$

$${}^{d} {h}_{b}^{″} [ i + 1 ] = \{ \begin{matrix}{e}^{s {c}_{i + 1}} ℒ \{ b [ {c}_{i + 1} , T ] \} ( s ) , if {\hat{a}}_{i + 1} = 1 , \\ {e}^{s {c}_{i + 1}} ℒ \{ b [ {c}_{i + 1} , T ] \} ( s ) , if {\hat{a}}_{i + 1} = 0.\end{matrix} \qquad ( 8.191 )$$

The rest of this section applies these formulas to the four cases shown in FIG. 119 and derives an update formula for each case.

Case aa: In this case, both cᵢ and cᵢ₊₁ come from a. Therefore, only the first case of (8.190) and the first case of (8.191) apply. Also, in this case, cᵢ<cᵢ₊₁ because spikes on a don't coincide. Moreover, the truncated spike train b[cᵢ, cᵢ₊₁) is empty. This leads to the following expression:

$$\begin{matrix}{}^{d} {h}_{b}^{″} [ i + 1 ] = {e}^{s {c}_{i + 1}} ℒ \{ b [ {c}_{i + 1} , T ] \} ( s ) \\ = {e}^{s {c}_{i + 1}} ( ℒ \{ b [ {c}_{i} , T ] \} ( s ) - \\ = {e}^{s {c}_{i + 1}} {e}^{- s {c}_{i}} ( {e}^{s {c}_{i}} ℒ \{ b [ {c}_{i} , T ] \} ( s ) ) \\ = {e}^{s ( {c}_{i + 1} - {c}_{i} )} {}^{d} {h}_{b}^{″} [ i ] .\end{matrix} \qquad ( 8.192 )$$

Case ab: In this case, cᵢ originates from a and cᵢ₊₁ originates from b. Therefore, we need to use the first case of (8.190) and the second case of (8.191). Using the linearity of the Laplace transform and the fact that b[cᵢ, cᵢ₊₁] contains only one spike at cᵢ₊₁, we can derive the following:

$$\begin{matrix}{}^{d} {h}_{b}^{″} [ i + 1 ] = {e}^{s {c}_{i + 1}} ℒ \{ b [ {c}_{i + 1} , T ] \} ( s ) \\ = {e}^{s {c}_{i + 1}} ( ℒ \{ b [ {c}_{i} , T ] \} ( s ) - ℒ \{ b [ {c}_{i} , {c}_{i + 1} ] \} ( s ) ) \\ = {e}^{s {c}_{i + 1}} ( ℒ \{ b [ {c}_{i} , T ] \} ( s ) - {e}^{- s {c}_{i + 1}} ) \\ = {e}^{s {c}_{i + 1}} {e}^{- s {c}_{i}} ( {e}^{s {c}_{i}} ℒ \{ b [ {c}_{i} , T ] \} ( s ) ) - {\underbrace{{e}^{s {c}_{i + 1}} {e}^{- s {c}_{i + 1}}}}_{1} \\ = {e}^{s ( {c}_{i + 1} - {c}_{i} )} {}^{d} {h}_{b}^{″} [ i ] - 1.\end{matrix} \qquad ( 8.193 )$$

This is the only case in which there could be a coincidence, i.e., it is possible that cᵢ=cᵢ₊₁. However, formula (8.193) holds even for coincident spikes. That is, the truncated spike train b[cᵢ, cᵢ₊₁] would contain only one spike at cᵢ₊₁, even if cᵢ=cᵢ₊₁.

Case ba: In this case, âᵢ=0 and âᵢ₊₁=1. This implies that only the second case of (8.190) and the first case of (8.191) apply. Using the fact that the truncated spike train b(cᵢ, cᵢ₊₁) contains no spikes, we can derive the following formula:

$$\begin{matrix}{}^{d} {h}_{b}^{″} [ i + 1 ] = {e}^{s {c}_{i + 1}} ℒ \{ b [ {c}_{i + 1} , T ] \} ( s ) \\ = {e}^{s {c}_{i + 1}} ( ℒ \{ b [ {c}_{i} , T ] \} ( s ) - ) \\ = {e}^{s {c}_{i + 1}} {e}^{- s {c}_{i}} ( {e}^{s {c}_{i}} ℒ \{ b [ {c}_{i} , T ] \} ( s ) ) \\ = {e}^{s ( {c}_{i + 1} - {c}_{i} )} {}^{d} {h}_{b}^{″} [ i ] .\end{matrix} \qquad ( 8.194 )$$

By the construction of c and â, the two spikes cannot coincide in this case, i.e., cᵢ<cᵢ₊₁. If they did coincide, then the spike from a would be listed first, which would be handled by the case ab.

Case bb: In this case, both cᵢ and cᵢ₊₁ originate from b. Thus, we can use the second case of (8.190) and the second case of (8.191) to derive the following update formula:

$$\begin{matrix}{}^{d} {h}_{b}^{″} [ i + 1 ] = {e}^{s {c}_{i + 1}} ℒ \{ b [ {c}_{i + 1} , T ] \} ( s ) \\ = {e}^{s {c}_{i + 1}} ( ℒ \{ b [ {c}_{i} , T ] \} ( s ) - ℒ \{ b [ {c}_{i} , {c}_{i + 1} ] \} ( s ) ) \\ = {e}^{s {c}_{i + 1}} ( ℒ \{ b [ {c}_{i} , T ] \} ( s ) - {e}^{- s {c}_{i + 1}} ) \\ = {e}^{s {c}_{i + 1}} {e}^{- s {c}_{i}} ( {e}^{s {c}_{i}} ℒ \{ b [ {c}_{i} , T ] \} ( s ) ) - {\underbrace{{e}^{s {c}_{i + 1}} {e}^{- s {c}_{i + 1}}}}_{1} \\ = {e}^{s ( {c}_{i + 1} - {c}_{i} )} {}^{d} {h}_{b}^{″} [ i ] - 1.\end{matrix} \qquad ( 8.195 )$$

In this case, cᵢ is strictly less than cᵢ₊₁, because, by definition, the spike train b does not contain duplicate spikes. Thus, b(cᵢ₊₁, T] is different from b(cᵢ, T].

Even though there are four cases, they reduce to only two update formulas that depend only on the origin of the most recent spike. If the most recent spike is from a, then the previous value of ^{d}h_{b}″ is multiplied by e^{s(c}^{ᵢ₊₁}^{-c}^{ᵢ}⁾. On the other hand, if it comes from b, then ^{d}h_{b}″[i] is multiplied by e^{s(c}^{ᵢ₊₁}^{-c}^{ᵢ}⁾ and 1 is subtracted from the result.

8.12.5 Deriving the Update Formulas for ^{d}M_{a,b} from the Model

This section shows how the update formulas for ^{d}M_{a,b} from FIG. 117 can be derived from the formulas in FIG. 118, which describe the state of the SSM model after each iteration. FIG. 118 states that the value of ^{d}M_{a,b} after the i-th iteration and after the (i+1)-st iteration can be expressed as follows:

$${d}^{} {M}_{a , b} [ i ] = ℒ \{ a ( {c}_{i} , T ] ★ b [ {c}_{i} , T ] \} ( s ) , \qquad ( 8.196 )$$

$${d}^{} {M}_{a , b} [ i + 1 ] = ℒ \{ a ( {c}_{i + 1} , T ] ★ b [ {c}_{i + 1} , T ] \} ( s ) . \qquad ( 8.197 )$$

Once again, we need to consider four cases, which depend on the origin of the two most recent spikes. These four cases are visualized in FIG. 119 and analyzed below.

Case aa: In this case, both cᵢ and cᵢ₊₁ originate from a. Thus, the truncated spike train b[cᵢ, cᵢ₊₁) is empty. Moreover, cᵢ<cᵢ₊₁, because the spikes in a cannot coincide. Therefore,

$$\begin{matrix}{d}^{} {M}_{a , b} [ i ] = ℒ \{ a ( {c}_{i} , T ] ★ b [ {c}_{i} , T ] \} ( s ) \\ = ℒ \{ a ( {c}_{i} , {c}_{i + 1} ] ★ b [ {c}_{i} , T ] \} ( s ) + ℒ \{ a ( {c}_{i + 1} , T ] ★ b [ {c}_{i} , T ] \} ( s ) \\ = ℒ \{ ( {c}_{i + 1} ) ★ b [ {c}_{i} , T ] \} ( s ) + + ℒ \{ a ( {c}_{i + 1} , T ] ★ b [ {c}_{i + 1} , T ] \} ( s ) \\ = + ℒ \{ ( {c}_{i + 1} ) ★ b [ {c}_{i + 1} , T ] \} ( s ) + {d}^{} {M}_{a , b} [ i + 1 ] \\ = {e}^{s {c}_{i + 1}} ℒ \{ b [ {c}_{i + 1} , T ] \} ( s ) + {d}^{} {M}_{a , b} [ i + 1 ] \\ = {}^{d} {h}_{b}^{″} [ i + 1 ] + {d}^{} {M}_{a , b} [ i + 1 ] .\end{matrix} \qquad ( 8.198 )$$

Rearranging the terms leads to the following update formula:

$${d}^{} {M}_{a , b} [ i + 1 ] = {d}^{} {M}_{a , b} [ i ] - {}^{d} {h}_{b}^{″} [ i + 1 ] . \qquad ( 8.199 )$$

Note that (8.198) used a property of the Laplace transform of the cross-correlation of a single spike and a spike train, which implies that:

$$ℒ \{ ( {c}_{i + 1} ) ★ b [ {c}_{i + 1} , T ] \} ( s ) = {e}^{s {c}_{i + 1}} ℒ \{ b [ {c}_{i + 1} , T ] \} ( s ) = {}^{d} {h}_{b}^{″} [ i + 1 ] . \qquad ( 8.2 )$$

Case ab: Suppose that cᵢ<cᵢ₊₁, i.e., there is no coincidence. Then, the truncated spike trains a(cᵢ, cᵢ₊₁] and b[cᵢ, cᵢ₊₁) are empty. Therefore,

$$\begin{matrix}{d}^{} {M}_{a , b} [ i ] = ℒ \{ a ( {c}_{i} , T ] ★ b [ {c}_{i} , T ] \} ( s ) \\ = + ℒ \{ a ( {c}_{i + 1} , T ] ★ b [ {c}_{i} , T ] \} ( s ) \\ = + ℒ \{ a ( {c}_{i + 1} , T ] ★ b [ {c}_{i + 1} , T ] \} ( s ) \\ = {d}^{} {M}_{a , b} [ i + 1 ] .\end{matrix} \qquad ( 8.201 )$$

By the construction of c and â, this is the only case in which there can be a coincidence, i.e., it is possible that cᵢ=cᵢ₊₁. Even if cᵢ=cᵢ₊₁, however, it would still be true that ^{d}M_{a,b}[i]=^{d}M_{a,b}[i+1], because the truncation intervals in (8.196) and (8.197) would be the same.

Case ba: In this case, cᵢ originates from a, cᵢ₊₁ originates from b, and there can be no coincidences, i.e., cᵢ is strictly less than cᵢ₊₁. Due to the construction of the common timeline, all coincidences are handled by the case ab. Thus, we can derive the following expression, which leads to the same update formula as in (8.199):

$$\begin{matrix}{}_{}^{d} {M}_{a , b}^{} [ i ] = ℒ \{ a ( {c}_{i} , T ] ★ b [ {c}_{i} , T ] \} ( s ) \\ = ℒ \{ a ( {c}_{i} , {c}_{i + 1} ] ★ b [ {c}_{i} , T ] \} ( s ) + ℒ \{ a ( {c}_{i + 1} , T ] ★ b [ {c}_{i} , T ] \} ( s ) \\ = ℒ \{ ( {c}_{i + 1} ) ★ b [ {c}_{i} , T ] \} ( s ) + + \\ ℒ \{ a ( {c}_{i + 1} , T ] ★ b [ {c}_{i + 1} , T ] \} ( s ) \\ = + ℒ \{ ( {c}_{i + 1} ) ★ b [ {c}_{i + 1} , T ] \} ( s ) + {}_{}^{d} {M}_{a , b}^{} [ i + 1 ] \\ = {e}^{s {c}_{i + 1}} ℒ \{ b [ {c}_{i + 1} , T ] \} ( s ) + {}_{}^{d} {M}_{a , b}^{} [ i + 1 ] \\ = {}_{}^{d} {h}_{b}^{″} [ i + 1 ] + {}_{}^{d} {M}_{a , b}^{} [ i + 1 ] .\end{matrix} \qquad ( 8.202 )$$

The two cancellations in this derivation can be explained as follows. Each spike in the interval a(cᵢ₊₁, T] follows every spike in the interval b[cᵢ, cᵢ₊₁). However, only spike pairs in which the spike from a(cᵢ₊₁, T] precedes or coincides with a spike from b[cᵢ, cᵢ₊₁) contribute to the value of [symbol] {a(cᵢ₊₁, T]*b[cᵢ, cᵢ₊₁)}(s). This implies that its value is zero. Similarly, cᵢ₊₁ follows every spike in b[cᵢ, cᵢ₊₁), which implies that [symbol] {(cᵢ₊₁)*b[cᵢ, cᵢ₊₁)}(s) is also equal to zero. This derivation uses the property [symbol] {(cᵢ₊₁)*b[cᵢ₊₁, T]}(s)=e^{sc}^{ᵢ₊₁} [symbol] {b[cᵢ₊₁, T]}(s).

Case bb: In the fourth case, both spikes come from b and there can be no coincidences. Using the fact that in this case a(cᵢ, cᵢ₊₁] is empty, we can derive the following update formula:

$$\begin{matrix}{}_{}^{d} {M}_{a , b}^{} [ i ] = ℒ \{ a ( {c}_{i} , T ] ★ b [ {c}_{i} , T ] \} ( s ) \\ = + ℒ \{ a ( {c}_{i + 1} , T ] ★ b [ {c}_{i} , T ] \} ( s ) \\ = + ℒ \{ a ( {c}_{i + 1} , T ] ★ b [ {c}_{i + 1} , T ] \} ( s ) \\ = {}_{}^{d} {M}_{a , b}^{} [ i + 1 ] .\end{matrix} \qquad ( 8.203 )$$

The second cancellation in this derivation is justified because the two truncated spike trains a(cᵢ₊₁, T] and b[cᵢ, cᵢ₊₁) don't overlap and therefore the Laplace transform of their cross-correlation is equal to zero.

Similarly to the update formulas for ^{d}h_{b}″, the four cases for ^{d}M_{a,b} collapse to just two update formulas. These formulas depend only on the origin of the most recent spike, i.e., they depend on âᵢ₊₁. The two formulas match the update formulas shown in FIG. 117.

8.12.6 At the End of Decoding Verification ^{d}h_{b}″ and ^{d}M_{a,b} are Equal to Zero

This section shows that at the end of the verification process the value of ^{d}h_{b}″ and the value of ^{d}M_{a,b} are equal to zero. In other words, this section shows that all four formulas in FIG. 118 evaluate to zero for i=J+K−1. In that case, âᵢ₊₁=â_{J+K} and cᵢ₊₁=c_{J+K}.

If â_{J+K}=1, then c_{J+K} comes from a. Therefore, the last spike on b occurs strictly before c_{J+K} (otherwise, the coincidence would be handled by the case ab). Therefore, the truncated spike train b[c_{J+K}, T] is empty. Thus,

$${}_{}^{d} {h}_{b}^{″} [ J + K ] = {e}^{s {c}_{J + K}} ℒ \{ b [ {c}_{J + K} , T ] \} ( s ) = 0 \qquad ( 8.204 )$$

Moreover, the truncated spike train a(c_{J+K}, T] is also empty. Thus,

$${}_{}^{d} {M}_{a , b}^{} [ J + K ] = ℒ \{ a ( {c}_{J + K} , T ] ★ b [ {c}_{J + K} , T ] \} ( s ) = 0. \qquad ( 8.205 )$$

If â_{J+K}=0, then b(c_{J+K}, T] is empty. Therefore,

$${}_{}^{d} {h}_{b}^{″} [ J + K ] = {e}^{s {c}_{J + K}} ℒ \{ b [ {c}_{J + K} , T ] \} ( s ) = 0. \qquad ( 8.206 )$$

The truncated spike train a(c_{J+K}, T] is empty in this case too. Thus,

$${}_{}^{d} {M}_{a , b}^{} [ J + K ] = ℒ \{ a ( {c}_{J + K} , T ] ★ b [ {c}_{J + K} , T ] \} ( s ) = 0. \qquad ( 8.207 )$$

### 8.13 The Decoding Verification Algorithm

The decoding verification procedure that was described in Section 8.12 can be implemented by an algorithm for which the run-time complexity is O(J+K), where J is the number of spikes in a and K is the number of spikes in b. If the verification is successful, then the two values returned by this algorithm should be equal to zero.

Once again, this is a verification algorithm, not a decoding algorithm, because the spike train a is given to the algorithm. A decoding algorithm would have to infer the spike train a. Also, this algorithm verifies only one element of the matrix. Because the computation is local and does not depend on any other matrix element, however, different instances of this algorithm can be run in parallel. For example, to verify the entire matrix, one instance of the algorithm could be run for each matrix element.

### 9 Continuous-Time Formulation for Weighted Spike Trains

This chapter extends the theory described in Chapter 8 so that it can be applied to weighted spike trains. These extensions are then used to state the SUV family of algorithms, which can be viewed as the continuous-time counterparts to the ZUV algorithms for discrete sequences.

### 9.1 Modeling Weighted Spikes and Weighted Spike Trains

Section 8.4 described how to model spikes and spike trains. In that case all spikes were alike. This section extends the theory so that it can handle spikes that are weighted differently.

In the previous case each spike was modeled with a shifted template function δₙ(t−t₀), which was defined as follows:

$${δ}_{n} ( t - {t}_{0} ) = \{ \begin{matrix}0 , & if t < {t}_{0} - \frac{1}{2 n} , \\ n , & if {t}_{0} - \frac{1}{2 n} \leq t \leq {t}_{0} + \frac{1}{2 n} , \\ 0 , & if t > {t}_{0} + \frac{1}{2 n}\end{matrix} . \qquad ( 9.1 )$$

In this chapter we will use the same template function, but it will be weighted differently for different spikes.

Let c be a complex scalar. Then, the weighted and shifted template function cδₙ(t−t₀) is defined as follows:

$$c {δ}_{n} ( t - {t}_{0} ) = \{ \begin{matrix}0 , & if t < {t}_{0} - \frac{1}{2 n} , \\ cn , & if {t}_{0} - \frac{1}{2 n} \leq t \leq {t}_{0} + \frac{1}{2 n} , \\ 0 , & if t > {t}_{0} + \frac{1}{2 n}\end{matrix} . \qquad ( 9.2 )$$

Note that in this definition the height of the template is scaled by c, but the width is not scaled. Therefore, the area under the curve is no longer equal to 1 if c≠1. Also, note that the scaled template could be complex, while the original one is always real. FIG. 120 illustrates the difference between the templates defined by equations (9.1) and (9.2).

To model a weighted spike train, we first need to introduce a notation for the weights that will be associated with each spike. Let v(t) be a weighting function and let b⁽ⁿ⁾(t) be the model for the spike train b=(b₁, b₂, . . . , b_{K}). We will use the notation (vb⁽ⁿ⁾)(t) to denote the spike train obtained after weighting b⁽ⁿ⁾ by v(t). The superscript n indicates that the template function δₙ(t−bₖ) is used to model each spike before it is scaled by v(t).

Definition 9.1. The model for the spike train b=(b₁, b₂, . . . , b_{K}) that is weighted by the function v(t) is the sequence of functions ((vb⁽¹⁾)(t), (vb⁽²⁾)(t), . . . , (vb⁽ⁿ⁾)(t), . . . , where

$$( {vb}^{( n )} ) ( t ) = v ( t ) {b}^{( n )} ( t ) = v ( t ) {\sum }_{k = 1}^{K} {δ}_{n} ( t - {b}_{k} ) = {\sum }_{k = 1}^{K} v ( t ) {δ}_{n} ( t - {b}_{k} ) , \qquad ( 9.3 )$$

for each n∈{1, 2, . . . }. In this notation b₁, b₂, . . . , b_{K} denote the times at which the individual spikes occur. It is assumed that the list of spikes is sorted in increasing order and that this list does not contain any duplicates.

Using a similar approach the spike train a=(a₁, a₂, . . . , a_{J}) that is weighted by the function u(t) can be defined as:

$$( u {a}^{( m )} ) ( t ) = {\sum }_{j = 1}^{J} u ( t ) {δ}_{m} ( t - {a}_{j} ) . \qquad ( 9.4 )$$

In this case the shifted template function is δₘ and it is defined as follows:

$${δ}_{m} ( t - {t}_{0} ) = \{ \begin{matrix}0 , & if t < {t}_{0} - \frac{1}{2 m} , \\ m , & if {t}_{0} - \frac{1}{2 m} \leq t \leq {t}_{0} + \frac{1}{2 m} , \\ 0 , & if t > {t}_{0} + \frac{1}{2 m}\end{matrix} . \qquad ( 9.5 )$$

### 9.2 Operations on Weighted Spike Trains

This section defines some operations on weighted spike trains. These are similar to the operations on spike trains defined in Section 8.5, but now the spike trains are weighted. As a result of this weighting, the notation and the formulas are slightly different. By default it will be assumed that all weighting functions are continuous functions.

### 9.2.1 The Laplace Transform of a Weighted Spike Train

Let a=(a₁, a₂, . . . , a_{J}) be a spike train and let u(t) be a complex function of a nonnegative real argument, i.e., u: [symbol] ₀⁺→ [symbol] . The value of the Laplace transform of the spike train a weighted by the function u(t) will be denoted by [symbol] ₐ^{(u)}(s). That is, the superscript is the weighting function, the subscript is the spike train, and s is the argument of the Laplace transform. Note that u(t) is not the unit step function (a.k.a., Heaviside function), which is denoted with H(t) in this document.

Definition 9.2. The Laplace transform of the spike train a=(a₁, a₂, . . . , a_{J}) that is weighted by the function u(t) is a function obtained by taking the limit of the sequence of Laplace transforms of functions in the model for the spike train a weighted by u(t). More formally,

$${ℒ}_{a}^{( u )} ( s ) = {lim}_{m \to ∞} ℒ \{ u {a}^{( m )} \} ( s ) = {lim}_{m \to ∞} {\int }_{0 -}^{∞} u ( t ) {a}^{( m )} ( t ) {e}^{- st} dt . \qquad ( 9.6 )$$

If the weighting function is continuous, then the value of the Laplace transform of the weighted spike train can be expressed in terms of the values of the weighting function at each of the spike times. This derivation is shown below.

$$\begin{matrix}{ℒ}_{a}^{( u )} ( s ) & = & {lim}_{m \to ∞} ℒ \{ u {a}^{( m )} \} ( s ) & ( Therorem 8.16 ) \\ & = & {lim}_{m \to ∞} {\int }_{0 -}^{∞} u ( t ) {a}^{( m )} ( t ) {e}^{- st} dt & \\ & = & {lim}_{m \to ∞} {\int }_{0 -}^{∞} {\sum }_{j = 1}^{J} u ( t ) {δ}_{m} ( t - {a}_{j} ) {e}^{- st} dt & \\ & = & {\sum }_{j = 1}^{J} {lim}_{m \to ∞} {\int }_{0 -}^{∞} {δ}_{m} ( t - {a}_{j} ) u ( t ) {e}^{- st} dt & \\ & = & {\sum }_{j = 1}^{J} {lim}_{m \to ∞} {\int }_{0 -}^{∞} H ( t - {0}^{-} ) {δ}_{m} ( t - {a}_{j} ) u ( t ) {e}^{- st} dt & \\ & = & {\sum }_{j = 1}^{J} H ( {a}_{j} - 0 ) ( {lim}_{m \to ∞} {\int }_{- ∞}^{∞} {δ}_{m} ( t - {a}_{j} ) u ( t ) {e}^{- st} dt ) & \\ & = & {\sum }_{j = 1}^{J} H ( {a}_{j} ) u ( {a}_{j} ) {e}^{- {sa}_{j}} . & ( 9.7 )\end{matrix}$$

If the spike train a is causal (i.e., if aⱼ≥0 for all j), then the Heaviside function in the previous expression will always be equal to 1 and the formula can be simplified as follows:

$${ℒ}_{a}^{( u )} ( s ) = {\sum }_{j = 1}^{J} u ( {a}_{j} ) {e}^{- {sa}_{j}} . \qquad ( 9.8 )$$

Furthermore, if the spike train a is causal and contains just one spike that occurs at time a₁, i.e., a=(a₁), then the formula reduces to the formula for the Laplace transform of a weighted Dirac's delta that is shifted to the right by a₁. In this case the expression is:

$$\begin{matrix}{ℒ}_{a}^{( u )} ( s ) = {ℒ}^{( u )} \{ δ ( t - {a}_{1} ) \} ( s ) \\ = {lim}_{m \to ∞} ℒ \{ u ( t ) {δ}_{m} ( t - {a}_{1} ) \} ( s ) \\ = {\sum }_{j = 1}^{1} u ( {a}_{j} ) {e}^{- {sa}_{j}} \\ = u ( {a}_{1} ) {e}^{- {sa}_{1}} .\end{matrix} \qquad ( 9.9 )$$

Similarly, if v(t) is a continuous weighting function and b=(b₁, b₂, . . . , b_{K}) is a spike train, then the Laplace transform of b weighted by v(t) is given by:

$${ℒ}_{b}^{( v )} ( s ) = {\sum }_{k = 1}^{K} H ( {b}_{k} ) v ( {b}_{k} ) {e}^{- s {b}_{k}} . \qquad ( 9.1 )$$

If the spike train b is causal, then this simplifies as follows:

$${ℒ}_{b}^{( v )} ( s ) = {\sum }_{k = 1}^{K} v ( {b}_{k} ) {e}^{- s {b}_{k}} . \qquad ( 9.11 )$$

Finally, if b is causal and has just one spike at time b₁, i.e., b=(b₁), then the formula reduces to:

$${ℒ}_{b}^{( v )} ( s ) = {ℒ}^{( v )} \{ δ ( t - {b}_{1} ) \} ( s ) = v ( {b}_{1} ) {e}^{- s {b}_{1}} . \qquad ( 9.12 )$$

### 9.2.2 The Cross-Correlation of Two Weighted Spike Trains

This section defines the cross-correlation of two weighted spike trains. In extends both the theory and the notation described in Section 8.5.2.

Let a=(a₁, a₂, . . . , a_{J}) be a spike train that has J spikes that occur at times a₁, a₂, . . . , a_{J}. Also, let u(t) be a weighting function. As described in Section 9.1 the weighted spike train can be expressed as the sum of weighted and shifted template functions δₘ. In other words,

$$\begin{matrix}( u {a}^{( m )} ) ( t ) = {\sum }_{j = 1}^{J} u ( t ) {δ}_{m} ( t - {a}_{j} ) , & for each m ∈ ℕ = \{ 1 , 2 , … \} .\end{matrix} \qquad ( 9.13 )$$

Similarly, if b=(b₁, b₂, . . . , b_{K}) is another spike train that is weighted by the function v(t), then the weighted spike train can be expressed as

$$\begin{matrix}( v {b}^{( n )} ) ( t ) = {\sum }_{k = 1}^{K} v ( t ) {δ}_{n} ( t - {b}_{k} ) , & for each n ∈ ℕ = \{ 1 , 2 , … \} ,\end{matrix} \qquad ( 9.14 )$$

where δₙ is also a shifted template function.

The following definition formally states the model for the cross-correlation of two weighted spike trains.

Definition 9.3. Let a=(a₁, a₂, . . . , a_{J}) and b=(b₁, b₂, . . . , b_{K}) be two spike trains and let u(t) and v(t) be two weighting functions. Then, the model for the cross-correlation of the spike train a weighted by the function u(t) and the spike train b weighted by the function v(t) is formed by the functions (ua^{(m)}*(vb⁽ⁿ⁾))(t), where m, n∈ [symbol] ={1, 2, . . . } such that

$$\begin{matrix}( ( u {a}^{( m )} ) ★ ( v {b}^{( n )} ) ) ( t ) = {\int }_{- ∞}^{∞} \overline{u ( τ ) {a}^{( m )} ( τ )} v ( τ + t ) {b}^{( n )} ( τ + t ) d τ \\ = {\int }_{- ∞}^{∞} \overline{u ( τ )} {a}^{( m )} ( τ ) v ( τ + t ) {b}^{( n )} ( τ + t ) d τ .\end{matrix} \qquad ( 9.15 )$$

Note that in (9.15) the conjugation over a^{(m)}(τ) can be dropped because it is modeled with a template function δₘ that is a real-valued function of a real argument. The conjugation over u̅(̅τ̅)̅, however, cannot be dropped because u may be a complex function of a real variable. This conjugation is one of the main differences between the formulas in this chapter and the formulas in Chapter 8. The other major difference, of course, is the presence of the weighting functions in all formulas in this chapter.

### 9.2.3 The Laplace Transform of the Cross-Correlation of two Weighted Spike Trains

The Laplace transform of the cross-correlation of the spike train a=(a₁, a₂, . . . , a_{J}) weighted by the function u(t) and the spike train b=(b₁, b₂, . . . , b_{K}) weighted by the function v(t) will be denoted with [symbol] ^{(u,v)}{a*b}(s) or with [symbol] _{a*b}^{(u,v)}(s) for short. As shown below, the result of this operation is defined as the iterated limit of the Laplace transform of the cross-correlation of ua^{(m)} and vb⁽ⁿ⁾ as the width of the template δₘ and the width of the template δₙ tend to zero.

Definition 9.4. Let a=(a₁, a₂, . . . , a_{J}) and b=(b₁, b₂, . . . , b_{K}) be two spike trains. Also, let u(t) and v(t) be two weighting functions such that u,v: [symbol] ₀⁺→ [symbol] , i.e., they are complex functions of a nonnegative real argument. Then, the Laplace transform of the cross-correlation of the spike train a weighted by the function u(t) and the spike train b weighted by the function v(t) is a function obtained by evaluating the iterated limit of the sequence of Laplace transforms of the cross-correlation functions specified in Definition 9.3 as n and m tend to infinity. The resulting function is denoted by [symbol] _{a*b}^{(u,v)}(s). More formally,

$$\begin{matrix}{ℒ}_{a ★ b}^{( u , v )} ( s ) = {lim}_{m \to ∞} {lim}_{n \to ∞} ℒ \{ ( u {a}^{( m )} ) ★ ( v {b}^{( n )} ) \} ( s ) \\ = {lim}_{m \to ∞} {lim}_{n \to ∞} {\int }_{{0}^{-}}^{∞} ( ( u {a}^{( m )} ) ★ ( v {b}^{( n )} ) ) ( t ) {e}^{- st} dt .\end{matrix} \qquad ( 9.16 )$$

Starting with this definition, we can derive a closed-form formula for the Laplace transform of the cross-correlation of two causal weighted spike trains. The first step is shown below:

$$\begin{matrix}{ℒ}_{a ★ b}^{( u , v )} ( s ) = {lim}_{m \to ∞} {lim}_{n \to ∞} {\int }_{{0}^{-}}^{∞} ( ( u {a}^{( m )} ) ★ ( v {b}^{( n )} ) ) ( t ) {e}^{- st} dt \\ = {lim}_{m \to ∞} {lim}_{n \to ∞} {\int }_{{0}^{-}}^{∞} ( {\int }_{- ∞}^{∞} \overline{u ( τ )} {a}^{( m )} ( τ ) v ( τ + t ) {b}^{( n )} ( τ + t ) d τ ) {e}^{- st} dt \\ = {lim}_{m \to ∞} {lim}_{n \to ∞} {\int }_{{0}^{-}}^{∞} ( {\int }_{- ∞}^{∞} ( {\sum }_{j = 1}^{J} \overline{u ( τ )} {δ}_{m} ( τ - {a}_{j} ) ) ( {\sum }_{k = 1}^{K} v ( τ + t ) {δ}_{n} ( τ + t - {b}_{k} ) ) d τ ) {e}^{- st} dt \\ = {lim}_{m \to ∞} {lim}_{n \to ∞} {\int }_{{0}^{-}}^{∞} ( {\sum }_{j = 1}^{J} {\sum }_{k = 1}^{K} {\int }_{- ∞}^{∞} \overline{u ( τ )} {δ}_{m} ( τ - {a}_{j} ) v ( τ + t ) {δ}_{n} ( τ + t - {b}_{k} ) d τ ) {e}^{- st} dt \\ = {\sum }_{j = 1}^{J} {\sum }_{k = 1}^{K} {lim}_{m \to ∞} {lim}_{n \to ∞} {\int }_{{0}^{-}}^{∞} {\int }_{- ∞}^{∞} \overline{u ( τ )} {δ}_{m} ( τ - {a}_{j} ) v ( τ + t ) {δ}_{n} ( τ + t - {b}_{k} ) {e}^{- st} d τ dt \\ = {\sum }_{j = 1}^{J} {\sum }_{k = 1}^{K} {lim}_{m \to ∞} {lim}_{n \to ∞} {\int }_{- ∞}^{∞} {\int }_{{0}^{-}}^{∞} \overline{u ( τ )} {δ}_{m} ( τ - {a}_{j} ) v ( τ + t ) {δ}_{n} ( τ + t - {b}_{k} ) {e}^{- st} d td τ \\ = {\sum }_{j = 1}^{J} {\sum }_{k = 1}^{K} {lim}_{m \to ∞} {lim}_{n \to ∞} {\int }_{- ∞}^{∞} \overline{u ( τ )} {δ}_{m} ( τ - {a}_{j} ) {\int }_{- ∞}^{∞} H ( t - {0}^{-} ) v ( τ + t ) {δ}_{n} ( τ + t - {b}_{k} ) {e}^{- st} d td τ \\ = {\sum }_{j = 1}^{J} {\sum }_{k = 1}^{K} {lim}_{m \to ∞} {\int }_{- ∞}^{∞} {δ}_{m} ( τ - {a}_{j} ) \overline{u ( τ )} {\underbrace{( {lim}_{n \to ∞} {\int }_{- ∞}^{∞} H ( t - {0}^{-} ) {δ}_{n} ( t - ( {b}_{k} - τ ) ) v ( τ + t ) {e}^{- st} d t )}}_{{f}_{k} ( τ )} d τ \\ = {\sum }_{j = 1}^{J} {\sum }_{k = 1}^{K} {lim}_{m \to ∞} {\int }_{- ∞}^{∞} {δ}_{m} ( τ - {a}_{j} ) \overline{u ( τ )} {f}_{k} ( τ ) d τ .\end{matrix} \qquad ( 9.17 )$$

The previous expression uses the short-hand notation fₖ(τ), which is defined as:

$${f}_{k} ( τ ) = {lim}_{n \to ∞} {\int }_{- ∞}^{∞} H ( t - {0}^{-} ) {δ}_{n} ( t - ( {b}_{k} - τ ) ) v ( τ + t ) {e}^{- st} d t . \qquad ( 9.18 )$$

To continue the derivation we will show that the limit of fₖ(τ) as τ→t₀ exists and is finite for each t₀∈ [symbol] . To do this, we will use the variable substitution \hat{τ}=bₖ−τ to derive the following closed-form expression for the value of this limit:

$$\begin{matrix}{lim}_{τ \to {t}_{0}} {f}_{k} ( τ ) = {lim}_{τ \to {t}_{0}} {lim}_{n \to ∞} {\int }_{- ∞}^{∞} H ( t - {0}^{-} ) {δ}_{n} ( t - ( {b}_{k} - τ ) ) v ( τ + t ) {e}^{- st} dt \\ = {lim}_{\hat{τ} \to ( {b}_{k} - {t}_{0} )} {lim}_{n \to ∞} {\int }_{- ∞}^{∞} H ( t - {0}^{-} ) {δ}_{n} ( t - \hat{τ} ) v ( {b}_{k} - \hat{τ} + t ) {e}^{- st} dt \\ = H ( ( {b}_{k} - {t}_{0} ) - 0 ) ( {lim}_{\hat{τ} \to ( {b}_{k} - {t}_{0} )} {\underbrace{( {lim}_{n \to ∞} {\int }_{- ∞}^{∞} {δ}_{n} ( t - \hat{τ} ) v ( {b}_{k} - \hat{τ} + t ) {e}^{- st} dt )}}_{v ( {b}_{k} - + ) {e}^{- s \hat{τ}}} ) \\ = H ( {b}_{k} - {t}_{0} ) ( {lim}_{\hat{τ} \to ( {b}_{k} - {t}_{0} )} v ( {b}_{k} ) {e}^{- s \hat{τ}} ) \\ = H ( {b}_{k} - {t}_{0} ) v ( {b}_{k} ) {e}^{- s ( {b}_{k} - {t}_{0} )} .\end{matrix} \qquad \begin{matrix}( Theorem 8.16 ) \\ ( 919. )\end{matrix}$$

Formula (9.19) moves the Heaviside function out of the integral and the two limits. It also uses Theorem 8.16, which can be applied to the inner limit because v(bₖ−\hat{τ}+t)e^{-st} is continuous. Finally, it evaluates the limit as τ→(bₖ−t₀) of the result from Theorem 8.16. This limit exists and is finite.

To get the formula for the value of [symbol] _{a*b}^{(u,v)}(s) we can combine (9.19) and (9.17) as shown below:

$$\begin{matrix}{ℒ}_{a ★ b}^{( u , v )} ( s ) = \begin{matrix}{\sum }_{j = 1}^{J} {\sum }_{k = 1}^{K} {lim}_{m \to ∞} {\int }_{- ∞}^{∞} {δ}_{m} ( τ - {a}_{j} ) \overline{u ( τ )} {f}_{k} ( τ ) d τ & ( Theorem 8.15 )\end{matrix} \\ = \begin{matrix}{\sum }_{j = 1}^{J} {\sum }_{k = 1}^{K} {lim}_{τ \to {a}_{j}} ( \overline{u ( τ )} {f}_{k} ( τ ) ) & ( Formula ( 9.19 ) )\end{matrix} \\ = \begin{matrix}{\sum }_{j = 1}^{J} {\sum }_{k = 1}^{K} H ( {b}_{k} - {a}_{j} ) \overline{u ( {a}_{j} )} v ( {b}_{k} ) {e}^{- s ( {b}_{k} - {a}_{j} )} . & ( 9.2 )\end{matrix}\end{matrix}$$

To summarize, the formula for the Laplace transform of the cross-correlation of two causal weighted spike trains is:

$${ℒ}_{a ★ b}^{( u , v )} ( s ) = {\sum }_{j = 1}^{J} {\sum }_{k = 1}^{K} H ( {b}_{k} - {a}_{j} ) \overline{u ( {a}_{j} )} v ( {b}_{k} ) {e}^{- s ( {b}_{k} - {a}_{j} )} . \qquad ( 9.21 )$$

In the special case when u(t)=1 and v(t)=1 this formula reduces to formula (8.46), which defines [symbol] _{a*b}(s), i.e., the Laplace transform of the cross-correlation of two unweighted spike trains. By extension, this formula also reduces to all the other special cases described in Section 8.5.3.

### 9.2.4 Some Additional Properties

Property 9.5. Let b=(b₁, b₂, . . . , b_{K}) be a spike train that has K spikes. Also, let v(t) be a weighting function that is defined as follows:

$$v ( t ) = {e}^{{s}_{0} t} , \qquad ( 9.22 )$$

where s₀ is a complex constant. Then,

$${ℒ}^{( v )} \{ b \} ( s ) = ℒ \{ b \} ( s - {s}_{0} ) . \qquad ( 9.23 )$$

Property 9.6. Let a=(a₁, a₂, . . . , a_{J}) and b=(b₁, b₂, . . . , b_{K}) be two causal spike trains that have J and K spikes, respectively. Also, let u(t) and v(t) be two weighting functions that are defined as follows:

$$u ( t ) = {e}^{- \overline{{s}_{0}} t} , \qquad ( 9.24 )$$

$$v ( t ) = {e}^{{s}_{0} t} , \qquad ( 9.25 )$$

where s₀ is a complex constant. Then,

$${ℒ}^{( u , v )} \{ a ★ b \} ( s ) = ℒ \{ a ★ b \} ( s - {s}_{0} ) . \qquad ( 9.26 )$$

Property 9.7. Let a=(a₁, a₂, . . . , a_{J}) and b=(b₁, b₂, . . . , b_{K}) be two causal spike trains that have J and K spikes, respectively. Also, let u(t) and v(t) be two weighting functions that are defined as follows:

$$u ( t ) = U {e}^{- \overline{{s}_{0}} t} , \qquad ( 9.27 )$$

$$v ( t ) = V {e}^{{s}_{0} t} , \qquad ( 9.28 )$$

where so, U, and V are complex constants. Then

$${ℒ}^{( u , v )} \{ a ★ b \} ( s ) = {ℒ}^{( U , V )} \{ a ★ b \} ( s - {s}_{0} ) . \qquad ( 9.29 )$$

### 9.3 Operations on Weighted and Truncated Spike Trains

This section extends the theory described in Section 8.6. The new formulas can be used with truncated spike trains that are also weighted. The truncation is still performed using two Heaviside functions and left- and right-limits for the bounds.

### 9.3.1 The Laplace Transform of a Weighted and Truncated Spike Train

Definition 9.8. Model for a weighted and truncated spike train. Let b=(b₁, b₂, . . . , b_{K}) be a spike train that contains K spikes, and let v(t) be a weighting function. Also, let t₁ and t₂ be two real numbers such that t₁≤t₂. The model for the weighted and truncated spike train is a sequence of functions ((vb_{[t}_{₁}_{,t}_{₂}_{]}⁽¹⁾)(t), (vb_{[t}_{₁}_{,t}_{₂}_{]}⁽²⁾)(t), . . . , (vb_{[t}_{₁}_{,t}_{₂}_{]}⁽ⁿ⁾(t), . . . ), where

$$( v {b}_{[ {t}_{1} , {t}_{2} ]}^{( n )} ) ( t ) = H ( t - {t}_{1}^{-} ) H ( {t}_{2}^{+} - t ) ( v {b}^{( n )} ) ( t ) , \qquad ( 9.3 )$$

for each n∈{1, 2, . . . }.
Definition 9.9. The Laplace transform of a weighted and truncated spike train. Let b=(b₁, b₂, . . . , b_{K}) be a spike train that has K spikes and let v(t) be a weighting function. Also, let t₁ and t₂ be two real numbers that determine the truncation interval such that t₁≤t₂. Then, the Laplace transform of the truncated spike train b[t₁, t₂] that is weighted by v(t) is a function that is obtained by taking the limit of the sequence of Laplace transforms of functions that are given by Definition 9.8. More formally,

$${ℒ}^{( v )} \{ b [ {t}_{1} , {t}_{2} ] \} ( s ) = {lim}_{n \to ∞} {ℒ}^{( v )} \{ {b}_{{[ {t}_{1} , {t}_{2} ]}^{}}^{( n )} \} = {lim}_{n \to ∞} {\int }_{0 -}^{∞} H ( t - {t}_{1}^{-} ) H ( {t}_{2}^{+} - t ) v ( t ) {b}^{( n )} ( t ) {e}^{- s t} dt . \qquad ( 9.31 )$$

Because each spike train is modeled as a sum of shifted and weighted template functions we can use Definition 9.9 to derive a closed-form expression for the Laplace transform of a weighted and truncated spike train in which the integral reduces to a sum. More formally,

$$\begin{matrix}{ℒ}^{( v )} \{ b [ {t}_{1} , {t}_{2} ] \} ( s ) = {lim}_{n \to ∞} {\int }_{0 -}^{∞} H ( t - {t}_{1}^{-} ) H ( {t}_{2}^{+} - t ) v ( t ) {b}^{( n )} ( t ) {e}^{- s t} dt \\ = {lim}_{n \to ∞} {\int }_{0 -}^{∞} H ( t - {t}_{1}^{-} ) H ( {t}_{2}^{+} - t ) v ( t ) ( {\sum }_{k = 1}^{K} {δ}_{n} ( t - {b}_{k} ) ) {e}^{- s t} d t \\ = {\sum }_{k = 1}^{K} {lim}_{n \to ∞} {\int }_{0 -}^{∞} H ( t - {t}_{1}^{-} ) H ( {t}_{2}^{+} - t ) v ( t ) {δ}_{n} ( t - {b}_{k} ) {e}^{- s t} d t \\ = {\sum }_{k = 1}^{K} {lim}_{n \to ∞} {\int }_{- ∞}^{∞} H ( t - {0}^{-} ) H ( t - {t}_{1}^{-} ) H ( {t}_{2}^{+} - t ) {δ}_{n} ( t - {b}_{k} ) v ( t ) {e}^{- s t} d t \\ = {\sum }_{k = 1}^{K} H ( {b}_{k} - 0 ) H ( {b}_{k} - {t}_{1} ) H ( {t}_{2} - {b}_{k} ) {\underbrace{( {lim}_{n \to ∞} {\int }_{- ∞}^{∞} {δ}_{n} ( t - {b}_{k} ) v ( t ) {e}^{- s t} d t )}}_{v ( {b}_{k} ) {e}^{- {sb}_{k}} , from Theorem 8.16} \\ = {\sum }_{k = 1}^{K} H ( {b}_{k} - {t}_{1} ) H ( {t}_{2} - {b}_{k} ) H ( {b}_{k} ) v ( {b}_{k} ) {e}^{- s {b}_{k}} .\end{matrix} \qquad ( 9.32 )$$

Formula (9.32) is for a general case in which t₁ and t₂ can have arbitrary values. In the special case when the spike train b is causal and t₁=0 and t₁=t if this formula simplifies as follows:

$$\begin{matrix}{ℒ}^{( v )} \{ b [ 0 , t ] \} ( s ) = {\sum }_{k = 1}^{K} {\underbrace{H ( {b}_{k} - 0 )}}_{1} H ( t - {b}_{k} ) {\underbrace{H ( {b}_{k} )}}_{1} v ( {b}_{k} ) {e}^{- s {b}_{k}} \\ = {\sum }_{k = 1}^{K} H ( t - {b}_{k} ) v ( {b}_{k} ) {e}^{- s {b}_{k}} .\end{matrix} \qquad ( 9.33 )$$

Another special case is when the spike train b is causal, the truncation interval is [t, T], and all spikes in b occur no later than time T. Now t₁=t and t₂=T and formula (9.32) reduces to:

$$\begin{matrix}{ℒ}^{( v )} \{ b [ t , T ] \} ( s ) = {\sum }_{k = 1}^{K} H ( {b}_{k} - t ) {\underbrace{H ( T - {b}_{k} )}}_{1} {\underbrace{H ( {b}_{k} )}}_{1} v ( {b}_{k} ) {e}^{- s {b}_{k}} \\ = {\sum }_{k = 1}^{K} H ( {b}_{k} - t ) v ( {b}_{k} ) {e}^{- s {b}_{k}} .\end{matrix} \qquad ( 9.34 )$$

Yet another special case can be derived from (9.34) after multiplying both sides by e^{st}. In other words,

$$\begin{matrix}{e}^{s t} {ℒ}^{( v )} \{ b [ t , T ] \} ( s ) = {e}^{s t} ( {\sum }_{k = 1}^{K} H ( {b}_{k} - t ) v ( {b}_{k} ) {e}^{- s {b}_{k}} ) \\ = {\sum }_{k = 1}^{K} H ( {b}_{k} - t ) v ( {b}_{k} ) {e}^{- s ( {b}_{k} - t )} .\end{matrix} \qquad ( 9.35 )$$

As described in Section 8.6.2 this formula can be viewed as a special case of the left-shift theorem for the Laplace transform when the input function is a weighted spike train. The shift in this case is equal to t.

### 9.3.2 The Laplace Transform of the Cross-Correlation of Two Weighted and Truncated Spike Trains

This section gives the formal definition for the Laplace transform of the cross-correlation of a pair of weighted and truncated spike trains. It extends the theory that was presented in Section 8.6.3 to handle weighted and truncated spike train as well.

Definition 9.10. Model for the cross-correlation of two weighted and truncated trains. Let a=(a₁, a₂, . . . , a_{J}) be a spike train that consists of J spikes and let b=(b₁, b₂, . . . , b_{K}) be another spike train that consists of K spikes. Also, let u(t) and v(t) be two weighting functions. Let t₁ and t₂ be two real numbers such that t₁<t₂. Finally, let τ₁ and τ₂ be two real numbers such that τ₁≤τ₂. The model for the cross-correlation of a[t₁, t₂] weighted by u(t) and b[τ₁, τ₂] weighted by v(t) is formed by the functions ((ua_{[t}_{₁}_{,t}_{₂}_{]}^{(m)})*(vb_{[τ}_{₁}_{, τ}_{₂}_{]}⁽ⁿ⁾))(t), where m and n are two positive integers. Each of these functions is defined by the following equation:

$$\begin{matrix}( ( u {a}_{[ {t}_{1} , {t}_{2} ]}^{( m )} ) ★ ( v {b}_{[ {τ}_{1 ,} {τ}_{2} ]}^{( n )} ) ) ( t ) = {\int }_{- ∞}^{∞} \overline{( u {a}_{[ {t}_{1} , {t}_{2} ]}^{( m )} ) ( τ )} ( v {b}_{[ {τ}_{1 ,} {τ}_{2} ]}^{( n )} ) ( τ + t ) d τ \\ = {\int }_{- ∞}^{∞} \overline{u ( τ )} {a}_{[ {t}_{1} , {t}_{2} ]}^{( m )} ( τ ) v ( τ + t ) {b}_{[ {τ}_{1} , {τ}_{2} ]}^{( n )} ( τ + t ) d τ .\end{matrix} \qquad ( 9.36 )$$

Definition 9.11. The Laplace transform of the cross-correlation of two weighted and truncated spike trains. Let a=(a₁, a₂, . . . , a_{J}) be a spike train that has J spikes and let b=(b₁, b₂, . . . , b_{K}) be another spike train that has K spikes. Let u(t) and v(t) be two weighting functions. Let t₁ and t₂ be two real numbers such that t₁≤t₂. Also, let τ₁ and τ₂ be a pair of real numbers such that τ₁≤τ₂. The Laplace transform of the cross-correlation of a[t₁, t₂] weighted by u(t) and b[τ₁, τ₂] weighted by v(t) is defined as the iterated limit of Laplace transforms of the functions in the model for the cross-correlation as m and n approach infinity. More formally,

$${ℒ}^{( u , v )} \{ a [ {t}_{1} , {t}_{2} ] ★ b [ {τ}_{1} , {τ}_{2} ] \} ( s ) = {lim}_{m \to ∞} {lim}_{n \to ∞} {\int }_{0 -}^{∞} ( ( u {a}_{[ {t}_{1} , {t}_{2} ]}^{( m )} ) ★ ( v {b}_{[ {τ}_{1} , {τ}_{2} ]}^{( n )} ) ) ( t ) {e}^{- s t} dt . \qquad ( 9.37 )$$

The previous definition can be used as a starting point to derive a closed-form formula for the Laplace transform of the cross-correlation of two weighted and truncated spike trains. To keep the formulas manageable, we will use Fₘ and Gₙ to denote two helper functions that are defined as follows:

$${F}_{m} ( {t}_{1} , {t}_{2} , {a}_{j} , t ) = H ( t - {t}_{1}^{-} ) H ( {t}_{2}^{+} - t ) {δ}_{m} ( t - {a}_{j} ) , \qquad ( 9.38 )$$

$${G}_{n} ( {τ}_{1} , {τ}_{2} , {b}_{k} , t ) = H ( t - {τ}_{1}^{-} ) H ( {τ}_{2}^{+} - t ) {δ}_{n} ( t - {b}_{k} ) . \qquad ( 9.39 )$$

Let L denote the value of the Laplace transform of the cross-correlation of a[t₁, t₂] and b[τ₁, τ₂] where the spike trains are weighted by u(t) and v(t), respectively. Using the helper functions Fₘ and Gₙ we can express L as follows:

$$\begin{matrix}L = {ℒ}^{( u , v )} \{ a [ {t}_{1} , {t}_{2} ] ★ b [ {τ}_{1} , {τ}_{2} ] \} ( s ) \\ = {lim}_{m \to ∞} {lim}_{n \to ∞} {\int }_{0 -}^{∞} ( ( u {a}_{[ {t}_{1} , {t}_{2} ]}^{( m )} ) ★ ( v {b}_{[ {t}_{1} , {t}_{2} ]}^{( n )} ) ) ( t ) {e}^{- s t} d t \\ = {lim}_{m \to ∞} {lim}_{n \to ∞} {\int }_{0 -}^{∞} {\int }_{- ∞}^{∞} \overline{u ( τ )} {a}_{[ {t}_{1} , {t}_{2} ]}^{( m )} ( τ ) v ( τ + t ) {b}_{[ {τ}_{1} , {τ}_{2} ]}^{( n )} ( τ + t ) {e}^{- s t} d τ d t \\ = {lim}_{m \to ∞} {lim}_{n \to ∞} {\int }_{0 -}^{∞} {\int }_{- ∞}^{∞} ( \overline{u ( τ )} {\sum }_{j = 1}^{J} {F}_{m} ( {t}_{1} , {t}_{2} , {a}_{j} , τ ) ) ( v ( τ + t ) {\sum }_{k = 1}^{K} {G}_{n} ( {τ}_{1} , {τ}_{2} , {b}_{k} , τ + t ) ) {e}^{- s t} d τ d t \\ = {lim}_{m \to ∞} {lim}_{n \to ∞} {\int }_{- ∞}^{∞} \overline{u ( τ )} {\sum }_{j = 1}^{J} {F}_{m} ( {t}_{1} , {t}_{2} , {a}_{j} , τ ) {\int }_{0 -}^{∞} v ( τ + t ) {\sum }_{k = 1}^{K} {G}_{n} ( {τ}_{1} , {τ}_{2} , {b}_{k} , τ + t ) {e}^{- s t} d t d τ \\ = {\sum }_{j = 1}^{J} {\sum }_{k = 1}^{K} {lim}_{m \to ∞} {lim}_{n \to ∞} {\int }_{- ∞}^{∞} \overline{u ( τ )} {F}_{m} ( {t}_{1} , {t}_{2} , {a}_{j} , τ ) {\int }_{0 -}^{∞} v ( τ + t ) {G}_{n} ( {τ}_{1} , {τ}_{2} , {b}_{k} , τ + t ) {e}^{- s t} d t d τ \\ = {\sum }_{j = 1}^{J} {\sum }_{k = 1}^{K} {lim}_{m \to ∞} {\int }_{- ∞}^{∞} \overline{u ( τ )} {F}_{m} ( {t}_{1} , {t}_{2} , {a}_{j} , τ ) {\underbrace{( {lim}_{n \to ∞} {\int }_{0 -}^{∞} v ( τ + t ) {G}_{n} ( {τ}_{1} , {τ}_{2} , {b}_{k} , τ + t ) {e}^{- s t} d t )}}_{{g}_{k} ( τ )} dτ \\ = {\sum }_{j = 1}^{J} {\sum }_{k = 1}^{K} {lim}_{m \to ∞} {\int }_{- ∞}^{∞} {F}_{m} ( {t}_{1} , {t}_{2} , {a}_{j} , τ ) \overline{u ( τ )} {g}_{k} ( τ ) d τ \\ = {\sum }_{j = 1}^{J} {\sum }_{k = 1}^{K} {lim}_{m \to ∞} {\int }_{- ∞}^{∞} H ( τ - {t}_{1}^{-} ) H ( {t}_{2}^{+} - τ ) {δ}_{m} ( τ - {a}_{j} ) \overline{u ( τ )} {g}_{k} ( τ ) d τ .\end{matrix} \qquad ( 9.4 )$$

The inner integral in the previous formula was replaced with gₖ(τ), which can be expanded as follows:

$$\begin{matrix}{g}_{k} ( τ ) = {lim}_{n \to ∞} {\int }_{0 -}^{∞} v ( τ + t ) {G}_{n} ( {τ}_{1} , {τ}_{2} , {b}_{k} , τ + t ) {e}^{- st} dt \\ = {lim}_{n \to ∞} {\int }_{- ∞}^{∞} H ( t - {0}^{-} ) {G}_{n} ( {τ}_{1} , {τ}_{2} , {b}_{k} , τ + t ) v ( τ + t ) {e}^{- st} dt \\ = {lim}_{n \to ∞} {\int }_{- ∞}^{∞} H ( t - {0}^{-} ) H ( ( τ + t ) - {τ}_{1}^{-} ) H ( {τ}_{2}^{+} - ( τ + t ) ) {δ}_{n} ( ( τ + t ) - {b}_{k} ) v ( τ + t ) {e}^{- st} dt \\ = {lim}_{n \to ∞} {\int }_{- ∞}^{∞} H ( t - ( {τ}_{1}^{-} - τ ) ) H ( ( {τ}_{2}^{+} + τ ) - t ) H ( t - {0}^{-} ) {δ}_{n} ( t - ( {b}_{k} - τ ) ) v ( τ + t ) {e}^{- st} dt .\end{matrix} \qquad ( 9.41 )$$

To continue the derivation we need to show that the limit of gₖ(τ) as τ→aⱼ exists and is finite, which is done below:

$$\begin{matrix}{lim}_{τ \to {a}_{j}} {g}_{k} ( τ ) = H ( ( {b}_{k} - {a}_{j} ) - ( {τ}_{1} - {a}_{j} ) ) H ( ( {τ}_{2} - {a}_{j} ) - ( {b}_{k} - {a}_{j} ) ) H ( ( {b}_{k} - {a}_{j} ) - 0 ) v ( {a}_{j} + ( {b}_{k} - {a}_{j} ) ) {e}^{- s ( {b}_{k} - {a}_{j} )} \\ = H ( {b}_{k} - - {τ}_{1} + ) H ( {τ}_{2} - - {b}_{k} + ) H ( {b}_{k} - {a}_{j} ) v ( + {b}_{k} - ) {e}^{- s ( {b}_{k} - {a}_{j} )} \\ = H ( {b}_{k} - {τ}_{1} ) H ( {τ}_{2} - {b}_{k} ) H ( {b}_{k} - {a}_{j} ) v ( {b}_{k} ) {e}^{- s ( {b}_{k} - {a}_{j} )} .\end{matrix} \qquad ( 9.42 )$$

Finally, we can derive the following formula:

$$\\ \begin{matrix}\begin{matrix}L = {ℒ}^{{}_{} ( u , v )} \{ a [ {t}_{1} , {t}_{2} ] ★ b [ {τ}_{1} , {τ}_{2} ] \} ( s ) \\ = {\sum }_{j = 1}^{J} {\sum }_{k = 1}^{K} {lim}_{m \to ∞} {\int }_{- ∞}^{∞} H ( τ - {t}_{1}^{{}_{} -} ) H ( {t}_{2}^{{}_{} +} - τ ) {δ}_{m} ( τ - {a}_{j} ) \overline{u ( τ )} {g}_{k} ( τ ) d τ \\ = {\sum }_{j = 1}^{J} {\sum }_{k = 1}^{K} H ( {a}_{j} - {t}_{1} ) H ( {t}_{2} - {a}_{j} ) ( {lim}_{τ \to {a}_{j}} ( \overline{u ( τ )} {g}_{k} ( τ ) ) ) \\ = {\sum }_{j = 1}^{J} {\sum }_{k = 1}^{K} H ( {a}_{j} - {t}_{1} ) H ( {t}_{2} - {a}_{j} ) H ( {b}_{k} - {τ}_{1} ) H ( {τ}_{2} - {b}_{k} ) H ( {b}_{k} - {a}_{j} ) \overline{u ( {a}_{j} )} v ( {b}_{k} ) {e}^{{}_{} - s ( {b}_{k} - {a}_{j} )} .\end{matrix} & ( 9.43 )\end{matrix}$$

Before we move on to the next topic we will derive two special cases of formula (9.43). In the first special case it is assumed that both a and b are causal spike trains that are truncated to the interval [0, t]. That is, t₁=τ₁=0 and t₂=τ₂=t. Then, formula (9.43) simplifies as shown below:

$$\\ \begin{matrix}{ℒ}^{{}_{} ( u , v )} \{ a [ 0 , t ] ★ b [ 0 , t ] \} ( s ) = {\sum }_{j = 1}^{J} {\sum }_{k = 1}^{K} {\underbrace{H ( {a}_{j} - 0 )}}_{1} H ( t - {a}_{j} ) {\underbrace{H ( {b}_{k} - 0 )}}_{1} H ( t - {b}_{k} ) H ( {b}_{k} - {a}_{j} ) \overline{u ( {a}_{j} )} v ( {b}_{k} ) {e}^{{}_{} - s ( {b}_{k} - {a}_{j} )} = {\sum }_{j = 1}^{J} {\sum }_{k = 1}^{K} H ( t - {a}_{j} ) H ( t - {b}_{k} ) H ( {b}_{k} - {a}_{j} ) \overline{u ( {a}_{j} )} v ( {b}_{k} ) {e}^{{}_{} - s ( {b}_{k} - {a}_{j} )} . & ( 9.44 )\end{matrix}$$

In the second special case it is assumed that all spikes in a and b occur no later than time T and that both trains are truncated to the interval [t, T]. In other words, t₁=τ₁=t and t₂=τ₂=T. Then, formula (9.43) simplifies as follows:

$$\\ \begin{matrix}{ℒ}^{{}_{} ( u , v )} \{ a [ t , T ] ★ b [ t , T ] \} ( s ) = {\sum }_{j = 1}^{J} {\sum }_{k = 1}^{K} H ( {a}_{j} - t ) {\underbrace{H ( T - {a}_{j} )}}_{1} H ( {b}_{k} - t ) {\underbrace{H ( T - {b}_{k} )}}_{1} H ( {b}_{k} - {a}_{j} ) \overline{u ( {a}_{j} )} v ( {b}_{k} ) {e}^{{}_{} - s ( {b}_{k} - {a}_{j} )} = {\sum }_{j = 1}^{J} {\sum }_{k = 1}^{K} H ( {a}_{j} - t ) H ( {b}_{k} - t ) H ( {b}_{k} - {a}_{j} ) \overline{u ( {a}_{j} )} v ( {b}_{k} ) {e}^{{}_{} - s ( {b}_{k} - {a}_{j} )} . & ( 9.45 )\end{matrix}$$

### 9.3.3 Modeling Reversed and Weighted Spike Trains

A reversed spike train can be obtained from a causal spike train by reversing the temporal order of its spikes in some interval. This was formally defined in Definition 8.25. This definition is slightly adjusted below to handle spike trains that are truncated to the interval [0, t] before the are reversed.

Definition 9.12. Truncated and reversed spike train. Let a=(a₁, a₂, . . . , a_{J}) be a causal spike train that has J spikes and let t be a nonnegative real number. The reversed spike train [symbol] [0, t] is obtained from a by truncating and reversing the times of the spikes on [0, t]. The model for [symbol] [0, t] is a sequence of functions ( [symbol] _{[0, t]}⁽¹⁾(τ), [symbol] _{[0, t]}⁽²⁾(τ), . . . , [symbol] _{[0, t]}⁽¹⁾(τ), . . . ), where each function [symbol] _{[0, t]}^{(m)}(τ) is obtained by reversing a^{(m)}(τ) on [0, t]. In other words,

$$\\ \begin{matrix}\overleftarrow{a} {}_{[ 0 , t ]}^{{}_{} ( m )} ( τ ) = H ( {t}^{{}_{} +} - τ ) {a}^{{}_{} ( m )} ( t - τ ) = {\sum }_{j = 1}^{J} H ( {t}^{{}_{} +} - τ ) {δ}_{m} ( t - τ - {a}_{j} ) , & ( 9.46 )\end{matrix}$$

for each m∈ [symbol] ⁺={1, 2, . . . }.
Definition 9.13. Reversed weighting function. Let u(τ) be a continuous weighting function and let t be a real number. Then, the reversed function [symbol] (τ) is defined as

$$\\ \begin{matrix}\overleftarrow{u} ( τ ) = u ( t - τ ) , & ( 9.47 )\end{matrix}$$

for each t such that t−τ∈domain(u).
Property 9.14. Let u(τ) be a weighting function and let [symbol] (τ) be the corresponding reversed weighting function. Also, let t be a real number. Then, reversing the function u on the interval [0, t] and conjugating it are commutative operations. That is,

$$\\ \begin{matrix}\overleftarrow{\overline{u}} ( τ ) = \overleftarrow{\overline{u}} ( τ ) . & ( 9.48 )\end{matrix}$$

The notation described below assumes that both the spike train and its weighting function are reversed on the same interval, i.e., [0, t]. This is necessary because the weights of the spikes need to be preserved after the reversal. The next definition states this more formally.

### Definition 9.15. Weighted, Truncated, and Reversed Spike Train.

Let a=(a₁, a₂, . . . , a_{J}) be a causal spike train that is weighted by the function u(t). Also, let t be a nonnegative real number. The model or a spike train that is truncated and reversed on the interval [0, t] is a sequence of functions (( [symbol] [symbol] _{[0, t]}⁽¹⁾)(τ), ( [symbol] [symbol] _{[0, t]}⁽²⁾)(τ), . . . , ( [symbol] [symbol] _{[0, t]}^{(m)}(τ), . . . ), where

$$\\ \begin{matrix}( {}_{} \overleftarrow{u} {}_{} \overleftarrow{a} {}_{[ 0 , t ]}^{{}_{} ( m )} ) ( τ ) = H ( {t}^{{}_{} +} - τ ) ( {}_{} \overleftarrow{u} {}_{} \overleftarrow{a} {}^{{}_{} ( m )} ) ( τ ) = H ( {t}^{{}_{} +} - τ ) u ( t - τ ) {a}^{{}_{} ( m )} ( t - τ ) , & ( 9.49 )\end{matrix}$$

for each m∈{1, 2, . . . }.
Property 9.16. The Laplace transform of a weighted, truncated, and reversed spike train. Let a=(a₁, a₂, . . . , a_{J}) be a causal spike train that is weighted by the function u(t) and let t≥0 be a real number. If this weighted and truncated spike train is reversed on the interval [0, t], then the Laplace transform of the resulting spike train can be expressed with the following formula:

$$\\ \begin{matrix}{ℒ}^{{}_{} ( \overleftarrow{u} )} \{ \overleftarrow{a} [ 0 , t ] \} ( s ) = {e}^{{}_{} - st} {ℒ}^{{}_{} ( u )} \{ a [ 0 , t ] \} ( - s ) . & ( 9.5 )\end{matrix}$$

Next, we will derive two special cases of Property 9.16. The first special case conjugates the weighting function, i.e., it uses ū instead of u. Thus,

$$\\ \begin{matrix}{ℒ}^{{}_{} ( {u}^{\overline{←}} )} \{ \overleftarrow{a} [ 0 , t ] \} ( s ) = {\sum }_{j = 1}^{J} H ( t - {a}_{j} ) \overline{u ( {a}_{j} )} {e}^{{}_{} - s ( t - {a}_{j} )} . & ( 9.51 )\end{matrix}$$

Extending this derivation leads to the following alternative expression:

$$\\ \begin{matrix}{ℒ}^{{}_{} ( {u}^{\overline{←}} )} \{ \overleftarrow{a} [ 0 , t ] \} ( s ) = {e}^{{}_{} - st} {ℒ}^{{}_{} ( \overline{u} )} \{ a [ 0 , t ] \} ( - s ) . & ( 9.52 )\end{matrix}$$

The second special case assumes that all spikes in a occur before time T, i.e., aⱼ≤T for all j. Under these conditions, formula (9.51) can be evaluated at time t=T to get the following result:

$$\\ \begin{matrix}{ℒ}^{{}_{} ( {u}^{\overline{←}} )} \{ \overleftarrow{a} [ 0 , T ] \} ( s ) = {\sum }_{j = 1}^{J} {\underbrace{H ( T - {a}_{j} )}}_{1} \overline{u ( {a}_{j} )} {e}^{{}_{} - s ( T - {a}_{j} )} = {\sum }_{j = 1}^{J} \overline{u ( {a}_{j} )} {e}^{{}_{} - s ( T - {a}_{j} )} . & ( 9.53 )\end{matrix}$$

Furthermore, using the fact that a=a[0, T] the expression in (9.52) can be simplified as follows:

$$\\ \begin{matrix}{ℒ}^{{}_{} ( {u}^{\overline{←}} )} \{ \overleftarrow{a} [ 0 , T ] \} ( s ) = {e}^{{}_{} - sT} {ℒ}^{{}_{} ( \overline{u} )} \{ a [ 0 , T ] \} ( - s ) = {e}^{{}_{} - sT} {ℒ}_{{}_{} a}^{{}_{} ( \overline{u} )} ( - s ) . & ( 9.54 )\end{matrix}$$

### 9.4 The Concatenation Theorem for Weighted Spike Trains

This section states the concatenation theorem for weighted spike trains. The theorem is an extension of the theorem for unweighted spike trains that was stated in Section 8.7.

### Theorem 9.17. The Concatenation Theorem for Weighted Spike Trains.

Let a=(a₁, a₂, . . . , a_{J}) be a spike train that has J spikes and let b=(b₁, b₂, . . . , b_{K}) be another spike train that has K spikes. It is assumed that both trains are causal and that the list of spike times in each train is sorted in increasing order and contains no duplicates, i.e., aⱼ≥0 for each j∈{1, 2, . . . , J}, bₖ≥0 for each k∈{1, 2, . . . , K}, aⱼ<aⱼ₊₁ for each j∈{1, 2, . . . , J−1}, and bₖ<bₖ+1 for each k∈{1, 2, . . . , K−1}. Also, let u(t) and v(t) be two weighting functions.
Let each of the two spike trains be split into two parts, where the time of the cut is denoted by C, which is a nonnegative real constant. Let a′ and a″ be the prefix and the suffix of the train a such that a′ contains the spikes in a that occur up to and including time C and a″ contains all remaining spikes from a that are not in a′. Similarly, let b be split into a prefix b′ and a suffix b″ such that b′ contains the spikes from b that occur strictly before time C and b″ contains all of the remaining spikes from b that are not in b′.
In other words, the spike train a is split into two spike trains a′ and a″ such that

$$\\ \begin{matrix}{a}^{{}_{} ′} = ( {a}_{1} , {a}_{2} , … , {a}_{p} ) , & ( 9.55 )\end{matrix}$$

$$\\ \begin{matrix}{a}^{{}_{} ″} = ( {a}_{p + 1} , {a}_{p + 2} , … , {a}_{J} ) , where & ( 9.56 )\end{matrix}$$

$$\\ \begin{matrix}p = max \{ j : {a}_{j} \leq C \} . & ( 9.57 )\end{matrix}$$

Using this formulation, the original spike train a can be recovered from the two slices a′ and a″ by concatenating the two lists of spike times, i.e., a=a′∥a″, where ∥ denotes concatenation.
In a similar way, the original spike train b is split into b′ and b″ such that

$$\\ \begin{matrix}{b}^{{}_{} ′} = ( {b}_{1} , {b}_{2} , … , {b}_{q} ) , & ( 9.58 )\end{matrix}$$

$$\\ \begin{matrix}{b}^{{}_{} ″} = ( {b}_{q + 1} , {b}_{q + 2} , … , {b}_{K} ) , where & ( 9.59 )\end{matrix}$$

$$\\ \begin{matrix}q = max \{ k : {b}_{k} < C \} . & ( 9.6 )\end{matrix}$$

Once again, by concatenating b′ and b″ we can recover the original spike train b, i.e., b=b′∥b″.
The four slices can also be expressed as follows:

$$\\ \begin{matrix}{a}^{{}_{} ′} ← a [ 0 , C ] , {a}^{{}_{} ″} ← a ( C , ∞ ) , {b}^{{}_{} ′} ← b [ 0 , C ) , {b}^{{}_{} ″} ← b [ C , ∞ ) . & ( 9.61 )\end{matrix}$$

Then, the concatenation theorem for weighted spike trains states that:

$${ℒ}_{a ★ b}^{( u , v )} ( s ) = {ℒ}_{{a}^{′} ★ {b}^{′}}^{( u , v )} ( s ) + {ℒ}_{{a}^{″} ★ {b}^{″}}^{( u , v )} ( s ) + \overline{{ℒ}_{{a}^{′}}^{( u )} ( - \overline{s} )} {ℒ}_{{b}^{″}}^{( v )} , ( s ) . \qquad ( 9.62 )$$

Note that in the statement of the theorem the two spike trains a and b are split slightly differently. The prefix a′ includes all spikes from a that fall in the closed interval [0, C] and the suffix a″ includes the remaining spikes from a that fall in (C, ∞). The train b, however, is split such that the prefix b′ includes all spikes from b that fall in the interval [0, C) and the suffix b″ includes all spikes from b that fall in the interval [C, ∞). This difference in the splits is intentional and its purpose it to eliminate the special cases that have to be considered otherwise if there are one or more spikes that occur exactly at time C.

### 9.4.1 Two Special Cases of the Concatenation Theorem

This section states two corollaries of the concatenation theorem for weighted spike trains. These corollaries cover special cases in which the splits of the two spike trains have certain properties.

Corollary 9.18 is a special case of the concatenation theorem for weighted spike trains when the two trains are split such that the suffix a″ is empty and the suffix b″ contains just one spike.

Corollary 9.18. When the suffix b″ contains just one spike and the suffix a″ is empty. Let a=(a₁, a₂, . . . , a_{J}) and b=(b₁, b₂, . . . , b_{K}) be two spike trains that unfold simultaneously in time such that aⱼ<b_{K} for each j∈{1, 2, . . . , J} and b_{K}=T. In other words, each spike in a precedes the last spike in b, which occurs exactly at time T. It is also assumed that a and b are causal, i.e., aⱼ≥0 and bₖ≥0 for each j∈{1, 2, . . . , J} and each k∈{1, 2, . . . , K}. Also, let u(t) and v(t) be two weighting functions.
Let the spike train a be divided into two spike trains a′ and a″ such that a′=a=(a₁, a₂, . . . , a_{J}) and a″=( ). That is, the suffix a″ is empty and contains no spikes and the prefix a′ contains all spikes from a. Also, let b be divided into b′ and b″, where b′=(b₁, b₂, . . . , b_{K-1}) and b″=(b_{K}). In other words, the suffix b″ contains just the last spike, which occurs at time T. Then,

$${ℒ}_{a ★ b}^{( u , v )} ( s ) = {ℒ}_{{a}^{′} ★ {b}^{′}}^{( u , v )} ( s ) + v ( {b}_{K} ) {ℒ}_{\overleftarrow{a}}^{( \overline{\overleftarrow{u}} )} ( s ) , \qquad ( 9.63 )$$

where [symbol] denotes a spike train obtained by reversing the spikes in a in the interval [0, T]. In other words, the time of the n-th spike in a [symbol] is given by

$${( \overleftarrow{a} )}_{n} = T - {a}_{J + 1 - n} , for n = 1 , 2 , … , J , \qquad ( 9.64 )$$

and the reversed spike train [symbol] is specified as follows:

$$\overleftarrow{a} = ( T - {a}_{J} , T - {a}_{J - 1} , … , T - {a}_{2} , T - {a}_{1} ) . \qquad ( 9.65 )$$

Note that in formula (9.63) the notation [symbol] (s) denotes the value of the Laplace transform at s of the reversed spike train [symbol] that is weighted by the reversed and conjugated function u, where [symbol] (t)=u̅(̅T̅−̅t̅)̅.

Corollary 9.19 is a special case of the concatenation theorem for weighted spike trains when the two trains are split such that the prefix a′ contains just one spike and the prefix b′ is empty.

Corollary 9.19. When the prefix a′ contains just one spike and the prefix b′ is empty. Let a=(a₁, a₂, . . . , a_{J}) and b=(b₁, b₂, . . . , b_{K}) be two spike trains such that a₁<bₖ for each k∈{1, 2, . . . , K}, i.e., the first spike in a occurs before all spikes in b. It is assumed that both spike trains are causal, i.e., aⱼ≥0 for each j∈{1, 2, . . . , J} and bₖ≥0 for each k∈{1, 2, . . . , K}. Let a be split into two non-overlapping spike trains a′=(a₁) and a″=(a₂, a₃, . . . , a_{J}). In other words, the prefix a′ consists of only the first spike in a and the suffix a″ contains the remaining spikes from a. Also, let b be split into b′ and b″ such that b′=( ) and b″=b=(b₁, b₂, . . . , b_{K}). That is, the prefix b′ is empty and the suffix b″ is equal to b. Furthermore, let u(t) and v(t) be two weighting functions. Then,

$${ℒ}_{a ★ b}^{( u , v )} ( s ) = \overline{u ( {a}_{1} )} {e}^{{sa}_{1}} {ℒ}_{b}^{( v )} ( s ) + {ℒ}_{{a}^{″} ★ {b}^{″}}^{( u , v )} ( s ) . \qquad ( 9.66 )$$

### 9.4.2 Special Cases of the Theorem for Weighted and Truncated Spike Trains

The concatenation theorem for weighted spike trains also applies to weighted and truncated spike trains. The following two corollaries of Theorem 9.17 are the mathematical justification for the SUV algorithms, which are described later in this chapter.

Corollary 9.20. Let x=(x₁, x₂, . . . , x_{J}) and y=(y₁, y₂, . . . , y_{K}) be two causal spike trains that are weighted by the functions u(t) and v(t), respectively. Then, for each integer n∈{2, 3, . . . , K}, the following is true:

$${ℒ}^{( u , v )} \{ x [ 0 , {y}_{n} ] ★ y [ 0 , {y}_{n} ] \} ( s ) = {ℒ}^{( u , v )} \{ x [ 0 , {y}_{n - 1} ] ★ y [ 0 , {y}_{n - 1} ] \} ( s ) + v ( {y}_{n} ) {ℒ}^{( \overline{\overleftarrow{u}} )} \{ \overleftarrow{x} [ 0 , {y}_{n} ] \} ( s ) , \qquad ( 9.67 )$$

where [symbol] [0, yₙ] is a spike train that is obtained by reversing x[0, yₙ] in the interval [0, yₙ]. That is,

$$\overleftarrow{x} [ 0 , {y}_{n} ] = ( {y}_{n} - {x}_{p} , {y}_{n} - {x}_{p - 1} , … , {y}_{n} - {x}_{1} ) , \qquad ( 9.68 )$$

where p=max{j: xⱼ≤yₙ}. Also, [symbol] (t)=u̅(̅y̅ₙ̅−̅t̅)̅.
Furthermore, in the special case when n=1, the following equation holds:

$${ℒ}^{( u , v )} \{ x [ 0 , {y}_{1} ] ★ y [ 0 , {y}_{1} ] \} ( s ) = v ( {y}_{1} ) {ℒ}^{( \overline{\overleftarrow{u}} )} \{ \overleftarrow{x} [ 0 , {y}_{1} ] \} ( s ) . \qquad ( 9.69 )$$

Corollary 9.21. Let x=(x₁, x₂, . . . , x_{J}) and y=(y₁, y₂, . . . , y_{K}) be two causal spike trains that are weighted by the functions u(t) and v(t), respectively. It is assumed that the spikes in x and y occur no later than time T, i.e., 0≤xⱼ≤T and 0≤yₖ≤T for all j∈{1, 2, . . . , J} and for all k∈{1, 2, . . . , K}. Then, for any integer m∈{1, 2, . . . , J−1} the following is true:

$${ℒ}^{( u , v )} \{ x [ {x}_{m} , T ] ★ y [ {x}_{m} , T ] \} ( s ) = \overline{u ( {x}_{m} )} {e}^{{sx}_{m}} {ℒ}^{( v )} \{ y [ {x}_{m} , T ] \} ( s ) + {ℒ}^{( u , v )} \{ x [ {x}_{m + 1} , T ] ★ y [ {x}_{m + 1} , T ] \} ( s ) . \qquad ( 9.7 )$$

Also, in the special case when m=J, the following equation holds:

$${ℒ}^{( u , v )} \{ x [ {x}_{J} , T ] ★ y [ {x}_{J} , T ] \} ( s ) = \overline{u ( {x}_{J} )} {e}^{{sx}_{J}} {ℒ}^{( v )} \{ y [ {x}_{J} , T ] \} ( s ) . \qquad ( 9.71 )$$

Finally, we will prove two properties that were used to prove Corollary 9.20 and Corollary 9.21, respectively.

Property 9.22. Let x=(x₁, x₂, . . . , x_{J}) and y=(y₁, y₂, . . . , y_{K}) be two causal spike trains that are weighted by the functions u(t) and v(t), respectively. Also, let x[0, t] and y[0, τ] be two truncated spike trains such that τ<t. Then,

$${ℒ}^{( u , v )} \{ x [ 0 , t ] ★ y [ 0 , τ ] \} ( s ) = {ℒ}^{( u , v )} \{ x [ 0 , τ ] ★ y [ 0 , τ ] \} ( s ) . \qquad ( 9.72 )$$

That is, the Laplace transform of the cross-correlation of x[0, t] weighted by u(t) and y[0, τ] weighted by v(t) is equal to the Laplace transform of the cross-correlation of x[0, τ] weighted by u(t) and y[0, τ] weighted by v(t). In other words, spikes from x that occur in the interval (τ, t] don't affect the result.
Property 9.23. Let x=(x₁, X₂, . . . , x_{J}) and y=(y₁, y₂, . . . , y_{K}) be two causal spike trains that are weighted by u(t) and v(t), respectively. Also, let the spikes in both trains occur no later than time T, i.e., 0≤xⱼ≤T and 0≤yₖ≤T for all j and for all k. Furthermore, let x[t, T] and y[τ, T] be two truncated spike trains such that τ<t. Then,

$${ℒ}^{( u , v )} \{ x [ t , T ] ★ y [ τ , T ] \} ( s ) = {ℒ}^{( u , v )} \{ x [ t , T ] ★ y [ t , T ] \} ( s ) . \qquad ( 9.73 )$$

In other words, the Laplace transform of the cross-correlation of x[t, T] weighted by u(t) and y[τ, T] weighted by v(t) is equal to the Laplace transform of the cross-correlation of x[t, T] weighted by u(t) and y[t, T] weighted by v(t). That is, the spikes from y that occur in the interval [τ, t) don't influence the result.

### 9.5 The SUV SSM Model

This section describes the SSM model for weighted spike trains, which is an extension of the model for non-weighted spike trains described in Section 8.8. To distinguish between the two models the new one will be called the SUV SSM model or simply the SUV model. This name comes from the three parameters of the model—s, u, and v—where s is the argument of the Laplace transform and u and v are two weighting functions. This is analogous to the ZUV model in the discrete-time case, which also had three parameters: z, u, and v.

The SUV model has three components: a matrix M and two vectors h′ and h″. The matrix is of size M′×M″, h′ is a column vector of size M′, and h″ is a row vector of size M″. Without loss of generality, the examples in this section will assume that M′=M″=2. FIG. 121 summarizes the notation for the elements of the three components in that case. The four spike trains from which these components are computed will be denoted with α, β, A, and B. Using our convention, the spike trains that correspond to the rows are labeled with Greek letters. Also, the spike trains that correspond to the columns are labeled with English letters.

### 9.5.1 The Model at the End of Encoding

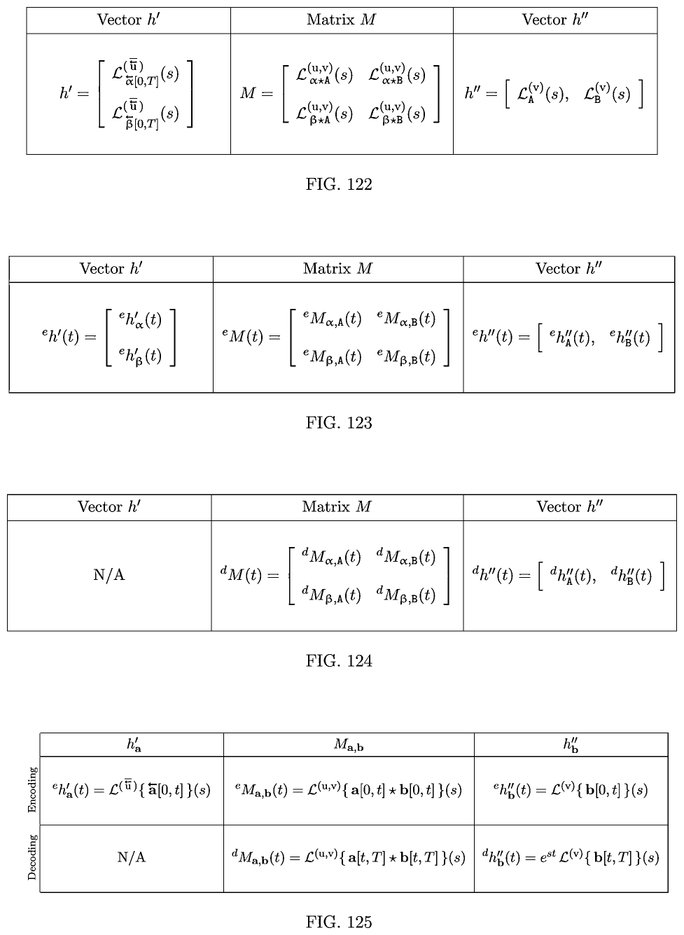

FIG. 122 shows the elements of the three components at the end of encoding, which is assumed to be at time T. Each element of the matrix is equal to the Laplace transform of the corresponding cross-correlation of two weighted spike trains. The spike train that corresponds to the row is weighted by u(t); the spike train that corresponds to the column is weighted by v(t). Each element of h′ is equal to the Laplace transform of the corresponding spike train, which has been reversed in the interval [0, T] and also weighted by the reversed and conjugated function [symbol] , where [symbol] (t)=u̅(̅T̅−̅t̅)̅. Each element of h″ is equal to the Laplace transform of the corresponding spike train weighted by v(t). All transforms are evaluated at s.

The formulas in FIG. 122 are expressed in terms of the Laplace transform. Using the Heaviside function these formulas can also be stated as shown below:

$${h}^{′} = [ \begin{matrix}{\sum }_{j = 1}^{\vert α \vert} & \overline{u ( {α}_{j} )} {e}^{- s ( T - {α}_{j} )} \\ {\sum }_{j = 1}^{\vert β \vert} & \overline{u ( {β}_{j} )} {e}^{- s ( T - {β}_{j} )}\end{matrix} ] , \qquad ( 9.74 )$$

$${h}^{′} = [ {\sum }_{k = 1}^{\vert A \vert} v ( {A}_{k} ) {e}^{- s {A}_{k}} , {\sum }_{k = 1}^{\vert B \vert} v ( {B}_{k} ) {e}^{- s {B}_{k}} ] , \qquad ( 9.75 )$$

$$M = [ \begin{matrix}{\sum }_{j = 1}^{\vert α \vert} {\sum }_{k = 1}^{\vert A \vert} H ( {A}_{k} - {α}_{j} ) \\ \overline{u ( {α}_{j} )} v ( {A}_{k} ) {e}^{- s ( {A}_{k} - {α}_{j} )} & {\sum }_{j = 1}^{\vert α \vert} {\sum }_{k = 1}^{\vert B \vert} H ( {B}_{k} - {α}_{j} ) \\ \overline{u ( {α}_{j} )} v ( {B}_{k} ) {e}^{- s ( {B}_{k} - {α}_{j} )} \\ {\sum }_{j = 1}^{\vert β \vert} {\sum }_{k = 1}^{\vert A \vert} H ( {A}_{k} - {β}_{j} ) \\ \overline{u ( {β}_{j} )} v ( {A}_{k} ) {e}^{- s ( {A}_{k} - {β}_{j} )} & {\sum }_{j = 1}^{\vert β \vert} {\sum }_{k = 1}^{\vert B \vert} H ( {B}_{k} - {β}_{j} ) \\ \overline{u ( {β}_{j} )} v ( {B}_{k} ) {e}^{- s ( {B}_{k} - {β}_{j} )}\end{matrix} ] . \qquad ( 9.76 )$$

These three expressions follow from formulas (9.53), (9.11) and (9.21), respectively.

### 9.5.2 The Model at a Specific Time During Encoding

The formulas described in the previous section express the elements of the model at the end of the encoding process, which is assumed to be at time T. This section presents another set of formulas that express these values at any time t prior to that.

FIG. 123 summarizes the notation that is used in this case. To denote that these are not the final values we will use the letter e in a superscript on the left, e.g., ^{e}h′, ^{e}h″, and ^{e}M.

Using the notation for weighted and truncated trains the components of the model can be expressed as follows:

$${e}^{} {h}^{′} ( t ) = [ \begin{matrix}{ℒ}^{( \overline{\overleftarrow{u}} )} \{ \overleftarrow{α} [ 0 , t ] \} ( s ) \\ {ℒ}^{( \overline{\overleftarrow{u}} )} \{ \overleftarrow{β} [ 0 , t ] \} ( s )\end{matrix} ] = [ \begin{matrix}{e}^{- st} \overline{{ℒ}^{( u )} \{ α [ 0 , t ] \} ( - \overline{s} )} \\ {e}^{- st} \overline{{ℒ}^{( u )} \{ β [ 0 , t ] \} ( - \overline{s} )}\end{matrix} ] [ \begin{matrix}{e}^{- s t} {ℒ}^{( \overline{u} )} \{ α [ 0 , t ] \} ( - s ) \\ {e}^{- s t} {ℒ}^{( \overline{u} )} \{ β [ 0 , t ] \} ( - s )\end{matrix} ] , \qquad ( 9.77 )$$

$${e}^{} {h}^{″} ( t ) = [ {ℒ}^{( v )} \{ A [ 0 , t ] \} ( s ) , {ℒ}^{( v )} \{ B [ 0 , t ] \} ( s ) ] , \qquad ( 9.78 )$$

$${e}^{} M ( t ) = [ \begin{matrix}{ℒ}^{( u , v )} \{ α [ 0 , t ] ★ A [ 0 , t ] \} ( s ) & {ℒ}^{( u , v )} \{ α [ 0 , t ] ★ B [ 0 , t ] \} ( s ) \\ {ℒ}^{( u , v )} \{ β [ 0 , t ] ★ A [ 0 , t ] \} ( s ) & {ℒ}^{( u , v )} \{ β [ 0 , t ] ★ B [ 0 , t ] \} ( s )\end{matrix} ] . \qquad ( 9.79 )$$

Note that all spike trains are truncated in the interval [0, t]. This suggests that the encoding can be accomplished with a single pass through all trains, which is what the SUV encoding algorithm does (see Section 9.8).

By adapting formulas (9.51), (9.33), and (9.44) the encoding formulas can also be stated in the following form:

$${e}^{} {h}^{′} ( t ) = [ \begin{matrix}{\sum }_{j = 1}^{\vert α \vert} H ( t - {α}_{j} ) \overline{u ( {α}_{j} )} {e}^{- s ( t - {α}_{j} )} \\ {\sum }_{j = 1}^{\vert β \vert} H ( t - {β}_{j} ) \overline{u ( {β}_{j} )} {e}^{- s ( t - {β}_{j} )}\end{matrix} ] , \qquad ( 9.8 )$$

$${e}^{} {h}^{″} ( t ) = [ {\sum }_{k = 1}^{\vert A \vert} H ( t - {A}_{k} ) v ( {A}_{k} ) {e}^{- s {A}_{k}} , {\sum }_{k = 1}^{\vert B \vert} H ( t - {B}_{k} ) v ( {B}_{k} ) {e}^{- s {B}_{k}} ] , \qquad ( 9.81 )$$

$${e}^{} M ( t ) = [ \begin{matrix}{}^{e} {M}_{α , A} ( t ) & {}^{e} {M}_{α , B} ( t ) \\ {}^{e} {M}_{β , A} ( t ) & {}^{e} {M}_{β , B} ( t )\end{matrix} ] , \qquad ( 9.82 )$$

$$where$$

$${e}^{} {M}_{α , A} ( t ) = \qquad ( 9.83 )$$

$${\sum }_{j = 1}^{\vert α \vert} {\sum }_{k = 1}^{\vert A \vert} H ( t - {α}_{j} ) H ( t - {A}_{k} ) H ( {A}_{k} - {α}_{j} ) \overline{u ( {α}_{j} )} v ( {A}_{k} ) {e}^{- s ( {A}_{k} - {α}_{j} )} ,$$

$${e}^{} {M}_{α , B} ( t ) = \qquad ( 9.84 )$$

$${\sum }_{j = 1}^{\vert α \vert} {\sum }_{k = 1}^{\vert B \vert} H ( t - {α}_{j} ) H ( t - {B}_{k} ) H ( {B}_{k} - {α}_{j} ) \overline{u ( {α}_{j} )} v ( {B}_{k} ) {e}^{- s ( {B}_{k} - {α}_{j} )} ,$$

$${e}^{} {M}_{β , A} ( t ) = \qquad ( 9.85 )$$

$${\sum }_{j = 1}^{\vert β \vert} {\sum }_{k = 1}^{\vert A \vert} H ( t - {β}_{j} ) H ( t - {A}_{k} ) H ( {A}_{k} - {β}_{j} ) \overline{u ( {β}_{j} )} v ( {A}_{k} ) {e}^{- s ( {A}_{k} - {β}_{j} )} ,$$

$${}^{e} {M}_{β , B} ( t ) = \qquad ( 9.86 )$$

$${\sum }_{j = 1}^{\vert β \vert} {\sum }_{k = 1}^{\vert B \vert} H ( t - {β}_{j} ) H ( t - {B}_{k} ) H ( {B}_{k} - {β}_{j} ) \overline{u ( {β}_{j} )} v ( {B}_{k} ) {e}^{- s ( {B}_{k} - {β}_{j} )} .$$

All of these formulas are mathematically correct, but from a computational point of view they are not very efficient. Section 9.7 derives iterative versions of these formulas that are used by the SUV encoding algorithm.

### 9.5.3 The Model at a Specific Time During Decoding

FIG. 124 summarizes the notation for the SUV model during decoding. Each element is expressed as a function of the current time t. To denote that these values are different from the values during encoding we will use a left superscript with the letter d, i.e., ^{d}M and ^{d}h″. Assuming that the spikes in all trains occur no later than time T, the matrix and the vector h″ can be expressed as follows:

$${}^{d} M ( t ) = [ \begin{matrix}{ℒ}^{( u , v )} \{ α [ t , T ] ★ A [ t , T ] \} ( s ) & {ℒ}^{( u , v )} \{ α [ t , T ] ★ B [ t , T ] \} ( s ) \\ {ℒ}^{( u , v )} \{ β [ t , T ] ★ A [ t , T ] \} ( s ) & {ℒ}^{( u , v )} \{ β [ t , T ] ★ B [ t , T ] \} ( s )\end{matrix} ] , \qquad ( 9.87 )$$

$${}^{d} {h}^{″} ( t ) = [ {e}^{st} {ℒ}^{( v )} \{ A [ t , T ] \} ( s ) , {e}^{st} {ℒ}^{( v )} \{ B [ t , T ] \} ( s ) ] . \qquad ( 9.88 )$$

Using formulas (9.45) and (9.35) the elements of M and h″ can also be stated as follows:

$${}^{d} M ( t ) = [ \begin{matrix}{}_{}^{d} {M}_{α , A}^{} ( t ) & {}_{}^{d} {M}_{α , B}^{} ( t ) \\ {}_{}^{d} {M}_{β , A}^{} ( t ) & {}_{}^{d} {M}_{β , B}^{} ( t )\end{matrix} ] , \qquad ( 9.89 )$$

$${}^{d} {h}^{″} ( t ) = [ {\sum }_{k = 1}^{\vert A \vert} H ( {A}_{k} - t ) v ( {A}_{k} ) {e}^{- s ( {A}_{k} - t )} , {\sum }_{k = 1}^{\vert B \vert} H ( {B}_{k} - t ) v ( {B}_{k} ) {e}^{- s ( {B}_{k} - t )} ] , \qquad ( 9.9 )$$

$$where$$

$$( 9.91 )$$

$${}_{}^{d} {M}_{α , A}^{} ( t ) = {\sum }_{j = 1}^{\vert α \vert} {\sum }_{k = 1}^{\vert A \vert} H ( {α}_{j} - t ) H ( {A}_{k} - t ) H ( {A}_{k} - {α}_{j} ) \overline{u ( {α}_{j} )} v ( {A}_{k} ) {e}^{- s ( {A}_{k} - {α}_{j} )} ,$$

$$( 9.92 )$$

$${}_{}^{d} {M}_{α , B}^{} ( t ) = {\sum }_{j = 1}^{\vert α \vert} {\sum }_{k = 1}^{\vert B \vert} H ( {α}_{j} - t ) H ( {B}_{k} - t ) H ( {B}_{k} - {α}_{j} ) \overline{u ( {α}_{j} )} v ( {B}_{k} ) {e}^{- s ( {B}_{k} - {α}_{j} )} ,$$

$$( 9.93 )$$

$${}_{}^{d} {M}_{β , A}^{} ( t ) = {\sum }_{j = 1}^{\vert β \vert} {\sum }_{k = 1}^{\vert A \vert} H ( {β}_{j} - t ) H ( {A}_{k} - t ) H ( {A}_{k} - {β}_{j} ) \overline{u ( {β}_{j} )} v ( {A}_{k} ) {e}^{- s ( {A}_{k} - {β}_{j} )} ,$$

$$( 9.94 )$$

$${}_{}^{d} {M}_{β , B}^{} ( t ) = {\sum }_{j = 1}^{\vert β \vert} {\sum }_{k = 1}^{\vert B \vert} H ( {β}_{j} - t ) H ( {B}_{k} - t ) H ( {B}_{k} - {β}_{j} ) \overline{u ( {β}_{j} )} v ( {B}_{k} ) {e}^{- s ( {B}_{k} - {β}_{j} )} .$$

### 9.5.4 The Formulas for an Abstract Element

For the sake of convenience and completeness, this section states the formulas for an abstract element of the matrix and the two vectors. Let a=(a₁, a₂, . . . , a_{J}) and b=(b₁, b₂, . . . , b_{K}) be two causal spike trains that contain J and K spikes, respectively. The matrix element that corresponds to this pair of spike trains will be denoted with M_{a,b}. The elements of the two vectors that correspond to M_{a,b} will be denoted with ha and h′b. Using this convention, the encoding and decoding formulas are stated below in two different ways. The first approach uses the Heaviside function. The second approach uses the Laplace transform notation.

At the end of encoding (i.e., at time T):

$${h}_{a}^{′} = {}_{}^{e} {h}_{a}^{′} ( T ) = {\sum }_{j = 1}^{J} \overline{u ( {α}_{j} )} {e}^{- s ( T - {a}_{j} )} , \qquad ( 9.95 )$$

$${h}_{b}^{″} = {}_{}^{e} {h}_{b}^{″} ( T ) = {\sum }_{k = 1}^{K} v ( {b}_{k} ) {e}^{- {sb}_{k}} , \qquad ( 9.96 )$$

$${M}_{a , b} = {}_{}^{e} {M}_{a , b}^{} ( T ) = {\sum }_{j = 1}^{J} {\sum }_{k = 1}^{K} H ( {b}_{k} - {a}_{j} ) \overline{u ( {α}_{j} )} v ( {b}_{k} ) {e}^{- s ( {b}_{k} - {a}_{j} )} . \qquad ( 9.97 )$$

At time t during encoding:

$${}_{}^{e} {h}_{a}^{′} ( t ) = {\sum }_{j = 1}^{J} H ( t - {a}_{j} ) \overline{u ( {α}_{j} )} {e}^{- s ( t - {a}_{j} )} , \qquad ( 9.98 )$$

$${}_{}^{e} {h}_{b}^{″} ( t ) = {\sum }_{k = 1}^{K} H ( t - {b}_{k} ) v ( {b}_{k} ) {e}^{- {sb}_{k}} , \qquad ( 9.99 )$$

$${}_{}^{e} {M}_{a , b}^{} ( t ) = {\sum }_{j = 1}^{J} {\sum }_{k = 1}^{K} H ( t - {a}_{j} ) H ( t - {b}_{k} ) \overline{u ( {α}_{j} )} v ( {b}_{k} ) {e}^{- s ( {b}_{k} - {a}_{j} )} . \qquad ( 9.1 )$$

At time t during decoding:

$$( 9.101 )$$

$${}_{}^{d} {M}_{a , b}^{} ( t ) = {\sum }_{j = 1}^{J} {\sum }_{k = 1}^{K} H ( {a}_{j} - t ) H ( {b}_{k} - t ) H ( {b}_{k} - {a}_{j} ) \overline{u ( {α}_{j} )} v ( {b}_{k} ) {e}^{- s ( {b}_{k} - {a}_{j} )} ,$$

$${}_{}^{d} {h}_{b}^{″} ( t ) = {\sum }_{k = 1}^{K} H ( {b}_{k} - t ) v ( {b}_{k} ) {e}^{- s ( {b}_{k} - t )} . \qquad ( 9.102 )$$

The same set of formulas can also be stated using the Laplace transform notation for weighted and truncated spike trains. FIG. 125 gives a visual summary of these formulas. In addition, these formulas are also stated below.

At the end of encoding (i.e., at time T):

$${h}_{a}^{′} = {}_{}^{e} {h}_{a}^{′} ( T ) = {ℒ}^{( \overline{\overleftarrow{u}} )} \{ \overleftarrow{a} [ 0 , T ] \} ( s ) = {e}^{- sT} \overline{{ℒ}^{( u )} \{ a \} ( - \overline{s} )} , \qquad ( 9.103 )$$

$${h}_{b}^{″} = {}_{}^{e} {h}_{b}^{″} ( T ) = {ℒ}^{( v )} \{ b \} ( s ) , \qquad ( 9.104 )$$

$${M}_{a , b} = {}_{}^{e} {M}_{a , b}^{} ( T ) = {ℒ}^{( u , v )} \{ a ★ b \} ( s ) . \qquad ( 9.105 )$$

At time t during encoding:

$${}_{}^{e} {h}_{a}^{′} ( t ) = {ℒ}^{( \overline{\overleftarrow{u}} )} \{ \overleftarrow{a} [ 0 , t ] \} ( s ) = {e}^{- st} \overline{{ℒ}^{( u )} \{ a [ 0 , t ] \} ( - \overline{s} )} , \qquad ( 9.106 )$$

$${}_{}^{e} {h}_{b}^{″} ( t ) = {ℒ}^{( v )} \{ b [ 0 , t ] \} ( s ) , \qquad ( 9.107 )$$

$${}_{}^{e} {M}_{a , b}^{} ( t ) = {ℒ}^{( u , v )} \{ a [ 0 , t ] ★ b [ 0 , t ] \} ( s ) . \qquad ( 9.108 )$$

At time t during decoding:

$${}_{}^{d} {M}_{a , b}^{} ( t ) = {ℒ}^{( u , v )} \{ a [ t , T ] ★ b [ t , T ] \} ( s ) , \qquad ( 9.109 )$$

$${}_{}^{d} {h}_{b}^{″} ( t ) = {e}^{st} {ℒ}^{( v )} \{ b [ t , T ] \} ( s ) . \qquad ( 9.11 )$$

### 9.6 Duality of the Matrix Representation

This section shows that the matrix values in the SUV model can be viewed in two different ways. The first view suggests how the matrix can be encoded. The second view suggests how it can be decoded. This duality is analogous to the one described in Section 8.9, but now the spike trains are weighted and as a result of this the formulas are slightly different.

At the end of the encoding process the value of the matrix element M_{a,b} is equal to:

$${M}_{a , b} = {\sum }_{j = 1}^{J} {\sum }_{k = 1}^{K} H ( {b}_{k} - {a}_{j} ) \overline{u ( {α}_{j} )} v ( {b}_{k} ) {e}^{- s ( {b}_{k} - {a}_{j} )} . \qquad ( 9.111 )$$

The two views of the matrix factor this expression in two different ways and express it in terms of ^{e}hₐ′(t) and ^{d}h_{b}″(t), which were defined as follows:

$${}_{}^{e} {h}_{a}^{′} ( t ) = {\sum }_{j = 1}^{J} H ( t - {a}_{j} ) \overline{u ( {α}_{j} )} {e}^{- s ( t - {a}_{j} )} , \qquad ( 9.112 )$$

$${}_{}^{d} {h}_{b}^{″} ( t ) = {\sum }_{k = 1}^{K} H ( {b}_{k} - t ) v ( {b}_{k} ) {e}^{- s ( {b}_{k} - t )} . \qquad ( 9.113 )$$

### 9.6.1 Encoding View

The matrix element M_{a,b} is encoded from two causal spike trains a and b. Because these spike trains contain a finite number of spikes we can change the order of the two sums in (9.111). This swap allows us to factor the expression as follows:

$$\begin{matrix}{M}_{a , b} = {\sum }_{j = 1}^{J} {\sum }_{k = 1}^{K} H ( {b}_{k} - {a}_{j} ) \overline{u ( {α}_{j} )} v ( {b}_{k} ) {e}^{- s ( {b}_{k} - {a}_{j} )} \\ = {\sum }_{k = 1}^{K} v ( {b}_{k} ) {\underbrace{( {\sum }_{j = 1}^{J} H ( {b}_{k} - {a}_{j} ) \overline{u ( {α}_{j} )} {e}^{- s ( {b}_{k} - {a}_{j} )} )}}_{{}_{}^{e} {h}_{a}^{′} ( {b}_{k} )} \\ = {\sum }_{k = 1}^{K} v ( {b}_{k} ) {}_{}^{e} {h}_{a}^{′} ( {b}_{k} ) .\end{matrix} \qquad ( 9.114 )$$

That is, the value of M_{a,b} can be represented as a weighted sum of the values of Cha at the times of the spikes in b. The corresponding weights in this sum are given by the values of the weighting function v, also at the spike times in b.

This type of factorization applies to any element of the matrix. If the matrix is of size 2×2, then it has the following form:

$$M = [ \begin{matrix}{\sum }_{k = 1}^{\vert A \vert} {v ( {A}_{k} )}^{e} {h}_{α}^{′} ( {A}_{k} ) & {\sum }_{k = 1}^{\vert B \vert} {v ( {B}_{k} )}^{e} {h}_{α}^{′} ( {B}_{k} ) \\ {\sum }_{k = 1}^{\vert A \vert} {v ( {A}_{k} )}^{e} {h}_{β}^{′} ( {A}_{k} ) & {\sum }_{k = 1}^{\vert B \vert} {v ( {B}_{k} )}^{e} {h}_{β}^{′} ( {B}_{k} )\end{matrix} ] . \qquad ( 9.115 )$$

### 9.6.2 Decoding View

Formula (9.111) can also be factored as a weighted sum of the values of ^{d}h_{b}″(t) at the times of the spikes in a. That is,

$$\begin{matrix}{M}_{a , b} = {\sum }_{j = 1}^{J} {\sum }_{k = 1}^{K} H ( {b}_{k} - {a}_{j} ) \overline{u ( {α}_{j} )} v ( {b}_{k} ) {e}^{- s ( {b}_{k} - {a}_{j} )} \\ = {\sum }_{j = 1}^{J} \overline{u ( {α}_{j} )} {\underbrace{( {\sum }_{k = 1}^{K} H ( {b}_{k} - {a}_{j} ) v ( {b}_{k} ) {e}^{- s ( {b}_{k} - {a}_{j} )} )}}_{{}_{}^{d} {h}_{b}^{″} ( {a}_{j} )} \\ = {\sum }_{j = 1}^{J} \overline{u ( {α}_{j} )} {}_{}^{d} {h}_{b}^{″} ( {a}_{j} ) .\end{matrix} \qquad ( 9.116 )$$

In this case, the weights are equal to u̅(̅a̅ⱼ̅)̅, i.e., the conjugated value of the weighting function u at the times of the spikes in a.

Once again, this factorization applies to all elements of the matrix. In particular, a 2×2 matrix can be expressed as follows:

$$M = [ \begin{matrix}{\sum }_{j = 1}^{\vert α \vert} \overline{u ( {α}_{j} )} {}_{}^{d} {h}_{a}^{″} ( {α}_{j} ) & {\sum }_{k = 1}^{\vert α \vert} \overline{u ( {α}_{j} )} {}_{}^{d} {h}_{B}^{″} ( {α}_{j} ) \\ {\sum }_{j = 1}^{\vert β \vert} \overline{u ( {β}_{j} )} {}_{}^{d} {h}_{A}^{″} ( {β}_{j} ) & {\sum }_{j = 1}^{\vert β \vert} \overline{u ( {β}_{j} )} {}_{}^{d} {h}_{B}^{″} ( {β}_{j} )\end{matrix} ] . \qquad ( 9.117 )$$

### 9.7 Derivation of the Iterative Encoding Formulas

This section derives the formulas that are used by the SUV encoding algorithm, which is described in Section 9.8. These formulas are iterative versions of formulas (9.106), (9.107), and (9.108).

9.7.1 Computing the a-th Element of the Vector h′

The iterative formula for computing the value of ^{e}hₐ′(aₘ) in terms of the value of ^{e}hₐ′(aₘ₋₁) can be derived using the additivity of the Laplace transform. Let a[0, aₘ] be a truncated spike train that contains the first m spikes from a. Then, a[0, aₘ] can be represented as the following sum:

$$a [ 0 , {a}_{m} ] = a [ 0 , {a}_{m - 1} ] + a ( {a}_{m - 1} , {a}_{m} ] . \qquad ( 9.118 )$$

Also, recall that the value of ^{e}hₐ′ at time t during encoding is given by the following formula:

$${}^{e} {h}_{a}^{′} ( t ) = {ℒ}^{( \overline{\overleftarrow{u}} )} \{ \overleftarrow{a} [ 0 , t ] \} ( s ) = {e}^{- s t} \overline{{ℒ}^{( u )} \{ a [ 0 , t ] \} ( - \overline{s} )} . \qquad ( 9.119 )$$

By setting t=aₘ into the previous formula and then using (9.118), the value of ^{e}hₐ′(aₘ) can be expressed in the following way:

$$( 9.12 )$$

$$\begin{matrix}{}^{e} {h}_{a}^{′} ( {a}_{m} ) = {ℒ}^{( \overline{\overleftarrow{u}} )} \{ \overleftarrow{a} [ 0 , {a}_{m} ] \} ( s ) \\ = {e}^{- {sa}_{m}} \overline{{ℒ}^{( u )} \{ a [ 0 , {a}_{m} ] \} ( - \overline{s} )} \\ = {e}^{- s {a}_{m}} \overline{{ℒ}^{( u )} \{ a [ 0 , {a}_{m - 1} ] \} ( - \overline{s} )} + {e}^{- s {a}_{m}} \overline{{ℒ}^{( u )} \{ {\underbrace{a ( {a}_{m - 1} , {a}_{m} ]}}_{δ ( t - {a}_{m} )} \} ( - \overline{s} )} \\ = {e}^{- s ( {a}_{m} - {a}_{m - 1} )} {\underbrace{{e}^{- s {a}_{m - 1}} \overline{{ℒ}^{( u )} \{ a [ 0 , {a}_{m - 1} ] \} ( - \overline{s} )}}}_{{}^{e} {h}_{a}^{′} ( {a}_{m - 1} )} + {\underbrace{{e}^{- s {a}_{m}} \overline{{e}^{- ( - \overline{s} {a}_{m} )}}}}_{1} \overline{u ( {a}_{m} )} \\ = {}^{e} {h}_{a}^{′} ( {a}_{m - 1} ) {e}^{- s ( {a}_{m} - {a}_{m - 1} )} + \overline{u ( {a}_{m} )} .\end{matrix}$$

The second formula expresses the value of ^{e}hₐ′ at the time of the n-th spike in b in terms of its value at the previous spike in a. Let p be the index of that previous spike on channel a, i.e., p=max{j: aⱼ≤bₙ}. In this case, the truncated spike train a[0, bₙ] can be expressed as follows:

$$a [ 0 , {b}_{n} ] = a [ 0 , {a}_{p} ] + a ( {a}_{p} , {b}_{n} ] . \qquad ( 9.121 )$$

Note that the slice a(aₚ, bₙ] is empty because by definition there are no spikes after aₚ and before bₙ. Thus, by setting t=bₙ into (9.119) we get:

$$\begin{matrix}{}^{e} {h}_{a}^{′} ( {b}_{n} ) = {ℒ}^{( \overline{\overleftarrow{u}} )} \{ \overleftarrow{a} [ 0 , {b}_{n} ] \} ( s ) \\ = {e}^{- {sb}_{n}} \overline{{ℒ}^{( u )} \{ a [ 0 , {b}_{n} ] \} ( - \overline{s} )} \\ = {e}^{- {sb}_{n}} \overline{{ℒ}^{( u )} \{ a [ 0 , {a}_{p} ] \} ( - \overline{s} )} + {e}^{- {sb}_{n}} \overline{{ℒ}^{( u )} \{ {\underbrace{a ( {a}_{p} , {b}_{n} ]}}_{0} \} ( - \overline{s} )} \\ = {e}^{- s ( {b}_{n} - {a}_{p} )} {\underbrace{{e}^{- s {a}_{p}} \overline{{ℒ}^{( u )} \{ a [ 0 , {a}_{p} ] \} ( - \overline{s} )}}}_{{}^{e} {h}_{a}^{′} ( {a}_{p} )} \\ = {}^{e} {h}_{a}^{′} ( {a}_{p} ) {e}^{- s ( {b}_{n} - {a}_{p} )} .\end{matrix} \qquad ( 9.122 )$$

To summarize, the formulas for updating ^{e}hₐ′ during encoding are:

$${}^{e} {h}_{a}^{′} ( {a}_{m} ) = {}^{e} {h}_{a}^{′} ( {a}_{m - 1} ) {e}^{- s ( {a}_{m} - {a}_{m - 1} )} + \overline{u ( {a}_{m} )} , \qquad ( 9.123 )$$

$${}^{e} {h}_{a}^{′} ( {b}_{n} ) = {}^{e} {h}_{a}^{′} ( {a}_{p} ) {e}^{- s ( {b}_{n} - {a}_{p} )} . \qquad ( 9.124 )$$

The reason for stating two formulas is that each of them is used for a different purpose. The first one is used to update ^{e}hₐ′ at the times of the spikes on channel a. The second one updates ^{e}hₐ′ at the times of the spikes on channel b. Section 9.7.4 merges these two formulas into a single formula by using a common timeline for the spikes on both channels.
9.7.2 Computing the b-th Element of the Vector h″

The value of ^{e}h_{b}″ at time t during the encoding process is given by formula (9.107), which is replicated below:

$${}^{e} {h}_{b}^{″} ( t ) = {ℒ}^{( v )} \{ b [ 0 , t ] \} ( s ) . \qquad ( 9.125 )$$

Our goal is to compute the value of ^{e}h_{b}″ incrementally, i.e., to compute ^{e}h_{b}″(bₙ) in terms of its previous value ^{e}h_{b}″(bₙ₋₁). To derive an iterative formula we can start by cutting the spike train b[0, bₙ] into two parts at time bₙ₋₁, i.e.,

$$b [ 0 , {b}_{n} ] = b [ 0 , {b}_{n - 1} ] + b ( {b}_{n - 1} , {b}_{n} ] . \qquad ( 9.126 )$$

Note that the second slice contains just one spike that is at time bₙ, and thus it can be represented with a shifted delta function. Then, formula (9.125) and the additivity of the Laplace transform can be used to derive the following:

$$\begin{matrix}{}^{e} {h}_{b}^{″} ( {b}_{n} ) = {ℒ}^{( v )} \{ b [ 0 , {b}_{n} ] \} ( s ) \\ = {\underbrace{{ℒ}^{( v )} \{ b [ 0 , {b}_{n - 1} ] \} ( s )}}_{{}^{e} {h}_{b}^{″} ( {b}_{n - 1} )} + {ℒ}^{( v )} \{ {\underbrace{b ( {b}_{n - 1 ,} {b}_{n} ]}}_{δ ( t - {b}_{n} )} \} ( s ) \\ = {}^{e} {h}_{b}^{″} ( {b}_{n - 1} ) {ℒ}^{( v )} \{ δ ( t - {b}_{n} ) \} ( s ) \\ = {}^{e} {h}_{b}^{″} ( {b}_{n - 1} ) + v ( {b}_{n} ) {e}^{- s {b}_{n}} .\end{matrix} \qquad ( 9.127 )$$

Thus, the iterative formula is:

$${}^{e} {h}_{b}^{″} ( {b}_{n} ) = {}^{e} {h}_{b}^{″} ( {b}_{n - 1} ) + v ( {b}_{n} ) {e}^{- s {b}_{n}} . \qquad ( 9.128 )$$

That is, the value of the b-th element of h″ at time bₙ during encoding is equal to the value of the same element at time bₙ₋₁ plus the product between the value of the weighting function v(t) at time bₙ multiplied by e^{-sb}^{ₙ}.
9.7.3 Computing the Matrix Element in the a-th Row and b-th Column

Let a=(a₁, a₂, . . . , a_{J}) and b=(b₁, b₂, . . . , b_{K}) be two causal spike trains that are weighted by the functions u(t) and v(t), respectively. The matrix element that is encoded from these two spike trains is denoted with M_{a,b}. Its value at time t during encoding is equal to:

$${e}^{} {M}_{a , b} ( t ) = {ℒ}^{( u , v )} \{ a [ 0 , t ] ★b [ 0 , t ] \} ( s ) . \qquad ( 9.129 )$$

That is, its value is equal to the Laplace transform at s of the cross-correlation of a and b, both of which are truncated at time t and weighted by u and v.

Because formula (9.129) is valid for any time t, we can set t to the time of the (n−1)-st spike in b, i.e., t=bₙ₋₁, to get the following: E

$${e}^{} {M}_{a , b} ( {b}_{n - 1} ) = {ℒ}^{( u , v )} \{ a [ 0 , {b}_{n - 1} ] ★b [ 0 , {b}_{n - 1} ] \} ( s ) . \qquad ( 9.13 )$$

Also, by setting t to the time of the n-th spike in b leads to:

$${e}^{} {M}_{a , b} ( {b}_{n} ) = {ℒ}^{( u , v )} \{ a [ 0 , {b}_{n} ] ★b [ 0 , {b}_{n} ] \} ( s ) . \qquad ( 9.131 )$$

Corollary 9.20 implies that (9.131) can be represented as the following sum:

$${\underbrace{{ℒ}^{( u , v )} \{ a [ 0 , {b}_{n} ] ★b [ 0 , {b}_{n} ] \} ( s )}}_{{e}^{} {M}_{a , b} ( {b}_{n} )} = {\underbrace{{ℒ}^{( u , v )} \{ a [ 0 , {b}_{n - 1} ] ★b [ 0 , {b}_{n - 1} ] \} ( s )}}_{{e}^{} {M}_{a , b} ( {b}_{n - 1} )} + v ( {b}_{n} ) {\underbrace{{ℒ}^{( \overline{\overleftarrow{u}} )} \{ \overleftarrow{a} [ 0 , {b}_{n} ] \} ( s )}}_{{}^{e} {h}_{a}^{′} ( {b}_{n} )} . \qquad ( 9.132 )$$

Corollary 9.20 also implies that, in the special case when t=b₁, the formula has the following form:

$${\underbrace{{ℒ}^{( u , v )} \{ a [ 0 , {b}_{1} ] ★b [ 0 , {b}_{1} ] \} ( s )}}_{{e}^{} {M}_{a , b} ( {b}_{1} )} = v ( {b}_{1} ) {\underbrace{{ℒ}^{( \overline{\overleftarrow{u}} )} \{ \overleftarrow{a} [ 0 , {b}_{1} ] \} ( s )}}_{{}^{e} {h}_{a}^{′} ( {b}_{1} )} , \qquad ( 9.133 )$$

Therefore, the value of the matrix element ^{e}M_{a,b} can be updated iteratively at the times of the spikes on channel b. The iterative update formula follows from (9.132) and (9.133), i.e.,

$${e}^{} {M}_{a , b} ( {b}_{n} ) = {e}^{} {M}_{a , b} ( {b}_{n - 1} ) + v ( {b}_{n} ) {}^{e} {h}_{a}^{′} ( {b}_{n} ) . \qquad ( 9.134 )$$

In other words, at the time of the n-th spike on channel b during encoding, the value of the matrix element M_{a,b} is equal to its previous value at the time of the (n−1)-st spike in b plus the value of the weighting function at the time of the n-th spike in b multiplied by the value of the a-th element of the vector h′ at the time of the n-th spike in b.

### 9.7.4 The Iterative Encoding Formulas for a Common Timeline

This section expresses the encoding formulas for a common timeline that combines the spike times from a and b. The formulas derived here are more suitable for an algorithmic implementation. The encoding algorithm is described in the next section.

Let a=(a₁, a₂, . . . , a_{J}) and b=(b₁, b₂, . . . , b_{K}) be two spike trains and let u(t) and v(t) be their corresponding weighting functions. Also, let c=(c₁, c₂, . . . , c_{J+K}) be a list of spike times that combines the spike times from a and b and sorts them in increasing order. Finally, let a=(â₁, â₂, . . . , â_{J+K}) be a binary array such that its i-th element is defined as follows:

$${\hat{a}}_{i} = \{ \begin{matrix}1 , & if {c}_{i} comes from a , \\ 0 , & if {c}_{i} comes from b .\end{matrix} \qquad ( 9.135 )$$

This array represents the origin of each spike. The elements of c and â can be computed using the algorithm described in Section 8.10.4. That algorithm merges the spike times in a and b to produce c. If an element of a is equal to an element of b, then it gives precedence to the spike from a.

The encoding algorithm assumes that the variables ^{e}hₐ′, ^{e}h_{b}″, and ^{e}M_{a,b}, which store the SSM model during encoding, are initialized to zero. More formally, the initial conditions are:

$${}^{e} {h}_{a}^{′} [ 0 ] = 0 , \qquad ( 9.136 )$$

$${}^{e} {h}_{b}^{″} [ 0 ] = 0 , \qquad ( 9.137 )$$

$${}^{e} {M}_{a , b} [ 0 ] = 0. \qquad ( 9.138 )$$

These formulas use the 0-th iteration counter to capture the initial conditions, i.e., they use an implicit c₀ that is at time t=0. Also, the formulas use square brackets instead of round brackets to indicate the iteration counter. This notation is useful for an algorithmic implementation, but it does not mean that time is discretized at regular intervals. Instead, the time is indexed at the times of the spikes. That is, the i-th index corresponds to an update that will be performed at time cᵢ (this is different from the ZUV model, in which the time is discretized).

The a-th element of the vector h′ is updated using the following formula:

$${}^{e} {h}_{a}^{′} [ i ] = {}^{e} {h}_{a}^{′} [ i - 1 ] {e}^{- s ( {c}_{i} - {c}_{i} - 1 )} + \{ \begin{matrix}\overline{u ( {c}_{i} )} , & if {\hat{a}}_{i} = 1 , \\ 0 , & if {\hat{a}}_{i} = 0 ,\end{matrix} \qquad ( 9.139 )$$

for each i∈{1, 2, . . . , J+K}.

The matrix element ^{e}M_{a,b} is updated using the following rule:

$${}^{e} {M}_{a , b} [ i ] = {}^{e} {M}_{a , b} [ i - 1 ] + \{ \begin{matrix}0 , & if {\hat{a}}_{i} = 1 , \\ v ( {c}_{i} ) {}^{e} {h}_{a}^{′} [ i ] , & if {\hat{a}}_{i} = 0 ,\end{matrix} \qquad ( 9.14 )$$

for each i∈{1, 2, . . . , J+K}. The two formulas imply that ^{e}hₐ′ is updated before its value is used to update the matrix element M_{a,b}.

The b-th element of the vector h″ is updated as follows:

$${}^{e} {h}_{b}^{″} [ i ] = {}^{e} {h}_{b}^{″} [ i - 1 ] + \{ \begin{matrix}0 , & if {\hat{a}}_{i} = 1 , \\ v ( {c}_{i} ) {e}^{- s {c}_{i}} , & if {\hat{a}}_{i} = 0 ,\end{matrix} \qquad ( 9.141 )$$

for each i∈{1, 2, . . . , J+K}.

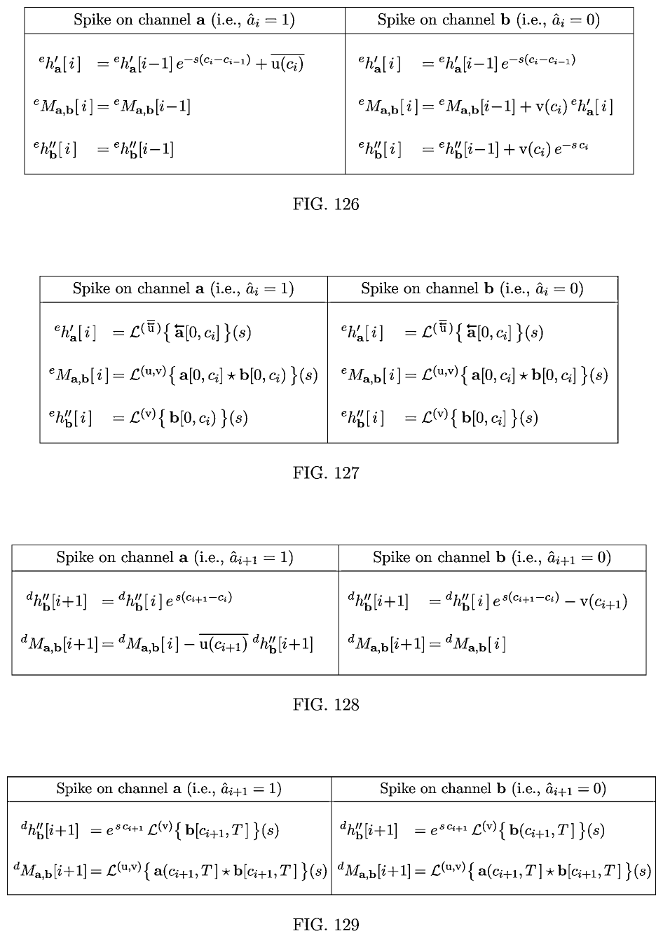

FIG. 126 presents the encoding formulas by splitting them into two columns. The formulas in the first column are used when the incoming spike is on channel a (i.e., âᵢ=1). The formulas in the second column are used when the current spike is on channel b (i.e., âᵢ=0). In each iteration, the algorithm uses the formulas from only one of these two columns.

If two spikes occur at the same time, i.e., when aⱼ=bₖ for some j and k, then they are processed individually in two separate iterations. The spike from channel a is processed first; the spike from channel b is processed in the next iteration. Because in the case of coincidence cᵢ=cᵢ₋₁, the second update does not affect ^{e}hₐ′, which remains unchanged since e^{-s(c}^{ᵢ}^{-c}^{_{i−1}}⁾=e⁰=1. Only ^{e}M_{a,b} and ^{e}h_{b}″ are updated in the second iteration.

FIG. 127 describes the mapping between the state of the variables of the encoding algorithm and the theoretical SUV model based on the Laplace transform. This mapping is applicable at the end of the i-th encoding iteration. Because the algorithm processes the spikes from a differently than the spikes from b, the mapping depends on the origin of the current spike. More specifically, if the spike cᵢ comes from a, then the right end of the interval b[0, cᵢ) is open. If the spike cᵢ comes from b, then the right end of the interval b[0, cᵢ] is closed. This difference allows the encoding algorithm to process pairs of coincident spikes accurately using two consecutive iterations.

The formulas in the previous subsections were formulated for split timelines, i.e., separately for a and b. When a and b are merged into c, however, there can be ambiguities when spikes on a and b coincide (e.g., ^{e}h_{b}″(aⱼ)≠^{e}h_{b}″(bₖ) even though aⱼ=bₖ). Defining the common timeline mapping as shown in FIG. 127 allows us to resolve these ambiguities.

### 9.8 The SUV Encoding Algorithm

This algorithm is based on the formulas for a common timeline given in Section 9.7.4. The common timeline c, however, is not explicitly computed by the algorithm. Instead, it is constructed by implicitly merging the spikes from a and b, which is possible because the spike times in both a and b are sorted. Only the two most recent spike times are preserved and stored in the variables tₚᵣₑᵥ and t. The boolean array â is not generated either because the algorithm needs only the relevant element of â, which is stored in the boolean variable spikeOnA. The computational complexity of the SUV encoding algorithm is O(J+K), where J is the number of spikes in a and K is the number of spikes in b.

The structure of the algorithm is similar to the algorithm for non-weighted spike trains, which was described in Section 8.11. Because the trains are now weighted, however, this requires some additional bookkeeping. The algorithm uses the helper variables û, \hat{v}, and ĝ to incrementally update the values of the exponential weights from their previous values. These updates use the following property of the exponential function: e^{x+y}=e^{x}e^{y} or, in this case, e^{-st}^{₁}=e^{-st}^{₀}e^{-s(t}^{₁}^{-t}^{₀}⁾. This is similar to the exponential updates in the ZUV algorithm, but now the time is no longer discretized.

In the previous sections, the weighting functions were stated in an abstract form, i.e., u(t) and v(t). The algorithm, however, needs to use concrete weighting functions. In this implementation, these functions are u(t)=U e^{-ut} and v(t)=V e^{-vt}, where the scaling constants U and V are assumed to be equal to 1. The parameters u and v determine the decay rates of the exponentials. Together with the parameter s, they form the three main arguments of the SUV encoding algorithm. The remaining arguments are two lists that represent the spike trains a and b. The algorithm returns the value of the matrix element M_{a,b} and the elements hₐ′ and h_{b}″ of the two vectors. If both u and v are real, then all conjugations in the formulas can be dropped and the algorithm does not need to handle them.

The algorithm computes only one element of the matrix. Because the encoding of each matrix element is independent of the other elements, the entire matrix can be computed in parallel by running one instance of the algorithm for each matrix element.

A small technical detail that is worth mentioning is how the algorithm handles coincident spikes on a and b. Suppose that aⱼ=bₖ for some j and k. In this case, the encoding formulas described in the previous section process the spike from a before the spike from b. More specifically, when aⱼ=bₖ, the spike on a at time aⱼ is processed in the first iteration and the spike on b at time bₖ is processed in the second iteration. This order can be enforced by the condition aⱼ≤bₖ. Because aⱼ=bₖ, however, this implies that t−tₚᵣₑᵥ=0 when bₖ is processed. In this case, all updates that use t−tₚᵣₑᵥ, in the exponent have no effect, i.e., they reduce to multiplication by 1. That is, the updates of M_{a,b} and h_{b}″ will use the previous values of hₐ′, \hat{v}, and ĝ.

### 9.9 Derivation of the Iterative Decoding Verification Formulas

This section derives iterative formulas for verifying the solution obtained by a decoding algorithm. The formulas suggest how the values of M_{a,b} and h_{b}″ can be gradually depleted down to zero. The formulas assume that the spike train a is available, which is not the case for decoding. Thus, these are verification formulas and not decoding formulas. A decoding algorithm has to infer the times of the spikes on a.

9.9.1 Updating the Matrix Element in the a-th Row and b-th Column

Let a=(a₁, a₂, . . . , a_{J}) and b=(b₁, b₂, . . . , b_{K}) be two causal spike trains such that all of their spikes occur no later than time T. At time t during decoding the matrix element M_{a,b} has the following value:

$${}^{d} {M}_{a , b} ( t ) = {ℒ}^{( u , v )} \{ a [ t , T ] ★ b [ t , T ] \} ( s ) . \qquad ( 9.142 )$$

This equation follows from formula (9.109) and is valid for any time t∈[0, T]. In particular, if we set t to the time of the m-th spike in a, i.e., t=aₘ, then we will get:

$${}^{d} {M}_{a , b} ( {a}_{m} ) = {ℒ}^{( u , v )} \{ a [ {a}_{m} , T ] ★ b [ {a}_{m} , T ] \} ( s ) . \qquad ( 9.143 )$$

The same expression can also be evaluated at t=aₘ₊₁, i.e., the time of the (m+1)-st spike in a, which leads to:

$${}^{d} {M}_{a , b} ( {a}_{m + 1} ) = {ℒ}^{( u , v )} \{ a [ {a}_{m + 1} , T ] ★ b [ {a}_{m + 1} , T ] \} ( s ) . \qquad ( 9.144 )$$

Corollary 9.21 allows us to rewrite (9.143) as the following sum:

$${\underbrace{{ℒ}^{( u , v )} \{ a [ {a}_{m} , T ] ★ b [ {a}_{m} , T ] \} ( s )}}_{{}^{d} {M}_{a , b} ( {a}_{m} )} = \overline{u ( {a}_{m} )} {\underbrace{{e}^{s {a}_{m}} {ℒ}^{( v )} \{ b [ {a}_{m} , T ] \} ( s )}}_{{}^{d} {h}_{b}^{″} ( {a}_{m} )} + {\underbrace{{ℒ}^{( u , v )} \{ a [ {a}_{m + 1} , T ] ★ b [ {a}_{m + 1} , T ] \} ( s )} .}_{{}^{d} {M}_{a , b} ( {a}_{m + 1} )} \qquad ( 9.145 )$$

That is, the value of M_{a,b} at the time of the (m+1)-st spike in a is equal to its previous value at the time of the m-th spike in a minus the value of h_{b}″ at the time of the m-th spike in a, times the conjugated value of the weighting function u, also at t=aₘ. In the special case when t=a_{J}, i.e., at the last iteration, Corollary 9.21 also implies that:

$${\underbrace{{ℒ}^{( u , v )} \{ a [ {a}_{J} , T ] ★ b [ {a}_{J} , T ] \} ( s )}}_{{}^{d} {M}_{a , b} ( {a}_{J} )} = \overline{u ( {a}_{J} )} {\underbrace{{e}^{s {a}_{J}} {ℒ}^{( v )} \{ b [ {a}_{J} , T ] \} ( s )}}_{{}^{d} {h}_{b}^{″} ( {a}_{J} )} . \qquad ( 9.146 )$$

The iterative formula follows from (9.145) after rearranging its terms:

$${}^{d} {M}_{a , b} ( {a}_{m + 1} ) = {}^{d} {M}_{a , b} ( {a}_{m} ) - \overline{u ( {a}_{m} )} {}^{d} {h}_{b}^{″} ( {a}_{m} ) . \qquad ( 9.147 )$$

9.9.2 Updating the b-th Element of the Vector h″

At time t during decoding the value of the b-th element of h″ is given by formula (9.110), which is replicated below:

$${}^{d} {h}_{b}^{″} ( t ) = {e}^{st} {ℒ}^{( v )} \{ b [ t , T ] \} ( s ) . \qquad ( 9.148 )$$

This formula is mathematically correct, but it does not show how to iteratively compute the value of h_{b}″. To derive an iterative formula, we will start by representing the truncated spike train b[bₙ, T] as the following sum:

$$b [ {b}_{n} , T ] = b [ {b}_{n} , {b}_{n + 1} ) + b [ {b}_{n + 1} , T ] . \qquad ( 9.149 )$$

In other words, we will split it into two non-overlapping pieces, where the cut is at the time of the (n+1)-st spike on channel b.

Now, if we set t=bₙ in (9.148) and then use (9.149) we will get the following expression:

$$\begin{matrix}{}^{d} {h}_{b}^{″} ( {b}_{n} ) = {e}^{s {b}_{n}} {ℒ}^{( v )} \{ b [ {b}_{n} , T ] \} ( s ) \\ = {e}^{s {b}_{n}} {ℒ}^{( v )} \{ {\underbrace{b [ {b}_{n} , {b}_{n + 1} )}}_{δ ( t - {b}_{n} )} \} ( s ) + {e}^{s {b}_{n}} {ℒ}^{( v )} \{ b [ {b}_{n + 1} , T \} ( s ) \\ = {\underbrace{{e}^{s {b}_{n}} {e}^{- s {b}_{n}}}}_{1} v ( {b}_{n} ) + {e}^{s ( {b}_{n} - {b}_{n + 1} )} {\underbrace{{e}^{s {b}_{n + 1}} {ℒ}^{( v )} \{ b [ {b}_{n + 1} , T ] \} ( s )}}_{{}^{d} {h}_{b}^{″} ( {b}_{n + 1} )} \\ = v ( {b}_{n} ) + {}^{d} {h}_{b}^{″} ( {b}_{n + 1} ) {e}^{s ( {b}_{n} - {b}_{n + 1} )} .\end{matrix} \qquad ( 9.15 )$$

Rearranging the terms in (9.150) leads to the following iterative formula:

$${}^{d} {h}_{b}^{″} ( {b}_{n + 1} ) = [ {}^{d} {h}_{b}^{″} ( {b}_{n} ) - v ( {b}_{n} ) ] {e}^{s ( {b}_{n + 1} - {b}_{n} )} . \qquad ( 9.151 )$$

Using a similar approach, we can derive another update rule for ^{d}h_{b}″, in this case, for the times of spikes on channel a. Let p be the index of the last spike in b that is strictly before the m-th spike on channel a, i.e., p=max{k: bₖ<aₘ}. Then, the truncated spike train b[bₚ, T] can be split into two spike trains by a cut at time aₘ as follows:

$$b [ {b}_{p} , T ] = b [ {b}_{p} , {a}_{m} ) + b [ {a}_{m} , T ] . \qquad ( 9.152 )$$

Note that the first slice contains just one spike so its Laplace transform reduces to the Laplace transform of a shifted and weighted delta function.

Setting t=bₚ in (9.148) and then using (9.152) leads to the following expression:

$$\begin{matrix}{}^{d} {h}_{b}^{″} ( {b}_{p} ) & = {e}^{{sb}_{p}} {ℒ}^{( v )} \{ b [ {b}_{p} , T ] \} ( s ) \\ & = {e}^{{sb}_{p}} {ℒ}^{( v )} \{ b {\underbrace{[ {b}_{p} , {a}_{m} )}}_{δ ( t - {b}_{p} )} \} ( s ) + {e}^{{sb}_{p}} {ℒ}^{( v )} \{ b [ {a}_{m} , T ] \} ( s ) \\ & = {e}^{{sb}_{p}} {ℒ}^{( v )} \{ δ ( t - {b}_{p} ) \} ( s ) + {e}^{s ( {b}_{p} - {a}_{m} )} {\underbrace{{e}^{{sa}_{m}} {ℒ}^{( v )} \{ b [ {a}_{m} , T ] \} ( s )}}_{{}^{d} {h}_{b}^{″} ( {a}_{m} )} \\ & = {\underbrace{{e}^{{sb}_{p}} {e}^{- {sb}_{p}}}}_{1} v ( {b}_{p} ) + {}^{d} {h}_{b}^{″} ( {a}_{m} ) {e}^{s ( {b}_{p} - {a}_{m} )} \\ & = v ( {b}_{p} ) + {}^{d} {h}_{b}^{″} ( {a}_{m} ) {e}^{s ( {b}_{p} - {a}_{m} )}\end{matrix} . \qquad ( 9.153 )$$

After rearranging the terms, we get the following iterative formula:

$${}^{d} {h}_{b}^{″} ( {a}_{m} ) = [ {}^{d} {h}_{b}^{″} ( {b}_{p} ) - v ( {b}_{p} ) ] {e}^{s ( {a}_{m} - {b}_{p} )} . \qquad ( 9.154 )$$

To summarize, the two iterative decoding verification formulas for the value of the b-th element of the vector h″ are:

$${}^{d} {h}_{b}^{″} ( {b}_{n + 1} ) = [ {}^{d} {h}_{b}^{″} ( {b}_{n} ) - v ( {b}_{n} ) ] {e}^{s ( {b}_{n + 1} - {b}_{n} )} , \qquad ( 9.155 )$$

$${}^{d} {h}_{b}^{″} ( {a}_{m} ) = [ {}^{d} {h}_{b}^{″} ( {b}_{p} ) - v ( {b}_{p} ) ] {e}^{s ( {a}_{m} - {b}_{p} )} . \qquad ( 9.156 )$$

The next section combines both of these formulas into one formula that uses a common timeline. This common timeline is denoted with c and it combines the spike times from a and b.

Finally, it is worth mentioning two special cases of formula (9.148). In the first case the value of t is equal to 0. This leads to the following expression:

$${}^{d} {h}_{b}^{″} ( 0 ) = {\underbrace{{e}^{s 0}}}_{1} {ℒ}^{( v )} \{ b [ 0 , T ] \} ( s ) = {ℒ}^{( v )} \{ b [ 0 , T ] \} ( s ) = {}^{e} {h}_{b}^{″} ( T ) . \qquad ( 9.157 )$$

In other words, the initial value of h_{b}″ at time 0 during decoding is equal to the final value of h_{b}″ at time T during encoding.

The second special case evaluates (9.148) at time t₁=min(a₁, b₁). In other words, t₁ is the time of the first spike on either channel a or channel b. In this case the formula reduces to:

$$\begin{matrix}{}^{d} {h}_{b}^{″} ( {t}_{1} ) & = {e}^{{st}_{1}} {ℒ}^{( v )} \{ b [ {t}_{1} , T ] \} ( s ) \\ & = {e}^{{st}_{1}} {ℒ}^{( v )} \{ b [ 0 , T ] \} ( s ) \\ & = {{e}^{{st}_{1}}}^{e} {h}_{b}^{″} ( T ) \\ & = {e}^{{st}_{1}} {}^{d} {h}_{b}^{″} ( 0 )\end{matrix} . \qquad ( 9.158 )$$

Therefore, the value of h_{b}″ should be multiplied by e^{st}^{₁} before the first time this element is used in the update formulas. This derivation uses the fact that t₁ is either the time of the first spike in b or is less than that. In both cases b[0, t₁) contains no spikes and its Laplace transform is equal to zero. Thus, the interval b[t₁, T] can be extended to b[0, T].

### 9.9.3 The Decoding Verification Formulas for a Common Timeline

This section restates the formulas for M_{a,b} and ^{d}h_{b}″ that were derived previously in a more suitable form for an algorithmic implementation. The rewritten formulas use a common timeline c, which combines the spike times from a and b.

Let a=(a₁, a₂, . . . , a_{J}) be a causal spike train that is weighted by the function u(t). Also, let b=(b₁, b₂, . . . , b_{K}) be another spike train that is weighted by the function v(t). Furthermore, let c=(c₁, c₂, . . . , c_{J+K}) be a list of spike times that combines the spike times from a and b and sorts them in increasing order.

The common timeline c can be constructed using the algorithm described in Section 8.10.4. That algorithm merges the sorted lists a and b. If two spikes coincide, then the spike from a precedes the spike from b. The algorithm also computes a binary array â=(â₁, â₂, . . . , â_{J+K}) that is used to trace each spike in c to its original spike train, which is either a or b. If a spike in c came from a, then the corresponding element of â is 1. On the other hand, if a spike in c came from b, then the value of the corresponding element of â is 0.

The initial conditions for the verification process can be stated as follows:

$${}^{d} {h}_{b}^{″} [ 0 ] = {}^{e} {h}_{b}^{″} [ J + K ] , \qquad ( 9.159 )$$

$${}^{d} {M}_{a , b} [ 0 ] = {}^{e} {M}_{a , b} [ J + K ] . \qquad ( 9.16 )$$

In other words, the initial values during decoding are equal to the final values during encoding.

Formula (9.150) and formula (9.153) can be combined into one formula as follows:

$${}^{d} {h}_{b}^{″} [ i ] = {}^{d} {h}_{b}^{″} [ i + 1 ] {e}^{s ( {c}_{i} - {c}_{i + 1} )} + \{ \begin{matrix}0 , & if {\hat{a}}_{i + 1} = 1 , \\ v ( {c}_{i + 1} ) , & if {\hat{a}}_{i + 1} = 0 .\end{matrix} \qquad ( 9.161 )$$

In this case, the updates are performed only for the spikes in b. The temporal order in formula (9.161), however, is reversed, i.e., ^{d}h_{b}″[i] is expressed in terms of ^{d}h_{b}″[i+1]. To fix this, we can rearrange it to et the following formula:

$${}^{d} {h}_{b}^{″} [ i + 1 ] = {}^{d} {h}_{b}^{″} [ i ] {e}^{s ( {c}_{i + 1} - {c}_{i} )} - \{ \begin{matrix}0 , & if {\hat{a}}_{i + 1} = 1 , \\ v ( {c}_{i + 1} ) , & if {\hat{a}}_{i + 1} = 0 ,\end{matrix} \qquad ( 9.162 )$$

where i∈{0, 1, 2, . . . , J+K−1}.

Equation (9.147) leads to the following update rule:

$${}^{d} {M}_{a , b} [ i + 1 ] = {}^{d} {M}_{a , b} [ i ] - \{ \begin{matrix}\overline{u ( {c}_{i + 1} )} {}^{d} {h}_{b}^{″} [ i + 1 ] , & if {\hat{a}}_{i + 1} = 1 , \\ 0 , & if {\hat{a}}_{i + 1} = 0 ,\end{matrix} \qquad ( 9.163 )$$

for each i∈{0, 1, 2, . . . , J+K−1}. As expected, the matrix element ^{d}M_{a,b} is updated only at the times of the spikes in a.

At the end of this process, the verification is successful if both the matrix element M_{a,b} and the vector element h_{b}″ contain zeros, i.e., ^{d}M_{a,b}[J+K]=0 and ^{d}h_{b}″[J+K]=0.

FIG. 128 summarizes the decoding verification formulas by grouping them into two columns. The first column lists the formulas that are used if the current spike came from channel a (i.e., âᵢ₊₁=1). The second column gives the update formulas for a spike on channel b (i.e., âᵢ₊₁=0). During each iteration, the verification algorithm uses only the formulas from one of these columns. If two spikes were emitted at the same time, i.e., if aⱼ=bₖ for some j∈{1, 2, . . . , J} and some k∈{1, 2, . . . , K}, then they are processed in two separate iterations: aⱼ is processed first and bₖ is processed second. In other words, the formulas in the first column have a priority over the formulas in the second column in the case of coincident spikes. Furthermore, in the second update the value of e^{s(c}^{ᵢ₊₁}^{-c}^{ᵢ}⁾ in the formula for h_{b}″ is equal to 1 because cᵢ₊₁=cᵢ. Therefore, this update reduces to subtracting v(cᵢ₊₁) from the current value of h_{b}″, which was updated in the previous iteration when the spike aⱼ was processed.

The ZUV decoding algorithm does not decay h″ before the initial iteration because the sequence indexing in the ZUV case is 0-based. Also, the multiplication in the ZUV algorithm is by z, but it can be viewed as a multiplication by z^{(t-t}^{ₚᵣₑᵥ}⁾, where t−tₚᵣₑᵥ is always equal to 1 except at the very beginning, i.e., when both t and tₚᵣₑᵥ are zero and the multiplication is then by z^{t-t}^{ₚᵣₑᵥ}=1. The ZUV code skips this degenerate update. The SUV algorithm, however, cannot skip this update because the time of the first spike may be different from zero.

FIG. 129 gives formulas for the state of the SUV model after the (i+1)-st iteration. The formulas in FIG. 128 can be derived from the formulas in FIG. 129 using a similar approach as in Section 8.12.4 and Section 8.12.5.

### 9.10 The Decoding Verification Algorithm

The SUV decoding verification algorithm can be stated for only one element of the matrix, which is encoded from a pair of spike trains that are denoted with a and b. The computational complexity of this algorithm is O(J+K), where J and K are the number of spikes in a and b, respectively. Thus, the complexity is the same for both the encoding algorithm and the verification algorithm.

The algorithm uses the weighting functions u(t)=Ue^{-ut} and v(t)=Ve^{-vt}. The scaling constants U and V are both equal to 1 in this case. Furthermore, if the parameters u and v are real, then there is no need for conjugation.

As in the encoding algorithm, if aⱼ=bₖ for some j and k, the algorithm performs two iterations to process this case. Precedence is given to the spike from a. During the second iteration, however, t−tₚᵣₑᵥ=0. Thus, the value of h″ from the previous iteration is used in the second update.

### 10 SUV Decoding Algorithms

The verification algorithm described in Chapter 9 showed that it is possible to ‘roll back’ the SUV model from its encoded state back to zero in linear time. The SUV verification algorithm, however, relies on knowing the times of the spikes in S′. This chapter states a decoding algorithm that does not use S′. It also analyzes some of the conditions under which S′ is decoded accurately.

### 10.1 The SUV Decoding Problem

The main difference between ZUV decoding and SUV decoding is that the SUV model decouples the times at which spikes are emitted from the times when h″ is decreased. The spikes are emitted at the times of the spikes on ^{d}S′, while the updates to h″ happen at the times of the spikes on ^{d}S″. In other words, the time at which a spike can be emitted is not constrained in the SUV model, in contrast to the ZUV model where it can only happen at integer multiples of the discretization interval. Thus, the SUV decoding algorithm has to operate in a space with more “degrees of freedom” compared to the space of discrete sequences in which the ZUV decoding algorithm operates.

Let α⁽¹⁾, α⁽²⁾, . . . , α^{(M′)} be the M′ spike trains in S′. Let A⁽¹⁾, A⁽²⁾, . . . , A^{(M″)} be the M″ spike trains in S″. The SUV decoding algorithm takes the encoded matrix M, the vector h″, and the spike trains A⁽¹⁾, A⁽²⁾, . . . , A^{(M″)} as its inputs and computes the spike trains α⁽¹⁾, α⁽²⁾, . . . , α^{(M′)}.

A useful property of the SUV decoding algorithm is its ability to handle cases when the spike trains in ^{d}S″ at decoding time may differ from their counterparts used for encoding the SUV model. It turns out that under certain constraints the SUV decoding algorithm can be robust to changing the spike times or deleting spikes in ^{d}S″.

The SUV decoding algorithm solves the problem “in real time”, i.e., it consumes the spikes in the ^{d}S″ channels in their chronological order and emits spikes on the ^{d}S′ channels as they are being decoded. That is, the algorithm does not use the spikes “from the future” in order to emit the spikes “in the present”. Only the data derived from the spikes “in the past” is used. In other words, there is no spike train buffering in the SUV decoding algorithm.

### 10.2 SUV Decoding for one Row of the Matrix

The SUV decoding algorithm uses a sequence of candidate spike times ψ=(ψ₁, ψ₂, . . . , ψ_{L}). The algorithm filters the times of candidate spikes to select only those that can be emitted in the decoded version of S′. This implies that the sequence ψ has to include all spike times in S′ to make accurate decoding possible. The sequence ψ can also include other candidate spike times.

This algorithm iterates through spike times similarly to the SUV verification algorithm. In contrast to the verification algorithm, however, the SUV decoding algorithm uses the ability to subtract h″ from m as a condition for selecting among candidates in the sequence ψ. In other words, unlike the verification algorithm, the decoding algorithm doesn't have a as one of its inputs. Instead, the decoding algorithm seeks to reconstruct a by filtering the possible spike times in ψ.

The computational complexity of the algorithm is O(\hat{K}+LM″). In this formula, L is the number of candidate spike times in the sequence ψ and \hat{K} is the number of all spikes in the collection of spike trains S″. Similarly to other formulas in this document, M″ is the size of the second alphabet. In this case, this is the number of spike trains in S″.

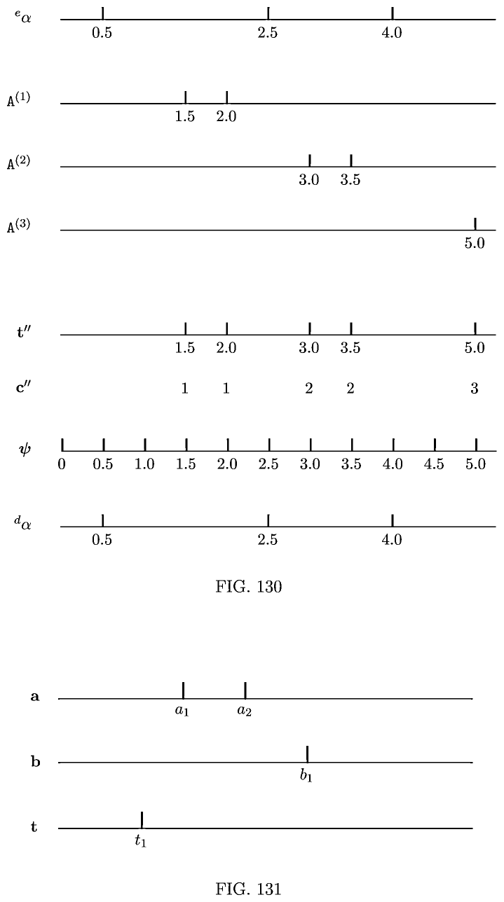

FIG. 130 gives an example that illustrates some of the input and internal variables of the algorithm. The model is encoded from ^{e}α and A=(A⁽¹⁾, A⁽²⁾, A⁽³⁾). The decoded spike train is denoted by ^{d}α. The list t″ stores the spike times of all spikes in A. For each spike in A, the list c″ stores the index of its original channel in A. The list ψ stores the possible candidate spike times for the output spikes.

When the algorithm is described in this form, it is necessary to build the vector ψ, which can be done using a merge sort of the N lists (i.e., spike trains), each of which is already sorted. The computational complexity is O(L N), where L is the total number of spikes in all spike trains in S. In distributed implementations or implementations in which the spikes come from an external input or device, this function may not be necessary.

### 10.3 SUV Decoding for a Complete Matrix

The SUV algorithm that decodes an entire matrix can use the same values of s, u, and v for all elements of the matrix. The computational complexity of this version of the algorithm is O(\hat{K}+LM′M″), where L is the total number of candidate spike times in the list ψ.

### 10.4 Alternative Version of SUV Decoding for a Complete Matrix

In an alternative version of the decoding algorithm, the parameters s, u, and v can be customized for each element of the matrix. In this case, the computational complexity is O((\hat{K}+\hat{L})M′M″), because the algorithm needs to update each matrix element for each incoming spike or candidate spike time. If all updates run in parallel, then the run-time complexity of this algorithm reduces to O(\hat{K}+\hat{L}).

10.5 Some Special Cases in which the Decoding is Accurate

This section focuses on several special cases of SUV decoding. The main goal is to describe when the SUV decoding algorithm can emit a spike. This decision is formalized using the sign of the difference between the value of the matrix element M_{a,b} and the value of ^{d}h_{b}″ weighted by the function u(t) at time t during decoding. This difference can be viewed as a strictly monotone function g(t). The proofs use the intermediate value theorem (from Calculus) to show that g(t) has only one zero in the decoding interval and that this zero is located at the correct decoding time. Theorem 10.1 covers the case when both spike trains consist of only one spike, i.e., a=(a₁) and b=(b₁). Theorem 10.2 extends this proof to the case when a=(a₁) and b=(b₁, b₂, . . . , b_{K}). In both cases it is assumed that a₁<b₁.

Theorem 10.1. Let a=(a₁) be a spike train and let b=(b₁) also be a spike train such that a₁<b₁. Let u(t) and v(t) be two continuous real weighting functions. Finally, let f(t) be the following function:

$$f ( t ) = u ( t ) {}^{d} {h}_{b}^{″} ( t ) = u ( t ) H ( {b}_{1} - t ) v ( {b}_{1} ) {e}^{- s ( {b}_{1} - t )} . \qquad ( 10.1 )$$

### That is,

$$f ( t ) = \{ \begin{matrix}u ( t ) y ( {b}_{1} ) {e}^{- s ( {b}_{1} - t )} , & if t \leq {b}_{1} , \\ 0 , & if t > {b}_{1} .\end{matrix} \qquad ( 10.2 )$$

Suppose that f(t) is strictly monotone on [0, b₁], i.e., for each t₁ [symbol] t₂ such that 0≤t₁<t₂≤b₁, f(t₁)<f(t₂) or f(t₁)>f(t₂). Then, f(t)=^{d}M_{a,b}(0) on t∈[0, b₁] if and only if t=a₁.

In other words, if there is only one spike on a and only one spike on b, and if the spike on b comes after the spike on a, then the function f(t)=u(t) ^{d}h_{b}″(t) crosses the value of ^{d}M_{a,b}(0) at exactly the right time during decoding, i.e., when t=a₁. The spike on b doesn't even have to be present during decoding in order to decode a₁ correctly. That is, if a spike on b arrives after a₁ or even if it doesn't arrive at all, then the decoding will still be accurate.

The following theorem extends the condition imposed on the function f(t) by Theorem 10.1 to the case when K>1. Once again, if a₁≤b₁, then the equation f(t)=^{d}M_{a,b}(0) has only one solution on [0, b₁] at t=a₁.

Theorem 10.2. Let a=(a₁) be a spike train that consists of a single spike that occurs at time a₁≥0. Let b=(b₁, b₂, . . . , b_{K}) be another spike train such that a₁≤b₁. Let u(t) and v(t) be two real weighting functions. Let s be a real number. Finally, let f (t) be the following function:

$$f ( t ) = u ( t ) {}^{d} {h}_{b}^{″} ( t ) = u ( t ) ( {\sum }_{k = 1}^{K} H ( {b}_{k} - t ) v ( {b}_{k} ) {e}^{- s ( {b}_{k} - t )} ) . \qquad ( 10.3 )$$

Suppose that f(t) is strictly monotone on [0, b₁]. That is,

$$f ( {t}_{1} ) > f ( {t}_{2} ) or f ( {t}_{1} ) < f ( {t}_{2} ) , for each {t}_{1} , {t}_{2} ∈ [ 0 , {b}_{1} ] s . t . {t}_{1} < {t}_{2} . \qquad ( 10.4 )$$

Then, the following equation

$${}^{d} {M}_{a , b} ( 0 ) - f ( t ) = 0 \qquad ( 10.5 )$$

has a unique solution on the interval t∈[0, b₁], which is located at t=a₁.

The SUV decoding algorithm uses the function u(t)=e^{-ut}. The algorithm allows spiking when both f(t) and ^{d}M_{a,b}(0) are positive and when it is possible to subtract f(t) from ^{d}M_{a,b} without making its value negative. This restricts (10.4) to only decreasing f (t), which maps to the inequality u>s in this case.

### 10.6 A Special Case of Incorrect Decoding

The following theorem describes a family of spike decoding problems where the function f(t) does not intersect the line ^{d}M_{a,b}(0) at t=a₁. In other words, this theorem can be viewed as a counter-example that shows that there are cases when the decoding algorithm can't decode the first spike accurately, even if f(t) is strictly decreasing.

Theorem 10.3. Let a=(a₁, a₂) and b=(b₁) be two spike trains and suppose that a₂<b₁. Let u(t) be a positive real function. Let f(t)=u(t) ^{d}h_{b}″(t)=u(t) H(b₁−t) v(b₁) e^{-s(b}^{₁}^{-t)}. Finally, suppose that f(t) strictly decreases on [0, b₁). Then, the equation

$${d}^{} {M}_{a , b} ( 0 ) - f ( t ) = 0 , \qquad ( 10.6 )$$

has at most one solution on t∈[0, b₁]. Furthermore, if t₁ is the value of this solution, then t₁<a₁.

FIG. 131 gives an example that illustrates the essence of Theorem 10.3. The function f(t) would intersect the line y=M_{a,b} at time t₁<a₁. Thus, a decoding algorithm that emits a spike when it becomes possible to subtract f(t) from M_{a,b} may generate a single spike too early. This example also shows that, in general, the decoding problem can be ill-posed. FIG. 131 illustrates this example.

### 10.7 Definitions for Interleaving

### Definition 10.4. Definition for a Collection of Spike Trains.

A collection of spike trains A=(A⁽¹⁾, A⁽²⁾ . . . , A^{(N)}) consists of N spike trains such that:

$${A}^{( 1 )} = ( {A}_{1}^{( 1 )} , {A}_{2}^{( 1 )} , … , {A}_{{K}_{1}}^{( 1 )} ) , \qquad ( 10.7 )$$

$${A}^{( 2 )} = ( {A}_{1}^{( 2 )} , {A}_{2}^{( 2 )} , … , {A}_{{K}_{2}}^{( 2 )} ) , \qquad ( 10.8 )$$

$$…$$

$${A}^{( N )} = ( {A}_{1}^{( N )} , {A}_{2}^{( N )} , … , {A}_{{K}_{N}}^{( N )} ) . \qquad ( 10.9 )$$

That is, K, denotes the number of spikes in the spike train A⁽ⁿ⁾ for each n∈{1, 2, . . . , N}. As usual, it is assumed that the spikes in each spike train are ordered in increasing order based on their arrival time and that them are no duplicate spikes, i.e., A₁⁽ⁿ⁾<A₂⁽ⁿ⁾< . . . <A_{K}_{ₙ}⁽ⁿ⁾ for each n.
Definition 10.5. Definition for Interleaving. A spike train α=(α₁, α₂, . . . , α_{J}) interleaves a collection of spike trains A=(A⁽¹⁾, A⁽²⁾, . . . , A^{(N)}) if and only if each spike in α precedes or follows every spike train in A. More formally, for each αⱼ and each A⁽ⁿ⁾=(A₁⁽ⁿ⁾), A₂⁽ⁿ⁾, . . . , A_{K}_{ₙ}⁽ⁿ⁾) the following condition holds:

$${α}_{j} ∈ [ 0 , {A}_{1}^{( n )} ] ⋃ ( {A}_{{K}_{n}}^{( n )} , ∞ ) , \qquad ( 10.1 )$$

for each j∈{1, 2, . . . , J} and each n∈{1, 2, . . . , N}.

The following theorem restates Definition 10.5 from the point of view of the collection A. This transforms (10.10) into three mutually exclusive inequalities.

Theorem 10.6. A spike train α=(α₁, α₂, . . . , α_{J}) with J spikes interleaves a collection of N spike trains A=(A⁽¹⁾, A⁽²⁾, . . . , A^{(N)}) if and only if for each n∈{1, 2, . . . , N} exactly one of the following three mutually exclusive inequalities holds:

$$0 \leq {A}_{{K}_{n}}^{( n )} < {α}_{1} , \qquad ( 10.11 )$$

$${α}_{j} \leq {A}_{1}^{( n )} < {A}_{{K}_{n}}^{( n )} < {α}_{j + 1} , for j ∈ \{ 1 , 2 , … , J - 1 \} , \qquad ( 10.12 )$$

$${α}_{J} \leq {A}_{1}^{( n )} . \qquad ( 10.13 )$$

The next lemma proves that the inequality A₁⁽ⁿ⁾≤αⱼ<A_{K}_{ₙ}⁽ⁿ⁾ complements the three inequalities in Theorem 10.6.

Lemma 10.7. Let a=(a₁, a₂, . . . , a_{J}) be a spike train and let b=(b₁, b₂, . . . , b_{K}) be another spike train. Then, one of the following two mutually exclusive conditions holds:

$$\begin{matrix}( 1 ) & \{ \begin{matrix}( 1. a ) & 0 \leq {b}_{K} < {a}_{1} , \\ ( 1. b ) & {a}_{j} \leq {b}_{1} < {b}_{K} < {a}_{j + 1} , for some j ∈ \{ 1 , 2 , … , J \} , \\ ( 1. c ) & {a}_{J} \leq {b}_{1} ,\end{matrix}\end{matrix} \qquad ( 10.14 )$$

$$\begin{matrix}( 2 ) & {b}_{1} < {a}_{j} \leq {b}_{K} , for some j ∈ \{ 1 , 2 , … , J \} .\end{matrix} \qquad ( 10.15 )$$

Definition 10.8. Interleaving of two collections of spike trains. A collection of spike trains α=(α⁽¹⁾, α⁽²⁾, . . . , α^{(M′)}) interleaves another collection of spike trains A=(A⁽¹⁾, A⁽²⁾, . . . , A^{(M″)}) if each spike train in a interleaves the collection of spike trains A (see Definition 10.5).
Definition 10.9. Sufficient interleaving between a spike train and a collection of spike trains. A spike train α=(α₁, α₂, . . . , α_{J}) with J spikes sufficiently interleaves a collection of M″ spike trains A=(A⁽¹⁾, A⁽²⁾, . . . , A^{(M″)}) if α interleaves A and the following two conditions hold:
1. 0<α_{J}<A₁⁽ⁿ⁾, for some n∈{1, 2, . . . , M″}.
2. For each j∈{1, 2, . . . , J−1} there is m∈{1, 2, . . . , M″} such that αⱼ≤A₁^{(m)}≤A_{K}_{ₘ}^{(m)}<αⱼ₊₁.

To summarize, interleaving means that for each spike train A⁽ⁿ⁾ in the collection A there is an inter-spike interval in a that contains all spikes from A⁽ⁿ⁾. Sufficient interleaving extends interleaving by also requiring each inter-spike interval in a to contain all spikes from at least one spike train in the collection A and also requiring at least one train in A to occur after the last spike in a.

Note that Definition 10.9 does not require any spikes in A to occur before a₁. That possibility, however, is not explicitly excluded by this definition. Definition 10.11 rules out this possibility.

Theorem 10.10. Let α=(α₁, α₂, . . . , α_{J}) be a spike train and let A=(A⁽¹⁾, A⁽²⁾, . . . , A^{(M″)}) be a collection of spike trains. If α sufficiently interleaves A, then M″≥J.
Definition 10.11. A spike train α=(α₁, α₂, . . . , α_{J}) minimally sufficiently interleaves a collection of spike trains A=(A⁽¹⁾, A⁽²⁾, . . . , A^{(M″)}) if α sufficiently interleaves A and J=M″.
Definition 10.12. Sufficient interleaving between two collections of spike trains. A collection of spike trains α=(a⁽¹⁾, a⁽²⁾, . . . , a^{(M′)}) sufficiently interleaves another collection of spike trains A=(A⁽¹⁾, A⁽²⁾, . . . , A^{(M″)}) if each spike train in a sufficiently interleaves the collection A.
Definition 10.13. A collection of spike trains α=(α⁽¹⁾, α⁽²⁾, . . . , α^{(M′)}) minimally sufficiently interleaves a collection of spike trains A=(A⁽¹⁾, A⁽²⁾, . . . , A^{(M″)}) if each spike train in α minimally sufficiently interleaves the collection A.
Definition 10.14. Let A=(A⁽¹⁾, A⁽²⁾, . . . , A^{(N)}) be a collection of spike trains. Its projection is a spike train r=(r₁, r₂, . . . , r_{\hat{K}}) that is obtained by merging all spikes in A and sorting their times in increasing order, where \hat{K}=K₁+K₂+ . . . +K_{N} and Kₙ denotes the number of spikes in A⁽ⁿ⁾ for each n∈{1, 2, . . . , N}. More formally,

$$r = Sort ( {A}_{1}^{( 1 )} , {A}_{2}^{( 1 )} , … , {A}_{{K}_{1}}^{( 1 )} , {A}_{1}^{( 2 )} , {A}_{2}^{( 2 )} , … , {A}_{{K}_{2}}^{( 2 )} , … , {A}_{1}^{( N )} , {A}_{2}^{( N )} , … , {A}_{{K}_{N}}^{( N )} ) . \qquad ( 10.16 )$$

### 10.8 Examples of Interleaving

This section gives several examples that help clarify the definitions from Section 10.7. In all examples the spike train α is shown in red; the collection of spike trains A is shown in blue.

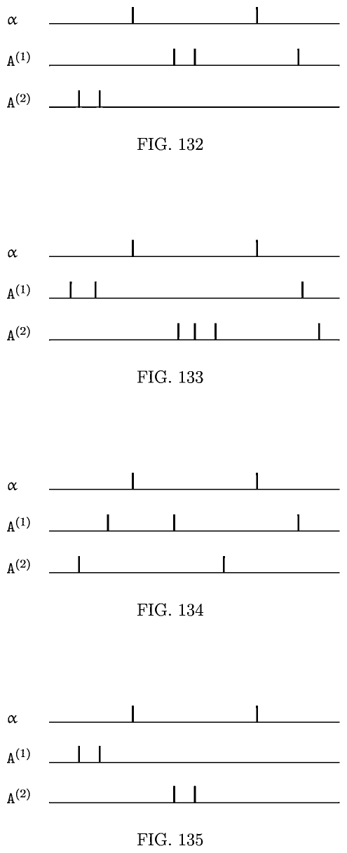

FIGS. 132, 133, 134 give examples of non-interleaving spike trains.

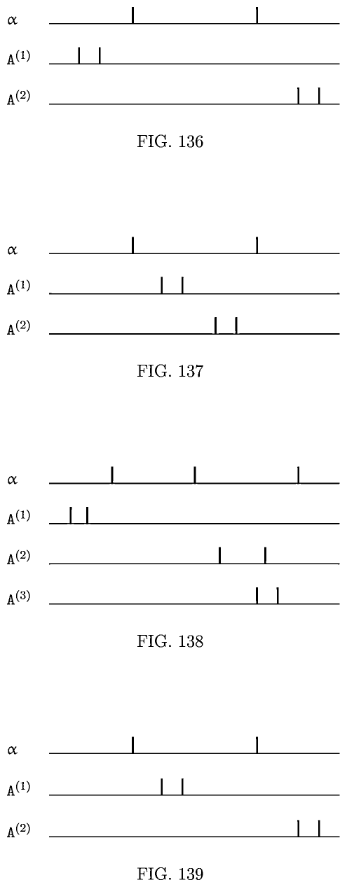

FIGS. 135, 136, 137, and 138 show examples of insufficient interleaving.

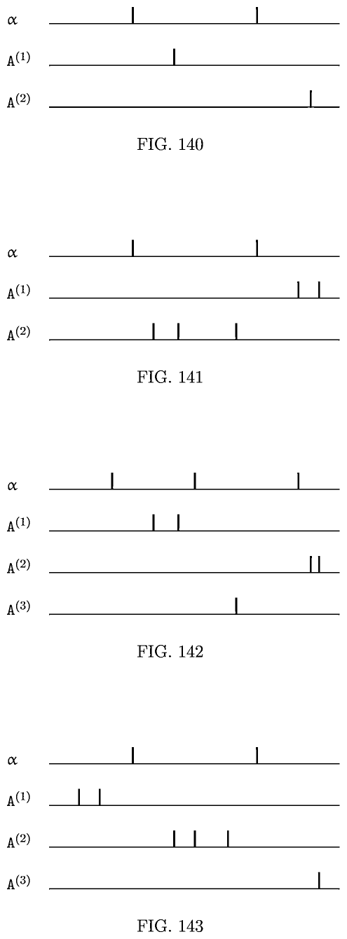

FIGS. 139, 140, 141, and 142 show examples of minimally sufficient interleaving.

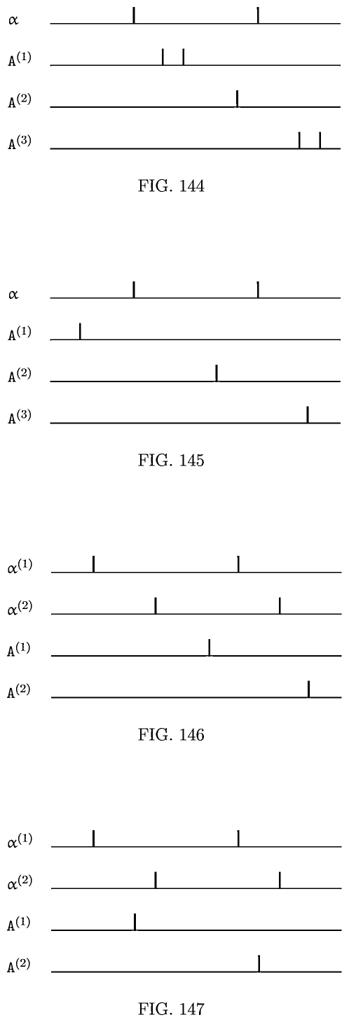

FIGS. 143, 144, 145 show example of sufficient but not minimally sufficient interleaving.

### 10.8.1 Interleaving Between Collections of Spike Trains

In all of the previous examples, α was a single spike train. In this subsection α is a collection of two spike trains, i.e., α=(α⁽¹⁾, α⁽²⁾). The examples given in FIGS. 146, 147, and 148 illustrate both sufficient and insufficient interleaving between two collections of spike trains.

### 10.9 Interleaving and Projections of Collections of Spike Trains

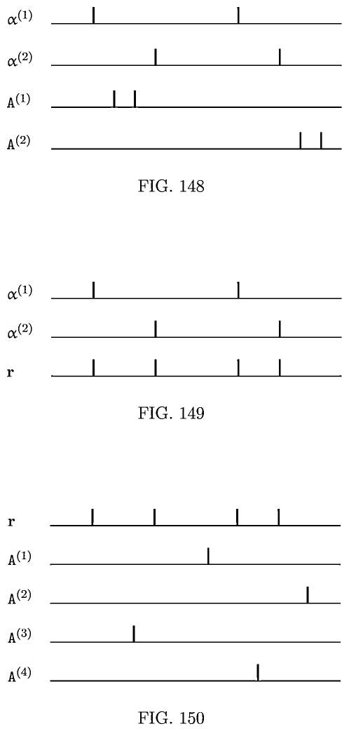

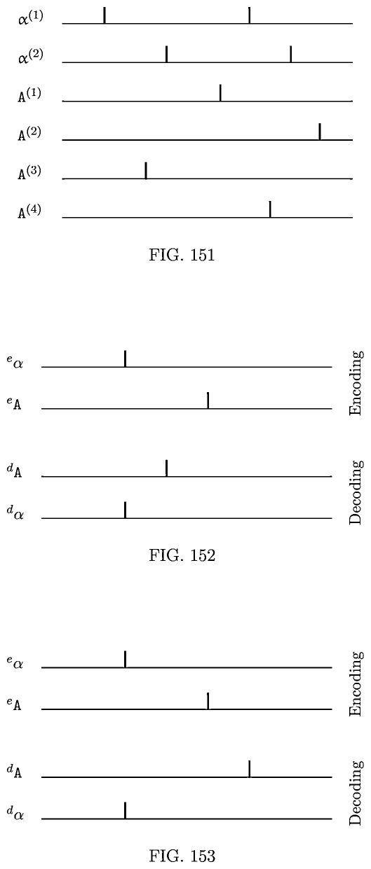

FIGS. 149, 150 give examples of projecting a collection of spike trains α to a spike train r. FIG. 151 shows the case when both the collection α=(α⁽¹⁾, α⁽²⁾) and its projected spike train r (not shown) sufficiently interleave the collection A=(A⁽¹⁾, A⁽²⁾, A⁽³⁾, A⁽⁴⁾).

Theorem 10.15. Let α=(α⁽¹⁾, α⁽²⁾, . . . , α^{(M′)}) be a collection of spike trains, where α^{(m)}=(α₁^{(m)}, α₂^{(m)}, . . . , α_{J}_{ₘ}^{(m)}) for each m∈{1, 2, . . . , M′}. Let A=(A⁽¹⁾, A⁽²⁾, . . . , A^{(M″)}) be another collection of spike trains, where A⁽ⁿ⁾=(A₁⁽ⁿ⁾, A₂⁽ⁿ⁾, . . . , A_{K}_{ₙ}⁽ⁿ⁾) for each n∈{1, 2, . . . , M″}. Finally, let r=(r₁, r₂, . . . , rⱼ) be a projection of α.
If the projected spike train r sufficiently interleaves A, then each of the spike trains in the collection α sufficiently interleaves A. The converse statement is false. That is, if each spike train in the collection α sufficiently interleaves A, the projection r may not sufficiently interleave A.

### 10.10 Sufficient Conditions for Accurate SUV Decoding

The following theorem states the conditions for accurate decoding of the first spike that apply when S′ consists of only one spike train α.

Theorem 10.16. Let u, v, and s be real numbers and suppose that u>s. Let α=(α₁, α₂, . . . , α_{J}) be a spike train and let A=(A⁽¹⁾, A⁽²⁾, . . . , A^{(M″)}) be a collection of spike trains. Suppose that the following three conditions hold:
1) The spike train α interleaves the collection A.
2) There is at least one spike train A^{(m)}∈A that falls into the interval [α₁, α₂). More formally,

$$∃ M ∈ \{ 1 , 2 , … , {M}^{″} \} such that {α}_{1} \leq {A}_{1}^{( m )} < {A}_{2}^{( m )} < … < {A}_{{K}_{m}}^{( m )} < {α}_{2} . \qquad ( 10.17 )$$

3) The list of candidate spike times ψ=(ψ₁, ψ₂, . . . , ψ_{N}) given to the SUV decoding algorithm includes α₁, i.e., α₁=ψₙ for some n∈{1, 2, . . . , N}.
   Then, the SUV decoding algorithm emits its first decoded spike at the correct time a₁.

The following lemma shows that other spike trains in A, i.e., spike trains that don't satisfy Condition 2 of Theorem 10.16, can't prevent the decoding of α₁ at the correct time. More specifically, the lemma shows that the decoding constraints associated with these trains become satisfied for some t<α₁. This lemma is used in the proofs of Theorem 10.16 and also in the proof of Theorem 10.19.

Lemma 10.17. Let u, v, and s be real numbers such that u>s. Let a=(a₁, a₂, . . . , a_{J}) be a spike train and let b=(b₁, b₂, . . . , b_{K}) be another spike train such that b_{K}≥a₁. Let f(t) be the following function:

$$f ( t ) = u ( t ) {}^{d} {h}_{b}^{″} ( t ) = u ( t ) ( {\sum }_{k = 1}^{K} H ( {b}_{k} - t ) v ( {b}_{k} ) {e}^{- s ( {b}_{k} - t )} ) , \qquad ( 10.18 )$$

where u(t)=e^{-ut} and v(t)=e^{-vt}. Also, let M_{a,b} be the matrix element computed from a and b by the SUV encoding algorithm, i.e.,

$${M}_{a , b} = {\sum }_{j = 1}^{J} {\sum }_{k = 1}^{K} u ( {a}_{j} ) v ( {b}_{k} ) H ( {b}_{k} - {a}_{j} ) {e}^{- s ( {b}_{k} - {a}_{j} )} . \qquad ( 10.19 )$$

### Then,

$$f ( {a}_{1} ) \leq {}^{d} {M}_{a , b} ( 0 ) . \qquad ( 10.2 )$$

The following theorem uses mathematical induction to generalize Theorem 10.16 from α₁ to all spikes in α. In other words, if α₁ is decoded correctly, then the next stage of the decoding algorithm can be viewed as decoding the first spike in the segment of α that starts with α₂. After α₂ is decoded correctly, this reasoning can be applied to α₃, then to α₄, and so forth until all of the spikes in α are decoded.

Theorem 10.18. Let ^{e}α=(^{e}α₁, ^{e}α₂, . . . , ^{e}α_{J}) be a spike train and let A=(A⁽¹⁾, A⁽²⁾, . . . , A^{(M″)}) be a collection of spike trains. Let u, v, and s be three real numbers. Suppose that u>s and that ^{e}α sufficiently interleaves A. Also, suppose that the vector of candidate times ψ includes every spike time in ^{e}α, i.e., ^{e}α⊆ψ in both ^{e}α and ψ are viewed as sets of real numbers.
Then, the SUV decoding algorithm will decode the train ^{e}α from the SUV model (M, h″) that was computed by the SUV encoding algorithm from ^{e}α and A, i.e., ^{d}α=^{e}α, where ^{d}α denotes a spike train generated by the decoding algorithm.

The following theorem shows that even if a subset of a collection of spike trains A is sufficiently interleaved by α, then the decoding will still be accurate. In other words, there can be some redundancy in A, provided that a subset of A is sufficiently interleaved by α.

Theorem 10.19. Let ^{e}α=(^{e}α₁, ^{e}α₂, . . . , ^{e}α_{J}) be a spike train and let A=(A⁽¹⁾, A⁽²⁾, . . . , A^{(M″)}) be a collection of spike trains. Also, let u, v, and s be three real numbers such that u>s. If a subset of A is sufficiently interleaved by ^{e}α, then the SUV decoding algorithm correctly decodes ^{e}α, i.e., ^{d}α=^{e}α, provided that ψ includes all spike times in ^{e}α.

In this formulation there could be spikes in A at t=0. These spikes don't affect the decoding, so they can be removed form A and the results proven in Theorem 10.18 and Theorem 10.19 still apply. This follows from the continuity of u(t) at zero. Depending on the shape of u(t), there could be other intervals within [0, α₁] on which spikes in A don't interfere with the decoding of α.

The following theorem generalizes Theorem 10.19 to collections of spike trains. More specifically, it states that if a subset of A is sufficiently interleaved by each train in the collection ^{e}α, then ^{e}α will be correctly decoded by the SUV decoding algorithm.

Theorem 10.20. Let ^{e}α=(^{e}α⁽¹⁾, ^{e}α⁽²⁾, . . . , ^{e}α^{(M′)}) be a collection of spike trains and let A=(A⁽¹⁾, A⁽²⁾, . . . , A^{(M″)}) be a collection of spike trains. Also, let u, v, and s be three real numbers and suppose that u>s. Finally, suppose that ψ includes the times of all spikes in ^{e}α.
If each spike train ^{e}α^{(p)} in the collection ^{e}α sufficiently interleaves a subset Â^{(p)} of the spike trains in A, then the SUV decoding algorithm correctly decodes the collection ^{e}α, i.e., ^{d}α=^{e}α.

The following theorem extends the inductive argument used in Theorem 10.19 to a more general class of problems where the collection of sequences ^{d}A given to the SUV decoding algorithm can deviate from the collection ^{e}A used for encoding. It turns out that accurate decoding is possible even in this case, provided that the spikes in ^{d}A don't occur too early with respect to ^{e}a. This theorem shows that sufficient interleaving makes the SUV decoding algorithm robust to delaying or even deleting spikes in ^{d}A.

Theorem 10.21. Let ^{e}α=(^{e}α₁, ^{e}α₂, . . . , ^{e}α_{J}) be a spike train and let ^{e}A=(^{e}A⁽¹⁾, ^{e}A⁽²⁾, . . . , ^{e}A^{(M″)}) be a collection of spike trains. Suppose that ^{e}α sufficiently interleaves ^{e}A. Let u, v, and s be three real numbers that specify the parameters for the encoded SUV model (m, h″). Suppose that u>s. Suppose that the SUV model is encoded using the spike trains ^{e}α and ^{e}A. That is,

$$m = ( {m}_{1} , {m}_{2} , … , {m}_{{M}^{″}} ) , \qquad ( 10.21 )$$

$${h}^{″} = ( {h}_{1}^{″} , {h}_{2}^{″} , … , {h}_{{M}^{″}}^{″} ) , \qquad ( 10.22 )$$

$$where$$

$${m}_{p} = {ℒ}^{( u , v )} \{ {}^{e} α★ {}^{e} {A}^{( p )} \} ( s ) , \qquad ( 10.23 )$$

$${h}_{p}^{″} = {ℒ}^{( v )} \{ {}^{e} {A}^{( p )} \} ( s ) , \qquad ( 10.24 )$$

for each p∈{1, 2, . . . , M″}. Finally, suppose that the list of candidate spike times ψ includes the times of all spikes in a.
Let ^{d}A=(^{d}A⁽¹⁾, ^{d}A⁽²⁾, . . . , ^{d}A^{(M″)}) be a collection of spike trains given to the SUV decoding algorithm. Suppose that each spike train ^{d}A^{(p)} in this collection satisfies one of the following two conditions:
1) ^{d}A^{(p)} is empty;
2) ^{d}A₁^{(p)}≥L(^{e}α, ^{e}A^{(p)}),
   where L(^{e}α, ^{e}A^{(p)}) denotes the time of the latest spike in ^{e}α that precedes ^{e}A₁^{(p)}. If no spikes in ^{e}α occur be ore ^{e}A₁^{(p)}, then L(^{e}α, ^{e}A₁^{(p)}=0. More formally,

$$L {(}^{e} α , {}^{e} {A}_{1}^{( p )} ) = max ( \{ {}^{e} {α}_{j} ∈ {}^{e} α : {}^{e} {α}_{j} \leq {}^{e} {A}_{1}^{( p )} \} ⋃ \{ 0 \} ) . \qquad ( 10.25 )$$

Then, the SUV decoding algorithm accurately decodes a from the model (m, h″), i.e.,

$${}^{d} α = {}^{e} α , \qquad ( 10.26 )$$

where ^{d}α=(^{d}α₁, ^{d}α₂, . . . , ^{d}α_{J}) is the spike train decoded by the SUV decoding algorithm.

Theorem 10.21 can be generalized to decoding collections of spike trains. That is, if the two conditions of Theorem 10.21 are satisfied for the projection ^{e}\hat{α} derived from a collection ^{e}α, then each spike train in the collection ^{e}α will be decoded accurately. The following theorem formally states this generalization.

Theorem 10.22. Let ^{e}α=(^{e}α⁽¹⁾, ^{e}α⁽²⁾, . . . , ^{e}α^{(M′)}) and ^{e}A=(^{e}A⁽¹⁾, ^{e}A⁽²⁾, . . . , ^{e}A^{(M″)}) be two collections of spike trains encoded by the SUV encoded algorithm. Suppose that ^{e}α sufficiently interleaves ^{e}A.
Let u, v, and s be three real numbers such that u>s. Let (M, h″) be the SUV model encoded from ^{e}α and ^{e}A. That is,

$${M}_{p , q} = {ℒ}^{( u , v )} \{ {}^{e} {α}^{( p )} ★ {}^{e} {A}^{( q )} \} ( s ) , \qquad ( 10.27 )$$

$${h}_{q}^{″} = {ℒ}_{{}^{e} {A}^{( q )}}^{( v )} ( s ) , \qquad ( 10.28 )$$

for each p∈{1, 2, . . . , M′} and each q∈{1, 2, . . . , M″}.
Let ^{e}\hat{α} be the projection of the collection ^{e}α. Let ^{d}A=(^{d}A⁽¹⁾, ^{d}A⁽²⁾, . . . , ^{d}A^{(M″)}) be a collection of spike trains given to the SUV decoding algorithm. Suppose that each spike train ^{d}A^{(q)}∈^{d}A satisfies one of the following two conditions:
1) ^{d}A^{(q)} is empty;
2) ^{d}A^{(q)}≥L(^{e}\hat{α}, ^{e}A₁^{(q)},
   where L(^{e}\hat{α}, A₁^{(q)}) is the time of the earliest spike in ^{e}\hat{α} that precedes ^{e}A₁^{(q)}. L(^{e}\hat{α}, ^{e}A₁^{(q)}) is zero if no such spikes exist in ^{e}\hat{α} More formally,

$$L ( {}^{e} \hat{α} , {}^{e} {A}_{1}^{( p )} ) = max ( \{ {(}^{e} {\hat{α}}_{j} ∈ {}^{e} \hat{α} : {}^{e} {\hat{α}}_{j} \leq {}^{e} {A}_{1}^{( p )} \} ⋃ \{ 0 \} ) . \qquad ( 10.29 )$$

Then, the SUV decoding algorithm accurately decodes ^{e}α from the model (M, h″), i.e.,

$${}^{d} α = {}^{e} α . \qquad ( 10.3 )$$

### 10.11 Examples of Robust Decoding in the Presence of Noise

This section gives several decoding examples in which ^{d}S″≠*S″ but ^{d}S′=*S′.

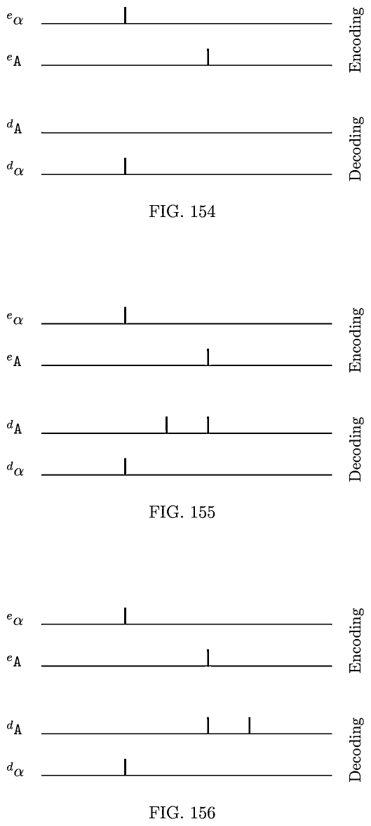

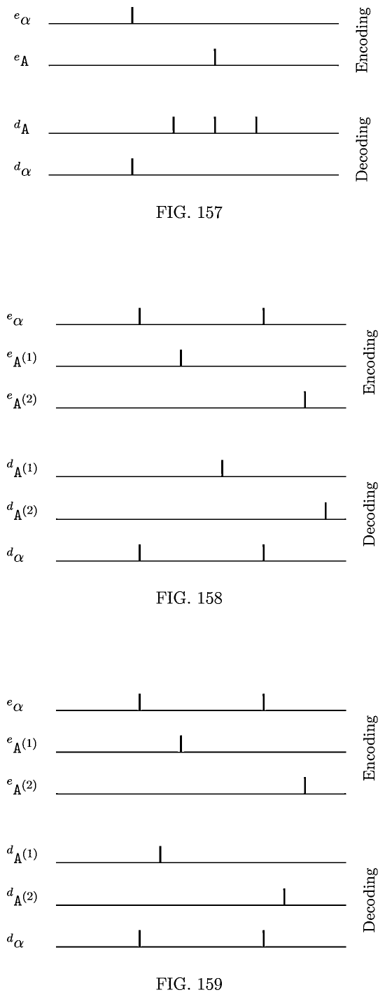

FIGS. 152, 153, 154, 155, 156, 157 show examples of perfect decoding in the presence of noise for the case when there is one spike train in S′ and one spike train in S″.

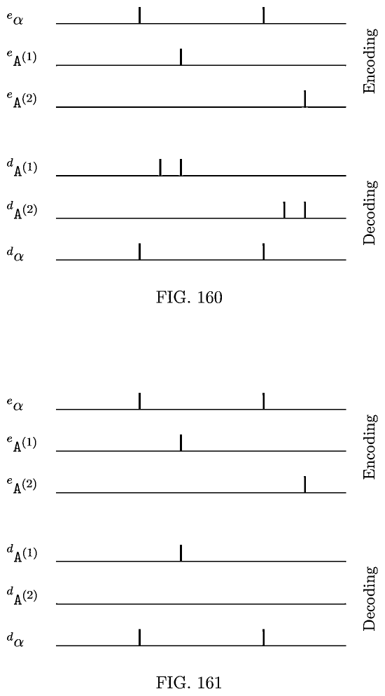

FIGS. 158, 159, 160, 161 give examples of perfect decoding in the presence of noise for the case when there is one spike train in S′ and two spike trains in S″.

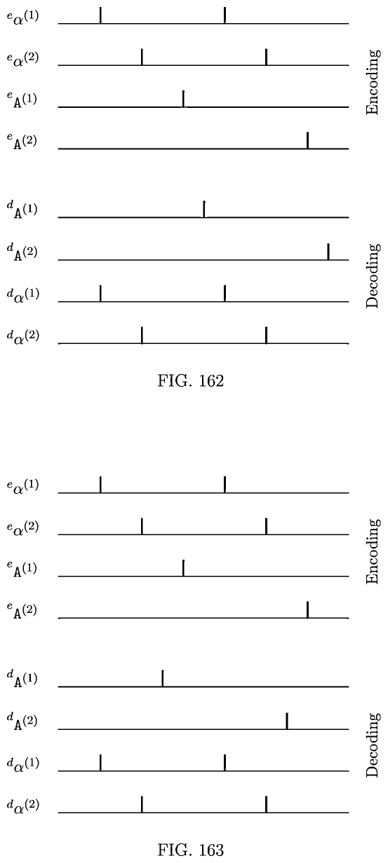

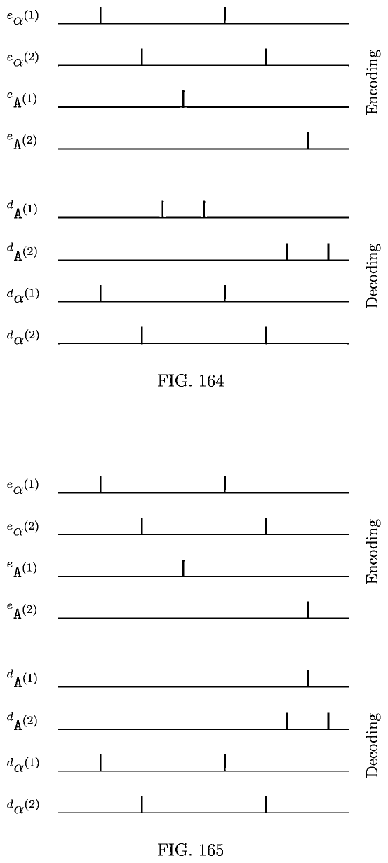

FIGS. 162, 163, 164, and 165 give examples of perfect decoding in the presence of notice for the case when there are two spike trains in S′ and two spike trains in S″.

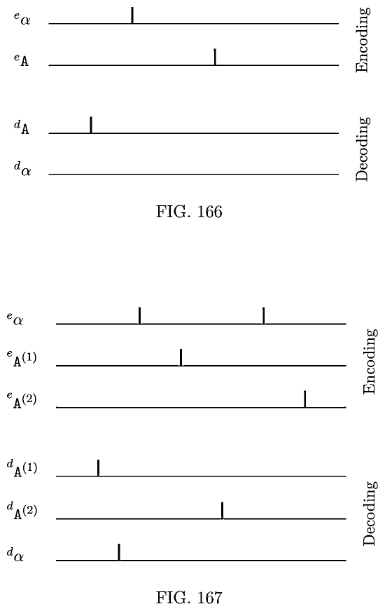

FIGS. 166 and 167 give two examples for the case when the decoding is imperfect. The decoding results may vary depending on b.

### 10.12 Summary

This chapter showed that the SUV decoding algorithm can accurately decode certain types of spike trains. More specifically, a sufficient interleaving condition was formulated and it was proven that it implies accurate decoding for certain combinations of the SUV model parameters. It was also shown that the sufficient interleaving condition can be generalized from decoding individual spike trains to decoding collections of spike trains.

Moreover, a theoretical investigation of the case in which the spikes used for the decoding differ from the spikes used for encoding suggests that the sufficient interleaving condition makes the SUV decoding algorithm robust to certain types of perturbations. That is, if the interleaving is sufficient and if the spikes used during decoding are delayed or even deleted with respect to their encoding counterparts, then the SUV decoding algorithm will decode the correct result.

Other extensions of SUV decoding are also possible. For example, instead of listing specific times, ψ can be a probability distribution for spiking within a specific time window.

### 11 Discrete- and Continuous-Time Theory Using Functionals

This chapter gives a theory from which the properties of single, dual, and exponential SSM matrices can be derived. The theory is built using real functions and functionals.

### 11.1 Functional Coupling and its Properties

As we will see in Section 11.3, the elements of SSM matrices can be expressed as applications of functionals, which are derived from the input sequences, to the arguments of a bivariate kernel function. The type of the resulting SSM matrix depends on the choice of the kernel function. This framework makes it possible to prove results that apply to single, dual, regular, and exponential SSM matrices by simply changing the kernel function, while the mapping from sequences to functionals remains the same.

Before we can derive these results, however, we need to introduce a higher-order function, Φ, that maps two functionals and a bivariate kernel function to a scalar. This scalar is obtained by applying the functionals to the two arguments of the kernel function. We will use the term functional coupling to refer to the higher-order function Φ and the term coupling value to refer to the scalar. These terms are formally defined below.

Definition 11.1. A functional coupling is a higher-order function Φ(φ, ψ, k), where φ and ψ are two functionals, and k is a bivariate real function. The function Φ is defined by the following formula:

$$Φ ( φ , ψ , k ) = φ [ x ] ( ψ [ y ] k ( x , y ) ) , \\ where φ , ψ ∈ \{ \{ ℝ ↦ ℝ \} ↦ ℝ \} and k ∈ \{ {ℝ}^{2} ↦ ℝ \} . \qquad ( 11.1 )$$

The value attained by Φ for a specific triple of its arguments is called a coupling value. The bivariate function k is called a kernel.

In other words, Φ is a function from D_{Φ} to [symbol] , where D_{Φ} is the Cartesian product of the set of all functionals (repeated twice) and the set of all bivariate functions. More formally,

$${D}_{Φ} = \{ \{ ℝ ↦ ℝ \} ↦ ℝ \} \times \{ \{ ℝ ↦ ℝ \} ↦ ℝ \} \times \{ {ℝ}^{2} ↦ ℝ \} . \qquad ( 11.2 )$$

The domain of Φ consists of all triples (φ, ψ, k) in D_{Φ} such that the right-hand side of (11.1) is well-defined. More formally,

$$( Φ ) = \{ ( φ , ψ , k ) ∈ {D}_{Φ} s . t . ψ ( k ◦ {𝔭}_{x , 2} ) = f ( x ) ∈ domain ( φ ) \} , \qquad ( 11.3 )$$

where p_{x,2} is the adapter function p_{x,2}(y)=(x, y) for each y∈ [symbol] .

The remainder of this section gives sufficient conditions for certain invariants of a functional coupling. In particular, it states the sufficient conditions for invariance with respect to reflection and the sufficient conditions for invariance with respect to translation.

Proposition 11.2. Let φ and ψ be two functionals and let k(x, y) be a kernel function. Furthermore, suppose that the following two conditions hold:
i) the kernel k(x, y) is invariant under changing the order of its two arguments and changing their signs, i.e.,

$$k ( x , y ) = k ( - y , - x ) ; \qquad ( 11.4 )$$

ii) φ[x] and ψ[y] commute for the kernel k(x, y), i.e.,

$$φ [ x ] ( ψ [ y ] k ( x , y ) ) = ψ [ y ] ( φ [ x ] k ( x , y ) ) . \qquad ( 11.5 )$$

Then, the value of φ(φ, ψ, k) is not affected if the functionals are reflected and their positions in the functional coupling are swapped. More formally,

$$Φ ( ℛψ , ℛφ , k ) = Φ ( φ , ψ , k ) . \qquad ( 11.6 )$$

The following proposition states that a translation invariant kernel makes the functional coupling invariant with respect to the translation operator on functionals.

Proposition 11.3. Let u, v∈ [symbol] and let the kernel function k∈{ [symbol] } be invariant with respect to translating its arguments (x, y) by (u, v). More formally,

$$k ( x , y ) = k ( x + u , y + v ) for all ( x , y ) ∈ domain ( k ) . \qquad ( 11.7 )$$

Then, the functional coupling Φ is invariant with respect to the corresponding translation operators on functionals, i.e.,

$$Φ ( {𝒯}_{u} φ , {𝒯}_{v} ψ , k ) = Φ ( φ , ψ , k ) . \qquad ( 11.8 )$$

### 11.2 Representing Discrete Sequences Using Functionals

Let S=S₁S₂ . . . S_{T} be a sequence of length T drawn from the alphabet Γ={c₁, c₂, . . . , c_{M}}, i.e., Sᵢ∈Γ for each i∈{1, 2, . . . , T}. To simplify the notation we will assume that the characters c₁, c₂, . . . , c_{M} are sorted in alphabetical order or in some other fixed order. Using this assumption we can refer to the i-th character in the alphabet, cᵢ, simply by using its alphabetical index, which is equal to i. Thus, the character sequence S can also be represented as an integer sequence or as a vector s=(s₁, s₂, . . . , s_{T}) of length T that consists of the alphabetical indices of all characters in S. More formally,

$$s = ( {s}_{1} , {s}_{2} , … , {s}_{T} ) ∈ {\{ 1 , 2 , … , M \}}^{T} , \\ such that {S}_{j} = {c}_{{s}_{j}} , for each j ∈ \{ 1 , 2 , … , T \} . \qquad ( 11.9 )$$

Let ω(s, φ) denote a function that maps a vector s to a vector of M functionals such that the i-th functional in the resulting vector is derived from all occurrences of the i-th alphabet character in the sequence S by adding shifted instances of the “template” functional φ. In other words,

$$ω ( s , φ ) = ( {ω ( s , φ )}_{1} , {ω ( s , φ )}_{2} , … , {ω ( s , φ )}_{M} ) , \qquad ( 11.1 )$$

where each element ω(s, φ)ᵢ is a functional that is defined using the following formula:

$${ω ( s , φ )}_{i} = {\sum }_{p = 1}^{T} {δ}_{{s}_{p} i} \cdot ( {𝒯}_{p} φ ) , i ∈ \{ 1 , 2 , … , M \} . \qquad ( 11.11 )$$

In the previous expression, δₛ_{ⱼ}ᵢ denotes the Kronecker's delta, i.e.,

$${δ}_{ab} = \{ \begin{matrix}1 , & if a = b , \\ 0 , & if a \neq b .\end{matrix}$$

Also, in (11.11) it is assumed that zero times a functional evaluates to zero. More formally,

$$( {δ}_{{s}_{p} i} \cdot ( {𝒯}_{p} φ ) ) f = \{ \begin{matrix}( {T}_{p} φ ) f , & if {s}_{p} = i , \\ 0 , & if {s}_{p} \neq i .\end{matrix} \qquad ( 11.12 )$$

The properties of discrete SSM matrices can be derived from the special case in which φ=δ. In this case, the Dirac's delta is used to represent an instance of each character in the sequence. For this special case, the definition of ω(s, φ)=ω(s, δ) has the following form:

$$w ( s , δ ) = ( {ω ( s , δ )}_{1} , {ω ( s , δ )}_{2} , … , {ω ( s , δ )}_{M} ) , \qquad ( 11.13 )$$

$$where$$

$${ω ( s , δ )}_{i} = {\sum }_{p = 1}^{T} {δ}_{{s}_{p} i} \cdot ( {𝒯}_{p} δ ) , i ∈ \{ 1 , 2 , … , M \} . \qquad ( 11.14 )$$

Note that there are two deltas now: the first one is the Dirac's delta, which is denotes with δ and is a functional that returns the value of its argument function at 0. The second one is the Kronecker's delta, which is denoted with δ and is a function that returns either zero or one, depending on its two arguments that are traditionally placed in the subscript. We will use different fonts to distinguish between these two deltas.

In the vector ω(s, δ), the value of the i-th functional ω(s, δ)ᵢ when applied to an argument function f is equal to the sum of the values of f evaluated at specific points that correspond to the indices of the character cᵢ in the sequence S. More formally,

$$( {ω ( s , δ )}_{i} ) f = ( {\sum }_{p = 1}^{T} {δ}_{{s}_{p} i} \cdot ( {𝒯}_{p} δ ) ) f = {\sum }_{p = 1}^{T} ( {δ}_{{s}_{p} i} \cdot ( {𝒯}_{p} δ ) ) f = {\sum }_{p = 1}^{T} {δ}_{{s}_{p} i} \cdot ( ( {𝒯}_{p} δ ) f ) = {\sum }_{p = 1}^{T} {δ}_{{s}_{p} i} \cdot ( δ ( {𝒯}_{p} f ) ) = {\sum }_{p = 1}^{T} {δ}_{{s}_{p} i} \cdot ( {𝒯}_{p} f ) ( 0 ) = {\sum }_{p = 1}^{T} {δ}_{{s}_{p} i} \cdot f ( {t}_{p} ( 0 ) ) = {\sum }_{p = 1}^{T} {δ}_{{s}_{p} i} \cdot f ( p ) . \qquad ( 11.15 )$$

### 11.3 Expressing SSM Matrices Using Functional Coupling

This section shows that the elements of an SSM matrix can be expressed as functional coupling values using the sequence representation shown in (11.13). Different types of matrices (e.g., regular versus exponential) can be obtained by simply using a different kernel function.

Let S′=S₁′S₂′ . . . S_{T}′ be a sequence of length T drawn from the alphabet Γ′={a₁, a₂, . . . , a_{M′}} and let S″=S₁″S₂″ . . . S_{T}″ be a sequence of length T drawn from the alphabet Γ″={b₁, b₂, . . . , b_{M″}}. Let s′ and s″ be two integer vectors that contain the alphabetical indices of the characters in S′ and S″, respectively. These two vectors are defined similarly to (11.9), i.e.,

$${s}^{′} = ( {s}_{1}^{′} , {s}_{2}^{′} , {s}_{T}^{′} ) ∈ {\{ 1 , 2 , … , {M}^{′} \}}^{T} such that {S}_{i}^{′} = {a}_{{s}_{i}^{′}} , for each j ∈ \{ 1 , 2 , … , T \} , \qquad ( 11.16 )$$

$${s}^{″} = ( {s}_{1}^{″} , {s}_{2}^{″} , {s}_{T}^{″} ) ∈ {\{ 1 , 2 , … , {M}^{″} \}}^{T} such that {S}_{j}^{″} = {b}_{{s}_{j}^{″}} , for each j ∈ \{ 1 , 2 , … , T \} . \qquad ( 11.17 )$$

Two vectors of functionals, ω(s′, δ) and ω(s″, δ), will be used to represent the two sequences. To shorten the formulas, ω′ will be used as a shorthand notation for ω(s′, δ) and ω″ will be used as a shorthand notation for ω(s″, δ). In other words,

$${ω}^{′} = ω ( {s}^{′} , δ ) and {ω}_{i}^{′} = {ω ( {s}^{′} , δ )}_{i} for each i ∈ \{ 1 , 2 , … , {M}^{′} \} , \qquad ( 11.18 )$$

$${ω}^{″} = ω ( {s}^{″} , δ ) and {ω}_{j}^{″} = {ω ( {s}^{″} , δ )}_{j} for each j ∈ \{ 1 , 2 , … , {M}^{″} \} , \qquad ( 11.19 )$$

A similar shorthand notation will be used when there is only one sequence S:

$$ω = ω ( s , δ ) and {ω}_{i} = {ω ( s , δ )}_{i} for each i ∈ \{ 1 , 2 , … , M \} . \qquad ( 11.2 )$$

The coupling value φ(ωᵢ′, ωⱼ″, k) obtained for a pair of these functionals can be expressed in terms of the values of the kernel function on the grid {1, 2, . . . , T}×{1, 2, . . . , T} as follows:

$$Φ ( {ω}_{i}^{′} , {ω}_{j}^{″} , k ) = {ω}_{i}^{′} [ x ] ( {ω}_{j}^{″} [ y ] k ( x , y ) ) = {ω}_{i}^{′} [ x ] ( {\sum }_{q = 1}^{T} {δ}_{{s}_{q}^{″} j} \cdot k ( x , q ) ) = {\sum }_{p = 1}^{T} {δ}_{{s}_{p}^{′} i} ( {\sum }_{q = 1}^{T} {δ}_{{s}_{q}^{″} j} \cdot k ( p , q ) ) = {\sum }_{p = 1}^{T} {\sum }_{q = 1}^{T} {δ}_{{s}_{p}^{′} i} \cdot {δ}_{{s}_{q}^{″} j} \cdot k ( p , q ) , \qquad ( 11.21 )$$

where i∈{1, 2, . . . , M′} and j∈{1, 2, . . . , M″}.

### 11.3.1 Regular SSM Matrices

The following proposition shows that each element of a dual SSM matrix is equal to the coupling value of the functionals that correspond to its row and column. For regular matrices (i.e., ones with integer elements) the kernel function selects pairs of indices (p, q) from the two sequences such that p≤q.

Proposition 11.4. Let D be the dual SSM matrix for the sequences S′ and S″. Then, the value of a matrix element in the i-th row and the j-th column is given by the following formula:

$${D}_{i j} = Φ ( {ω}_{i}^{′} , {ω}_{j}^{″} , {k}_{D} ) for each i ∈ \{ 1 , 2 , … , {M}^{′} \} and j ∈ \{ 1 , 2 , … , {M}^{′′} \} , \\ where \qquad ( 11.22 )$$

$${k}_{D} ( x , y ) = \{ \begin{matrix}1 , & if x \leq y , \\ 0 , & if x > y .\end{matrix} \qquad ( 11.23 )$$

The following corollary is a special case of Proposition 11.4 for “single-band” SSM matrices with integer elements.

Corollary 11.5. Let X be the single-band SSM matrix for the sequence S, which is of length T and is drawn from the alphabet Γ={c₁, c₂, . . . , c_{M}}. Then, each element of this matrix can be expressed as follows:

$${X}_{i j} = Φ ( {ω}_{i} , {ω}_{j} , {k}_{D} ) , for each i , j ∈ \{ 1 , 2 , … , M \} .$$

### 11.3.2 Exponential SSM Matrices

This section derives the analogs of (11.22) and (11.24) for exponential SSM matrices. In this case, we will use the kernel k_{E}, which is defined as follows:

$${k}_{E} ( x , y ) = \{ \begin{matrix}{2}^{- ( y - x )} , & if x \leq y , \\ 0 , & if x > y .\end{matrix} \qquad ( 11.25 )$$

The following proposition shows how dual exponential SSM matrices can be expressed using functional coupling.

Proposition 11.6. Let S′ and S″ be two sequences of length T. Also let s′ and s″ be two integer vectors of length T that contain the alphabetical indices of the characters in S′ and S″. Each element of the dual exponential SSM matrix D^{(E)}(S′, S″) is equal to the coupling value for the corresponding functionals in ω′ and ω″ with k_{E} used as a kernel function. More formally, the matrix element in the i-th row and the j-th column can be expressed as follows:

$${{D}^{( E )} ( {S}^{′} , {S}^{″} )}_{i j} = Φ ( {ω}_{i}^{′} , {ω}_{j}^{″} , {k}_{E} ) , for each i ∈ \{ 1 , 2 , … , {M}^{′} \} and j ∈ \{ 1 , 2 , … , {M}^{′′} \} . \qquad ( 11.26 )$$

By swapping S′ and S″ a similar result can be obtained for the other dual matrix D^{(E)}(S″, S′). In other words, the element in row j and column i of that matrix can be represented with the following coupling value:

$${{D}^{( E )} ( {S}^{″} , {S}^{′} )}_{ji} = Φ ( {ω}_{j}^{″} , {ω}_{i}^{′} , {k}_{E} ) , \qquad ( 11.27 )$$

for each j∈{1, 2, . . . , M″}, and each i∈{1, 2, . . . , M′}.

The following corollary is a special case of Proposition 11.6 for the single-band case.

Corollary 11.7. Each element of the single exponential SSM matrix X^{(E)} can be expressed by coupling the corresponding functionals in w as shown below:

$${X}_{i j}^{( E )} = Φ ( {ω}_{i} , {ω}_{j} , {k}_{E} ) , for i , j ∈ \{ 1 , 2 , … , M \} . \qquad ( 11.28 )$$

### 11.4 Functionals for Reversed Sequences

Let f∈{ [symbol] } be a function such that domain(f)⊆[1, T]. Also, let g∈{ [symbol] } be a function that is obtained by reversing f on [1, T], i.e., g(x)=f(T+1−x) for each x such that T+1−x∈domain(f). The function g can be expressed as follows: g=( [symbol] ₊₁∘ [symbol] )f. In other words, reversing a function is equivalent to reflecting and translating it appropriately. This section shows that this idea can be extended to functionals that are derived from discrete sequences using (11.10) if the “template” functional is invariant with respect to reflection.

Let S=S₁S₂ . . . S_{T} be a sequence of length T and let [symbol] denote the sequence obtained by reversing the sequence S. In other words, [symbol] ∈Γ={c₁, c₂, . . . , c_{M}} such that

$$\overleftarrow{S} = {S}_{T} {S}_{T - 1} … {S}_{1} , where {\overleftarrow{S}}_{j} = {S}_{T + 1 - j} , for each j ∈ \{ 1 , 2 , … , T \} . \qquad ( 11.29 )$$

Similarly, let [symbol] denote a vector obtained by reversing the vector s=(s₁, s₂, . . . , s_{T}), which was defined in (11.9). In other words,

$$\overleftarrow{s} = ( {s}_{T} , {s}_{T - 1} , … , {s}_{1} ) , where {\overleftarrow{s}}_{j} = {s}_{T + 1 - j} , for each j ∈ \{ 1 , 2 , … , T \} . \qquad ( 11.3 )$$

Given a “template” functional φ, we can use (11.11) to derive the following formula for the functional that represents occurrences of cᵢ in [symbol] :

$${ω ( \overleftarrow{s} , φ )}_{i} = {\sum }_{p = 1}^{T} {δ}_{{\overleftarrow{s}}_{p} i} \cdot ( {𝒯}_{p} φ ) . \qquad ( 11.31 )$$

Using (11.30) as an index conversion formula, the right-hand side of (11.31) can be rewritten in terms of the elements of s instead of the elements of [symbol] :

$${ω ( \overleftarrow{s} , φ )}_{i} = {\sum }_{p = 1}^{T} {δ}_{{s}_{T + 1 - p} i} \cdot ( {𝒯}_{p} φ ) . \qquad ( 11.32 )$$

Changing the index variable from p to q=T+1−p changes the previous equation as follows:

$${ω ( \overleftarrow{s} , φ )}_{i} = {\sum }_{q = 1}^{T} {δ}_{{s}_{q} i} \cdot ( {𝒯}_{T + 1 - q} φ ) . \qquad ( 11.33 )$$

Because [symbol] _{T+1-q}= [symbol] _{T+1}∘ [symbol] _{-q}, it is possible to “pull” [symbol] _{T+1} out of the sum, i.e.,

$${ω ( \overleftarrow{s} , φ )}_{i} = {\sum }_{q = 1}^{T} {δ}_{{s}_{q} i} ( ( {𝒯}_{T + 1} ◦ {𝒯}_{- q} ) φ ) = {𝒯}_{T + 1} ( {\sum }_{q = 1}^{T} {δ}_{{s}_{q} i} \cdot ( {𝒯}_{- q} φ ) ) . \qquad ( 11.34 )$$

The following proposition gives the formula for ω( [symbol] , φ)ᵢ in terms of [symbol] , [symbol] , and ω(s, [symbol] φ)ᵢ, which is possible because [symbol] _{-q}∘ [symbol] = [symbol] ∘ [symbol] _{q} and because [symbol] is its own inverse.

Proposition 11.8. The functional ω( [symbol] , φ)ᵢ, which is derived from occurrences of the i-th character in the reversed sequence [symbol] using the template functional φ, can be obtained by reflecting and then translating the functional ω(s, [symbol] φ)ᵢ by T+1. The functional ω(s, [symbol] φ) is derived from occurrences of the same character in the original sequence S using the functional [symbol] φ, which is obtained by reflecting the template functional φ. More formally,

$${ω ( \overleftarrow{s} , φ )}_{i} = ( {𝒯}_{T + 1} ◦ ℛ ) ω {( s , ℛφ )}_{i} , for each i ∈ \{ 1 , 2 , … , M \} . \qquad ( 11.35 )$$

Corollary 11.9. Let φ be a functional that is invariant under reflection, i.e., [symbol] φ=φ. Then,

$${ω ( \overleftarrow{s} , φ )}_{i} = ( {𝒯}_{T + 1} ◦ ℛ ) ω {( s , φ )}_{i} , for each i ∈ \{ 1 , 2 , … , M \} . \qquad ( 11.36 )$$

For the special case of Corollary 11.9 in which φ=δ, equation (11.36) can be rewritten as:

$${ω ( \overleftarrow{s} , φ )}_{i} = ( {𝒯}_{T + 1} ◦ ℛ ) ω {( s , δ )}_{i} , for each i ∈ \{ 1 , 2 , … , M \} . \qquad ( 11.37 )$$

### 11.5 Expressing ZUV Matrices Using Coupled Functionals

This section shows how to express the elements of a ZUV matrix using coupled functionals. The kernel function k^{(zuv)} that can be used to do this mapping is shown below:

$${k}^{( zuv )} ( x , y ) = H ( y - x ) {u}^{- x} {v}^{- y} {𝓏}^{- ( y - x )} , \qquad ( 11.38 )$$

where H(y−x) denotes the Heaviside function, i.e.,

$$H ( y - x ) = \{ \begin{matrix}1 , & if y \geq x , \\ 0 , & if y < x .\end{matrix} \qquad ( 11.39 )$$

The following theorem uses k^{(zuv)} as a kernel function for coupling functionals derived from character sequences to denote the elements of a ZUV matrix.

Theorem 11.10. Let S′ be a character sequence of length T that is drawn from the alphabet Γ′={c₁′, c₂′, . . . , C_{M′}′} of size M′. Let S″ be another character sequence of length T that is drawn from the alphabet Γ″={c₁″, c₂″, . . . , c_{M′}″} of size M″. Let ω′=(ω₁′, ω₂′, . . . , ω_{M′}′) be a collection of functionals that was derived from S′ as described in (11.10) using Dirac's delta as the template functional. Similarly, let ω″=(ω₁″, ω₂″, . . . , ω_{M″}″) be a collection of functionals derived from S″, also using Dirac's delta as the template functional.
Then, each element of the ZUV matrix encoded from S′ and S″ is equal to the functional coupling between the corresponding functionals in ω′ and ω″ that uses the kernel function k^{(zuv)}. More formally, for each i∈{1, 2, . . . , M′} and each j∈{1, 2, . . . , M″},

$${M}_{{a}^{( i )} , {b}^{( j )}}^{( zuv )} = {{𝒵}^{+}}_{{a}^{( i )} * {b}^{( j )}}^{( u , v )} + ( 𝓏 ) = Φ ( {ω}_{i}^{′} , {ω}_{j}^{″} , {k}^{( zuv )} ) , \qquad ( 11.4 )$$

where a⁽ⁱ⁾=(a₀⁽ⁱ⁾, a₁⁽ⁱ⁾, a₂⁽ⁱ⁾, . . . , a_{T-1}⁽ⁱ⁾) is a binary sequence that indicates the occurrences of cᵢ′ in S′ and b^{(j)}=(b₀⁽ⁱ⁾, b₁⁽ⁱ⁾, b₂⁽ⁱ⁾, . . . , b_{T-1}⁽ⁱ⁾) is a binary sequence that indicates the occurrences of cⱼ″ in S″. That is,

$${a}_{p}^{( i )} = \{ \begin{matrix}1 , & if {S}_{p}^{′} = {c}_{i}^{′} , \\ 0 , & if {S}_{p}^{′} \neq {c}_{i}^{′} ,\end{matrix} for each p ∈ \{ 0 , 1 , 2 , … , T - 1 \} \qquad ( 11.41 )$$

$${b}_{q}^{( j )} = \{ \begin{matrix}1 , & if {S}_{q}^{″} = {c}_{j}^{″} , \\ 0 , & if {S}_{q}^{″} \neq {c}_{j}^{″} ,\end{matrix} for each q ∈ \{ 0 , 1 , 2 , … , T - 1 \} . \qquad ( 11.42 )$$

### 11.6 Expressing SUV Matrices Using Coupled Functionals

The elements of an SUV matrix can also be expressed using coupled functionals. Because spike times may not be integer, however, this requires deriving both a suitable functional representation of spike trains and stating the kernel function for the SUV model.

The following definition shows how to map spike trains to functionals. This is accomplished by representing each spike in the train using shifted Dirac's deltas and summing over all spikes in the train. The resulting linear functional is a suitable mathematical representation of a spike train for the SUV mapping.

Definition 11.11. Let a=(a₁, a₂, . . . , a_{J}) be a spike train, where aⱼ specifies the time of the j-th spike for each j∈{1, 2, . . . , J}. A functional ψₐ that represents the spike train a is defined using the following formula:

$${ψ}_{a} = {\sum }_{j = 1}^{J} {𝒯}_{{a}_{j}} δ , \qquad ( 11.43 )$$

where δ is Dirac's delta.

If ψₐ is applied to a function f(t), then the result is equal to the sum over the values of f at the times of the spikes in a. More formally,

$${ψ}_{a} = ( {\sum }_{j = 1}^{J} {𝒯}_{{a}_{j}} δ ) f = {\sum }_{j = 1}^{J} ( {𝒯}_{{a}_{j}} δ ) f = {\sum }_{j = 1}^{J} δ ( {𝒯}_{{a}_{j}} f ) = {\sum }_{j = 1}^{J} ( {𝒯}_{{a}_{j}} f ) ( 0 ) = {\sum }_{j = 1}^{J} f ( {t}_{{a}_{j}} ( 0 ) ) = {\sum }_{j = 1}^{J} f ( {a}_{j} ) . \qquad ( 11.44 )$$

Let k^{(suv)}(x, y) be the following kernel function:

$${k}^{( suv )} ( x , y ) = H ( y - x ) {e}^{- ux} {e}^{- vy} {e}^{- s ( y - x )} , \qquad ( 11.45 )$$

which is parametrized by the real numbers u, v, and s. In this formula, the term H(y−x) is the Heaviside function. That is,

$$H ( y - x ) = \{ \begin{matrix}1 , & if y \geq x , \\ 0 , & if y < x .\end{matrix} \qquad ( 11.46 )$$

Using the Heaviside function ensures that k^{(suv)}(x, y)=0 when y<x.

The following theorem shows how to derive the formulas for the matrix element M_{a,b}^{(suv)} in the SUV model using the coupling operator Φ with functionals that represent the spike trains a and b and the kernel k^{(suv)}.

Theorem 11.12. Let a=(a₁, a₂, . . . , a_{J}) and b=(b₁, b₂, . . . , b_{K}) be two spike trains. Let ψₐ be a functional that represents a and let ψ_{b} be a functional that represents b, i.e.,

$${ψ}_{a} = {\sum }_{j = 1}^{J} {𝒯}_{{a}_{j}} δ , {ψ}_{b} = {\sum }_{k = 1}^{K} {𝒯}_{{b}_{k}} δ . \qquad ( 11.47 )$$

### Then,

$${M}_{a , b}^{( suv )} = {ℒ}_{a * b}^{( u , v )} ( s ) = Φ ( {ψ}_{a} , {ψ}_{b} , {k}^{( suv )} ) . \qquad ( 11.48 )$$

### 11.7 Summary

This chapter introduced a framework for studying and analyzing the properties of SSM matrices. It also introduced a notation that makes it possible to prove the properties of different types of SSM models. Using functionals enables the exploration of models in which the sampling process is imperfect, e.g., models where the sampling may require a certain amount of time to complete and concurrent samples can interfere or overlap with each other. These processes can be modeled using integral operators with narrow gaussian kernels or other narrowly localized kernels. This approach can also work with continuous-time sequences and can be applied to spike trains.

The template functional determines how each item in the sequence is represented. For example, using Dirac's delta as a template functional leads to a spike-based representation. Dirac's delta, however, is not the only possible template functional that can be used in this model. Certain properties of the templates may be used as necessary conditions for specific features of the model. For example, some features of the SSM model are preserved if the template functional is symmetric (i.e., invariant with respect to reflecting its argument function). Moreover, some properties of the SSM model are retained even if different template functionals are used for the two encoded sequences, if these templates commute.

The kernel function determines how each element of the encoded matrix is calculated. For example, using the kernel function k^{(zuv)} with a spike-based representation derived from character sequences leads to ZUV matrices. Similarly, using the kernel function k(v) with functionals derived from spike trains leads to SUV matrices.

All references, including publications, patent applications, and patents cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) is to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

A portion of the disclosure of this patent document contains material which is subject to copyright protection. The copyright owner has no objection to the facsimile reproduction by anyone of the patent document or the patent disclosure, as it appears in the Patent and Trademark Office patent file or records, but otherwise reserves all copyright rights whatsoever.

© 2017, 2018 Alexander Stoytchev and Vladimir Sukhoy. All Rights Reserved.

What is claimed is:

1. A method of pattern matching, comprising the steps of:
receiving a first collection of signals \hat{B};
scaling at least one signal \hat{b} of the first collection of signals \hat{B} with at least one weighting function \hat{v} selected from a first collection weighting functions \hat{V}, wherein \hat{v} is not always equal to 1;
decoding a plurality of previously encoded SSM models using the first collection of signals \hat{B};
matching the first collection of signals \hat{B} to a subset of the plurality of previously encoded SSM models based on the outcomes from the step of decoding.

2. The method of claim 1, wherein the step of matching is based on the lengths of signals decoded from the previously encoded SSM models.

3. The method of claim 1, wherein at least one SSM model that is quiescent for a period of time during the step of decoding is excluded from the subset of the previously encoded SSM models during the step of matching.

4. The method of claim 1, wherein the subset of the plurality of previously encoded SSM models is empty or consists of one previously encoded SSM model or consists of more than one previously encoded SSM model.

5. The method of claim 1, wherein the step of decoding further comprises decoding a plurality of SSM models in parallel.

6. The method of claim 1, wherein the step of matching further comprises matching in parallel the first collection of signals \hat{B} to a subset of the plurality of previously encoded SSM models.

7. The method of claim 1, further comprising the steps of:
receiving a second collection of signals Â;
scaling at least one signal â of the second collection of signals Â with at least one weighting function û selected from a second collection of weighting functions Û, wherein û is not always equal to 1.

8. The method of claim 7, wherein the step of matching further comprises the step of comparing the collection of signals decoded from at least one previously encoded SSM model with the second collection of signals Â.

9. A system, comprising:
an input device for receiving a data input;
a processor coupled to the input device, the processor configured to convert the data input into a first collection of signals Â and a second collection of signals \hat{B} and to scale at least one signal in Â and \hat{B} using a weighting function that is not always equal to 1;
a memory device configured to store a plurality of known SSM models representing a plurality of previously encoded data inputs;
wherein the processor is configured to decode at least one known SSM model using a second collection of signals \hat{B} and matches the data input to a subset of the plurality of known SSM models.

10. The system of claim 9, wherein the first collection of signals Â is empty.

11. The system of claim 9, wherein the matching is based on the lengths of signals decoded from the subset of the plurality of known SSM models.

12. The system of claim 9, wherein the processor is further configured to encode the date input and wherein the memory device is further configured to store the SSM model encoded from the date input among the plurality of known SSM models, and wherein at least one SSM model is encoded from a third collection of signals A and a fourth collection of signals B;
wherein at least one signal a of the third collection of signals A is scaled by at least one weighting function u selected from a third collection of weighting functions U and at least one signal b of the fourth collection of signals B is scaled by at least one weighting function v selected from a fourth collection of weighting functions V, wherein at least one of the weighting functions u or v is not always equal to 1.

13. The system of claim 9, wherein the first collection of signals Â represents a first sequence Ŝ′, the second collection of signals \hat{B} represents a second sequence Ŝ″.

14. The system of claim 13, wherein the length of at least one sequence is at least 3.

15. The system of claim 13, wherein the processor performs O(\hat{T} \hat{M}′ \hat{M}″) or fewer primitive operations when decoding a known SSM model, wherein \hat{T} is the length of the second sequence Ŝ″, \hat{M}′ is the number of signals in the first collection of signals Â, and \hat{M}″ is the number of signals in the second collection of signals \hat{B}.

16. The system of claim 9, wherein the second collection of signals \hat{B} includes at least one spike train, wherein the processor is configured to perform O(\hat{T} \hat{M}′ \hat{M}″) or fewer primitive operations when decoding at least one known SSM model, wherein \hat{T} is the total number of spikes in the second collection of signals \hat{B}, wherein \hat{M}′ is the number of spike trains in the second collection of signals \hat{B}, and wherein \hat{M}″ is the number of spike trains decoded from the SSM model by the processor.

17. The system of claim 9, wherein the system is at least one of a personal computer (PC), a system comprising a graphics processing unit (GPU), a system comprising a field-programmable gate array (FPGA), a system-on-a-chip (SoC), or a system comprising an application-specific integrated circuit (ASIC).

18. The system of claim 9, configured for automatic speech recognition, wherein the data input is an audio input, wherein the plurality of previously encoded data inputs represents a plurality of known spoken words, and wherein the processor selects a subset of known words based on the outcomes of decoding.

19. The system of claim 9, configured for computer vision, wherein the data input is a visual image, wherein the plurality of previously encoded data inputs represents a plurality of known visual images, and wherein the processor selects a subset of known visual images based on the outcomes of decoding.

20. The system of claim 19, wherein the visual image is an image of an object and wherein the system is configured for visual object recognition.

21. The system of claim 9, wherein the visual image is an image of a face and wherein the system is configured for face recognition.

22. The system of claim 9, configured for interactive object recognition, wherein the data input is derived from sensorimotor modalities received by at least one robot while it performs at least one exploratory behavior on at least one object, wherein a plurality of previously known SSM models represents a plurality of known objects, and wherein a subset of known objects is selected based on the outcomes of decoding.

23. The system of claim 13, wherein character sequences are derived from DNA sequences, amino acid sequences, or both DNA and amino acid sequences.

24. The system of claim 9, wherein the system is further capable of selecting its next data input based on the collections of signals generated by the processor during decoding.

25. The method of claim 24, wherein the system is configured to be used as an associative memory.

26. The system of claim 25, wherein the system is configured to perform at least one of sequence prediction, sequence completion, or error correction.

27. The system of claim 9, wherein the processor comprises a plurality of parallel processors.

28. The system of claim 12, wherein at least one known SSM model comprises a matrix M, a first vector h′, and the elements of the second vector h″.

29. The system of claim 28, wherein the elements of the matrix M, the elements of the first vector h′, and the elements of the second vector h″ are distributed or replicated or both distributed and replicated across a plurality of computational units.

30. The system of claim 28, wherein at least one element M_{a,b} of the matrix M is capable of being expressed as a unilateral z-transform of the cross-correlation of the scaled signal a of a third collection of signals A and the scaled signal b of a fourth collection of signals B, wherein at least one element h′ₐ of the first vector h′ is capable of being expressed as a unilateral z-transform of the reverse of the scaled signal a, wherein at least one element h″_{b} of the vector h″ is capable of being expressed as a unilateral z-transform of the scaled signal b, and wherein the three unilateral z-transforms are computed for a complex parameter z.

31. The system of claim 28, wherein at least one element M_{a,b} of the matrix M is capable of being expressed as a Laplace transform of the cross-correlation of the scaled signal a of the third collection of signals A and the scaled signal b of the fourth collection of signals B, wherein at least one element h′ₐ of the first vector h′ is capable of being expressed as a Laplace transform of the reverse of the scaled signal a, and wherein at least one element h″_{b} of the vector h″ is capable of being expressed as a Laplace transform of the scaled signal b, and wherein the three Laplace transforms are computed for a complex parameter s.

32. The system of claim 28, wherein the first vector h′ is not stored after completing encoding.

33. The system of claim 12, wherein the computational complexity of encoding an SSM model is O(TM′), wherein the fourth collection of signals B represents a sequence S″, wherein T is the length of S″, and wherein M′ is the number of signals in the third collection of signals A.

34. The system of claim 9, wherein the second collection of signals \hat{B} represents a sequence Ŝ″, wherein the computational complexity of decoding the SSM model is O(\hat{T} \hat{M}′ \hat{M}″), wherein \hat{T} is the length of the sequence Ŝ″, \hat{M}′ is the number of signals in the first collection of signals Â, and \hat{M}″ is the number of signals in the second collection of signals \hat{B}.
